(19) United States
(12) Patent Application Publication
(10) Pub. No.: US 2022/0195071 A1
(43) Pub. Date: Jun. 23, 2022

(54) IMMUNOTHERAPEUTIC COMPOSITIONS AND USE THEREOF

(71) Applicant: Torque Therapeutics,Inc., Cambridge, MA (US)

(72) Inventor: Douglas Jones, Bellevue, WA (US)

(21) Appl. No.: 17/599,948

(22) PCT Filed: Mar. 30, 2020
(86) PCT No.: PCT/US2020/025844

§ 371 Date: Sep. 29, 2021

Related U.S. Application Data
(60) Provisional application No. 62/826,923, filed on Mar. 29, 2019.
(60) Provisional application No. 62/881,300, filed on Jul. 31, 2019.
(60) Provisional application No. 62/884,540, filed on Aug. 8, 2019.
(60) Provisional application No. 62/930,363, filed on Nov. 4, 2019.

(51) Int. Cl. C07K 17/04; A61K 47/69; A61P 35/00

(52) U.S. Cl. CPC C07K 17/04; B82Y 5/00; A61P 35/00; A61K 47/6901

(57) ABSTRACT

Combination therapies for the treatment of cancer comprising an immunostimulatory fusion molecules that include an immune cell targeting moiety and a cytokine molecule; and an immune cell loaded with protein nanogels that include a reversibly crosslinked cytokine molecule and a polymer, pharmaceutical and formulations thereof, and methods of using and making the same, are disclosed.

### CROSS-REFERENCE TO RELATED APPLICATIONS

This application claims priority to and the benefit of U.S. Provisional Application Nos. 62/826,923 filed Mar. 29, 2019; 62/881,300 filed Jul. 31, 2019; 62/884,540 filed Aug. 8, 2019 and 62/930,363 filed Nov. 4, 2019; each of the foregoing of which is incorporated herein by reference in its entirety.

### SEQUENCE LISTING

The ASCII text file submitted herewith via EFS-Web, entitled “174285_011704_sequence.txt” created on Mar. 30, 2020, having a size of 231,284 bytes, is hereby incorporated by reference in its entirety.

### FIELD

The present disclosure relates generally to immunotherapeutic compositions. Methods for making and using the same are also provided.

### BACKGROUND

The potential for immunotherapy to treat cancer and other diseases and disorder has yet to be fully realized. Many of the early successions the treatment of cancer were single agent therapies that are successful in only a small percentage of patients, with frequent occurrences of relapse. Development of combination therapies using a cellular therapy—e.g., TIL, TCR of CAR-T cells—in combination with immunotherapeutics, can be unpredictable and prone to increased toxicity and narrower efficacy ranges. Developing combination therapies, even when based on existing drugs, has indeed proven challenging. For example, early efforts in combination therapies for incorporating IL-12 or IL-15 with T cells included at least three studies that were terminated, two of which cited toxicity (see NCT01236573, NCT01369888, and NCT01457131).

When used in cancer therapy, cytokines, such as IL-12 and IL-15, can act as immunomodulatory agents that have anti-tumor effects and which can increase the immune response towards some types of tumors. However, rapid blood clearance and lack of tumor specificity require systemic administration of high doses of the cytokine in order to achieve a concentration of the cytokine at the tumor site and other relevant tissues (e.g., lymph nodes and spleen) sufficient to activate an immune response or have an anti-tumor effect. These high levels of systemic cytokine can lead to severe toxicity and adverse reactions. Both IL-12 and IL-15 have been shown to induce substantial toxicity as single agents.

Thus, the need still exists for cytokine compositions and combination therapies with improved properties, e.g., having greater therapeutic effectiveness and a reduction in the number and severity of the side effects of these products (e.g., toxicity, destruction of non-tumor cells, among others).

### SUMMARY

Novel immunotherapies for treating diseases, such as cancer, with drug combinations are provided herein. Such novel combination therapies have been discovered to exhibit unexpected improvements in reduced toxicity by, e.g., minimizing doses (synergistic potency) and/or outcomes by, e.g., escalating effect (synergistic efficacy). Indeed, while each drug of the inventive combinations individually produce similar or signature effects but, when administered in combination, display greatly enhanced effects. Such enhanced effect is great than that which would have been predicted or expected by the drugs' individual potencies. As such, the combined effect is not only synergistic but also surprising and unexpected. Different methods and tools a may be used for evaluating syngistic effects of drug combinations according to the invention. See, for example, Tallarida, R., Quantitative Methods for Assessing Drug Synergism, Genes & Cancer, 2(11) 1003-1008, 2011; Meyer, C., et al., Quantifying Drug Combination Synergy Along Potency and Efficacy Axes, Cell Systems, 8, 97-108, Feb. 2, 2019; and Ianevski, A., SynergyFinder: a Web Application for Analyzing Drug Combination Dose-Response Matrix Data, Bioinformatics, 33(15), 2413-15, 2017; each of the foregoing of which is incorporated herein by reference in its entirety. Various reference models for producing comparative interaction- or combination-indices to quantify observed effects like zero-interaction, synergism or antagonism in drug combinations are discussed in, for example, Schindler, M. Theory of Synergistic Effects: Hill-type Response Surfaces as ‘Null-interaction’ Models for Mixtures, Theoretical Biology and Medical Modelling, 14:15, 2017, which is incorporated by reference herein, in its entirety.

More particularly, the present disclosure provides, inter alia, a therapeutic (e.g., cancer immunotherapy) composition comprising: a first immune cell having a surfaced loaded with a plurality of protein nanogels and a second immune cell having a surfaced loaded with a plurality of immunostimulatory fusion molecules (IFMs, used interchangeably with “tethered fusion” or TF). In some embodiments, the first immune cell and the second immune cell are the same cell, i.e., the protein nanogels and the IFMs can be co-loaded on a single cell. In some embodiments, the first immune cell and the second immune cell are different cells, wherein the two cells (or populations of cells) can be administer together, or serially (with or without some amount of time in between). In another embodiment, an IFM may be delivered in “free” form—i.e., in solution, unattached to a cell. Such free delivery may be administered systemically or intratumoral and may be delivered concurrently with the nanogel-loaded immune cell, or before or after the nanogel-loaded immune cell. The administration of the nanogel-loaded cells and/or the IFM (in cell-bound and/or free form) may be repeated administration. It has been discovered that such co-administration of a nanogel and an IFM produces unexpected and surprising synergy. With the synergistic effect, greater efficacy at lower doses (for one or both compounds) and/or reduced toxicity levels can be achieved. There can be an wider dosing window with a greater span between efficacy and toxicity—i.e., there is a wider range in which to optimize dosage for efficacy before the maximal, undesired level of toxicity is reached.

Specifically, it has been surprisingly discovered that the anti-tumor activity of IL-15 nanogel (a multimer comprising chemically crosslinked IL-15/IL-15 Rα/Fc heterodimers (IL15-Fc) and a polymer) and IL-12 tethered fusion (single-chain IL-12p70 fused to a humanized anti-CD45 Fab) surface-loaded on cells, when combined, produces an improved therapeutic profile with a synergistic effect rather than merely additive. That is, there is a statistically significant difference (increase) in the efficacy of the combination of IL-15 nanogel and IL-12 tethered fusion, compared to what would have been expected if the efficacy were purely additive.

In some embodiments, the protein nanogels can each include a plurality of therapeutic protein monomers reversibly cross-linked to one another via a plurality of biodegradable cross-linkers. In some embodiments, the protein nanogel has a size between 30 nm and 1000 nm in diameter measured by dynamic light scattering. In some embodiments, the cross-linker degrades, after administration into a subject in need thereof, under physiological conditions so as to release the therapeutic protein monomers from the protein nanogel. In some embodiments, the protein nanogel further comprises a surface modification such as polycation so as to allow the protein nanogel to associate with the first immune cell.

In some embodiments, the therapeutic protein monomers can include one or more cytokine molecules and/or one or more costimulatory molecules, wherein:
(i) the one or more cytokine molecules are selected from IL-15, IL-2, IL-7, IL-10, IL-12, IL-18,
IL-21, IL-23, IL-4, IL-1alpha, IL-1beta, IL-5, IFNgamma, TNFa, IFNalpha, IFNbeta, GM-CSF, or GCSF; and
(ii) the one or more costimulatory molecules are selected from CD137, OX40, CD28, GITR, VISTA, anti-CD40, or CD3.

In some embodiments, the cross-linker can be a degradable or hydrolysable linker. In some embodiments, the degradable linker is a redox responsive linker. Exemplary linkers as well as methods of making and using various linkers (e.g., to make nanogels) are disclosed in PCT Application No. PCT/US2018/049594 U.S. Publication No. 2017/0080104, U.S. Pat. No. 9,603,944, and U.S. Publication No. 2014/0081012, each of which is incorporated herein by reference in its entirety.

In certain embodiments, each IFM can be engineered to contain an immunostimulatory cytokine molecule and a targeting moiety (e.g., an antibody or an antigen-binding fragment thereof) having an affinity to an antigen on the surface of the immune cell, wherein the immunostimulatory cytokine molecule is operably linked to targeting moiety. Exemplary IFMs (also referred to as “tethered fusion” or TF) are disclosed in PCT International Publication Nos. WO 2019/010219 and WO 2019/010222, each incorporated herein by reference in its entirety.

In some embodiments, the immunostimulatory cytokine molecule is selected from one or more of IL-15, IL-2, IL-6, IL-7, IL-12, IL-18, IL-21, IL-23, or IL-27 or variant forms thereof. The antigen can be selected from one or more of CD45, CD4, CD8, CD3, CD11a, CD11b, CD11c, CD18, CD25, CD127, CD19, CD20, CD22, HLA-DR, CD197, CD38, CD27, CD196, CXCR3, CXCR4, CXCR5, CD84, CD229, CCR1, CCR5, CCR4, CCR6, CCR8, CCR10, CD16, CD56, CD137, OX40, or GITR.

In one embodiment, the IFM contains IL-12, e.g., single-chain IL-12p70 fused to a humanized anti-CD45 Fab. The single-chain IL-12p70 can contain IL-12B and IL-12A joined by flexible linker. In one example, “IL-12 tethered fusion” (single-chain IL-12p70 fused to a humanized anti-CD45 Fab) can be recombinantly expressed, purified, and then tethered onto immune cells expressing CD45 such as T cells to form T cells with surface-loaded immune agonists.

In various embodiments, the first and second immune cell can be provided and administered separately (e.g., sequentially) to a patient in need of, e.g., cancer immunotherapy. In some embodiments the immune cells can be from a population of T cells that have been enriched or trained to possess specificity against one or more tumor-associated antigens (TAAs).

Another aspect relates to a method for providing cancer immunotherapy, comprising administering to a patient in need thereof a plurality of immune cells each loaded with a first plurality of protein nanogels and a second plurality of immunostimulatory fusion molecules (IFMs).

A further aspect relates to a method for providing cancer immunotherapy, comprising: administering to a patient in need thereof a first plurality of immune cells each loaded with a plurality of protein nanogels; and administering to the patient a second plurality of immune cells each loaded with a plurality of immunostimulatory fusion molecules (IFMs).

In various embodiments, the cancer immunotherapy is for treatment of a cancer selected from breast, prostate, lung, ovarian, cervical, skin, melanoma, colon, stomach, liver, esophageal, kidney, throat, thyroid, pancreatic, testicular, brain, and bone cancer, leukemia, chronic lymphocytic leukemia, basal cell carcinoma, biliary tract cancer, bladder cancer, brain and central nervous system (CNS) cancer, choriocarcinoma, colorectal cancer, connective tissue cancer, endometrial cancer, eye cancer, head and neck cancer, gastric cancer, intraepithelial neoplasm, larynx cancer, lymphoma; neuroblastoma; lip, tongue, mouth and pharynx cancer; retinoblastoma; rhabdomyosarcoma; rectal cancer; sarcoma; skin cancer; thyroid cancer; and uterine cancer.

Still another aspect relates to a method for inducing the synergistic expansion of human CD8⁺ T cells in a human immunotherapeutic regimen, said regimen consisting of co-administering at least two immune agonists, the first immune agonist comprising a T cell loaded with an IL-12 tethered fusion, and the second immune agonist comprising a T cell loaded with an IL-15 nanogel, wherein the co-administration of such immune agonists results in a synergistic expansion of said human CD8⁺ T cells. In some embodiments, the T cell loaded with the IL-12 tethered fusion, the T cell loaded with the IL-15 nanogel, or both T cells, are specific to one or more tumor-associated antigens.

In some embodiments, the tumor-associated antigen is one expressed by a cancer selected from breast, prostate, lung, ovarian, cervical, skin, melanoma, colon, stomach, liver, esophageal, kidney, throat, thyroid, pancreatic, testicular, brain, and bone cancer, leukemia, chronic lymphocytic leukemia, basal cell carcinoma, biliary tract cancer, bladder cancer, brain and central nervous system (CNS) cancer, choriocarcinoma, colorectal cancer, connective tissue cancer, endometrial cancer, eye cancer, head and neck cancer, gastric cancer, intraepithelial neoplasm, larynx cancer, lymphoma; neuroblastoma; lip, tongue, mouth and pharynx cancer; retinoblastoma; rhabdomyosarcoma; rectal cancer; sarcoma; skin cancer; thyroid cancer; and uterine cancer.

In some embodiments, the IL-12 tethered fusion comprises a humanized anti-CD45 antibody or an antibody fragment selected from a Fab, F(ab′)₂, Fd, and a Fv. In some embodiments, the IL-15 nanogel comprises a plurality of crosslinked IL-15-Fc fusion protein monomers.

Also provided herein is a method for the treatment of cancer, comprising the concurrent administration to a mammal in need thereof a synergistic, therapeutically effective amount of two immune agonists, the first immune agonist comprising a T cell loaded with an IL-12 tethered fusion, and the second immune agonist comprising a T cell loaded with an IL-15 nanogel. In some embodiments, said cancer is a solid tumor. In some embodiments, said cancer treatment further comprises an anti-proliferative cytotoxic agent either alone or in combination with radiation therapy.

In some embodiments, the first and second immune agonists are administered in a ratio of either immune agonists to the other immune agonists of 1:1, 1:2, 1:3, 1:4 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:30, 1:35, 1:40, 1:45, 1:50, 1:55, 1:60, 1:65, 1:70; 1:75, 1:80, 1:85, 1:90, 1:95, 1:100, 1:120, 1:130, 1:140, 1:150, 1:160, 1:170, 1:180, 1; 190, 1:200, 1:500, 1:1000. 1:5000, 1:10,000, 1:100,000, 2:3, 3:4, 2:5, 3:5, 3:10, 7:10, 9:10, 2:15, 4:15, 6:15, 7:15, 8:15, 11:15, 13:15, 14:15, 3:20, 7:20, 9:20, 11:20, 13:20, 17:20, 19:20, 1:25, 2:25, 4:25, 6:25, 7:25, 8:25, 10:25, 11:25, 12:25, 13:25, 14:25, 16:25, 17:25, 18:25, 19:25, 21:25, 22:25, 23:25, or 24:25.

In some embodiments, at least one of the first and second immune agonists is administered in a dosage of about 20 million cells/m², 40 million cells/m², 100 million cells/m², 120 million cells/m², 200 million cells/m², 360 million cells/m², 600 million cells/m², 1 billion cells/m², 1.5 billion cells/m², 10⁶ cells/m², about 5×10⁶ cells/m², about 10⁷ cells/m², about 5×10⁷ cells/m², about 10⁸ cells/m², about 5×10⁸ cells/m², about 10⁹ cells/m², about 5×10⁹ cells/m², about 10¹⁰ cells/m², about 5×10¹⁰ cells/m², or about 10¹¹ cells/m².

In one aspect, an immunostimulatory fusion molecule is provided, comprising:
(a) an immunostimulatory cytokine molecule; and
(b) an immune cell targeting moiety comprising an antigen-binding fragment of an antibody having an affinity to an antigen on the surface of a target immune cell,
wherein the immunostimulatory cytokine molecule is operably linked to the antigen-binding fragment.

In another aspect, an immunostimulatory fusion molecule is provided, comprising:

(a) an immunostimulatory cytokine molecule; and

(b) an immune cell targeting moiety comprising an antibody having an antigen-binding site specific for an antigen on the surface of a target immune cell, wherein the antibody comprises a light chain having a C-terminus and an N-terminus, and a heavy chain having a C-terminus and an N-terminus, wherein the light chain is linked to the heavy chain by a disulfide bond,
wherein the immunostimulatory cytokine molecule is operably linked to the antibody at the C-terminus of the light chain, the N-terminus of the light chain, or the N-terminus of the heavy chain portion.

In another aspect, an immunostimulatory fusion molecule is provided, comprising:

(a) an IL-12 molecule; and

(b) a T cell targeting moiety comprising a Fab fragment having an antigen-binding site specific for a CD45 cell surface receptor;

wherein the Fab fragment and the IL-12 molecule are operably linked together as a fusion molecule.

In some embodiments, the immune cell targeting moiety targets a T cell selected from an effector T cell, a CD4+ T cell, a CD8+ T cell, and a CTL. In some embodiments, the antigen is a CD45 receptor expressed on the cell surface of the T cell. In some embodiments, the immune cell targeting moiety comprises a Fab fragment, F(ab′)2, Fv, a single chain Fv of anti-CD45 antibodies BC8, 4B2, GAP8.3 or 9.4, or humanized version of any of the foregoing. In some embodiments, the immunostimulatory cytokine molecule comprises an IL-12, a single chain IL-12, a subunit of IL-12, or a variant form any of the foregoing. The immunostimulatory fusion molecule can further include a single-chain Fv having an affinity to an antigen on the surface of the target immune cell, wherein optionally the single-chain Fv has an affinity to the same antigen as the antigen-binding fragment. In some embodiments, the single-chain Fv has an affinity to a different antigen than the antigen-binding fragment. In some embodiments, the antigen-binding fragment is a Fab fragment, which optionally comprises a light chain and a heavy chain fragment optionally linked by a disulfide bond, and wherein the immunostimulatory cytokine molecule is operably linked to the Fab fragment at a C-terminus of the light chain, an N-terminus of the light chain, a C-terminus of the heavy chain fragment, or an N-terminus of the heavy chain fragment.

In some embodiments, the immunostimulatory cytokine molecule is operably linked to the antigen-binding fragment by a linker. In some embodiments, the linker is selected from a cleavable linker, a non-cleavable linker, a peptide linker, a flexible linker, a rigid linker, a helical linker, and a non-helical linker, e.g., a peptide linker comprising a Gly and a Ser. In some embodiments, the peptide linker is a (GGGS)_{N}(SEQ ID NO: 124) or (GGGGS)_{N}(SEQ ID NO: 125) linker, wherein _{N} indicates the number of repeats of the motif and is an integer selected from 1-10.

In some embodiments, the antigen-binding fragment has an affinity to a CD45 receptor and comprises:

(a) a light chain variable amino acid sequence corresponding to the variable domain in the antibody portion of the amino acid sequence shown in SEQ ID NO: 82, or an amino acid sequence at least 85%, 90%, 95%, or higher identity to the variable domain of SEQ ID NO: 82; and/or

(b) a heavy chain variable amino acid sequence corresponding to the variable domain of amino acid sequence shown in SEQ ID NO: 79, or an amino acid sequence at least 85%, 90%, 95%, or higher identity to the variable domain of SEQ ID NO: 79.

In some embodiments, the cytokine molecule comprises an IL-12 molecule having an amino acid sequence corresponding to the amino acid sequence shown in SEQ ID NO: 50, or an amino acid sequence at least 85%, 90%, 95%, or higher identity to the cytokine portion of SEQ ID NO: 50.

In some embodiments, the cytokine molecule comprises a single-chain IL-12 molecule having an IL-12A subunit linked to an IL-12B subunit through a linker having an amino acid sequence corresponding to the amino acid sequence shown in SEQ ID NO: 70, or an amino acid sequence at least 85%, 90%, 95%, or higher identity to the cytokine portion of SEQ ID NO: 70.

In some embodiments, the linker comprises a peptide linker having an amino acid sequence corresponding to the amino acid sequence in SEQ ID NO: 36, or an amino acid sequence at least 85%, 90%, 95%, or higher identity to the cytokine portion of SEQ ID NO: 36.

In some embodiments, the single-chain Fv has an amino acid sequence corresponding to the Fv portion of SEQ ID NO: 80, or an amino acid sequence at least 85%, 90%, 95%, or higher identity to the Fv portion of SEQ ID NO: 80.

In some embodiments, the Fab fragment comprises a light chain having a variable domain (VL) and a constant domain (CL) and a heavy chain fragment having a variable domain (VH) and a constant domain (CH1), wherein the light chain and heavy chain fragment are optionally linked by a disulfide bond, and wherein the light chain and heavy chain fragment each comprise a C-terminus and an N-terminus. In some embodiments, the IL-12 molecule is operably linked to the C-terminus or the N-terminus of the light chain or the heavy chain fragment.

In some embodiments, the immunostimulatory fusion molecule further comprises a peptide linker having a first terminus fused to the IL-12 molecule and a second terminus is fused to the Fab fragment, thereby operably linking the IL-12 molecule and the Fab fragment.

Also provided herein is an isolated nucleic acid molecule encoding any one of the immunostimulatory fusion molecule disclosed herein.

Also provided herein is a vector comprising one or more nucleic acids encoding a polypeptide corresponding to the amino acid sequence of SEQ ID NO: 36, 50, 70, 79, 80, or 82, or an amino acid sequence at least 85%, 90%, 95%, or higher identity to SEQ ID NO: 36, 50, 70, 79, 80, or 82.

Also provided herein is a host cell comprising the nucleic acid molecule or the vector disclosed herein.

A further aspect relates to a modified immune cell comprising:

(a) an immunostimulatory fusion molecule comprising
(i) an immunostimulatory cytokine molecule; and
(ii) an immune cell targeting moiety having an affinity to a cell surface antigen; and

(b) a target immune cell expressing or otherwise displaying the cell surface antigen,

wherein the immunostimulatory fusion molecule is bound to the surface of the immune cell through interaction with the cell surface antigen.

Another aspect relates to a modified immune cell comprising a healthy and/or non-malignant immune cell and the immunostimulatory fusion molecule disclosed herein bound thereto.

Another aspect relates to a method of preparing modified immune cells, comprising:

(a) providing a population of immune cells; and

(b) incubating the immunostimulatory fusion molecule of disclosed herein with the population of immune cells so as to permit targeted binding of the immunostimulatory fusion molecule thereto, thereby producing a population of immune cells having immunostimulatory fusion molecules bound on the cell surface.

Another aspect relates to a composition for use in immune cell therapy, the composition comprising:

(a) a plurality of immunostimulatory fusion molecules, each fusion molecule comprising
(i) an immunostimulatory cytokine molecule; and
(ii) an immune cell targeting moiety having an affinity to a cell surface antigen of a T cell;

(b) a population of T cells expressing or otherwise displaying the cell surface antigen, wherein the plurality of immunostimulatory fusion molecules are bound to the surface of the T cells through interaction with the cell surface antigen; and

(c) a pharmaceutically acceptable carrier, excipient, or stabilizer.

Another aspect relates to a pharmaceutical composition comprising the immunostimulatory fusion molecule disclosed herein and a pharmaceutically acceptable carrier, excipient, or stabilizer.

Also provided herein is a method for the treatment of cancer in a human subject, the method comprising administering to the human subject a cell therapeutic composition, the composition comprising:

(a) a plurality of immunostimulatory fusion molecules, each fusion molecule comprising
(i) an immunostimulatory cytokine molecule; and
(ii) an immune cell targeting moiety having an affinity to a cell surface antigen of a T cell; and

(b) a population of T cells that homes to a cancer cells or a tissue in which cancer cells exist, and wherein the T cells express the cell surface antigen,

wherein the plurality of immunostimulatory fusion molecules are bound to the surface of the T cells, and wherein the cytokine molecule acts in vivo upon the population of T cells and/or other immune cells in the human subject to stimulate an immune response against the cancer.

In some embodiments, the population of T cells comprise primary T cells, expanded primary T cells, T cells derived from PBMC cells, T cells derived from cord blood cells, T cells autologous to the human subject, T cells allogeneic to the human subject, genetically-engineered T cells, CAR-T cells, effector T cells, activated T cells, CD8+ T cells, CD4+ T cells, and/or CTLs. In some embodiments, the cell therapeutic composition is administered to the human subject in a cell therapy course selected from an adoptive cell therapy, CAR-T cell therapy, engineered TCR T cell therapy, an antigen-trained T cell therapy, or an enriched antigen-specific T cell therapy.

In some embodiments, the cytokine molecule is IL-12 and/or IL-15. The immune cell targeting moiety can comprise an antibody or antigen-binding fragment thereof that binds to CD45.

In some embodiments, the immune cell is a healthy and/or non-malignant immune cell. In various embodiments, the IFM can further include a linker for operably linking the targeting moiety and the cytokine molecule. For example, the linker can be selected from: a cleavable linker, a non-cleavable linker, a peptide linker, a flexible linker, a rigid linker, a helical linker, or a non-helical linker, preferably a peptide linker that optionally comprises Gly and Ser, wherein preferably the peptide linker is a (GGGS)_{N} or (GGGGS)_{N} linker, wherein _{N} indicates the number of repeats of the motif and is an integer selected from 1-10.

Also provided herein is a pharmaceutical composition comprising the IFM and/or protein nanogels disclosed herein and a pharmaceutically acceptable carrier, excipient, or stabilizer.

Another aspect relates to a modified immune cell (e.g., for a cell therapy), comprising a healthy and/or non-malignant immune cell and the IFM and/or protein nanogels disclosed herein bound or targeted thereto.

In some embodiments, the cell therapy can be used for treating a cancer, preferably a solid tumor cancer or a hematological cancer.

In various embodiments, the cell therapy can be selected from an adoptive cell therapy, CAR-T cell therapy, engineered TCR T cell therapy, a tumor infiltrating lymphocyte therapy, an antigen-trained T cell therapy, an enriched antigen-specific T cell therapy or NK cell therapy. In certain embodiments, the plurality of healthy and/or non-malignant immune cells are autologous to the subject.

In some embodiments, the immune stimulating moiety is a cytokine molecule. In certain embodiments, the cytokine molecule includes a cytokine, e.g., includes a cytokine chosen from one or more of IL-2, IL-6, IL-7, IL-9, IL-12, IL-15, IL-18, IL-21, IL-23, or IL-27, including variant forms thereof (e.g., a cytokine derivative, a complex comprising the cytokine molecule with a polypeptide, e.g., a cytokine receptor complex, and other agonist forms thereof). In one embodiment, the cytokine molecule is an IL-15 molecule.

In some embodiments, the immune cell targeting moiety is capable of binding to an immune cell surface target, thereby targeting the immune stimulating moiety to the immune cell, e.g., an immune effector cell (e.g., a lymphocyte). Without wishing to be bound by theory, binding of the immune cell targeting moiety to the immune cell surface target is believed to increase the concentration, e.g., the concentration over time, of the immune stimulating moiety, e.g., cytokine molecule, with its corresponding receptor, e.g., a cytokine receptor, on the surface of the immune cell, e.g., relative to the association of the free cytokine molecule with its cytokine receptor. In some embodiments, the immune cell surface target is abundantly present on the surface of an immune cell (e.g., outnumbers the number of receptors for the cytokine molecule present on the immune cell surface). In some embodiments, the immune cell targeting moiety can be chosen from an antibody molecule or a ligand molecule that binds to an immune cell surface target, e.g., a target chosen from CD4, CD8, CD11a, CD19, CD20 or CD45. In one embodiment, the immune cell targeting moiety comprises an antibody molecule or a ligand molecule that binds to CD45. In embodiments, the targeting moiety is believed to specifically deliver and/or increase the concentration of the cytokine molecule to the surface of an immune cell, thereby resulting in one or more of increased localization, distribution and/or enhancing the cell surface availability of the cytokine molecule. In embodiments, the IFM does not substantially interfere with the signaling function of the cytokine molecule. Such targeting effect results in localized and prolonged stimulation of proliferation and activation of the immune cells, thus inducing the controlled expansion and activation of an immune response.

Accordingly, in one aspect, the disclosure provides an immunostimulatory fusion molecule (IFM) comprising an immune stimulating moiety (e.g., a cytokine molecule, an agonist of a costimulatory molecule, or an inhibitor of a negative immune regulator), and an immune cell targeting moiety.

In some embodiments, the immune stimulating moiety, e.g., the cytokine molecule, is connected to, e.g., covalently linked to, the immune cell targeting moiety (e.g., directly or indirectly, e.g., via a peptide linker). In some embodiments, the immune cell targeting moiety of the IFM binds to a surface target, e.g., surface receptor, on an immune cell, e.g., an immune effector cell. In embodiments, the IFM associates, e.g., links together, the immune stimulating moiety, e.g., the cytokine molecule, and the immune cell targeting moiety to the immune cell, e.g., the effector immune cell. In some embodiments, the IFM increases the concentration of the cytokine molecule of the IFM (e.g., the concentration of the cytokine molecule of the IFM over time, e.g., a specified period of time) on the surface of the immune cell. In embodiments, the increased concentration of the cytokine molecule of the IFM on the surface of the immune cell results in one or more of: (i) increased localization (e.g., level) of the cytokine molecule of the IFM to the immune cell surface, e.g., relative to the free cytokine molecule; (ii) enhanced cell surface availability (e.g., concentration (e.g., level or amount) and/or duration of exposure) of the cytokine molecule of the IFM, e.g., relative to the free cytokine molecule; (iii) increased cytokine signaling in a targeted population of immune cells, e.g., a population of cells expressing a preselected surface target, e.g., a surface target as described herein, e.g., relative to the free cytokine molecule; (iv) prolongs cytokine signaling in the targeted cell population (e.g., increases the duration of cytokine signaling by at least 8 hours, e.g, 24 hours), e.g., relative to the free cytokine molecule; (v) causes immunostimulation; (vi) increases immune cell activation of and/or expansion, e.g., of the targeted population of immune cells; or (vii) shows reduced side effects, e.g., a lower systemic toxicity, compared to the free cytokine molecule. In some embodiments, the IFM changes, e.g., increases, any of (i)-(vii) to a greater extent than the free cytokine molecule, e.g., by at least 8 hours, e.g., 24 hours. In one embodiment, the cytokine molecule is an IL-15 molecule as described herein, and the immune cell targeting moiety is an anti-CD45 antibody molecule, e.g., an antibody or antibody fragment that binds to CD45 as described herein.

In a related aspect, the disclosure provides a composition, e.g., an IFM, comprising a cytokine molecule coupled to, e.g., fused to, an immune cell targeting moiety. In embodiments, the immune cell targeting moiety binds to a target or a receptor on the immune cell. In embodiments, the immune cell targeting moiety includes, or is, an antibody molecule, e.g., an antibody or an antibody fragment, e.g., an anti-CD45 antibody molecule (e.g., an IgG, a Fab, scFv), that binds a CD45 receptor on a cell, e.g., an immune cell (e.g., an immune effector cell, such as a lymphocyte). In embodiments, the composition, e.g., an IFM, associates, e.g., links together, the cytokine molecule and the immune cell targeting moiety to the immune cell, e.g., the effector immune cell. In embodiments, the anti-CD45 antibody binding to the cell increases the association of the IL-15 molecule with the cell and improves one or more of IL-12 signaling, immunostimulation, over time, e.g., relative to a free IL-12 molecule (an IL-12 molecule not found in the composition). In embodiments, the signaling and/or immunostimulation occurs over a period of time, e.g., minutes, hours, days e.g., by at least 8 hours, e.g., 24 hours.

In another aspect, the disclosure provides a particle, e.g., a nanoparticle, that comprises an immune agonist as described herein, e.g., nanoparticle that comprises a protein (e.g., a protein nanogel as described herein). In one embodiment, the particle comprises the same immune agonist In other embodiments, the particle comprises one or more different types of immune agonist.

Compositions, e.g., pharmaceutical compositions, comprising the IFMs and/or the nanogels disclosed herein, are also disclosed. In embodiments, the pharmaceutical compositions further include a pharmaceutically acceptable carrier, excipient, or stabilizer.

The IFMs and protein nanogels described herein can be administered directly to a subject suffering from the disorder to be treated (e.g., cancer) via e.g., intravenous or subcutaneous administration. In some embodiments, the immune cell targeting moiety of the IFM and protein nanogel delivers the cytokine molecules to the surface of an immune cell, thereby increasing the concentration of the cytokine molecules at the surface of the immune cell. In embodiments, the IFM and nanogel results in one or more of: localizes the distribution and/or enhances the cell surface availability of the cytokine molecule, thereby activating and/or stimulating the immune cell.

In other embodiments, the IFMs described herein can be administered in combination with an immune cell therapy in order to activate and/or stimulate the immune cell therapy either in vivo or in vitro. For example, an IFM described herein may be co-administered with a cell based therapy to a subject suffering from the disorder to be treated (e.g., cancer) via e.g., intravenous or subcutaneous administration. In other embodiments, a cell therapy is pulsed in vitro with an IFM described herein prior to administration. In some embodiments, the cell therapy is chosen from an adoptive cell therapy, CAR-T cell therapy, engineered TCR T cell therapy, a tumor infiltrating lymphocyte therapy, an antigen-trained T cell therapy, or an enriched antigen-specific T cell therapy.

Additional features and embodiments of any of the IFMs, compositions, nanoparticles, methods, uses, nucleic acids, vectors, and host cells, disclosed herein include one or more of the following.

In some embodiments, the immune stimulating moiety, e.g., the cytokine molecule, is functionally linked, e.g., covalently linked (e.g., by chemical coupling, genetic or protein fusion, noncovalent association or otherwise) to the immune cell targeting moiety. For example, the immune stimulating moiety can be covalently coupled indirectly, e.g., via a linker to the immune cell targeting moiety. In embodiments, the linker is chosen from: a cleavable linker, a non-cleavable linker, a peptide linker, a flexible linker, a rigid linker, a helical linker, or a non-helical linker. In some embodiments, the linker is a peptide linker. The peptide linker can be 5-20, 8-18, 10-15, or about 8, 9, 10, 11, 12, 13, 14, 15-20, 20-25, or 25-30 amino acids long. In some embodiments the peptide linker can be 30 amino acids or longer; e.g., 30-35, 35-40, 40-50 50-60 amino acids long. In some embodiments, the peptide linker comprises Gly and Ser, e.g., a linker comprising the amino acid sequence (Gly₃-Ser)ₙ or (Gly₄-Ser)ₙ, wherein n indicates the number of repeats of the motif, e.g., n=1, 2, 3, 4 or 5 (e.g., a (Gly₃-Ser)₂ or (Gly₄Ser)₂, or a (Gly₃-Ser)₃ or a (Gly₄Ser)₃ linker). In some embodiments, the linker comprises the amino acid sequence of SEQ ID NO: 36, 37, 38, or 39, or an amino acid sequence substantially identical thereto (e.g., having 1, 2, 3, 4, or 5 amino acid substitutions). In one embodiment, the linker comprises an amino acid sequence GGGSGGGS (SEQ ID NO: 37). In another embodiment, the linker comprises amino acids derived from an antibody hinge region. In certain embodiments the linker comprises amino acids derived from the hinge regions of IgG1, IgG2, IgG3, IgG4, IgGM, or IgGA antibodies. In embodiments, the linker comprises amino acids derived from an IgG hinge region, e.g., an IgG1, IgG2 or IgG4 hinge region. For example, the linker comprises a variant amino acid sequence from an IgG hinge, e.g., a variant having one or more cysteines replaced, e.g., with serines.

In other embodiments, the linker is a non-peptide, chemical linker. For example, the immune stimulating moiety is covalently coupled to the immune cell targeting moiety by crosslinking. Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). In yet other embodiments, the immune stimulating moiety is directly covalently coupled to the immune cell targeting moiety, without a linker. In yet other embodiments, the immune stimulating moiety and the immune cell targeting moiety of the IFM are not covalently linked, e.g., are non-covalently associated.

In other embodiments, the linker can be a protein or a fragment or derivative thereof, e.g., human albumin or an Fc domain, or a fragment or derivative thereof. In some embodiments, the immune cell targeting moiety is linked to the N-terminus and the immune stimulating moiety is linked to the C-terminus.

In other embodiments, the linker non-covalently associates the immune cell targeting moiety to the immune stimulating moiety. For example, the linker comprises a dimerization domain, e.g., a coiled coil or a leucine zipper.

Other features, objects, and advantages of the disclosure will be apparent from the description and drawings, and from the claims.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

### BRIEF DESCRIPTION OF THE DRAWINGS

FIG. 1 depicts exemplary fusion proteins of the present disclosure combining a cytokine and an immunoglobulin moiety for cell-surface targeting and stimulation.

FIGS. 2A-2D illustrate 4 exemplary constructs comprising anti-CD45 antibody and IL-12 (also referred to as “anti-CD45-IL12-TF” or “αCD45-IL12-TF” or “IL12-TF”).

FIG. 3 show anti-CD45-IL12-TF supports strong cell loading of IL-12 and strong surface persistence.

FIGS. 4A-4B shows schematic depicting tethered fusions can signal in cis, trans and by transfer to target cells, and shows activation of STAT4 phosphorylation in loaded (“cis”) non-loaded target cells (“trans” and “transferred”) by IL-12 tethered fusion.

FIG. 5A shows tumor growth, mouse weight change, survival (up to day 100 post ACT).

FIG. 5B shows Pmel cells carrying a surrogate IL12-TF lead candidate induce transient lymphopenia of transferred and endogenous immune cells.

FIGS. 5C-5D show proliferation (via KI67 positivity) of circulating endogenous CD8 T cells.

FIG. 5E shows endogenous NK cell proliferation (via Ki67 positivity) and activation (via CD69 positivity).

FIG. 6 shows IL12-TF augments tumor-specific T cell therapy when either pre-loaded onto adoptively transferred T cells or when solubly co-administered.

FIG. 7A shows tumor growth curves following single or multiple doses of tumor-specific T cells carrying IL12-TFs.

FIG. 7B shows survival from single or multiple doses of tumor-specific T cells carrying IL12-TFs.

FIG. 7C shows IL12-TFs enhance tumor-specific T cell expansion and engraftment in vivo.

FIG. 7D shows body weight changes following treatment with one or two doses of tumor-specific T cells carrying IL12-TFs.

FIG. 8 shows IFN-γ plasma levels following ACT with Pmel carrying one of two IL12-TFs.

FIG. 9 shows CXCL10 plasma levels following ACT with Pmel carrying one of two IL12-TFs.

FIGS. 10A-10D shows specific binding of CD8-targeted IFMs comprising wild-type or mutated IL-15 to CD8 T cells in vivo and activity of CD8-targeted IFMs on circulating CD4 T, CD8 T, and NK cells.

FIGS. 11A-11C shows toxicity of IL-15 following increasing dose or dosing schedule, compared with safety of CD8-targeted IFMs comprising wild-type or mutated IL-15 variants. * indicates after second dosing.

FIGS. 12A-12B shows anti-tumor efficacy and body weight changes from dose escalation with IL-12, a CD8-targeted IL-12 IFM, or two different CD45-targeted IL-12 IFMs.

FIG. 13A: IL-15 nanogel provides autocrine cytokine stimulation.

FIG. 13B: IL-12 tethered fusion construct and surface-loading of T cells.

FIG. 14: Study timeline.

FIG. 15: Anti-tumor activity of the combination of IL-15 nanogel-loaded PMEL T cells (DP-15 PMEL; 10×10⁶) co-administered with IL-12 tethered fusion-loaded PMEL T cells (DP-12 PMEL) dosed at 1 (left panel), 2.5 (center panel) or 5×10⁶ cells (right panel). The activity of IL-12 tethered fusion-loaded PMEL T cells (DP-12 PMEL; 1, 2.5 or 5×10⁶) and of the combination controls, where IL-15 nanogel-loaded PMEL T cells (DP-15 PMEL; 10×10⁶) were co-administered with PMEL T cells (1, 2.5 or 5×10⁶), are also shown.

FIG. 16: Changes in body weight relative to treatment start (Day 0) for the different treatment groups.

FIG. 17: left panel: Spleen weights at Day 4 post dose. At Day 4 post-dose, 4-5 mice/group were euthanized for gross pathology evaluation and spleen weights were recorded. **=p<0.01; ***=p<0.001; ****=p<0.0001.

FIG. 17, right panel: Spleen weights at Day 4 post dose. At Day 4 post-dose, 4-5 mice/group were euthanized for gross pathology evaluation and spleen weights were recorded.

FIG. 18A: Phenotype of transferred PMEL T cells over time. Blood samples were collected at Day 4, 7, 11, 16, 23, 30 and 37, and stained for flow cytometry evaluation. Transferred PMEL T cells were identified through CD90.1 staining PMEL T cells were subdivided into four different populations based on CD44 and CD62L staining profile: Effector T cells (Teff; CD44− CD62L−), naïve/stem cell memory T cells (Tn/scm; CD44− CD62L+), Effector memory T cells (Tem; CD44+ CD62L−), and central memory T cells (Tcm; CD44+ CD62L+).

FIG. 18B: PMEL T cells co-loaded with IL-12 tethered fusion and IL-15 nanogel or loaded with IL-12 tethered fusion of IL-15 nanogel only were co-cultured with B16-F10 melanoma cells at a low effector:target ratio (1:10). B16-F10 melanoma cells growth (left most), PMEL T cells proliferation (center left), numbers of activated PMEL T cells (CD25+ CD69+, measured by flow cytometry) (center right) and PMEL T cells phenotype (right most) were evaluated. For phenotype evaluation, PMEL T cells were subdivided into four different populations based on CD44 and CD62L staining profile: Effector T cells (Teff; CD44− CD62L−), naïve/stem cell memory T cells (Tn/scm; CD44− CD62L+), Effector memory T cells (Tem; CD44+ CD62L−), and central memory T cells (Tcm; CD44+ CD62L+).

FIG. 19: Top row-MART-1 T cell numbers quantified by flow cytometry with CountBright quantification beads. IL-12 tethered fusion loading (blue curves) promotes cell survival above MART-1 only, and IL-15 nanogel loading (green curves) promotes 2-fold expansion. Combining immune agonists IL-12 tethered fusion and IL-15 nanogel by combining (mixed, orange) or co-loading. Bottom row-Counts of antigen reactive (tetramer positive) cells on Day 6 show increased antigen reactivity with the surface-loaded immune agonists.

FIG. 20: Live cell colorimetric reporter assay shows cytotoxicity of MART-1 targeted T cells alone at higher E:T ratio, and increased cytotoxicity of MART-1 targeted T cells at lower E:T ratios and later time points. IL-12 tethered fusion-loaded T cells (blue) and combined (mixed, orange) IL-12 tethered fusion and IL-15 nanogel-loaded T cells show similar increases in cytotoxicity in this assay. DP-12=IL-12 tethered fusion-loaded, DP-15=IL-15 nanogel-loaded, CTL=Effector MART-1-targeted T cells.

FIG. 21: Day 6 MART-1 T cells had effector memory phenotypes (CD45RO+ CCR7−) and MTCs were highly activated (CD25+ CD69+). IL-15 nanogel-loaded T cells (left) and combined (right) IL-12 tether fusion- and IL-15 nanogel-loaded T cells show similar phenotypes.

FIG. 22: Interferon-gamma (IFNg) measured by ELISA at Days 1, 3, and 6 is increased. IL-12 tethered fusion-loaded T cells (blue) and combined (mixed, orange) IL-12 tethered fusion and IL-12 tethered fusion-loaded T cells show similar increases in cytotoxicity in this assay. E:T=Effector:Target.

FIG. 23A: Left-MART-1 T cell numbers quantified by flow cytometry with CountBright quantification beads show usable numbers of T cells at Day 3. E:T=Effector:Target. Right-re-challenge live cell colorimetric reporter assay shows cytotoxicity of MART-1 targeted T cells improves with combined (mixed, orange) IL-12 tethered fusion and IL-12 tethered fusion loaded T cells compared to singly loaded T cells. (green, blue).

FIG. 23B: IL-12 tethered fusion drives cytotoxicity of Pmel cells. Co-load treatment improves cytotoxicity of IL15 nanogel-loaded Pmel cells. As shown in FIG. 23B, complete tumor elimination was achieved in IL-12 tethered fusion and co-load groups by Day2. IL-12 tethered fusion drives IFNg production and cytotoxic activities. Tumor outgrowth was observed in control and IL-15 nanogel group by Day5.

FIG. 23C: Co-load mediated target cell cytotoxicity at low E:T ratio. As shown in FIG. 23C, IL-15 nanogel loses long-term cytotoxicity advantage as the E:T ratio decreases. IL15 nanogel+IL12 TF co-load condition shows induced persistent cytotoxicity advantage over mono-therapy.

FIG. 23D: Combo IL-15 nanogel+IL-12 TF: improved activity relative to individual agents. As shown in FIG. 23D, IL-15 nanogel, IL-12 TF, and antigen presentation showed surprising enhancement of PMEL T cells long term persistence in circulation. Co-load (15M) and combination group (IL-15 nanogel 10M+IL-12TF 5M) show comparable anti-tumor activity. Combination groups show improved activity compared to the individual agents.

FIG. 23E: Combination treatment enables persistent cell expansion of antigen-specific cells and enhances cytotoxicity. As shown in FIG. 23E, IL-15 nanogel rescues antigen-specific cell expansion from IL-12 TF loaded MTCs. IL-12 TF drives IFNg production and enhances cytotoxicity in IL-15 nanogel loaded cells.

FIG. 23F: Beneficial synergistic effect was observed on co-loaded cells at low level of IL-15 nanogel and IL-12 TF. As shown in FIG. 23F, determining the optimal loading doses of IL-15 nanogel and IL-12 TF for co-load samples, lower doses of each monotherapy might be enough to reach the same synergistic effect.

FIG. 23G: Combo and co-load show improved activity relative to IL-12 TF and IL-15 nanogel at same total cell numbers (15 M). * IIL-12 TF 15M group: variability is driven by 1 mouse w earlier tumor escape than others.

FIG. 24 shows an embodiment in which a combination DC pool is created by combining conventional mature DCs loaded directly with 15mer peptides and preloaded DCs that present 6-15mer peptides.

FIG. 25 shows the interrogation of MTCs trained against TAA using a combination process for binding to PRAME-derived 9mer and 10mer peptide via peptide-loaded MHC tetramers (MTC binding to a pool of the four PRAME tetramers is shown at left). The population of tetramer-binding, CD8 cells is highlighted. CD8 reactivity to the individual peptides is shown at right.

FIG. 26 shows HPLC of cross-linked IL-15^{N72D}/sushi-Fc protein nanogel functionalized with polyK30 on BioSep4000 size-exclusion chromatography column.

FIG. 27 depicts protein nanogel association with CD8 T cells. CD8 T cells were associated with IL-15^{N72D}/sushi-Fc protein nanogels containing 3% in weight of Alexa-647 conjugated IL-15^{N72D}/sushi-Fc. CD8 T cells were frozen in FBS+5% DMSO overnight. Upon thawing, CD8 T cells were cultured in IL-2 containing media (20 ng/ml) and their Alexa-647 fluorescence measured at the indicated time points by flow cytometry.

FIG. 28 depicts T cell expansion analysis. CD8 T cells were conjugated (right group) or not (left group) with IL-15^{N72D}/sushi-Fc Nanogels before freezing in FBS+5% DMSO overnight. Upon thawing, both groups were cultured in IL-2 containing media (20 ng/ml) and the number of live cells was measured after 4 hours (gray bars) and on day 2 (black bars) by flow cytometry. Complete media for this experiment was IMDM (Lonza), Glutamaxx (Life Tech), 20% FBS (Life Tech), 2.5 ug/ml human albumin (Octapharma), 0.5 ug/ml Inositol (Sigma).

FIGS. 29A-29B show T-cell expansion analysis. In FIG. 29A, CD3 T cells were associated (rightmost group) or not (3 leftmost groups) with IL-15^{N72D}/sushi-Fc protein nanogels before freezing in serum-free media (Bambanker) for 2 weeks. Upon thawing, first group was cultured in complete media (Media only), second group was cultured in IL-2 containing (20 ng/ml) complete media (IL-2 (soluble)), third group was cultured in IL-15^{N72D}/sushi-Fc containing (0.6 ug/ml) complete media (IL-15^{N72D}/sushi-Fc (0.6 ug/ml)) and fourth group was cultured in complete media (IL-15^{N72D}/sushi-Fc Nanogels). The number of live cells was measured after 16 hours (gray bars) and on day 9 (black bars) by flow cytometry. In FIG. 29B, CD3 T cells were conjugated (rightmost group) or not (3 leftmost groups) with IL-15^{WT}/sushi-Fc Nanogels before freezing in serum-free media (Bambanker) overnight. Upon thawing, first group was cultured in complete media (Media only), second group was cultured in IL-2 containing (20 ng/ml) complete media (IL-2 (soluble)), third group was cultured in IL-15^{WT}/sushi-Fc containing (12 ug/ml) complete media (IL-15^{WT}/sushi-Fc (12 ug/ml)) and fourth group was cultured in complete media (IL-15^{WT}/sushi-Fc Nanogels). The number of live cells was measured on day 2 (light gray bars), on day 6 (dark gray bars) and on day 7 (black bars) by microscopy. Complete media for this experiment was IMDM (Lonza), Glutamaxx (Life Tech), 20% FBS (Life Tech), 2.5 ug/ml human albumin (Octapharma), 0.5 ug/ml Inositol (Sigma).

FIGS. 30A-30B depict NK-92 cell line and primary NK cell expansion analysis. In FIG. 30A, NK-92 cells were associated (2 rightmost groups) or not (3 leftmost groups) with IL-15^{WT}/sushi-Fc protein gels (Nanogels). First 4 groups were frozen in serum-free media (Bambanker) for 2 hours, fifth group was washed and cultured in complete media (IL-15^{WT}/sushi-Fc Nanogels (no freezing)). Upon thawing of 4 leftmost groups, first group was cultured in complete media (Media only), second group was cultured in IL-2 containing (20 ng/ml) complete media (IL-2 (soluble)), third group was cultured in IL-15^{WT}/sushi-Fc containing (12 ug/ml) complete media (IL-15^{WT}/sushi-Fc (12 ug/ml)) and fourth group was cultured in complete media (IL-15^{WT}/sushi-Fc Nanogels). The number of live cells was measured on day 1 (light gray bars), on day 5 (dark gray bars) and on day 6 (black bars) by microscopy. In FIG. 30B, primary NK cells were associated (rightmost group) or not (3 leftmost groups) with IL-15^{N72D}/sushi-Fc protein nanogel before freezing in serum-free media (Bambanker) for 2 weeks. Upon thawing, first group was cultured in complete media (Media only), second group was cultured in IL-2 containing (20 ng/ml) complete media (IL-2 (soluble)), third group was cultured in IL-15^{N72D}/sushi-Fc containing (0.6 ug/ml) complete media (IL-15^{N72D}/sushi-Fc (0.6 ug/ml)) and fourth group was cultured in complete media (IL-15^{N72D}/sushi-Fc Nanogels). The number of live cells was measured after 16 hours (gray bars) and on day 9 (black bars) by flow cytometry. Complete media for this experiment was Xvivo10 containing recombinant transferrin (Lonza), Glutamaxx (Life Tech), 5% human serum AB (Corning).

FIGS. 31A-31B depict T cell subset analysis. In FIG. 31A, CD3 T cells were associated (rightmost group) or not (3 leftmost groups) with IL-15N72D/sushi-Fc protein nanogels before freezing in serum-free media (Bambanker) for 2 weeks. Upon thawing, the first group was cultured in complete media (Media only), the second group was cultured in IL-2 containing (20 ng/ml) complete media (IL-2 (soluble)), the third group was cultured in IL-15N72D/sushi-Fc containing (0.6 ug/ml) complete media (IL-15N72D/sushi-Fc (0.6 ug/ml)) and the fourth group was cultured in complete media (IL-15N72D/sushi-Fc Nanogels). After 9 days in culture, CD3 T cells were analyzed by flow cytometry for expression of subset (FIG. 31A) and activation (FIG. 31B) markers.

FIGS. 32A-32B depict T cell potency analysis. In FIG. 32A, CD3 T cells were associated (rightmost group) or not (3 leftmost groups) with IL-15^{N72D}/sushi-Fc protein nanogels (before freezing in serum-free media (Bambanker) for 2 weeks. Upon thawing, the first group was cultured in complete media (Media only), the second group was cultured in IL-2 containing (20 ng/ml) complete media (IL-2 (soluble)), the third group was cultured in IL-15^{N72D}/sushi-Fc containing (0.6 ug/ml) complete media (IL-15^{N72D}/sushi-Fc (0.6 ug/ml)) and the fourth group was cultured in complete media (IL-15^{N72D}/sushi-Fc Nanogels). After 1 day in culture, CD3 T cells were co-cultured with target cells (Daudi) at different effector to target (E:T) ratios. Killing of target cells was measured by flow cytometry after 16 hours. Statistical significance was calculated by 2-way ANOVA with Tukey's multiple comparison test. *: p<0.05; **: p<0.01. FIG. 32B shows measurements of IFNg release from same cells as in FIG. 32A. Complete media for this experiment was IMDM (Lonza), Glutamaxx (Life Tech), 20% FBS (Life Tech), 2.5 ug/ml human albumin (Octapharma), 0.5 ug/ml Inositol (Sigma).

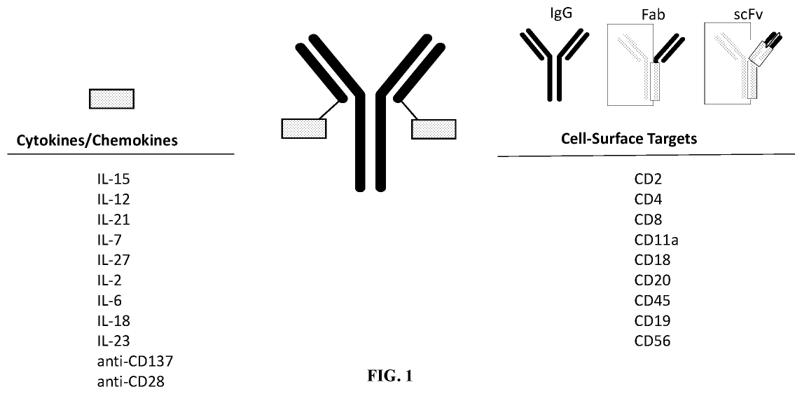

### DETAILED DESCRIPTION

The present disclosure provides, inter alia, compositions and related methods of use of immunostimulatory fusion molecules or IFMs. An “IFM” as described herein includes an immune stimulating moiety, e.g., a cytokine molecule (e.g., a biologically active cytokine), and an immune cell targeting moiety, e.g., an antibody molecule (e.g. an antibody or antibody fragment) capable of binding to an immune cell, e.g., an immune effector cell. In embodiments, the immune stimulating moiety and the immune cell targeting moiety are functionally linked (e.g., by chemical coupling, genetic fusion, noncovalent association or otherwise). In some embodiments, the immune cell targeting moiety is capable of binding to an immune cell surface target, thereby targeting the immune stimulating moiety, e.g., cytokine molecule, to the immune cell, e.g., an immune effector cell (e.g., a lymphocyte).

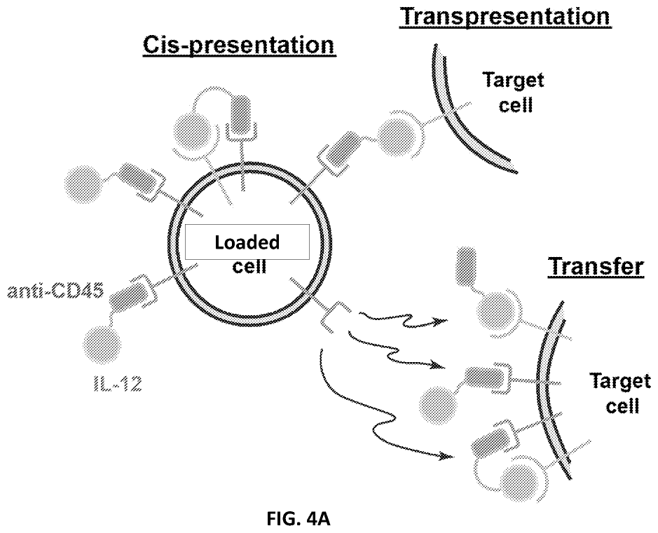

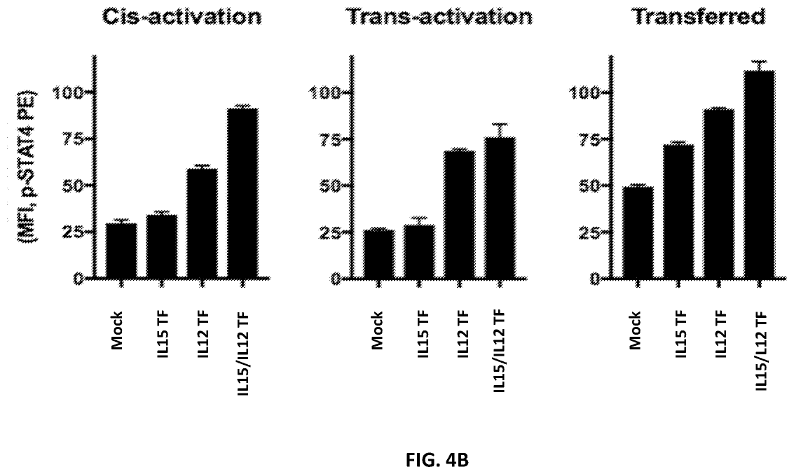

Without wishing to be bound by theory, binding of the immune cell targeting moiety to the immune cell surface target is believed to increase the concentration, e.g., the concentration over time, on the surface of the immune stimulating moiety, e.g., cytokine molecule, with its corresponding receptor, e.g., a cytokine receptor, on the immune cell, e.g., relative to the association of the free cytokine molecule with its cytokine receptor. This can result in an immune effect on the immune cell itself bound by the IFMs (autocrine signaling), and/or or on another (e.g., neighboring) immune cell (paracrine signaling). Advantageously, compared to other therapeutics such as soluble cytokines, armored CAR-T (with cytokines) and nanogels (onto immune cells), the tethered fusions of the present disclosure can provide balanced, dual autocrine and paracrine activity, combining the benefits of both sufficiently high activity and low toxicity. In contrast, delivery of soluble cytokines while providing systemic activity, is known for its high toxicity. Armored CAR-T can locally secrete cytokines that provide paracrine and systemic activity, as well as systemic toxicities. Nanogels such as those described in, e.g., U.S. Publication No. 2017/0080104, U.S. Pat. No. 9,603,944. U.S. Publication No. 2014/0081012, and PCT Application Nos. PCT/US2017/037249 and PCT/US2018/049596 (each incorporated herein by reference in its entirety), are capable of providing highly localized autocrine activity, which may be desirable for certain cytokines (e.g., IL-15). However, for cytokines (e.g., IL-12) where paracrine activity is needed, the tethered fusions disclosed herein can be a sweet spot for balanced autocrine and paracrine activity. As shown in FIG. 4A, tethered fusions can signal in cis once tethered (or loaded) onto an immune cell, in trans to a neighboring target immune cell, and by transfer to target immune cells that are not in close proximity to the original surface-loaded cells.

In embodiments, the immune cell targeting moiety results in an increase in one or more of: binding, availability, activation and/or signaling of the immune stimulating moiety on the immune cell, e.g., over a specified amount of time. In embodiments, the IFM does not substantially interfere with the signaling function of the cytokine molecule. Such targeting effect results in localized and prolonged stimulation of proliferation and activation of the immune cells, thus inducing the controlled expansion and activation of an immune response. The IFMs disclosed herein offer several advantages over art-known cytokines, including reduced side effects, e.g., a lower systemic toxicity, while retaining the immunostimulatory bioactivity (e.g., signaling activity and/or potency) of the cytokine molecule.

Prior disclosures of immunocytokines-antibody-cytokine fusion proteins are typically designed to target disease antigens (e.g., tumor associated antigens e.g., cell membrane antigens and extracellular matrix components) via their antibody components in order to potentiate effector functions through their cytokine components. (Clin. Pharmacol. 2013; 5(Suppl 1): 29-45. Thomas List and Dario Neri. Published online 2013 Aug. 20. doi: 10.2147/CPAA.S49231 PMCID: PMC3753206.) Exemplary barriers to the therapeutic use of cytokines relate to their short serum half-life and limited bioavailability. High doses of cytokines can overcome these barriers, but result in dose-limiting toxicities. Consequently, most cytokines require protein engineering approaches to reduce toxicity and increase half-life. Specific strategies include PEGylation, antibody complexes and fusion protein formats, and mutagenesis. (Antibodies 2013, 2, 426-451; doi:10.3390/antib2030426 Rodrigo Vazquez-Lombardi Brendan Roome and Daniel Christ.)

The present disclosure provides, inter alia, fusion proteins as a covalent conjugate of a cytokine and a targeting moiety which functions to target the fusion protein to an immune cell (e.g., healthy and/or non-malignant) with a particular composition of receptors. Fusing a pro-inflammatory cytokine to a targeting moiety, preferably an antibody or antibody fragment (e.g. single chain Fv, Fab, IgG), directs the fusion protein to a cell of interest and enhances cell surface availability of the cytokine. Cells of interest include, inter alia, immune cells, especially lymphocytes, and preferably T-cells (e.g., total CD3 T cells, CD4 T cells, or CD8 T cells), and can include other cell types. In some embodiments, the fusion proteins can activate a subset of CD8 T cells. Cells of interest, including immune cells, can be in vivo (e.g., in a subject), in vitro or ex vivo (e.g., a cell based therapy).

In some embodiments, the immune cell surface target is abundantly present on the surface of an immune cell (e.g., outnumbers the number of receptors for the cytokine molecule present on the immune cell surface). In some embodiments, the immune cell targeting moiety can be chosen from an antibody molecule or a ligand molecule that binds to an immune cell surface target, e.g., a target chosen from CD4, CD8, CD18, CD11a, CD11b, CD11c, CD19, CD20 or CD45. In one embodiment, the immune cell targeting moiety comprises an antibody molecule or a ligand molecule that binds to CD45.

CD45 is an example of an abundant receptor. CD45 is also known as leukocyte common antigen, is a type I transmembrane protein present on hematopoietic cells except erythrocytes that assists in cell activation (see e.g., Altin, J G, Immunol Cell Biol. 1997 October; 75(5):430-45)). Other receptors of the targeting moiety of the IFM are ideally maintained on the cell surface and are resistant to internalization by the cell (e.g. persistent receptors). An example of an abundant and persistent receptor is CD45. Alternatively, receptors of the targeting moiety may be constitutively turned over, e.g. internalized by the cell and recycled back to the surface thus allowing significant binding opportunities for the fusion protein, despite their dynamic internalization (recycling receptors). CD22 is an example of a recycling receptor.

Expression levels of cytokine receptors can vary based on a variety of factors, including the (i) cell type, and (ii) the activation state of the cell. In embodiments, the expression level can impact one or more of cytokine signal transduction, signal strength and duration. In one embodiment, the receptors expressed on the immune cell surface are present in an effective ratio whereby the number of receptors to the targeting moiety is in excess of the number of receptors to the cytokine component, on the cell surface. Such an effective ratio is realized when the targeting moiety receptors are persistent; or alternatively; when their cell surface density is effectively maintained by a recycling mechanism which restores the receptors to the cell's surface and consequently permits binding opportunities for the targeting component in excess of the cytokine component. In the case of receptors that are recycled (e.g. internalized and returned to the cell surface), antibody receptors will be present in an effective ratio to allow binding opportunities for the targeting moiety in excess of binding opportunities for the cytokine component of the protein. Such an effective ratio allows cytokine localization to the cell surface and consequently increases the time and availability of the cytokine to bind its own cell-surface receptor (despite the dynamic presence, internalization and return to the surface of the targeting receptor).

In some cases, regulation of signaling initiated by plasma membrane receptors is coupled to endocytosis. Internalization of activated receptors is a means for signal attenuation, but also regulates the duration of receptor signaling and signaling output specificity (reviewed in Barbieri, P. P. Di Fiore, S. Sigismund. Endocytic control of signaling at the plasma membrane Curr. Opin. Cell Biol., 39 (2016), pp. 21-27). Endosomes can serve as mobile signaling platforms facilitating formation of multiprotein signaling assemblies and consequently enabling efficient signal transduction in space and time. Some signaling events, e.g. cytokine-signaling events, initiated at the plasma membrane may continue from endosomal compartments.

IFMs of the disclosure can confer improved biological activity of agonistic cytokines in general, and of IL-15, IL-7, IL-21, and IL-12p70 in particular. Other agonistic cytokines include IL-2, IL-6, and IL-27. In some embodiments, the cytokine molecule includes a pro-inflammatory cytokine, e.g., includes a cytokine chosen from one or more of IL-2, IL-6, IL-7, IL-12, IL-15, IL-21 or IL-27, including variant forms thereof (e.g., a cytokine derivative, a complex comprising the cytokine molecule with a polypeptide, e.g., a cytokine receptor complex, and other agonist forms thereof). In one embodiment, the cytokine molecule includes IL-15 and/or IL-12 (in one IFM or two IFMs).

In one exemplary embodiment, the immune cell targeting moiety of the IFM is derived from an anti-CD45 antibody molecule and the cytokine molecule is interleukin-15 optionally complexed to the sushi domain of the IL-15 receptor alpha subunit (αCD45-IL15 and αCD45-IL15/sushi). In another exemplary embodiment, the immune cell targeting moiety of the IFM is derived from an anti-CD45 antibody molecule and the cytokine molecule is interleukin-12 (αCD45-IL12). The αCD45-IL15/sushi IFM and αCD45-IL12 IFM can be used together in, e.g., a combination therapy.

In another embodiment, the IFMs can be tethered to different cell surface molecules, e.g., an IFM in which the immune cell targeting moiety is derived from an antibody targeting an abundant or persistence cell surface receptor other than CD45, e.g., a target chosen from CD4, CD8, CD18, CD11a, CD11b, CD11c, CD19, or CD20. The IFMs can contain cytokines such as interleukin-15 optionally complexed to the sushi domain of the IL-15 receptor alpha subunit and/or interleukin-12. The IFMs can be used together in, e.g., a combination therapy with αCD45-IL15, αCD45-IL15/sushi, or αCD45-IL12.

In another embodiment, IFMs comprising additional cytokines tethered to the same or different cell surface receptors are used together, e.g., in a combination therapy. The immune cell targeting moiety can be chosen from an antibody molecule or a ligand molecule that binds to an immune cell surface target, e.g., a target chosen from CD4, CD8, CD18, CD11a, CD11b, CD11c, CD19, CD20 or CD45, and a pro-inflammatory cytokine, e.g., includes a cytokine chosen from one or more of IL-2, IL-6, IL-7, IL-12, IL-15, IL-21 or IL-27, including variant forms thereof. In some embodiments combinations of two different IFMs are used. In other embodiments combinations of three different IFMs are used. In other embodiments combinations of more than three IFMs are used.

Therapeutic uses for the fusion proteins of the disclosure include, inter alia, (1) as agents for specific delivery of therapeutic proteins via receptor mediated binding of receptors unique to specific cells (e.g., CD4 or CD8); (2) as ex vivo agents to induce activation and expansion of isolated autologous and allogenic cells prior to reintroduction to a patient; for example, in T cell therapies including ACT (adoptive cell transfer) and also with other important immune cell types, including for example, B cells, tumor infiltrating lymphocytes, NK cells, antigen-specific CD8 T cells, T cells genetically engineered to express chimeric antigen receptors (CARs) or CAR-T cells, T cells genetically engineered to express T-cell receptors specific to an tumor antigen, tumor infiltrating lymphocytes (TILs), and/or antigen-trained T cells (e.g., T cells that have been “trained” by antigen presenting cells (APCs) displaying antigens of interest, e.g. tumor associated antigens (TAA)); and, (3) as in vivo agents for administration to deliver cytokines used to support expansion of cells used in cell therapies, including ACT.

As such, a pharmaceutical composition comprising the IFM of the present disclosure and a pharmaceutically acceptable carrier, excipient, or stabilizer can be used to deliver therapeutic proteins to a subject in need thereof. A modified immune cell, comprising a healthy and/or non-malignant immune cell and the IFM of the present disclosure bound or targeted thereto, is also provided. Such modified immune cell can be prepared in vitro or in vivo.

The present disclosure also provides a method of in vitro preparation of modified immune cells, comprising: providing a plurality of healthy and/or non-malignant immune cells; and incubating the IFM of the present disclosure with the plurality of healthy and/or non-malignant immune cells so as to permit targeted binding of the IFM thereto, thereby producing a plurality of modified immune cells.

Also provided herein is a method of providing a cell therapy, comprising: providing a plurality of healthy and/or non-malignant immune cells; incubating the IFM of the present disclosure with the plurality of healthy and/or non-malignant immune cells so as to permit targeted binding of the IFM thereto, thereby producing a plurality of modified immune cells; and administering the plurality of modified immune cells to a subject in need thereof. In some embodiments, the cell therapy is administered in the absence of pre-conditioning of the subject, wherein said pre-conditioning comprises CPX (cyclophosphamide) or other lymphodepletion conditioning chemotherapy. The elimination of pre-conditioning is advantageous as it is well known that pre-conditioning with chemotherapy agents can cause systemic high toxicity to all cells, including healthy cells, weaken the immune system and induce many undesirable side effects.

In some embodiments, provided herein is a co-load of IL-15 nanogel and IL-12 TF, which enables Pmel antigen-specific expansion following antigen encounter, promotes long term Pmel activation and IFNg production even at low effector:target ratio, and/or supports persistent antigen-specific target cytotoxicity.

In some embodiments, provided herein is a IL-15 nanogel and IL-12 TF combination therapy, which enables antigen-specific cell expansion upon antigen encounter, enhances IFNg production, maintains memory phenotypes and activation states, following antigen encounter, and/or enhances long term, antigen-specific target cytotoxicity.

In some embodiments, provided herein is a IL-15 nanogel and IL-12 TF combination therapy, which enables MART-1 antigen-specific expansion upon antigen encounter, enhances IFNg production, maintains effector memory phenotypes and activation states, following antigen encounter, and/or supports antigen-specific target cytotoxicity.

### Definitions

Certain terms are defined herein below. Additional definitions are provided throughout the application.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

The term “autologous” refers to any material derived from the same individual to whom it is later to be re-introduced into the individual. The term “allogeneic” refers to any material derived from a different animal of the same species as the individual to whom the material is introduced.

“Antibody” or “antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In an embodiment, an antibody molecule comprises an antigen binding or functional fragment of a full length antibody, or a full length immunoglobulin chain. For example, a full-length antibody is an immunoglobulin (Ig) molecule (e.g., IgG) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)2 fragments, and single chain variable fragments (scFvs). The terms “Fab” and “Fab fragment” are used interchangeably and refer to a region that includes one constant and one variable domain from each heavy and light chain of the antibody, i.e., V_{L}, C_{L}, V_{H}, and C_{H}1.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a macromolecule, including all proteins or peptides. In some embodiments, an antigen is a molecule that can provoke an immune response, e.g., involving activation of certain immune cells and/or antibody generation. Antigens are not only involved in antibody generation. T cell receptors also recognized antigens (albeit antigens whose peptides or peptide fragments are complexed with an MHC molecule). Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site” or “antigen-binding fragment” or “antigen-binding portion” (used interchangeably herein) of an antibody molecule refers to the part of an antibody molecule, e.g., an immunoglobulin (Ig) molecule such as IgG, that participates in antigen binding. In some embodiments, the antigen-binding site is formed by amino acid residues of the variable (V) regions of the heavy (H) and light (L) chains Three highly divergent stretches within the variable regions of the heavy and light chains, referred to as hypervariable regions, are disposed between more conserved flanking stretches called “framework regions” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4. Variable light chain (VL) CDRs are generally defined to include residues at positions 27-32 (CDR1), 50-56 (CDR2), and 91-97 (CDR3). Variable heavy chain (VH) CDRs are generally defined to include residues at positions 27-33 (CDR1), 52-56 (CDR2), and 95-102 (CDR3). One of ordinary skill in the art would understand that the loops can be of different length across antibodies and the numbering systems such as the Kabat or Chotia control so that the frameworks have consistent numbering across antibodies.

In some embodiments, the antigen-binding fragment of an antibody (e.g., when included as part of the fustion molecule of the present disclosure) can lack or be free of a full Fc domain. In certain embodiments, an antibody-binding fragment does not include a full IgG or a full Fc but may include one or more constant regions (or fragments thereof) from the light and/or heavy chains. In some embodiments, the antigen-binding fragment can be completely free of any Fc domain. In some embodiments, the antigen-binding fragment can be substantially free of a full Fc domain. In some embodiments, the antigen-binding fragment can include a portion of a full Fc domain (e.g., CH2 or CH3 domain or a portion thereof). In some embodiments, the antigen-binding fragment can include a full Fc domain. In some embodiments, the Fc domain is an IgG domain, e.g., an IgG1, IgG2, IgG3, or IgG4 Fc domain. In some embodiments, the Fc domain comprises a CH2 domain and a CH3 domain.

As used herein, a “cytokine molecule” refers to full length, a fragment or a variant of a naturally-occurring, wild type cytokine (including fragments and functional variants thereof having at least 10% of the activity of the naturally-occurring cytokine molecule). In embodiments, the cytokine molecule has at least 30, 50, or 80% of the activity, e.g., the immunomodulatory activity, of the naturally-occurring molecule. In embodiments, the cytokine molecule further comprises a receptor domain, e.g., a cytokine receptor domain, optionally, coupled to an immunoglobulin Fc region. In other embodiments, the cytokine molecule is coupled to an immunoglobulin Fc region.

The term “co-administration” in the present invention refers to the administration of different immune agonist moieties, such as an IL-12 tether fusion-loaded T cell and an IL-15 nanogel-loaded T cell under conditions such that the entities, e.g., the IL-12 immune agonist and the IL-15 immune agonist and elicit a synergistic effect in at least one desired parameter such as synergistic potency and/or synergistic efficacy. The moieties may be administered in the same or different compositions which if separate are administered proximate to one another, e.g., within 24 hours of each other, or within about 1-8 hours of one another, and or with 1-4 hours of each other or close to simultaneous administration. The relative amounts are dosages that achieve the desired synergism.

The term, “combination therapy” embraces administration of each agent or therapy in a sequential manner in a regiment that will provide beneficial effects of the combination, and co-administration of these agents or therapies in a substantially simultaneous manner, such as in a single composition having a fixed ratio of these active agents or in multiple, separate compositions for each agent. Combination therapy also includes combinations where individual elements may be administered at different times and/or by different routes but which act in combination to provide a beneficial effect by co-action or pharmacokinetic and pharmacodynamics effect of each agent or tumor treatment approaches of the combination therapy.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. The term “immune cell” includes immune effector cells described herein “Immune cell” also refers to modified versions of cells involved in an immune response, e.g. modified NK cells, including NK cell line NK-92 (ATCC cat. No. CRL-2407), haNK (an NK-92 variant that expresses the high-affinity Fc receptor FcγRIIIa (158V)) and taNK (targeted NK-92 cells transfected with a gene that expresses a CAR for a given tumor antigen), e.g., as described in Klingemann et al. supra.

“CD45,” also known as leukocyte common antigen, refers to human CD45 protein and species, isoforms, and other sequence variants thereof. Thus, CD45 can be the native, full-length protein or can be a truncated fragment or a sequence variant (e.g., a naturally occurring isoform, or recombinant variant) that retains at least one biological activity of the native protein. CD45 is a receptor-linked protein tyrosine phosphatase that is expressed on leukocytes, and which plays an important role in the function of these cells (reviewed in Altin, J G (1997) Immunol Cell Biol. 75(5):430-45, incorporated herein by reference). For example, the extracellular domain of CD45 is expressed in several different isoforms on T cells, and the particular isoform(s) expressed depends on the particular subpopulation of cell, their state of maturation, and antigen exposure. Expression of CD45 is important for the activation of T cells via the TCR, and that different CD45 isoforms display a different ability to support T cell activation.

“Immune effector cell,” as that term is used herein, refers to a cell that is involved in an immune response, e.g., in the promotion of an immune effector response. Examples of immune effector cells include, but are not limited to, T cells, e.g., CD4T cells, CD8 T cells, alpha T cells, beta T cells, gamma T cells, and delta T cells; B cells; natural killer (NK) cells; natural killer T (NKT) cells; dendritic cells; and mast cells. In some embodiments, the immune cell is an immune cell (e.g., T cell or NK cell) that comprises, e.g., expresses, a Chimeric Antigen Receptor (CAR), e.g., a CAR that binds to a cancer antigen. In other embodiments, the immune cell expresses an exogenous high affinity Fc receptor. In some embodiments, the immune cell comprises, e.g., expresses, an engineered T-cell receptor. In some embodiments, the immune cell is a tumor infiltrating lymphocyte. In some embodiments the immune cells comprise a population of immune cells and comprise T cells that have been enriched for specificity for a tumor-associated antigen (TAA), e.g. enriched by sorting for T cells with specificity towards MHCs displaying a TAA of interest, e.g. MART-1. In some embodiments immune cells comprise a population of immune cells and comprise T cells that have been “trained” to possess specificity against a TAA by an antigen presenting cell (APC), e.g. a dendritic cell, displaying TAA peptides of interest. In some embodiments, the T cells are trained against a TAA chosen from one or more of MART-1, MAGE-A4, NY-ESO-1, SSX2, Survivin, or others. In some embodiments the immune cells comprise a population of T cells that have been “trained” to possess specificity against a multiple TAAs by an APC, e.g. a dendritic cell, displaying multiple TAA peptides of interest. In some embodiments, the immune cell is a cytotoxic T cell (e.g., a CD8 T cell). In some embodiments, the immune cell is a helper T cell, e.g., a CD4 T cell.

The term “effector function” or “effector response” refers to a specialized function of a cell. Effector function of a T cell, for example, may be cytolytic activity or helper activity including the secretion of cytokines.

“Cytotoxic T lymphocytes” (CTLs) as used herein refer to T cells that have the ability to kill a target cell. CTL activation can occur when two steps occur: 1) an interaction between an antigen-bound MHC molecule on the target cell and a T cell receptor on the CTL is made; and 2) a costimulatory signal is made by engagement of costimulatory molecules on the T cell and the target cell. CTLs then recognize specific antigens on target cells and induce the destruction of these target cells, e.g., by cell lysis. In some embodiments, the CTL expresses a CAR. In some embodiments, the CTL expresses an engineered T-cell receptor.

The compositions and methods of the present disclosure encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5 The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid (e.g., SEQ ID NO: 1) molecules of the disclosure. BLAST protein searches can be performed with the XBLAST program, score=50, wordlength=3 to obtain amino acid sequences homologous to protein molecules of the disclosure. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov. It is understood that the molecules of the present disclosure may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “functional variant” or “variant” or “variant form” in the context of a polypeptide refers to a polypeptide that is capable of having at least 10% of one or more activities of the naturally-occurring sequence. In some embodiments, the functional variant has substantial amino acid sequence identity to the naturally-occurring sequence, or is encoded by a substantially identical nucleotide sequence, such that the functional variant has one or more activities of the naturally-occurring sequence.

The term “molecule” as used herein can refer to a polypeptide or a nucleic acid encoding a polypeptide, as indicated by the context. This term includes full length, a fragment or a variant of a naturally-occurring, wild type polypeptide or nucleic acid encoding the same, e.g., a functional variant, thereof. In some embodiments, the variant is a derivative, e.g., a mutant, of a wild type polypeptide or nucleic acid encoding the same.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “parent polypeptide” refers to a wild-type polypeptide and the amino acid sequence or nucleotide sequence of the wild-type polypeptide is part of a publicly accessible protein database (e.g., EMBL Nucleotide Sequence Database, NCBI Entrez, ExPasy, Protein Data Bank and the like).

The term “mutant polypeptide” or “polypeptide variant” or “mutein” refers to a form of a polypeptide, wherein its amino acid sequence differs from the amino acid sequence of its corresponding wild-type (parent) form, naturally existing form or any other parent form. A mutant polypeptide can contain one or more mutations, e.g., replacement, insertion, deletion, etc. which result in the mutant polypeptide.

The term “corresponding to a parent polypeptide” (or grammatical variations of this term) is used to describe a polypeptide of the present disclosure, wherein the amino acid sequence of the polypeptide differs from the amino acid sequence of the corresponding parent polypeptide only by the presence of at least amino acid variation. Typically, the amino acid sequences of the variant polypeptide and the parent polypeptide exhibit a high percentage of identity. In one example, “corresponding to a parent polypeptide” means that the amino acid sequence of the variant polypeptide has at least about 50% identity, at least about 60%, at least about 70%, at least about 80%, at least about 90%, at least about 95% or at least about 98% identity to the amino acid sequence of the parent polypeptide. In another example, the nucleic acid sequence that encodes the variant polypeptide has at least about 50% identity, at least about 60%, at least about 70%, at least about 80%, at least about 90%, at least about 95% or at least about 98% identity to the nucleic acid sequence encoding the parent polypeptide.

The term “introducing (or adding etc.) a variation into a parent polypeptide” (or grammatical variations thereof), or “modifying a parent polypeptide” to include a variation (or grammatical variations thereof) do not necessarily mean that the parent polypeptide is a physical starting material for such conversion, but rather that the parent polypeptide provides the guiding amino acid sequence for the making of a variant polypeptide. In one example, “introducing a variant into a parent polypeptide” means that the gene for the parent polypeptide is modified through appropriate mutations to create a nucleotide sequence that encodes a variant polypeptide. In another example, “introducing a variant into a parent polypeptide” means that the resulting polypeptide is theoretically designed using the parent polypeptide sequence as a guide. The designed polypeptide may then be generated by chemical or other means.

According to the present invention, a target “immune cell” is a nucleated cell, e.g., a nucleated cell as described herein below. In more particular embodiments, the immune cell, e.g., an immune effector cell, (e.g., an immune cell chosen from a lymphocyte, T cell, B cell, or a Natural Killer cell), or a hematopoietic stem cell). In embodiments, the immune cell comprises a lymphocyte. In embodiments, the immune cell comprises a T cell. In embodiments, the immune cell comprises a B cell. In embodiments, the immune cell comprises a Natural Killer (NK) cell. In embodiments, the immune cell comprises a hematopoietic stem cell. In some embodiments, the immune cell is an immune cell (e.g., T cell or NK cell) that comprises, e.g., expresses, a Chimeric Antigen Receptor (CAR), e.g., a CAR that binds to a cancer antigen. In some embodiments, the immune cell comprises, e.g., expresses an engineered T-cell receptor. In some embodiments, the immune cell is a tumor infiltrating lymphocyte. In some embodiments, the immune cell is a cytotoxic T cell (e.g., a CD8 T cell). In some embodiments, the immune cell is a regulatory T-cell (“Treg”). In some embodiments, the immune cell is a population of immune effector cells, e.g., a population of immune effector cells chosen from one or more of: T cells, e.g., CD4 T cells, CD8 T cells, alpha T cells, beta T cells, gamma T cells, and delta T cells; B cells; natural killer (NK) cells; natural killer T (NKT) cells; or dendritic cells. In embodiments, the immune cell, e.g., the immune effector cell, displays a cell surface receptor that binds the immune cell targeting moiety. In embodiments, the immune cell is an immune cell acquired from a patient, e.g., a patient's blood. In other embodiments, the immune cell is an immune cell acquired from a healthy donor. In embodiments, the immune cell is an immune cell from an embryonic stem cell and/or an iPSC cell. In some embodiments, the immune cell is a cell line, e.g., a stable or an immortalized cell line.

Various aspects of the disclosure are described in further detail below. Additional definitions are set out throughout the specification.

### Cytokine Molecules

Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells, including those of both the innate (monocytes, macrophages, dendritic cells) and adaptive (T- and B-cells) immune systems. Cytokines can be classified into two groups: pro- and anti-inflammatory. Pro-inflammatory cytokines, including IFN-γ, IL-1, IL-6 and TNF-α, are predominantly derived from the innate immune cells and Th1 cells. Anti-inflammatory cytokines, including IL-10, IL-4, IL-13 and IL-5, are synthesized from Th2 immune cells.

In some embodiments, the cytokine molecule of the IFM and/or the protein nanogel includes an immunomodulatory cytokine, e.g., a pro-inflammatory cytokine or an anti-inflammatory cytokine. In some embodiments, the cytokine is a member of the common γ-chain (γc) family of cytokines. In some embodiments, the cytokine molecule comprises a cytokine chosen from one or more of interleukin-15 (IL-15), interleukin-1, e.g., interleukin-1 alpha (IL-1α) or interleukin-1 beta (IL-1β), interleukin-2 (IL-2), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin-13 (IL-13), interleukin-18 (IL-18), interleukin-21 (IL-21), interleukin-23 (IL-23), interleukin-27 (IL-27), interleukin-35 (IL-35), IFNγ, TNFα, IFNα, IFNβ, GM-CSF, or GCSF, including variant forms thereof (e.g., a cytokine derivative, a complex comprising the cytokine molecule with a polypeptide, e.g., a cytokine receptor complex, and other agonist forms thereof). In some embodiments, the cytokine molecule is a pro-inflammatory cytokine molecule chosen from an IL-1, IL-2, IL-6, IL-12, IL-15, IL-18, IL-21, IL-23, or IL-27 cytokine molecule. In some embodiments, the cytokine molecule is an anti-inflammatory cytokine molecule chosen from an IL-4, IL-10, IL-13, IL-35 cytokine molecule. In some embodiments, the cytokine molecule is chosen from IL-2, IL-6, IL-7, IL-12, IL-15, IL-21 or IL-27, including variant forms thereof (e.g., a cytokine derivative, a complex comprising the cytokine molecule with a polypeptide, e.g., a cytokine receptor complex, and other agonist forms thereof, e.g., a non-neutralizing anti-cytokine antibody molecule). In some embodiments, the cytokine molecule is a superagonist (SA), e.g., as described herein. For example, the superagonist can have increased cytokine activity, e.g., by at least 10%, 20%, or 30%, compared to the naturally-occurring cytokine. In some embodiments, the cytokine molecule is a monomer or a dimer. In embodiments, the cytokine molecule further comprises a receptor or a fragment thereof, e.g., a cytokine receptor domain.

The present disclosure provides, inter alia, IFMs (e.g., IFM polypeptides) and/or protein nanogels, that include, e.g., are engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin.

In embodiments, the cytokine molecule is full length, a fragment or a variant of a cytokine, e.g., a cytokine comprising one or more mutations. In some embodiments the cytokine molecule comprises a cytokine chosen from interleukin-15 (IL-15), interleukin-1 alpha (IL-1 alpha), interleukin-1 beta (IL-1 beta), interleukin-2 (IL-2), interleukin-4 (IL-4), interleukin-5 (IL-5), interleukin-7 (IL-7), interleukin-12 (IL-12), interleukin-18 (IL-18), interleukin-21 (IL-21), interleukin-23 (IL-23), interferon (IFN) α, IFN-β, IFN-γ, tumor necrosis factor alpha, GM-CSF, GCSF, or a fragment or variant thereof, or a combination of any of the aforesaid cytokines. In other embodiments, the cytokine molecule is chosen from interleukin-2 (IL-2), interleukin-12 (IL-12), interleukin-15 (IL-15), interleukin-18 (IL-18), interleukin-21 (IL-21), or interferon gamma, or a fragment or variant thereof, or a combination of any of the aforesaid cytokines. The cytokine molecule can be a monomer or a dimer. In embodiments, the cytokine molecule further comprises a receptor domain, e.g., a cytokine receptor domain.

In some embodiments the cytokine or growth factor molecule can be a Treg inhibitory molecule selected from one or more of Ifn-γ, IL-1α, IL-1β, IL-6, IL-12, IL-21, IL-23, IL-27 or TNF-α. Ifn-γ promotes Treg fragility, and can reduce suppression in the tumor microenvironment. IL-1 and IL-6, IL-21 and IL-23 can induce Tregs to produce pro-inflammatory IL-17 and/or convert Tregs to Th17 T cell subset. IL-12 promotes Ifn-γ production in Tregs, leading Treg fragility and a general pro-immunogenic environment. TNF-α both impairs Treg development and reduces the function of existing Tregs. Thus, these cytokines can impair Treg development, reduce Treg function or induce Treg trans-differentiation into immune activating cells.

In the context of cancer, where it is desired to reduce Treg activity, one or more of the Treg inhibitory cytokines can be delivered systemically via Treg-specific IFMs in order to reduce Treg suppression globally. Bi-specific targeting (e.g., CD4:CD25, CD4:NRP1, CD4:CD39) can be used to direct systemically injected IFMs to the Treg cells in vivo. This concept of targeting a specific cell population is described in the Examples. These IFMs then reduce Treg numbers and/or function, and drive them to a pro-inflammatory or immunogenic state.

Additionally, Treg-specific IFMs can be loaded onto anti-tumor immune cells ex vivo and delivered to the Tregs via trans or paracrine signaling. In this case, the anti-tumor cells create a local anti-suppressive environment by increasing the local concentration of Treg inhibitory cytokines. This can promote local as opposed to global Treg dysfunction.

In one embodiment, the cytokine molecule comprises a wild type cytokine, e.g., a wild type, e.g., human amino acid sequence. In other embodiments, the cytokine molecule comprises an amino acid sequence substantially identical to the wild-type cytokine sequence, e.g., the human cytokine sequence. In some embodiments, the cytokine molecule comprises an amino acid sequence at least 95% to 100% identical, or having at least 1, 2, 3, 4, 5, 6, 7, 8, 9, or 10 or more amino acid alterations (e.g., substitutions, deletions, or insertions, e.g., conservative substitutions) relative to a wild-type cytokine sequence, e.g., a human cytokine sequence. In embodiments, the cytokine molecule comprises no more than five, ten or fifteen alterations (e.g., substitutions, deletions, or insertions, e.g., conservative substitutions) relative to the wild-type cytokine sequence, e.g., the human cytokine sequence.

Exemplary cytokine amino acid sequences are disclosed herein, for example, the amino acid of IL-15 is provided as, e.g., SEQ ID NO:10 and SEQ ID NO:40; the amino acid of IL-12A is provided as, e.g., SEQ ID NO:46 and SEQ ID NO:47; the amino acid of IL-12B is provided as, e.g., SEQ ID NO:48 and SEQ ID NO:49; exemplary fusions of IL-12A and IL-12B are disclosed as e.g., SEQ ID NO:50 and SEQ ID NO:51. Any of the cytokine sequences disclosed herein and substantially identical sequences (e.g., at least 90%, 95% or higher sequence identity) can be used in the IFM disclosed herein.

### IL-12 Molecules

Interleukin-12 (IL-12) is a heterodimeric cytokine composed of p35 and 00 subunits which are encoded by 2 separate IL-12A and IL-12B, respectively. IL-12 is involved in the differentiation of naive T cells into Th1 cells. It is known as a T cell-stimulating factor, which can stimulate the growth and function of T cells. It stimulates the production of interferon-gamma (IFN-γ) and tumor necrosis factor-alpha (TNF-α) from T cells and natural killer (NK) cells, and reduces IL-4 mediated suppression of IFN-γ. T cells that produce IL-12 have a coreceptor, CD30, which is associated with IL-12 activity.

IL-12 plays an important role in the activities of NK cells and T lymphocytes. IL-12 mediates enhancement of the cytotoxic activity of NK cells and CD8 cytotoxic T lymphocytes. There also seems to be a link between IL-2 and the signal transduction of IL-12 in NK cells. IL-2 stimulates the expression of two IL-12 receptors, IL-12R-β1 and IL-12R-β2, maintaining the expression of a critical protein involved in IL-12 signaling in NK cells. Enhanced functional response is demonstrated by IFN-γ production and killing of target cells.

IL-12 also has anti-angiogenic activity, which means it can block the formation of new blood vessels. It does this by increasing production of interferon gamma, which in turn increases the production of a chemokine called inducible protein-10 (IP-10 or CXCL10). IP-10 then mediates this anti-angiogenic effect. Because of its ability to induce immune responses and its anti-angiogenic activity, there has been an interest in testing IL-12 as a possible anti-cancer drug. There is a link that may be useful in treatment between IL-12 and the diseases psoriasis & inflammatory bowel disease.

IL-12 binds to the IL-12 receptor, which is a heterodimeric receptor formed by IL-12R-β1 and IL-12R-β2. IL-12R-β2 is considered to play a key role in IL-12 function, since it is found on activated T cells and is stimulated by cytokines that promote Th1 cells development and inhibited by those that promote Th2 cells development. Upon binding, IL-12R-β2 becomes tyrosine phosphorylated and provides binding sites for kinases, Tyk2 and Jak2. These are important in activating critical transcription factor proteins such as STAT4 that are implicated in IL-12 signaling in T cells and NK cells.

IL-12 is a potent cytokine with the potential to reshape the anti-inflammatory environment in solid tumors. However, its clinical utility has been limited by severe toxicities both from soluble administration or from adoptively transferred T cells engineered to secrete IL-12. The tethered fusion (TF) disclosed herein enables improved control of cytokine dose and biodistribution. In in vitro model systems, the IL12-TF cytokine provides persistent loading of IL-12 on the surface of T cells and sustained T cell activation and signaling downstream of the IL-12 receptors. In turn, this can activate innate and adaptive immunity.

The IL-12 in the tethered fusion can be in the form of a single chain containing both the IL-12A and IL-12B subunits. In some embodiments, the IL-12 can be present as a non single-chain (i.e., as a heterodimer of IL-12A and IL-12B, which is the natural form of IL-12). For example, the TF can be made by co-expression of three protein subunits (Fab heavy chain, Fab light-chain w/IL-12A (or IL-12B), and IL-12B (or IL-12A).

### IL-15 Molecules

In some embodiments of the IFM and/or nanogel, the cytokine molecule is an IL-15 molecule, e.g., a full length, a fragment or a variant of IL-15, e.g., human IL-15. In embodiments, the IL-15 molecule is a wild-type, human IL-15, e.g., having the amino acid sequence of SEQ ID NO: 10. In other embodiments, the IL-15 molecule is a variant of human IL-5, e.g., having one or more amino acid modifications.

In some embodiments, the IL-15 variant comprises, or consists of, a mutation at position 45, 51, 52, or 72, e.g., as described in US 2016/0184399. In some embodiments, the IL-15 variant comprises four, five, or six or more mutations.

In some embodiments, the IL-15 variant comprises, or consists of, one or more mutations at amino acid position 8, 10, 61, 64, 65, 72, 101, or 108 (in reference to the sequence of human IL-15, SEQ ID NO: 11). In some embodiments the IL-15 variant possesses increased activity as compared with wild-type IL-15. In some embodiments the IL-15 variant possesses decreased activity as compared with wild-type IL-15. In some embodiments the IL-15 variant possesses approximately two-fold, four-fold, ten-fold, 20-fold, 40-fold, 60-fold, 100-fold, or more than 100-fold decreased activity as compared with wild-type IL-15. In some embodiments, the mutation is chosen from D8N, K10Q, D61N, D61H, E64H, N65H, N72A, N72H, Q101N, Q108N, or Q108H (in reference to the sequence of human IL-15, SEQ ID NO: 11). As those of ordinary skill in the art would realize, any combination of the positions can be mutated. In some embodiments, the IL-15 variant comprises two or more mutations. In some embodiments, the IL-15 variant comprises three or more mutations. In some embodiments, the IL-15 variant comprises four, five, or six or more mutations. In some embodiments the IL-15 variant comprises mutations at positions 61 and 64. In some embodiments the mutations at positions 61 and 64 are D61N or D61H and E64Q or E64H. In some embodiments the IL-15 variants comprises mutations at positions 61 and 108. In some embodiments the mutations at positions 61 and 108 are D61N or D61H and Q108N or Q108H.

In embodiments, the cytokine molecule further comprises a receptor domain, e.g., a cytokine receptor domain. In one embodiment, the cytokine molecule comprises an IL-15 receptor, or a fragment thereof (e.g., an IL-15 binding domain of an IL-15 receptor alpha) as described herein. In some embodiments, the cytokine molecule is an IL-15 molecule, e.g., IL-15 or an IL-15 superagonist as described herein. As used herein, a “superagonist” form of a cytokine molecule shows increased activity, e.g., by at least 10%, 20%, 30%, compared to the naturally-occurring cytokine. An exemplary superagonist is an IL-15 SA. In some embodiments, the IL-15 SA comprises a complex of IL-15 and an IL-15 binding fragment of an IL-15 receptor, e.g., IL-15 receptor alpha or an IL-15 binding fragment thereof, e.g., as described herein. In other embodiments, the cytokine molecule further comprises a receptor domain, e.g., an extracellular domain of an IL-15R alpha, optionally, coupled to an immunoglobulin Fc or an antibody molecule. In embodiments, the cytokine molecule is an IL-15 superagonist (IL-15SA) as described in WO 2010/059253. In some embodiments, the cytokine molecule comprises IL-15 and a soluble IL-15 receptor alpha domain fused to an Fc (e.g., a sIL-15Ra-Fc fusion protein), e.g., as described in Rubinstein et al PNAS 103:24 p. 9166-9171 (2006).

The IL-15 molecule can further comprise a polypeptide, e.g., a cytokine receptor, e.g., a cytokine receptor domain, and a second, heterologous domain. In one embodiment, the heterologous domain is an immunoglobulin Fc region. In other embodiments, the heterologous domain is an antibody molecule, e.g., a Fab fragment, a FAB₂ fragment, a scFv fragment, or an affibody fragment or derivative, e.g. a sdAb (nanobody) fragment, a heavy chain antibody fragment. In some embodiments, the polypeptide also comprises a third heterologous domain. In some embodiments, the cytokine receptor domain is N-terminal of the second domain, and in other embodiments, the cytokine receptor domain is C-terminal of the second domain.

In other embodiments, the IL-15 molecule further comprises a receptor domain, e.g., an extracellular domain of an IL-15R alpha, optionally, coupled to an immunoglobulin Fc or an antibody molecule. In embodiments, the cytokine molecule is an IL-15 superagonist (IL-15SA) as described in WO 2010/059253. In some embodiments, the cytokine molecule comprises IL-15 and a soluble IL-15 receptor alpha domain fused to an Fc (e.g., a sIL-15Ra-Fc fusion protein), e.g., as described in Rubinstein et al PNAS 103:24 p. 9166-9171 (2006).

The IL-15 molecule can further comprise a polypeptide, e.g., a cytokine receptor, e.g., a cytokine receptor domain, and a second, heterologous domain. In one embodiment, the heterologous domain is an immunoglobulin Fc region. In other embodiments, the heterologous domain is an antibody molecule, e.g., a Fab fragment, a Fab₂ fragment, a scFv fragment, or an affibody fragment or derivative, e.g. a sdAb (nanobody) fragment, a heavy chain antibody fragment. In some embodiments, the polypeptide also comprises a third heterologous domain. In some embodiments, the cytokine receptor domain is N-terminal of the second domain, and in other embodiments, the cytokine receptor domain is C-terminal of the second domain.

The wild-type IL-15 Receptor alpha sequence and fragment and variants of this sequence are set out below.

Wild-type IL-15 Receptor alpha sequence (Genbank Acc. No. AAI21141.1): SEQ ID NO: 41.

Wild-type IL-15 Receptor alpha extracellular domain (portion of accession number Q13261): SEQ ID NO: 63.

Isoform CRA_d IL-15 Receptor alpha extracellular domain (portion of accession number EAW86418): SEQ ID NO: 64.

The wild-type IL-15 Receptor alpha sequence is provided above as SEQ ID NO: 41. IL-15 receptor alpha contains an extracellular domain, a 23 amino acid transmembrane segment, and a 39 amino acid cytoplasmic tail. The extracellular domain of IL-15 Receptor alpha is provided as SEQ ID NO: 63.

In other embodiments, an IL-15 agonist can be used. For example, an agonist of an IL-15 receptor, e.g., an antibody molecule (e.g., an agonistic antibody) to an IL-15 receptor, that elicits at least one activity of a naturally-occurring cytokine. In embodiments, the IL-15 receptor or fragment thereof is from human or a non-human animal, e.g., mammal, e.g., non-human primate.

The compositions and methods herein can comprise a portion of IL-15Rα, e.g., a Sushi domain of IL-15Rα. In some embodiments, a polypeptide comprises a Sushi domain and a second, heterologous domain. In some embodiments, the polypeptide also comprises a third heterologous domain. In some embodiments, the Sushi domain is N-terminal of the second domain, and in other embodiments, the Sushi domain is C-terminal of the second domain. In embodiments, the second domain comprises an Fc domain.

The wild-type IL-15 Receptor alpha sequence is provided as SEQ ID NO: 41. IL-15 receptor alpha contains an extracellular domain, a 23 amino acid transmembrane segment, and a 39 amino acid cytoplasmic tail. The sushi domain has been described in the literature including, e.g., Bergamaschi et al. (2008), JBC VOL. 283, NO. 7, pp. 4189-4199; Wei et al. (2001), Journal of Immunology 167:277-282; Schluns et al. (2004) PNAS Vol 110 (15) 5616-5621; US 2016/0184399 (the contents of each of which is incorporated by reference herein).

The extracellular domain of IL-15 Receptor alpha is provided as SEQ ID NO: 63. The extracellular domain of IL-15 Receptor alpha comprises a domain referred to as the sushi domain, which binds IL-15. The general sushi domain, also referred to as complement control protein (CCP) modules or short consensus repeats (SCR), is a protein domain found in several proteins, including multiple members of the complement system. The sushi domain adopts a beta-sandwich fold, which is bounded by the first and fourth cysteine of four highly conserved cysteine residues, comprising to a sequence stretch of approximately 60 amino acids (Norman, Barlow, et al. J Mol Biol. 1991 Jun. 20; 219(4):717-25). The amino acid residues bounded by the first and fourth cysteines of the sushi domain in IL-15Ralpha comprise a 62 amino acid polypeptide referred to as the minimal domain (SEQ ID NO: 52). Including additional amino acids of IL-15Ralpha at the N- and C-terminus of the minimal sushi domain, such as inclusion of N-terminal Ile and Thr and C-terminal Ile and Arg residues result in a 65 amino acid extended sushi domain (SEQ ID NO: 9).

A sushi domain as described herein may comprise one or more mutations relative to a wild-type sushi domain. For instance, residue 77 of IL-15Ra is leucine in the wild-type gene (and is underlined in SEQ ID NO: 41), but can be mutated to isoleucine (L77I). Accordingly, a minimal sushi domain comprising L77I (with the numbering referring to the wild-type IL-15Ra of SEQ ID NO: 41) is provided as SEQ ID NO: 65. An extended sushi domain comprising L77I (with the numbering referring to the wild-type IL-15Ra of SEQ ID NO: 41) is provided as SEQ ID NO: 66.

Minimal sushi domain, wild-type:
(SEQ ID NO: 52)
CPPPMSVEHADIWVKSYSLYSRERYICNSGFKRKAGTSSLTECVLNKATN
VAHWTTPSLKC
Extended sushi domain, wild-type:
(SEQ ID NO: 9)
ITCPPPMSVEHADIWVKSYSLYSRERYICNSGFKRKAGTSSLTECVLNKA
TNVAHWTTPSLKCIR
Minimal sushi domain, L77I:
(SEQ ID NO: 65)
CPPPMSVEHADIWVKSYSLYSRERYICNSGFKRKAGTSSLTECVINKATN
VAHWTTPSLKCI
Extended sushi domain, L77I:
(SEQ ID NO: 66)
ITCPPPMSVEHADIWVKSYSLYSRERYICNSGFKRKAGTSSLTECVINKA
TNVAHWTTPSLKCIR

In some embodiments, a sushi domain consists of 62-171 amino acids of SEQ ID NO: 63 or a sequence having at least 80%, 85%, 90%, 95%, 96%, 97%, 98%, or 99% identity thereto, and having IL-15 binding activity. In some embodiments, a sushi domain consists of 65-171 amino acids of SEQ ID NO: 63 or a sequence having at least 80%, 85%, 90%, 95%, 96%, 97%, 98%, or 99% identity thereto, and having IL-15 binding activity. In some embodiments, a sushi domain consists of up to 171 amino acids of SEQ ID NO: 63 or a sequence having at least 80%, 85%, 90%, 95%, 96%, 97%, 98%, or 99% identity thereto, and having IL-15 binding activity. In some embodiments, a sushi domain consists of 62-171, 62-160, 62-150, 62-140, 62-130, 62-120, 62-110, 62-100, 62-90, 62-80, 62-70, 65-171, 65-160, 65-150, 65-140, 65-130, 65-120, 65-110, 65-100, 65-90, 65-80, or 65-70 amino acids of SEQ ID NO: 63 or a sequence having at least 80%, 85%, 90%, 95%, 96%, 97%, 98%, or 99% identity thereto, and having IL-15 binding activity. In some embodiments, a sushi domain consists of 62-171, 62-160, 62-150, 62-140, 62-130, 62-120, 62-110, 62-100, 62-90, 62-80, 62-70, 65-171, 65-160, 65-150, 65-140, 65-130, 65-120, 65-110, 65-100, 65-90, 65-80, or 65-70 amino acids of SEQ ID NO: 63. In some embodiments, the sushi domain comprises, or consists of, an amino acid sequence of SEQ ID NO: 9 or SEQ ID NO: 52.

In some embodiments, a sushi domain consists of 62-171 amino acids of SEQ ID NO: 63 or a sequence having up to 1, 2, 3, 4, 5, 6, 7, 8, 9, 10, 15, 20, 25, 30, 35, or 40 modifications (e.g., substitutions) relative thereto, and having IL-15 binding activity. In some embodiments, a sushi domain consists of up to 171 amino acids of SEQ ID NO: 63 or a sequence having up to 1, 2, 3, 4, 5, 6, 7, 8, 9, 10, 15, 20, 25, 30, 35, or 40 modifications (e.g., substitutions) relative thereto, and having IL-15 binding activity. In some embodiments, a sushi domain consists of 62-171, 62-160, 62-150, 62-140, 62-130, 62-120, 62-110, 62-100, 62-90, 62-80, 62-70, 65-171, 65-160, 65-150, 65-140, 65-130, 65-120, 65-110, 65-100, 65-90, 65-80, or 65-70 amino acids of SEQ ID NO: 63 or a sequence having up to 1, 2, 3, 4, 5, 6, 7, 8, 9, 10, 15, 20, 25, 30, 35, or 40 modifications (e.g., substitutions) relative thereto, and having IL-15 binding activity.

In some embodiments, a sushi domain comprises at least 62 amino acids of SEQ ID NO: 63 or a sequence having at least 80%, 85%, 90%, 95%, 96%, 97%, 98%, or 99% identity thereto, wherein the sequence comprises an L77I mutation relative to wild-type IL-15Ra, and having IL-15 binding activity. In some embodiments, a sushi domain comprises at least 65 amino acids of SEQ ID NO: 63 or a sequence having at least 80%, 85%, 90%, 95%, 96%, 97%, 98%, or 99% identity thereto, wherein the sequence comprises an L77I mutation relative to wild-type IL-15Ra, and having IL-15 binding activity. In some embodiments, a sushi domain comprises a portion of SEQ ID NO: 63 or a sequence having at least 80%, 85%, 90%, 95%, 96%, 97%, 98%, or 99% identity thereto, wherein the sequence comprises an L77I mutation relative to wild-type IL-15Ra, and having IL-15 binding activity. In some embodiments, the sushi domain comprises an amino acid sequence of SEQ ID NO: 9 or SEQ ID NO: 52.

In some embodiments, a sushi domain comprises at least 62 amino acids of SEQ ID NO: 63 or a sequence having up to 1, 2, 3, 4, 5, 6, 7, 8, 9, 10, 15, 20, 25, 30, 35, or 40 modifications (e.g., substitutions) relative thereto, wherein the sequence comprises an L77I mutation relative to wild-type IL-15Ra, and having IL-15 binding activity. In some embodiments, a sushi domain comprises a portion of SEQ ID NO: 66 or a sequence having up to 1, 2, 3, 4, 5, 6, 7, 8, 9, 10, 15, 20, 25, 30, 35, or 40 modifications (e.g., substitutions) relative thereto, wherein the sequence comprises an L77I mutation relative to wild-type IL-15Ra, and having IL-15 binding activity.

In embodiments, the sushi domain comprises at least 10, 20, 30, 40, 50, 60, 62, 65, 70, 80, 90, 100, 110, 120, 130, 140, 150, or 160 consecutive amino acids of SEQ ID NO: 63, or a sequence having an L77I mutation relative thereto. In embodiments, the sushi domain consists of 10-20, 20-30, 30-40, 40-50, 50-60, 60-70, 70-80, 80-90, 90-100, 100-110, 110-120, 120-130, 130-140, 140-150, 150-160, or 160-170 consecutive amino acids of SEQ ID NO: 63, or a sequence having an L77I mutation relative thereto.

In embodiments, the sushi domain is a sushi domain from human or a non-human animal, e.g., mammal, e.g., non-human primate.

In some embodiments, the polypeptide can have a second, heterologous domain, e.g., an Fc domain or a Fab domain.

In some embodiments, the polypeptide comprising the IL-15 receptor or fragment thereof comprises an Fc domain. In embodiments, the Fc domain is an effector-attenuated Fc domain, e.g., a human IgG2 Fc domain, e.g., a human IgG2 Fc domain of SEQ ID NO: 54 or an amino acid sequence having at least 80%, 85%, 90%, 95%, 96%, 97%, 98%, or 99% identity thereto.

In embodiments, the effector-attenuated Fc domain has reduced effector activity, e.g., compared to a wild-type IgG1 Fc domain, e.g., compared to a wild-type IgG1 Fc domain of SEQ ID NO: 67. In some embodiments, effector activity comprises antibody-dependent cellular toxicity (ADCC). In embodiments, the effector activity is reduced by 10%, 20%, 30%, 40%, 50%, 60%, 70%, 80%, 90%, 95%, or 99% in an ADCC assay, e.g., compared to a wild-type IgG1 Fc domain of SEQ ID NO: 67. In some embodiments, effector activity comprises complement dependent cytotoxicity (CDC). In embodiments, the effector activity is reduced by 10%, 20%, 30%, 40%, 50%, 60%, 70%, 80%, 90%, 95%, or 99% in a CDC assay such as a CDC assay described in Armour et al., “Recombinant human IgG molecules lacking Fc gamma receptor I binding and monocyte triggering activities.” Eur J Immunol (1999) 29:2613-24″ e.g., compared to a wild-type IgG1 Fc domain of SEQ ID NO: 67.

In some embodiments, the Fc domain comprises an IgG1 Fc domain of SEQ ID NO: 67 or an amino acid sequence having at least 80%, 85%, 90%, 95%, 96%, 97%, 98%, or 99% identity thereto.

In some embodiments, the Fc domain comprises an IgG2 constant region of SEQ ID NO: 68 or fragment thereof, or an amino acid sequence having at least 80%, 85%, 90%, 95%, 96%, 97%, 98%, or 99% identity thereto.

In some embodiments, the Fc domain comprises an IgG2 Da Fc domain of SEQ ID NO: 55 or an amino acid sequence having at least 80%, 85%, 90%, 95%, 96%, 97%, 98%, or 99% identity thereto. In embodiments, the Fc domain comprises one or both of A330S and P331S mutations using Kabat numbering system. In embodiments, the Fc domain is one described in Armour et al. “Recombinant human IgG molecules lacking Fc gamma receptor I binding and monocyte triggering activities.” Eur J Immunol (1999) 29:2613-24.

In some embodiments, the Fc domain has dimerization activity. In yet other embodiments, the Fc domain is an IgG domain, e.g., an IgG1, IgG2, IgG3, or IgG4 Fc domain. In one embodiments, the Fc domain comprises a CH2 domain and a CH3 domain. In some embodiments, the nanoparticle comprises a protein having a sequence of SEQ ID NO: 56 (sushi-IgG2 Da-Fc) or an amino acid sequence having at least 80%, 85%, 90%, 95%, 96%, 97%, 98%, or 99% identity thereto.

In some embodiments, the IFM that comprises a sushi domain described herein (e.g., in SEQ ID NO: 9) and an Fc domain described herein, e.g., an IgG2 Fc domain (e.g., SEQ ID NO: 54 or an amino acid sequence having at least 80%, 85%, 90%, 95%, 96%, 97%, 98%, or 99% identity thereto). In some embodiments, the IFM comprises a sushi domain of SEQ ID NO: 9 and an Fc domain described herein, e.g., an IgG1 Fc domain, e.g., an Fc domain of SEQ ID NO: 67 or an amino acid sequence having at least 80%, 85%, 90%, 95%, 96%, 97%, 98%, or 99% identity thereto. In some embodiments, the IFM comprises a sushi domain of SEQ ID NO: 52 and an IgG2 Fc domain, e.g., an Fc domain of SEQ ID NO: 54 or an amino acid sequence having at least 80%, 85%, 90%, 95%, 96%, 97%, 98%, or 99% identity thereto. In some embodiments, the IFM comprises a sushi domain of SEQ ID NO: 52 and an IgG1 Fc domain, e.g., an Fc domain of SEQ ID NO: 67 or an amino acid sequence having at least 80%, 85%, 90%, 95%, 96%, 97%, 98%, or 99% identity thereto.

In some embodiments, the IFM comprises a sushi domain of SEQ ID NO: 9 and an IgG2 Da Fc domain of SEQ ID NO: 56 or an amino acid sequence having at least 80%, 85%, 90%, 95%, 96%, 97%, 98%, or 99% identity thereto. In some embodiments, the IFM comprises a sushi domain of SEQ ID NO: 52 and an IgG2 Da Fc domain of SEQ ID NO: 56 or an amino acid sequence having at least 80%, 85%, 90%, 95%, 96%, 97%, 98%, or 99% identity thereto. In some embodiments, the IFM comprises a sushi-IgG2 Da-Fc protein having a sequence of SEQ ID NO: 56 or an amino acid sequence having at least 80%, 85%, 90%, 95%, 96%, 97%, 98%, or 99% identity thereto.

In embodiments, the Sushi domain is a Sushi domain from human or a non-human animal, e.g., mammal, e.g., non-human primate.

In embodiments, the composition comprises, e.g., the nanoparticle comprises, an IL-15 complex, the IL-15 complex comprising an IL-15 molecule complexed, e.g., covalently or noncovalently, with a polypeptide, wherein the polypeptide comprises a first domain comprising:
i) at least 62 amino acids of the wild-type IL-15 Receptor alpha extracellular domain of SEQ ID NO: 63, wherein the longest contiguous IL-15 receptor alpha sequence of the polypeptide is no more than 171 amino acids in length, or a sequence having at least 80%, 85%, 90%, 95%, 96%, 97%, 98%, or 99% identity thereto, and having IL-15 binding activity;
ii) at least 62 amino acids of SEQ ID NO: 63, wherein the longest contiguous IL-15 receptor alpha sequence of the polypeptide is no more than 171 amino acids in length, or a sequence that differs by no more than 1, 2, 3, 4, or 5 amino acids from the corresponding sequence of SEQ ID NO: 63, and having IL-15 binding activity;
iii) at least 62 amino acids of SEQ ID NO: 63, wherein the longest contiguous IL-15 receptor alpha sequence of the polypeptide is no more than 171 amino acids in length, and having IL-15 binding activity;
iv) an active fragment, e.g., an IL-15 binding fragment, of the minimal sushi domain and no more than 171 contiguous amino acid residues of SEQ ID NO: 63, or a sequence having at least 80%, 85%, 90%, 95%, 96%, 97%, 98%, or 99% identity thereto;
v) an active fragment, e.g., an IL-15 binding fragment, of the minimal sushi domain and no more than 171 contiguous amino acid residues of SEQ ID NO: 63, or a sequence that differs by no more than 1, 2, 3, 4, or 5 amino acids from the corresponding sequence of SEQ ID NO: 63;
vi) an active fragment, e.g., an IL-15 binding fragment, of the minimal sushi domain and no more than 171 contiguous amino acid residues of SEQ ID NO: 63;
vii) an active fragment, e.g., an IL-15 binding fragment, of the extended sushi domain and no more than 171 contiguous amino acid residues of SEQ ID NO: 63, or a sequence having at least 80%, 85%, 90%, 95%, 96%, 97%, 98%, or 99% identity thereto;
viii) an active fragment, e.g., an IL-15 binding fragment, of the extended sushi domain and no more than 171 contiguous amino acid residues of SEQ ID NO: 63, or a sequence that differs by no more than 1, 2, 3, 4, or 5 amino acids from the corresponding sequence of SEQ ID NO: 63;
ix) an active fragment, e.g., an IL-15 binding fragment, of the extended sushi domain and no more than 171 contiguous amino acid residues of SEQ ID NO: 63; or
x) at least 62 amino acids of SEQ ID NO: 63, or a sequence having at least 80%, 85%, 90%, 95%, 96%, 97%, 98%, or 99% identity thereto, having IL-15 binding activity, and wherein amino acid 77 (with numbering referring to the wild-type IL-15 receptor alpha of SEQ ID NO: 41) is isoleucine;
xi) at least 62 amino acids of SEQ ID NO: 63, or a sequence that differs by no more than 1, 2, 3, 4, or 5 amino acids from the corresponding sequence of SEQ ID NO: 63, having IL-15 binding activity, and wherein amino acid 77 is isoleucine;
xii) at least 62 amino acids of SEQ ID NO: 63 having IL-15 binding activity, and wherein amino acid 77 is isoleucine;
xiii) an active fragment, e.g., an IL-15 binding fragment, of the minimal or extended sushi domain, or a sequence having at least 80%, 85%, 90%, 95%, 96%, 97%, 98%, or 99% identity thereto, wherein amino acid 77 is isoleucine;
xiv) an active fragment, e.g., an IL-15 binding fragment, of the minimal or extended sushi domain, or a sequence that differs by no more than 1, 2, 3, 4, or 5 amino acids from the corresponding sequence of SEQ ID NO: 63, wherein amino acid 77 is isoleucine; or
xv) an active fragment, e.g., an IL-15 binding fragment, of the minimal or extended sushi domain, wherein amino acid 77 is isoleucine; and

optionally, a second, heterologous domain, e.g., an Fc domain or a Fab domain.

In some embodiments, the polypeptide comprising the IL-15 receptor or fragment thereof comprises an Fc domain. In embodiments, the Fc domain is an effector-attenuated Fc domain, e.g., a human IgG2 Fc domain, e.g., a human IgG2 domain of SEQ ID NO: 54 or an amino acid sequence having at least 80%, 85%, 90%, 95%, 96%, 97%, 98%, or 99% identity thereto.

(SEQ ID NO: 54)
ERKCCVECPPCPAPPVAGPSVFLFPPKPKDTLMISRTPEVTCVVVDVSHE
DPEVQFNWYVDGVEVHNAKTKPREEQFNSTFRVVSVLTVVHQDWLNGKEY
KCKVSNKGLPAPIEKTISKTKGQPREPQVYTLPPSREEMTKNQVSLTCLV
KGFYPSDIAVEWESNGQPENNYKTTPPMLDSDGSFFLYSKLTVDKSRWQQ
GNVFSCSVMHEALHNHYTQKSLSLSPGK

In embodiments, the effector-attenuated Fc domain has reduced effector activity, e.g., compared to a wild-type IgG1 Fc domain, e.g., compared to a wild-type IgG1 Fc domain of SEQ ID NO: 53. In some embodiments, effector activity comprises antibody-dependent cellular toxicity (ADCC). In embodiments, the effector activity is reduced by 10%, 20%, 30%, 40%, 50%, 60%, 70%, 80%, 90%, 95%, or 99% in an ADCC assay, e.g., compared to a wild-type IgG1 Fc domain of SEQ ID NO: 53. In some embodiments, effector activity comprises complement dependent cytotoxicity (CDC). In embodiments, the effector activity is reduced by 10%, 20%, 30%, 40%, 50%, 60%, 70%, 80%, 90%, 95%, or 99% in a CDC assay such as a CDC assay described in Armour et al., “Recombinant human IgG molecules lacking Fc gamma receptor I binding and monocyte triggering activities.” Eur J Immunol (1999) 29:2613-24″ e.g., compared to a wild-type IgG1 Fc domain of SEQ ID NO: 53.

In some embodiments, the Fc domain comprises an IgG1 Fc domain of SEQ ID NO: 53 or an amino acid sequence having at least 80%, 85%, 90%, 95%, 96%, 97%, 98%, or 99% identity thereto.

(SEQ ID NO: 53)
EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK

In some embodiments, the Fc domain comprises an IgG2 constant region of SEQ ID NO: 68 or fragment thereof, or an amino acid sequence having at least 80%, 85%, 90%, 95%, 96%, 97%, 98%, or 99% identity thereto.

(SEQ ID NO: 68)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK

In some embodiments, the Fc domain comprises an IgG2 Da Fc domain of SEQ ID NO: 55 or an amino acid sequence having at least 80%, 85%, 90%, 95%, 96%, 97%, 98%, or 99% identity thereto. In embodiments, the Fc domain comprises one or both of A330S and P331S mutations using Kabat numbering system. In embodiments, the Fc domain is one described in Armour et al. “Recombinant human IgG molecules lacking Fc gamma receptor I binding and monocyte triggering activities.” Eur J Immunol (1999) 29:2613-24.

(SEQ ID NO: 55; IgG2Da-Fc)
ERKCCVECPPCPAPPVAGPSVFLFPPKPKDTLMISRTPEVTCVVVDVSHE
DPEVQFNWYVDGVEVHNAKTKPREEQFNSTFRVVSVLTVVHQDWLNGKEY
KCKVSNKGLPssIEKTISKTKGQPREPQVYTLPPSREEMTKNQVSLTCLV
KGFYPSDIAVEWESNGQPENNYKTTPPMLDSDGSFFLYSKLTVDKSRWQQ
GNVFSCSVMHEALHNHYTQKSLSLSPGK

In some embodiments, the Fc domain has dimerization activity. In other embodiments, the Fc domain is an IgG domain, e.g., an IgG1, IgG2, IgG3, or IgG4 Fc domain. In one embodiment, the Fc domain comprises a CH2 domain and a CH3 domain. In some embodiments, the nanoparticle comprises a protein having a sequence of SEQ ID NO: 56 or an amino acid sequence having at least 80%, 85%, 90%, 95%, 96%, 97%, 98%, or 99% identity thereto.

(SEQ ID NO: 56; sushi-IgG2Da-Fc)
ITCPPPMSVEHADIWVKSYSLYSRERYICNSGFKRKAGTSSLTECVLNKA
TNVAHWTTPSLKCIRERKCCVECPPCPAPPVAGPSVFLFPPKPKDTLMIS
RTPEVTCVVVDVSHEDPEVQFNWYVDGVEVHNAKTKPREEQFNSTFRVVS
VLTVVHQDWLNGKEYKCKVSNKGLPSSIEKTISKTKGQPREPQVYTLPPS
REEMTKNQVSLTCLVKGFYPSDIAVEWESNGQPENNYKTTPPMLDSDGSF
FLYSKLTVDKSRWQQGNVFSCSVMHEALHNHYTQKSLSLSPGK

In some embodiments, the nanoparticle comprises a sushi domain described herein (e.g., in SEQ ID NO: 9) and an Fc domain described herein, e.g., an IgG2 Fc domain (e.g., SEQ ID NO: 54 or an amino acid sequence having at least 80%, 85%, 90%, 95%, 96%, 97%, 98%, or 99% identity thereto). In some embodiments, the nanoparticle comprises a sushi domain of SEQ ID NO: 9 and an Fc domain described herein, e.g., an IgG1 Fc domain, e.g., an Fc domain of SEQ ID NO: 53 or an amino acid sequence having at least 80%, 85%, 90%, 95%, 96%, 97%, 98%, or 99% identity thereto. In some embodiments, the nanoparticle comprises a sushi domain of SEQ ID NO: 52 and an IgG2 Fc domain, e.g., an Fc domain of SEQ ID NO: 54 or an amino acid sequence having at least 80%, 85%, 90%, 95%, 96%, 97%, 98%, or 99% identity thereto. In some embodiments, the nanoparticle comprises a sushi domain of SEQ ID NO: 52 and an IgG1 Fc domain, e.g., an Fc domain of SEQ ID NO: 53 or an amino acid sequence having at least 80%, 85%, 90%, 95%, 96%, 97%, 98%, or 99% identity thereto.

In some embodiments, the nanoparticle comprises a sushi domain of SEQ ID NO: 9 and an IgG2 Da Fc domain of SEQ ID NO: 55 or an amino acid sequence having at least 80%, 85%, 90%, 95%, 96%, 97%, 98%, or 99% identity thereto. In some embodiments, the nanoparticle comprises a sushi domain of SEQ ID NO: 52 and an IgG2 Da Fc domain of SEQ ID NO: 55 or an amino acid sequence having at least 80%, 85%, 90%, 95%, 96%, 97%, 98%, or 99% identity thereto. In some embodiments, the nanoparticle comprises a sushi-IgG2 Da-Fc protein having a sequence of SEQ ID NO: 56 or an amino acid sequence having at least 80%, 85%, 90%, 95%, 96%, 97%, 98%, or 99% identity thereto.

In embodiments, the IL-15 molecule is a molecule described in International Application WO2017/027843, which is herein incorporated by reference in its entirety.

In some embodiments the IL-15 molecule is IL-15SA. The combination of human IL-15 with soluble human IL-15Ra generates a complex termed IL-15 superagonist (IL-15SA) that possesses greater biological activity than human IL-15 alone.

Soluble human IL-15Ra, as well as truncated versions of the extracellular domain, has been described, e.g., in (Wei et al., 2001, J. of Immunol. 167: 277-282). The amino acid sequence of human IL-15Ra is set forth in SEQ ID NO: 2 herein. Accordingly, some aspects of the disclosure relate to IL-15SA comprising a complex of human IL-15 and soluble human IL-15R molecules. In some aspects of the disclosure, IL-15SA comprises a complex of human IL-15 and soluble human IL-15Ra comprising all or a portion of the extracellular domain, without the transmembrane or cytoplasmic domain. In some aspects of the disclosure, IL-15SA comprises a complex of human IL-15 and soluble human iL-15Ra comprising the full extracellular domain or a truncated form of the extracellular domain which retains IL-15 binding activity. Some aspects of the disclosure relate to IL-15SA comprising a complex of human IL-15 and soluble human IL-15Ra comprising a truncated form of the extracellular domain which retains IL-15 binding activity, such as amino acids 1-60, 1-61, 1-62, 1-63, 1-64 or 1-65 of human IL-15Ra. In some aspects of the disclosure, IL-15SA comprises a complex of human IL-15 and soluble human IL-15Ra comprising a truncated form of the extracellular domain which retains IL-15 binding activity, such as amino acids 1-80, 1-81, 1-82, 1-83, 1-84 or 1-85 of human IL-15Ra. In some aspects of the disclosure, 1L-15SA comprises a complex of human IL-15 and soluble human IL-15Ra comprising a truncated form of the extracellular domain which retains IL-15 binding activity, such as amino acids 1-180, 1-181, or 1-182 of human IL-15Ra.

Some aspects of the disclosure relate to 1L-15SA comprising a complex of human IL-15 and soluble human IL-15Ra comprising a truncated form of the extracellular domain which retains IL-15 binding activity and comprises a Sushi domain. Truncated forms of soluble human IL-15Ra which retain IL-15 activity and comprise a Sushi domain are useful in IL-15SA of the present disclosure.

Mutant forms of human IL-15 have been described, e.g., in Zhu et al, 2009 J of Immunol. 183:3598. Accordingly, the present disclosure provides any of the foregoing IL-15SA complexes in which human IL-15 is wild-type or mutant IL-15 comprising one or more mutations (e.g., one or more amino acid substitutions, additions or deletions). An exemplary IL-15 mutant having increased biological activity relative to wild-type IL-15 for use in the IL-15SA of the present disclosure comprises an asparagine to aspartic acid substitution at amino acid 72 (N72D).

In any of the foregoing embodiments, the present disclosure relates to a complex comprising soluble human IL-15Ra expressed as a fusion protein, such as an Fc fusion as described herein (e.g., human IgG1 Fc), with IL-15. In some embodiments, IL-15SA comprises a dimeric human IL-15RaFc fusion protein (e.g., human IgG1 Fc) complexed with two human IL-15 molecules.

In some embodiments an IL-15SA cytokine complex comprises an IL-15 molecule comprising an amino acid sequence set forth in SEQ ID NO: 10 or SEQ ID NO: 11 herein. In some embodiments, an IL-15SA cytokine complex comprises an IL-15 molecule comprising an amino acid sequence set forth in SEQ ID NO: 4 or SEQ ID NO: 5 of International Application WO2017/027843, which are herein incorporated by reference. In some embodiments, an IL-15SA cytokine complex comprises a soluble IL-15Ra molecule comprising a sequence of SEQ ID NO: 9, SEQ ID NO: 52, SEQ ID NO: 65, or SEQ ID NO: 66 herein. In some embodiments, an IL-15SA cytokine complex comprises a soluble IL-15Ra molecule comprising a sequence of SEQ ID NO: 63, SEQ ID NO: 9 or SEQ ID NO: 52 of International Application WO2017/027843, which are herein incorporated by reference.

In some embodiments the IL-15SA is a cytokine complex comprising a dimeric IL-15RaFc fusion protein complexed with two IL-15 molecules. In some embodiments, IL-15-SA comprises a dimeric IL-15RaSu(Sushi domain)/Fc (SEQ ID NO: 13) and two IL-15N72D (SEQ ID NO: 11) molecules. In embodiments, the IL-15SA comprises ALT-803, as described in US20140134128, incorporated herein by reference. In some embodiments, the IL-15SA comprises a dimeric IL-15RaSu/Fc molecule (SEQ ID NO: 13) and two IL-15 molecules (SEQ ID NO: 10).

In some embodiments, the IL-15SA comprises a soluble IL-15Ra molecule (e.g., SEQ ID NO: 9 or SEQ ID NO: 52) and two IL-15 molecules (e.g., SEQ ID NO: 10 or SEQ ID NO: 11).

### Protein Nanogel/Nanoparticles

Compositions, e.g., nanoparticles (e.g., nanogels) can comprise one or more proteins (e.g., biologically-active proteins, e.g., therapeutic proteins) disclosed herein. Exemplary nanoparticles (e.g., nanogels), and methods of making the same, are described in International published application WO 2010/059253, entitled “Methods and Compositions for Localized Agent Delivery” and International published application WO 2015/048498, entitled “Carrier-Free Biologically-Active Protein Nanoparticles,” the contents of both applications are hereby entirely incorporated by reference. A “particle” as used herein, comprises a plurality of (e.g., at least 2) proteins, e.g., a plurality of cytokine molecules as described herein. In some embodiments, the particles are nanoparticles having a diameter of a range from 1-1000 nanometers (nm). In some embodiments, the diameter of the nanoparticle ranges in size from 20-750 nm, or from 20-500 nm, or from 20-250 nm. In some embodiments, the diameter ranges in size from 50-750 nm, or from 50-500 nm, or from 50-250 nm, or from about 100-300 nm. In some embodiments, the diameter of the nanoparticle is about 100, about 150, about 200 nm, about 250 nm, or about 300 nm. In embodiments, the nanoparticles are substantially spherical.

In embodiments, the nanoparticle has an average hydrodynamic diameter (e.g., measured by dynamic light scattering) between 30 nm and 1200 nm, between 40 nm and 1,100 nm, between 50 nm and 1,000 nanometer, between such as 50-500 nm, more typically, between 70 and 400 nm.

In some embodiments, the nanoparticles comprise, or consist of, a nanogel, e.g., a described herein. In embodiments, the proteins in the nanogels are coupled, e.g., covalently coupled or crosslinked to each other and/or a second component of the particle (e.g., the proteins reversibly linked through a degradable linker). In embodiments, the proteins are present in a polymer or silica, e.g., in a polymer-based or silica shell. In embodiments, the nanoparticle includes a nanoshell as described herein.

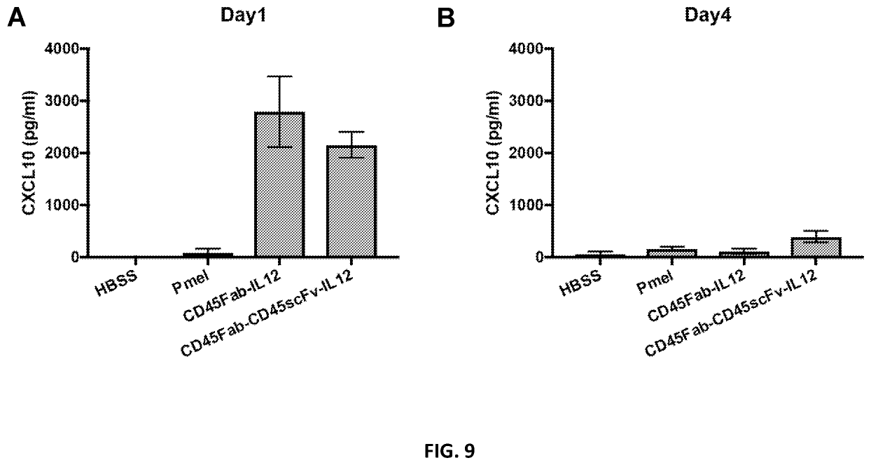

In embodiments, the protein is reversibly linked through a degradable linker to a functional group or polymer, or “reversibly modified.” The nanoshell can be formed, in some embodiments, by polymerizing functional groups (e.g., silanes) of a protein conjugate with a crosslinker (e.g., silane-PEG-silane) in the presence of a catalyst (e.g., NaF). An example of a protein nanoparticle is a “protein nanogel,” which refers to a plurality of proteins crosslinked (e.g., reversibly and covalently crosslinked) to each other through a degradable linker (see, e.g., FIG. 9A of WO2015/048498). In some embodiments, proteins of a nanogel are crosslinked (e.g., reversibly and covalently crosslinked) to a polymer (e.g., a hydrophilic polymer such as polyethylene glycol (PEG); see, e.g., FIG. 9A of WO2015/048498). The polymer, in some embodiments, may be crosslinked to the surface of the nanogel (e.g., to proteins exposed at the surface of the nanogel).

The size of a protein nanogel may be determined at least two ways: based on its “dry size” and based on its “hydrodynamic size.” The “dry size” of a protein nanogel refers to the diameter of the nanogel as a dry solid. The “hydrodynamic size” of a protein nanogel refers to the diameter of the nanogel as a hydrated gel (e.g., a nanogel in an aqueous buffer). The dry size of a nanogel may be determined, for example, by transmission electron microscopy, while the hydrodynamic size of the nanogel may be determined, for example, by dynamic light scattering.

In some embodiments, the dry size of a nanogel is less than 400 nm. In some embodiments, the dry size of a nanogel is less than 300 nm, less than 200 nm, less than 100 nm, less than 80 nm, less than 75 nm, less than 70 nm, less than 65 nm, or less than 60 nm. In some embodiments, the dry size of a nanogel is 40 to 90 nm, 40 to 80 nm, 40 to 70 nm, 40 to 60 nm, 50 to 90 nm, 60 to 80 nm, 50 to 70 nm, or 50 to 60 nm. In some embodiments, the dry size of a nanogel is 40 nm, 45 nm, 50 nm, 55 nm, 60 nm, 65 nm, 70 nm, 75 nm, 80 nm, 85 nm, 90 nm or 95 nm.

In some embodiments, the average dry size of a nanoparticle (e.g., nanogel) within a plurality of nanoparticles is less than 400 nm. In some embodiments, the average dry size of a nanoparticle within such a plurality varies by no more than 5% or 10%. In some embodiments, the average dry size of a nanoparticle (e.g., nanogel) within a plurality of nanoparticles is less than 300 nm, less than 200 nm, less than 100 nm, less than 80 nm, less than 75 nm, less than 70 nm, less than 65 nm, or less than 60 nm. In some embodiments, the average dry size of a nanoparticle (e.g., nanogel) within a plurality of nanoparticles is 40 to 90 nm, 40 to 80 nm, 40 to 70 nm, 40 to 60 nm, 50 to 90 nm, 60 to 80 nm, 50 to 70 nm, or 50 to 60 nm. In some embodiments, the dry size of a nanogel is 40 nm, 45 nm, 50 nm, 55 nm, 60 nm, 65 nm, 70 nm, 75 nm, 80 nm, 85 nm, 90 nm or 95 nm.

In some embodiments, the average hydrodynamic size of a nanoparticle (e.g., nanogel) within a plurality of nanoparticles is less than 1000 nm. In some embodiments, the average hydrodynamic size of a nanoparticle within such a plurality has a polydispersity index as measured by dynamic light scattering of less than 0.35. In some embodiments, the average hydrodynamic size of a nanoparticle (e.g., nanogel) within a plurality of nanoparticles is less than 500 nm, less than 400 nm, less than 300 nm, less than 200 nm, or less than 100 nm. In some embodiments, the average hydrodynamic size of a nanoparticle (e.g., nanogel) within a plurality of nanoparticles is 400 to 500 nm, 300 to 400 nm, 200 to 300 nm, 100 to 200 nm, 50 to 100 nm.

In some embodiments, the dry size of the biologically-active protein-polymer nanogels is less than 300 nm in diameter. For example, the dry size of the biologically-active protein-polymer nanogels may be 50-200 nm in diameter. In some embodiments, protein nanogels of a plurality, as provided herein, are of similar dry size (e.g., where 70% of the nanogels are within 10%, 20%, 30%, 40%, 50% or 100% diameter of each other and have a polydispersity index as measured by dynamic light scattering of less than 0.35).

In some embodiments, the hydrodynamic size of the biologically-active protein-polymer nanogels is less than 150 nm in diameter. For example, the hydrodynamic size of the biologically-active protein-polymer nanogels may be 50-100 nm in diameter. In some embodiments, protein nanogels of a plurality, as provided herein, are of similar hydrodynamic size (e.g., where 70% of the nanogels are within 10%, 20%, 30%, 40%, 50% or 100%, diameter of each other and have a polydispersity index as measured by dynamic light scattering of less than 0.35).

In some embodiments, nanoparticles are provided in a dry, solid form, such as a lyophilized form. In other embodiments, nanoparticles are provided in a hydrated form, such as in aqueous or otherwise liquid solution. In other embodiments, nanoparticles are provided in a frozen form.

In some embodiments, proteins of the nanoparticles are reversibly linked to each other through a degradable linker such that under physiological conditions, the linker degrades and releases the intact, biologically-active protein. In other embodiments, proteins of nanoparticles are reversibly linked to functional groups through a degradable linker such that under physiological conditions, the linker degrades and releases the intact, biologically-active protein. In each instance, the proteins are considered to be reversibly modified, as described below.

A protein that is “reversibly linked to another protein” herein refers to a protein that is attached (e.g., covalently attached) to another protein through a degradable linker. Such proteins are considered to be linked (e.g., crosslinked) to each other through the degradable linker. In some embodiments, nanoparticles (e.g., nanogels) contain a single (e.g., single type of) biologically-active protein (e.g., IL-15, IL-15-Fc, IL-15 Fab fragment, IL-2, or IL-2-Fc, a Fab fragment, a FAB₂ fragment, a scFv fragment, or an affibody fragment or derivative, e.g. a sdAb (nanobody) fragment, a heavy chain antibody fragment etc.), while in other embodiments, nanoparticles contain more than one (e.g., 2, 3, 4, 5 or more) of biologically-active protein (e.g., a combination of different proteins such as IL-2 and IL-15 (or IL-15SA) or IL-15 and IL-21). For example, a protein nanogel may contain a combination of Protein A and Protein B, wherein Protein A is linked to Protein A, Protein A is linked to Protein B and/or Protein B is linked to Protein B.

A protein that is “reversibly linked to a functional group,” or a protein that is “reversibly modified,” herein refers to a protein that is attached (e.g., covalently attached) to a functional group through a degradable linker. Such a protein may be referred to herein as a “protein conjugate” or a “reversibly modified protein conjugate”—the terms may be used interchangeably herein. It should be understood that proteins and polymers each contain functional groups to which a protein can be linked via a reversible linker (e.g., degradable linker such as a disulfide linker). Examples of protein conjugates and reversibly modified proteins, as provided herein, include without limitation, a protein reversibly linked (e.g., via a degradable linker) to another protein, a protein reversibly linked to a polymer, and a protein reversibly linked to another functional group. It should be understood that the term “protein” includes fusion proteins.

### Degradable Linkers

Suitable degradable linkers, e.g., crosslinkers, for the nanoparticles described herein can contain, for example, two N-hydroxysuccinimide (NHS) ester groups joined together by a flexible disulfide-containing linker that is sensitive to a reductive physiological environment, or a hydrolysable linker that is sensitive to an acidic physiological environment (pH<7, for example, a pH of 4-5, 5-6, or 6- to less than 7, e.g., 6.9), or a protease sensitive linker that is sensitive to one or more enzymes present in biological media such as proteases in a tumor microenvironment such a matrix metalloproteases present in a tumor microenvironment or in inflamed tissue (e.g. matrix metalloproteinase 2 (MMP2) or matrix metalloproteinase 9 (MMP9)). A crosslinker sensitive to a reductive physiological environment is, for example, a crosslinker with disulfide containing linker that will react with amine groups on proteins by the presence of NHS groups which cross-link the proteins into high density protein nanogels. The cross-linker used in the Examples herein includes Bis[2-(N-succinimidyl-oxycarbonyloxy)ethyl] disulfide.

In some embodiments, the degradable linker comprises at least one N-hydroxysuccinimide ester. In some embodiments, the degradable linker is a redox responsive linker. In some embodiments, the redox responsive linker comprises a disulfide bond. In some embodiments, the degradable linkers provided herein, comprise at least one N-hydroxysuccinimide ester, which is capable of reacting with proteins at neutral pH (e.g., about 6 to about 8, or about 7) without substantially denaturing the protein. In some embodiments, the degradable linkers are “redox responsive” linkers, meaning that they degrade in the presence of a reducing agent (e.g., glutathione, GSH) under physiological conditions (e.g., 20-40° C. and/or pH 4-8), thereby releasing intact protein from the compound to which it is reversibly linked. An example of a degradable linker for use in accordance with the present disclosure is the following:

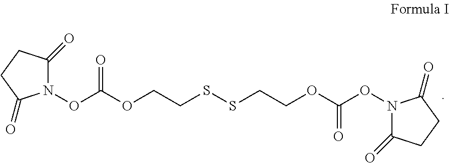 [SMILES: O=C(OCCSSCCOC(=O)ON1C(=O)CCC1=O)ON1C(=O)CCC1=O]

The linker of Formula I contains a disulfide, which is cleaved in the presence of a reducing agent. For example, under physiological conditions, the disulfide bond of the linker of Formula I is cleaved by glutathione.

Proteins may be linked (e.g., covalently linked) to a degradable linker through any terminal or internal—NH₂ functional group (e.g., side chain of a lysine). Thus, an intermediate species formed during the reversible modification of a protein with a degradable linker of Formula I is the following:

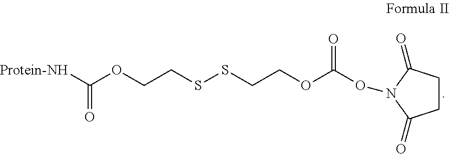

Also provided herein are reversibly modified protein conjugates that comprise Formula III:

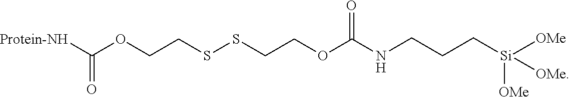

The linkers may be conjugated to the protein of interest at an amine group such as a terminal amine or an internal amine Internal amines include side chain amines such as lysine amines.

The disclosure further encompasses protein conjugates comprising Formula III:

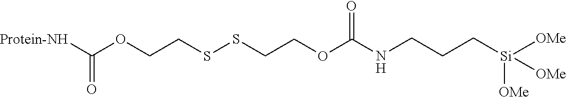

In addition, the disclosure further provides protein conjugates comprising Formula IV:

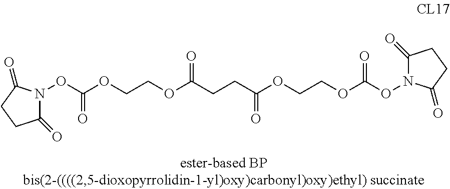 [SMILES: O=C(CCC(=O)OCCOC(=O)ON1C(=O)CCC1=O)OCCOC(=O)ON1C(=O)CCC1=O]

Silica-based nanoparticles with a high incorporation efficiency (e.g., >˜90%) and with high protein drug loading efficiency (e.g., >˜80%) are formed by the polymerization of proteins that are reversibly modified with silane. Thus, provided herein are nanoparticles formed by the polymerization of protein conjugates of Formula III with crosslinkers such as, for example, silane-PEG-silane polymers.

In other embodiments, the proteins can be linked by an enzyme-sensitive linker. In embodiments, the linker is degraded or hydrolyzed through the action of an enzyme (e.g., a protease or esterase). In some embodiments, the linker comprises a substrate peptide that is cleaved, e.g., activated, by an enzyme chosen from matrix metalloprotease MMP-1, MMP-2, MMP-3, MMP-8, MMP-9, MMP-14, plasmin, PSA, PSMA, CATHEPSIN D, CATHEPSIN K, CATHEPSIN S, ADAM10, ADAM12, ADAMT5, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, or TACE. In embodiments, the linker includes a substrate sequence disclosed in U.S. Patent Application No. 2015/0087810, U.S. Pat. Nos. 8,541,203, 8,580,244. In some embodiments, the linker comprises a sequence disclosed in one of the following articles: van Kempen, et al. Eur Cancer (2006) 42:728-734; Desnoyers, L. R. et al. Sci Transl Med (2013) 5:207; Rice, J. J. et al. Protein Sci (2006) 15:825-836; Boulware, K. T. and Daugherty, P. S. Proc Natl Acad Sci USA (2006) 103:7583-7588; and Eckhard, U et al Matrix Biol (2015) doi: 10.1016/j.matbio.2015.09.003 (epub). The contents of any of the publications referenced herein are hereby expressly incorporated by reference.

Other linkers useful in the nanogels of instant invention are described in PCT Application Nos. PCT/US2018/049594 and PCT/US2018/049596, the disclosure of each of which is incorporated herein in its entirety.

Reversibly modified proteins provided herein can, in some embodiments, be formed or self-assemble into various nanoparticles including, without limitation, protein-hydrophilic polymer conjugates (e.g., reversibly modified with PEG), protein-hydrophobic polymer conjugates (e.g., reversibly modified PLA or PLGA), bulk crosslinked protein hydrogels, crosslinked protein nanogel particles, protein nanocapsules with different shell materials (e.g., silica), protein-conjugated nanoparticles (e.g., liposome, micelle, polymeric nanoparticles, inorganic nanoparticles), e.g., as described in WO2015/048498. Likewise, proteins crosslinked to each other, as provided herein, in some embodiments, can be formed or can self-assemble into protein nanoparticles.

In some embodiments, protein nanoparticles (e.g., protein nanogels, including protein-polymer nanogels) contain carrier proteins or other carrier molecules. Carrier proteins typically facilitate the diffusion and/or transport of different molecules, and can increase stability of the nanoparticles and/or increase stability of the nanoparticle on the cell surface, and/or increases affinity of the nanoparticle to the cell surface. It should be understood that the term “carrier protein,” as used herein, refers to a protein that does not adversely affect a biologically-active protein of a protein nanoparticle. In some embodiments, a carrier protein is an inert protein. In some embodiments, the carrier protein or carrier molecules are chosen from albumin, protamine, chitosan carbohydrates, heparan-sulfate proteoglycans, natural polymers, polysaccharides, dextramers, cellulose, fibronectin, collagen, fibrin, or proteoglycans. Thus, in some embodiments, carrier proteins are not biologically active.

In some embodiments, provided herein is a monodispersed plurality of biologically-active protein-polymer particles, e.g., nanoparticles, e.g., nanogels. In embodiments, the proteins of the nanogels are reversibly and covalently crosslinked to each other through a degradable linker, and wherein proteins of the nanogels are crosslinked to a polymer. In some embodiments, the polymer is crosslinked to the surface of a nanogel (and, thus, is considered to be surface-conjugated).

In some embodiments, a nanoparticle (e.g., nanogel) comprises, consists of, or consists essentially of (a) one or more biologically-active proteins reversibly and covalently crosslinked to each other through a degradable linker (e.g., disulfide linker) and (b) polymers crosslinked to surface-exposed proteins of the nanogel (e.g., reversibly and covalently crosslinked through a degradable linker). In some embodiments, the weight percentage of proteins crosslinked to each other is greater than 75% w/w (e.g., greater than 80%, 85% or 90% w/w) of the nanogel.

A plurality of nanogels is considered to be “monodispersed” in a composition (e.g., an aqueous or otherwise liquid composition) if the nanogels have similar size (e.g., diameter) relative to each other, for example the polydispersity index measured by dynamic light scattering is less than 0.35, more preferably less than 0.3, such as less than 0.25 or less than 0.2. Nanogels of a plurality may be considered to have the same size relative to each other if the sizes among the nanogels in the plurality vary by no more than 5%-10%.

Other aspects of the present disclosure provide nanogels comprising a polymer and at least 75% (e.g., about 80%) w/w of proteins that are reversibly and covalently crosslinked to each other through a degradable linker. In some embodiments, the degradable linker is a redox responsive linker, such as, for example, a disulfide linker (e.g., Formula I).

Yet other aspects of the present disclosure provide methods of producing a plurality of biologically-active protein nanogels, the methods comprising (a) contacting a protein with a degradable linker (e.g., a disulfide linker) under conditions that permit reversible covalent crosslinking of proteins to each other through the degradable linker, thereby producing a plurality of protein nanogels, and (b) contacting the protein nanogels with a polymer (e.g., polyethylene glycol) under conditions that permit crosslinking of the polymer to proteins of the protein nanogels, thereby producing a plurality of biologically-active protein-polymer nanogels.

In some embodiments, the conditions of (a) include contacting the protein with the degradable linker in an aqueous buffer at a temperature of 4° C. to 25° C. In some embodiments, the conditions of (a) include contacting the protein with the degradable linker in an aqueous buffer for 30 minutes to one hour. In some embodiments, the conditions of (b) include contacting the protein nanogels with the polymer in an aqueous buffer at a temperature of 4° C. to 25° C. In some embodiments, the conditions of (b) include contacting the protein nanogels with the polymer in an aqueous buffer for 30 minutes to one hour. In some embodiments, the aqueous buffer comprises phosphate buffered saline (PBS).

In some embodiments, the conditions of (a) do not include contacting the protein with the degradable linker at a temperature of greater than 30° C. In some embodiments, the conditions of (b) do not include contacting the protein nanogels with the polymer at a temperature of greater than 30° C.

In some embodiments, the conditions of (a) do not include contacting the protein with the degradable linker in an organic solvent (e.g., alcohol). In some embodiments, the conditions of (b) do not include contacting the protein nanogels with the polymer in an organic solvent.

In some embodiments, the protein is a cytokine, growth factor, antibody or antigen. For example, the protein may be a cytokine molecule as described herein. In some embodiments, the cytokine molecule is an IL-12 molecule. In some embodiments, the cytokine molecule is an IL-15 molecule, e.g., IL-15 or IL-15SA.

In some embodiments, the degradable linker is a redox responsive linker. In some embodiments, the redox responsive linker comprises a disulfide bond. In some embodiments, the degradable linker comprises, or consists of, Formula I.

In some embodiments, the polymer is a hydrophilic polymer. The hydrophilic polymer, in some embodiments, comprises polyethylene glycol (PEG). For example, the hydrophilic polymer may be a 4-arm PEG-NH₂ polymer.

In some embodiments, the concentration of the protein in the aqueous buffer is 10 mg/mL to 50 mg/mL (e.g., 10, 15, 20, 25, 30, 35, 40, 45 or 50 mg/mL).

In some embodiments, the weight percentage of protein (e.g., biologically-active protein, crosslinked protein) in the biologically-active protein-polymer nanogels is at least 75%. In some embodiments, the weight percentage of protein in the biologically-active protein-polymer nanogels is at least 80%. In some embodiments, the weight percentage of protein in the biologically-active protein-polymer nanogels is at least 85%. In some embodiments, the weight percentage of protein in the biologically-active protein-polymer nanogels is at least 90%.

In some embodiments, the protein, under physiological conditions, is released in its native conformation from the nanogel and is biologically active. In some embodiments, the specific activity of the released protein is at least than 50% (e.g., at least 55%, 60%, 65%, 70%, 75%, 80%, 85%, 90%, 95%, 96%, 97%, 98%, 99% or 100%) of the specific activity of the protein before it was crosslinked to another protein through a degradable linker.

Some aspects of the disclosure provide proteins reversibly linked through a degradable linker to a polymerizable functional group. Such proteins are considered herein to be reversibly modified proteins.

In some embodiments, the polymerizable functional group comprises silane and/or a crosslinkable polymer. In some embodiments, the crosslinkable polymer comprises poly(ethylene oxide), polylactic acid and/or poly(lactic-co-glycolic acid). In some embodiments, the proteins are reversibly linked through a degradable linker to silane.

Other aspects of the disclosure provide pluralities of any reversibly modified protein described herein. In some embodiments, reversibly modified proteins in such pluralities are crosslinked.

Yet other embodiments of the disclosure provide nanoparticles that comprise a polymer and at least 50% w/w of a protein that is reversibly linked through a degradable linker to a polymerizable functional group. “w/w” here means weight of protein to weight of nanoparticle (e.g., nanogel). A “polymerizable functional group,” as used herein, refers to a group of atoms and bonds that can chemically react to form a polymer chain or network. A “polymer” refers to a chain or network of repeating units or a mixture of different repeating units. As used herein, a polymer is itself a functional group. Examples of polymerizable functional groups for use in accordance with the disclosure include, without limitation, silane, ethylene oxide, lactic acid, lactide, glycolic acid, N-(2-hydroxypropyl)methacrylamide, silica, poly(ethylene oxide), polylactic acid, poly(lactic-co-glycolic acid), polyglutamate, polylysine, cyclodextrin and dextran chitosan. Other polymerizable functional groups are contemplated and may be used in accordance with the disclosure. It should be understood, however, that a “polymer,” as used herein, is not a protein (is a non-protein), peptide (is a non-peptide) or amino acid (is a non-amino acid).

The term “polymer” encompasses “co-polymer.” That is, a polymer may comprise a mixture of different functional groups (e.g., silane-PEG-silane), including shorter polymers or co-polymers. The functional groups are typically polymerized under protein-compatible, neutral conditions. Thus, in some embodiments, polymerization of the functional groups occurs in an at least partially aqueous solution at about pH 6 to about pH 8. For example, polymerization of the functional groups can occur at pH 6, pH 6.5, pH 7, pH 7.5 or pH 8. In some embodiments, polymerization of the functional groups occurs at about pH 7.

In some embodiments, the polymerization reaction is catalyzed by sodium fluoride, potassium fluoride or any other soluble fluoride.

Exemplary polymers that can be reversibly linked to proteins and/or used to form nanoparticles (e.g., nanocapsules, nanogels, hydrogels) include, without limitation, aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), and poly(lactide-co-caprolactone), and natural polymers such as alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. Other polymers are contemplated and may be used in accordance with the disclosure.

In some embodiments, proteins are reversibly linked to hydrophilic polymers such as, for example, polyethylene glycol (PEG), polyethylene glycol-b-poly lysine (PEG-PLL), and/or polyethylene glycol-b-poly arginine (PEG-PArg).

Some embodiments of the present disclosure involve nanoparticles (e.g., nanogels) comprising on their surface a polycation. A polycation is a molecule or chemical complex having positive charges at several sites. Generally, polycations have an overall positive charge. Examples of polycations for use in accordance with the present disclosure include, without limitation, polylysine (poly-L-lysine and/or poly-D-lysine), poly(argininate glyceryl succinate) (PAGS, an arginine-based polymer), polyethyleneimine, polyhistidine, polyarginine, protamine sulfate, polyethylene glycol-b-polylysine (PEG-PLL), or polyethylene glycol-g-polylysine.

In some embodiments, a polycation is added to the surface of a nanogel. In some embodiments, a polycation (e.g., polyethylene glycol-b-polylysine or PEG-PLL) is added to the surface of a nanogel. In some embodiments the polycation is polyethylene glycol-b-polylysine. In some embodiments the polycation is added to a nanogel with or without an anti-CD45 antibody.

In embodiments, the nanoparticle comprises polyK30. In embodiments, the nanoparticle comprises polyethylene glycol (PEG), polyethylene glycol-b-poly lysine (PEG-PLL), or polyethylene glycol-b-poly arginine (PEG-PArg). In embodiments, the nanoparticle comprise polyK200.

In embodiments, the nanoparticle comprises at least one polymer, cationic polymer, or cationic block co-polymer on the nanoparticle surface. In some embodiments, the cationic polymer comprises poly-lysine, e.g., polyK30 or polyK200. In some embodiments, the poly-lysine is poly-L-lysine. In embodiments, the poly-lysine has an average length of 20-30, 30-40, 40-50, 50-100, 100-150, 150-200, 200-250, 250-300, 300-400, or 400-500 amino acids.

In embodiments, the nanoparticle comprises polyethylene glycol (PEG). In embodiments, the PEG has a molecular weight of 1-2, 2-3, 3-4, 4-5, 5-6, 6-7, 7-8, 8-9, or 9-10 kD.

In some embodiments, the nanoparticle comprises a cationic block co-polymer comprising PEG (e.g., PEG5k) and poly-lysine, e.g., polyK30 or polyK200. In embodiments the cationic block co-polymer comprises PEG5k-polyK30 or PEG5k-polyK200.

Without wishing to be bound by theory, in some embodiments, a nanoparticle comprising a low molecular weight poly-lysine (e.g., having an average length of 10-150, 20-100, 20-80, 20-60, 20-40, or about 30 amino acids) shows superior properties to a nanoparticle comprising a higher molecular weight poly-lysine (eg., having an average length of about 200 amino acids). The superior properties can be, e.g., low toxicity, low aggregation, or high cell loading, or any combination thereof.

In embodiments, the nanoparticle comprising the low molecular weight poly-lysine shows low toxicity to T cells, e.g., as assayed by quantifying the number of live T cells after freezing and thawing, e.g., using the method of the Examples. In embodiments, low toxicity comprises cells that expand at least 1.2-fold, 1.4-fold, 1.6-fold, 1.8-fold, 2-fold, 2.5-fold, 3-fold, 4-fold, or 5-fold after freezing and thawing.

In embodiments, the nanoparticle comprising the low molecular weight poly-lysine shows low aggregation, e.g., as measured by dynamic light scattering, e.g., as described in the Examples. In embodiments, low aggregation comprises a population of nanoparticles having a size of about 80 nm (e.g., 70-90 nm, 60-100 nm, or 50-150 nm).

In embodiments, the nanoparticle comprising the low molecular weight poly-lysine shows high cell loading, as measured by mean fluorescent intensity (MFU) of nanoparticles being loaded onto activated naïve T cells, e.g., as described in the Examples. In embodiments, high cell loading comprises a MFU at least 2, 5, 10, 20, 50, 100, 200, or 500 times greater than a control nanoparticle that is otherwise similar but has polyK200.

In other embodiments, proteins are reversibly linked to hydrophobic polymers such as, for example, polylactic acid (PLA) and/or polylactic-co-glycolic acid) (PLGA). These protein-hydrophobic polymer conjugates can, in some embodiments, self-assemble into nanoparticles.

The protein conjugates of the present disclosure, in some embodiments, may be crosslinked to form a hydrogel network, nanogel particle, or protein nanogel, e.g., as described in WO2015/048498 and WO2017/027843, all of which are herein considered to be “nanoparticles.”

In some embodiments, the polymerizable functional group comprises silane and/or a crosslinkable polymer. In some embodiments, the crosslinkable polymer comprises poly(ethylene oxide), polylactic acid and/or poly(lactic-co-glycolic acid).

In some embodiments, the nanoparticles comprise at least 75% w/w of a protein that is reversibly linked to a polymerizable functional group. In some embodiments, the nanoparticles comprise at least 80% w/w of a protein that is reversibly linked to a polymerizable functional group. Also contemplated herein are nanoparticles that comprise about 50% w/w to about 90% w/w of a protein that is reversibly linked to a polymerizable functional group. For example, in some embodiments, a nanoparticle may have about 50% w/w, about 55% w/w, about 60% w/w, about 65% w/w, about 70% w/w, about 75% w/w, about 80% w/w, about 85% w/w, or about 90% w/w of a protein that is reversibly linked to a polymerizable functional group.

Still other aspects of the disclosure provide methods of producing a nanoparticle, the methods comprising modifying a protein with a degradable linker and polymerizable functional groups, and polymerizing the polymerizable functional groups with a crosslinker and soluble fluoride.

In some embodiments, the polymerizable functional group comprises silane and/or a crosslinkable polymer. In some embodiments, the crosslinkable polymer comprises poly(ethylene oxide), polylactic acid and/or polylactic-co-glycolic acid).

In some embodiments, the soluble fluoride is sodium fluoride. In some embodiments, the soluble fluoride is potassium fluoride.

In some embodiments, the nanoparticles comprise one or more reactive group on their surface. In embodiments, the one or more reactive groups on their exterior surface can react with reactive groups on nucleated cells (e.g., T cells). Exemplary nanoparticle reactive groups include, without limitation, thiol-reactive maleimide head groups, haloacetyl (e.g., iodoacetyl) groups, imidoester groups, N-hydroxysuccinimide esters, pyridyl disulfide groups, and the like. These reactive groups react with groups on the nucleated cell surface and, thus, the nanoparticles are bound to the cell surface. In embodiments, when surface modified in this manner, the nanoparticles are intended for use with specific carrier cells having “complementary” reactive groups (i.e., reactive groups that react with those of the nanoparticles). In some embodiments, the nanoparticles will not integrate into the lipid bilayer that comprises the cell surface. Typically, the nanoparticles will not be significantly phagocytosed (or substantially internalized) by the nucleated cells.

In some embodiments, the reactive group is a maleimide, rhodamine or IR783 reactive group.

In embodiments, the IL-15 molecule is a molecule described in PCT International Application Publication No. WO2017/027843, which is herein incorporated by reference in its entirety.

### Immunostimulatory Fusion Molecules/Tethered Fusions

In certain embodiments, the IFM can be represented with the following formula in an N to C terminal orientation: R1-(optionally L1)-R2 or R2-(optionally L1)-R1; wherein R1 comprises an immune cell targeting moiety, L1 comprises a linker (e.g., a peptide linker described herein), and R2 comprises an immune stimulating moiety, e.g., a cytokine molecule.

In some embodiments, the immune stimulating moiety, e.g., the cytokine molecule, is connected to, e.g., covalently linked to, the immune cell targeting moiety.

In some embodiments, the immune stimulating moiety, e.g., the cytokine molecule, is functionally linked, e.g., covalently linked (e.g., by chemical coupling, fusion, noncovalent association or otherwise) to the immune cell targeting moiety. For example, the immune stimulating moiety can be covalently coupled indirectly, e.g., via a linker to the immune cell targeting moiety.

In embodiments, the linker is chosen from: a cleavable linker, a non-cleavable linker, a peptide linker, a flexible linker, a rigid linker, a helical linker, or a non-helical linker. In some embodiments, the linker is a peptide linker. The peptide linker can be 5-20, 8-18, 10-15, or about 8, 9, 10, 11, 12, 13, 14, or 15 amino acids long. In some embodiments, the peptide linker comprises Gly and Ser, e.g., a linker comprising the amino acid sequence (Gly₄-Ser)ₙ, wherein n indicates the number of repeats of the motif, e.g., n=1, 2, 3, 4 or 5 (e.g., a (Gly₄Ser)₂ or a (Gly₄Ser)₃ linker). In some embodiments, the linker comprises the amino acid sequence of SEQ ID NO: 36, 37, 38, or 39, or an amino acid sequence substantially identical thereto (e.g., having 1, 2, 3, 4, or 5 amino acid substitutions). In one embodiment, the linker comprises an amino acid sequence GGGSGGGS (SEQ ID NO: 37). In another embodiment, the linker comprises amino acids from an IgG4 hinge region, e.g., amino acids DKTHTSPPSPAP (SEQ ID NO: 38).

In still another embodiment, the cleavable linker is configured for cleavage by an enzyme, such as a protease (e.g., pepsin, trypsin, thermolysine, matrix metalloproteinase (MMP), a disintegrin and metalloprotease (ADAM; e.g. ADAM-10 or ADAM-17)), a glycosidase (e.g., α-, β-, γ-amylase, α-, β-glucosidase or lactase) or an esterase (e.g. acetyl cholinesterase, pseudo cholinesterase or acetyl esterase). Other enzymes which may cleave the cleavable linker include urokinase plasminogen activator (uPA), tissue plasminogen activator (tPA), granzyme A, granzyme B, lysosomal enzymes, cathepsins, prostate-specific antigen, Herpes simplex virus protease, cytomegalovirus protease, thrombin, caspase, and interleukin 1 beta converting enzyme.

Still another example is over-expression of an enzyme, e.g., proteases (e.g., pepsin, trypsin), in the tissue of interest, whereby a specifically designed peptide linker will be cleaved in upon arrival at the tissue of interest. Illustrative examples of suitable linkers in this respect are Gly-Phe-Ser-Gly (SEQ ID NO: 105), Gly-Lys-Val-Ser (SEQ ID NO: 106), Gly-Trp-Ile-Gly (SEQ ID NO: 107), Gly-Lys-Lys-Trp (SEQ ID NO: 108), Gly-Ala-Tyr-Met (SEQ ID NO: 109).

In still another example, over-expression of an enzyme, e.g. of glycosidases (e.g. α-amylase), in the tissue of interest, causes a specifically designed carbohydrate linker to be cleaved upon arrival at the tissue of interest. Illustrative examples of suitable linkers in this respect are −(α-1-4-D-Glucose)n- where n≥4.

The cleavable linker may include a total of from 2 to 60 atoms, such as from 2 to 20 atoms. The cleavable linker may include amino acid residues, and may be a peptide linkage, e.g., of from 1 to 30, or from 2 to 10, amino acid residues. In one variant, the cleavable linker B consists of from 1 to 30, such as from 2 to 10, or from 2 to 8, or from 3 to 9, or from 4-10, amino acids. For pH sensitive linkers, the number of atoms is typically from 2 to 50, such as from 2-30.

In some embodiments of the invention, the linker includes an aminocaproic acid (also termed aminohexanoic acid) linkage or a linkage composed of from 1 to 30, or from 2 to 10 carbohydrate residues.

The linker may, besides the substrate peptide, contain connectors, involved in the bond or bonds with the therapeutic protein. Such connectors may each consist of one amino acid residue or of an oligopeptide containing from 2 to 10, such as from 3 to 9, or from 4 to 8, or from 2 to 8, amino acid residues. The amino acid residue or oligopeptide as the connectors may, if present, bind to both ends of the substrate peptide, or may bind only to one end of the substrate peptide so as to represent one of the structures. Types of one amino acid usable as the connector(s), and amino acid residues constituting an oligopeptide usable as the connector(s) are not particularly limited, and one amino acid residue of an arbitrary type, or an arbitrary oligopeptide containing, e.g., from 2 to 8 of the same or different amino acid residues of arbitrary types can be used. Examples of the oligopeptide usable as the connector(s) include, for example, connectors that are rich in Gly amino acids. Other organic moieties can also be used as connectors.

In yet other embodiments, the immune stimulating moiety is directly covalently coupled to the immune cell targeting moiety, without a linker.

In yet other embodiments, the immune stimulating moiety and the immune cell targeting moiety of the IFM are not covalently linked, e.g., are non-covalently associated.

Exemplary formats for fusion of a cytokine molecule to an antibody molecule, e.g., an immunoglobulin moiety (Ig), for example an antibody (IgG) or antibody fragment (Fab, scFv and the like) can include a fusion to the amino-terminus (N-terminus) or carboxy-terminus (C-terminus) of the antibody molecule, typically, the C-terminus of the antibody molecule. In one embodiment, a cytokine-Ig moiety fusion molecule comprising a cytokine polypeptide, cytokine-receptor complex, or a cytokine-receptor Fc complex joined to an Ig polypeptide, a suitable junction between the cytokine polypeptide chain and an Ig polypeptide chain includes a direct polypeptide bond, a junction having a polypeptide linker between the two chains; and, a chemical linkage between the chains A typical junction is a flexible linker composed of small Gly4Ser linker (GGGGS)_{N}, where _{N} indicates the number of repeats of the motif. (GGGGS)₂ and (GGGGS)₃ are preferred embodiments of linkers for use in the fusion constructs of the disclosure.

Exemplary immunostimulatory fusion molecules described herein can comprise the amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO: 70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO:87, SEQ ID NO:88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO:97, SEQ ID NO:98, SEQ ID NO: 99, SEQ ID NO: 100, SEQ ID NO: 101, SEQ ID NO: 102, SEQ ID NO: 103, SEQ ID NO: 104, or an amino acid sequence substantially identical thereto (e.g., an amino acid sequence at least 85%, 90%, 95%, 96%, 97%, 98%, 99% or higher identity to any of the aforesaid amino acid sequences).

Described herein are exemplary immunostimulatory fusion molecules (or portion thereof) of the present disclosure. It should be noted that in certain scFv the arrangement is VH-linker-VL. However, the VL-linker-VH arrangement can also be used without affecting

Additional sequences that can be included in the IFM of the present disclosure are shown in Table 4 below. In some embodiments, the IFM comprises a constant lamba or lamda region. Exemplary constant lamba or lamda regions include SEQ ID NOS: 74-78. In various embodiments, the IFMs described herein can comprise one or more of the amino acid sequences of SEQ ID NOS: 1-104, or an amino acid sequence substantially identical thereto (e.g., an amino acid sequence at least 70%, 75%, 80%, 85%, 90%, 95%, 96%, 97%, 98%, 99% or higher identity to any one of SEQ ID NOS: 1-104, or having at least 1, 2, 3, 4, 5, 6, 7, 8, 9, or 10 or more amino acid alterations (e.g., substitutions, deletions, or insertions, e.g., conservative substitutions) relative to any one of SEQ ID NOS: 1-104. In embodiments, the IFM comprises no more than five, ten or fifteen alterations (e.g., substitutions, deletions, or insertions, e.g., conservative substitutions) relative to any one of SEQ ID NOS: 1-104.

TABLE 4
Additional Sequences

| SEQ ID | | |
|---|---|---|
| NO: | Name | Description/Comments |
| 9 | IL-15Rα-sushi (also | Sushi domain from wild-type IL-15Rα. |
| | referred as sushi) | |
| 10 | IL15-WT | Wild-type IL-15, corresponds to mature polypeptide from full |
| | | IL-15 sequence described in SEQ ID NO: 40. |
| 11 | IL15-N72D | IL-15 containing N72D mutation |
| 12 | sushiL77I-Fc | Sushi domain from IL-15Rα fused to IgG1-Fc; sushi domain |
| | | contains L77I mutation |
| 13 | sushi-Fc | Sushi domain from IL-15Rα fused to IgG1-Fc |
| 33 | Leader-1 | Leader sequence (e.g. signal sequence, signal peptide) used for |
| | | antibody light-chain, IL-15, N-terminal IL-15 fusions, and N- |
| | | terminal fusion of sushi to Fab-light-chain |
| 34 | Leader-2 | Leader sequence (e.g. signal sequence, signal peptide) used for |
| | | antibody heavy-chain |
| 35 | IL-15Rα leader sequence | Leader sequence used for sushi and sushi-Fc constructs. |
| 36 | Linker-1 (L1) | (G45)3 linker |
| 40 | Human IL-15 full sequence | Genbank Accession No. CAA62616.1 |
| 41 | Human IL-15Rα full | Genbank Accession No. AAI21141.1 |
| | sequence | |
| 46 | IL-12A full sequence (also | Genbank Accession No. P29459 |
| | referred to as IL-12p35) | |
| 47 | IL-12A mature sequence | |
| 48 | IL-12B full sequence (also | Genbank Accession No. P29460 |
| | referred to as IL-12p40) | |
| 49 | IL-12B mature sequence | |
| 50 | scIL-12p70-BA | Synthetic sequence; IL-12B and IL-12A joined by flexible |
| | | linker |
| 51 | scIL-12p70-AB | Synthetic sequence; IL-12A and IL-12B joined by flexible |
| | | linker |
| 52 | Minimal sushi domain | |
| 53 | IgG1-Fc | Fc domain (CH2 and CH3 domains) from human IgG1 |
| 54 | IgG2-Fc | Fc domain (CH2 and CH3 domains) from human IgG2 |
| 55 | IgG2Da-Fc | Fc domain from human IgG2 containing two point mutations |
| 56 | sushi-IgG2Da-Fc (also | Sushi domain from IL-15Rα fused to IgG2Da-Fc |
| | referred to as sushi-Fc2Da) | |
| 59 | 9.4 heavy-chain variable | |
| | domain | |
| 60 | 9.4 light-chain variable | |
| | domain | |
| 70 | Linker-5 (L5) | (G35)4 linker, e.g., as described above in SEQ ID NOs: 50 |
| | | and 51 |
| 71 | Linker-6 (L6) | (G45)4 linker |
| 72 | scIL-12p70-BA-L6 | Synthetic sequence; IL-12B and IL-12A joined by linker L5. |
| 73 | scIL-12p70-AB-L5 | Synthetic sequence; IL-12A and IL-12B joined by linker L5. |
| 79 | HC-h9.4Fab | Heavy-chain of a humanized anti-CD45 antibody; contains |
| | | humanized 9.4 (h9.4) heavy-chain variable domain and the |
| | | CH1 domain from human IgG1 |
| 80 | HC-h9.4Fab-h9.4scFv | Heavy-chain of a humanized anti-CD45 antibody linked to a |
| | | humanized anti-CD45 scFv; contains variable domain from |
| | | h9.4 heavy-chain and the CH1 domain from human IgGl. An |
| | | h9.4 scFv is genetically fused to the Fab heavy chain C- |
| | | terminus using a flexible linker (Linker-1, SEQ ID NO: 36). |
| 82 | LC-h9.4Fab-scIL-12p70 | Light-chain of a humanized anti-CD45 antibody; contains |
| | | variable domain from h9.4 light-chain and human constant |
| | | kappa domain, a wild-type single-chain human IL-12p70 |
| | | (SEQ ID NO: 50) genetically fused to antibody light-chain C- |
| | | terminus using a flexible linker (Linker-1, SEQ ID NO: 36); |
| | | single-chain human IL-12p70 comprises a genetic fusion of |
| | | human IL-12A and IL-12B using a flexible linker (Linker-5; |
| | | SEQ ID NO: 70). |
| 91 | h9.4 scFv | An scFv comprising a humanized 9.4 antibody; heavy-chain |
| | | and light-chain variable domains are genetically fused using a |
| | | flexible linker (Linker-6; SEQ ID NO: 71). |
| 102 | LC-h9.4Fab-scIL-12p70AB | Light-chain of a humanized anti-CD45 antibody; contains |
| | | variable domain from h9.4 light-chain and human constant |
| | | kappa domain; contains a single-chain human IL-12p70 (SEQ |
| | | ID NO: 51) genetically fused to antibody light-chain C- |
| | | terminus using a flexible linker (Linker-1, SEQ ID NO: 36); |
| | | single-chain human IL-12p70 comprises a genetic fusion of |
| | | human IL-12A and IL-12B using a flexible linker (Linker-5; |
| | | SEQ ID NO: 70). |
| 103 | LC-h9.4Fab-IL-12A | Light-chain of a humanized anti-CD45 antibody; contains |
| | | variable domain from h9.4 light-chain and human constant |
| | | kappa domain; contains a human IL-12A (SEQ ID NO: 47) |
| | | genetically fused to antibody light-chain C-terminus using a |
| | | flexible linker (Linker-1, SEQ ID NO: 36). |
| 104 | LC-h9.4Fab-IL-12B | Light-chain of a humanized anti-CD45 antibody; contains |
| | | variable domain from h9.4 light-chain and human constant |
| | | kappa domain; contains a human IL-12B (SEQ ID NO: 49) |
| | | genetically fused to antibody light-chain C-terminus using a |
| | | flexible linker (Linker-1, SEQ ID NO: 36). |

### Protein Variants

Full length polypeptides and variants thereof are described below. Full-length IL-15 sequence (SEQ ID NO: 40) is taken from Genbank Accession No. CAA62616.1; mature IL-15 is devoid of the signal sequence and is defined in SEQ ID NO: 10. Full-length IL-15Rcc (SEQ ID NO: 41) is taken from Genbank Accession No. AAI21141.1. The sushi domain of IL-15Rcc (IL-15Rcc-sushi) is given by SEQ ID NO: 9. A minimal sushi domain encompassing the first and fourth cysteines and the intervening amino acids (SEQ ID NO: 52) have also been described elsewhere and are plausible substitutes. Similarly, optional N-terminal additions to the minimal sushi domain comprising the native Thr or Ile-Thr and/or optional C-terminal additions to the minimal sushi domain comprising Ile or Ile-Arg residues are also plausible.

Protein variants described below specify protein subunit names and SEQ ID NOs corresponding to the mature proteins. Each protein subunit was recombinantly expressed with an N-terminal signal peptide to facilitate secretion from the expressing cell. The native IL-15Rcc signal peptide (SEQ ID NO: 35) was used for sushi, sushi-L77I-Fc, and sushi-Fc. The leader sequence in SEQ ID NO: 33 was used to support secretion of antibody light-chains, IL15-WT, N-terminal IL-15 fusions, IL15-N72D, and N-terminal fusion of sushi to Fab-light-chain. The leader sequence in SEQ ID NO: 34 was used to support secretion of antibody heavy chains.

For “heavy-chain” and “light-chain” nomenclature used herein the standard naming system for antibodies. For example, in an antibody Fab fragment both chains have approximately the same molecular mass, but the heavy-chain is referred to as the chain of the Fab fragment corresponding to the heavy-chain in the full-length antibody (e.g. containing the variable heavy-chain and CH1 domains). Further, in the case of cytokine fusions to the light-chain of a Fab fragment, the light-chain would actually have a larger molecular mass than the heavy-chain due to the cytokine fusion; for consistency, however, the standard naming convention is maintained in which the variable light-chain domain and constant kappa domain and cytokine fusion comprise the “light-chain” while the variable heavy-chain domain and CH1 domain comprises the “heavy-chain”.

In some embodiments, the IFM comprises h9.4Fab-scIL-12p70 or h9.4Fab-h9.4scFv-scIL-12p70, as defined below:

Protein name: h9.4Fab-scIL-12p70

This protein was made by coexpression of two subunits: HC-h9.4Fab (SEQ ID NO: 79) and LC-h9.4Fab-scIL-12p70 (SEQ ID NO: 82). The resulting protein comprises a fusion of a single-chain human IL-12p70 to the C-terminus of h9.4 Fab fragment light-chain using Linker-1 (SEQ ID NO: 36). The h9.4 Fab is an anti-human CD45R antibody Fab fragment comprising variable-heavy and variable-light chain domains (VH and VL) from h9.4 and constant domains from human (human constant kappa domain and human IgG1-CH1 domain).

Protein name: h9.4Fab-h9.4scFv-scIL-12p70

This protein was made by coexpression of two subunits: HC-h9.4Fab-h9.4scFv (SEQ ID NO: 80) and LC-h9.4Fab-scIL-12p70 (SEQ ID NO: 82). The resulting protein comprises a fusion of a single-chain human IL-12p70 to the C-terminus of h9.4 Fab fragment light-chain using Linker-1 (SEQ ID NO: 36) and a fusion of an h9.4 scFv to the h9.4 Fab fragment heavy-chain using Linker-1.

Immune Cell Targeting Moieties

In certain embodiments, the immunostimulatory fusion molecules disclosed herein include an immune cell targeting moiety. The immune cell targeting moiety can be chosen from an antibody molecule (e.g., an antigen binding domain as described herein), a receptor or a receptor fragment, or a ligand or a ligand fragment, or a combination thereof. In some embodiments, the immune cell targeting moiety associates with, e.g., binds to, an immune cell (e.g., a molecule, e.g., antigen, present on the surface of the immune cell). In certain embodiments, the immune cell targeting moiety targets, e.g., directs the immunostimulatory fusion molecules disclosed herein to an immune (e.g., a lymphocyte, e.g., a T cell).

In some embodiments, the immune cell targeting moiety is chosen from an antibody molecule (e.g., a full antibody (e.g., an antibody that includes at least one, and preferably two, complete heavy chains, and at least one, and preferably two, complete light chains), or an antigen-binding fragment (e.g., a Fab, F(ab′)2, Fv, a single chain Fv, a single domain antibody, a diabody (dAb), a bivalent antibody, or bispecific antibody or fragment thereof, a single domain variant thereof, or a camelid antibody)), non-antibody scaffold, or ligand that binds to the CD45 receptor.

In some embodiments, the immune cell targeting moiety targets the IFM to persistent, abundant, and/or recycling cell surface receptors and molecules expressed on the surface of the immune cell. These receptors/molecules include, e.g., CD45 (via, e.g., BC8 (ACCT: HB-10507), 9.4 (ATTC: HB-10508), GAP8.3 (ATTC: HB-12), monoclonal antibodies), CD8 (via OKT8 monoclonal antibody), the transmembrane integrin molecules CD11a (via MHM24 monoclonal antibody) or CD18 (via chimeric1B4 monoclonal antibody). In other preferred embodiments, the targeting moiety is directed to a marker selected from the group consisting of CD4, CD8, CD11a, CD18, CD19, CD20, and CD22. In some embodiments, the immune cell targeting moiety is chosen from an antibody molecule, e.g., an antigen binding domain, non-antibody scaffold, or ligand that binds to CD45, CD4, CD8, CD3, CD11a, CD11b, CD11c, CD25, CD127, CD137, CD19, CD20, CD22, HLA-DR, CD197, CD38, CD27, CD196, CXCR3, CXCR4, CXCR5, CD84, CD229, CCR1, CCR5, CCR4, CCR6, CCR8, or CCR10.

In some embodiments, the immune cell targeting moiety of the IFM includes an antibody molecule or a ligand that selectively binds to an immune cell surface target, e.g., an immune cell surface receptor. In some embodiments, the immune cell surface target or receptor can have one, two, three or more of the following properties: (i) is abundantly present on the surface of an immune cell (e.g., outnumbers the number of receptors for the cytokine molecule present on the immune cell surface); (ii) shows a slow downregulation, internalization, and/or cell surface turnover, e.g., relative to the receptors activated by the cytokine of the IFM; (iii) is present on the surface of the immune cell for a prolonged period of time, e.g., relative to the receptors activated by the cytokine of the IFM; or (iv) once internalized is substantially recycled back to the cell surface, e.g., at least 25%, 50%, 60%, 70%, 80%, 90% or more of the immune cell surface target is recycled back to the cell surface.

In some embodiments, the immune cell targeting moiety of the IFM binds to a recycling cell surface receptor. Without being bound by theory, it is believed that binding to the recycling cell surface receptor mediates internalization of the receptor and the IFM. For example, the IFM internalized along with the receptor may be sequestered into early endosomes and subsequently recycled back to the cell surface, instead of advancing to subsequent degradation (e.g. via either clathrin-mediated and clathrin-independent endocytosis). The return of the IFM/receptor to the cell surface can improve cytokine signaling by restoring the cytokine molecule of the IFM to the cell surface, thus increasing the time and availability of the cytokine molecule to bind its own cell-surface receptor. Additionally, signaling events that are initiated at the surface membrane by binding of a fusion protein of the disclosure may continue from endosomal compartments.

In some embodiments, the immune cell surface target or receptor is present on the surface of an immune cell, but not present on a cancer or tumor cell, e.g., a solid tumor or hematological cancer cell. In some embodiments, the immune cell surface target or receptor is predominantly present on the surface of an immune cell compared to its presence on a cancer or tumor cell, e.g., is present at least 5:1, 10:1, 15:1, 20:1 higher ratio on the immune cell relative to the cancer or tumor cell.

In some embodiments, the immune cell targeting moiety of the IFM binds to a receptor expressed on a cell (e.g., an immune cell), e.g. the surface membrane of the cell, and further the cell also expresses a cytokine receptor (e.g., a receptor to the cytokine molecule of the IFM).

In some embodiments, the immune cell targeting moiety of the IFM can be chosen from an antibody molecule or a ligand molecule that binds to an immune cell surface target, e.g., a target chosen from CD16, CD45, CD4, CD8, CD3, CD11a, CD11b, CD11c, CD18, CD25, CD127, CD56, CD19, CD20, CD22, HLA-DR, CD197, CD38, CD27, CD137, OX40, GITR, CD56, CD196, CXCR3, CXCR4, CXCR5, CD84, CD229, CCR1, CCR5, CCR4, CCR6, CCR8, or CCR10. In some embodiments, the immune cell targeting moiety binds to CD4, CD8, CD11a, CD18, CD20, CD56, or CD45. In other embodiments, the immune cell surface target is chosen from CD19, CD20, or CD22. In one embodiment, the immune cell targeting moiety comprises an antibody molecule or a ligand molecule that binds to CD45 (also interchangeably referred to herein as “CD45 receptor” or “CD45R”). In some embodiments, the target is CD45 (e.g., a CD45 isoform chosen from CD45RA, CD45RB, CD45RC or CD45RO). In embodiments, CD45 is primarily expressed on T cells. For example, CD45RA is primarily expressed on naïve T cells; CD45RO is primarily expressed on activated and memory T cells.

In other embodiments, the immune cell targeting moiety of the IFM comprises an antibody molecule (e.g., an antigen binding domain), a receptor molecule (e.g., a receptor, a receptor fragment or functional variant thereof), or a ligand molecule (e.g., a ligand, a ligand fragment or functional variant thereof), or a combination thereof, that binds to the immune cell target or receptor.

In some embodiments, the antibody molecule of the immune cell targeting moiety of the IFM comprises a full antibody (e.g., an antibody that includes at least one, and preferably two, complete heavy chains, and at least one, and preferably two, complete light chains), or an antigen-binding fragment (e.g., a Fab, F(ab′)2, Fv, a single chain Fv, a single domain antibody, a diabody (dAb), a bivalent antibody, or bispecific antibody or fragment thereof, a single domain variant thereof, or a camelid antibody)) that binds to the immune cell target or receptor.

The heavy chain constant region of the antibody molecule can be chosen from IgG1, IgG2, IgG3, or IgG4, or a fragment thereof, and more typically, IgG1, IgG2 or IgG4. In some embodiments, the Fc region of the heavy chain can include one or more alterations, e.g., substitutions, to increase or decrease one or more of: Fc receptor binding, neonatal-Fc receptor binding, antibody glycosylation, the number of cysteine residues, effector cell function, complement function, or stabilize antibody formation (e.g., stabilize IgG4). For example, the heavy chain constant region for an IgG4, e.g., a human IgG4, can include a substitution at position 228 (e.g., a Ser to Pro substitution) (see e.g., Angal, S, King, D J, et al. (1993) Mol Immunol 30:105-108 (initially described as S241P using a different numbering system); Owens, R, Ball, E, et al. (1997) Immunotechnology 3:107-116).

The antibody molecule of the immune cell targeting moiety of the IFM can bind to the target antigen with a dissociation constant of less than about 100 nM, 50 nM, 25 nM, 10 nM, e.g., less than 1 nM (e.g., about 10-100 pM). In embodiments, the antibody molecule binds to a conformational or a linear epitope on the antigen. In certain embodiments, the antigen bound by the antibody molecule of the immune cell targeting moiety is stably expressed on the surface of the immune cell. In embodiments, the antigen is a cell surface receptor that is more abundant on the cell surface relative to a receptor for the cytokine molecule of the IFM on the cell surface.

In some embodiments, the immune cell targeting moiety is chosen from an antibody molecule (e.g., a full antibody (e.g., an antibody that includes at least one, and preferably two, complete heavy chains, and at least one, and preferably two, complete light chains), or an antigen-binding fragment (e.g., a Fab, F(ab′)2, Fv, a single chain Fv, a single domain antibody, a diabody (dAb), a bivalent antibody, or bispecific or multispecific antibody or fragment thereof, a single domain variant thereof, or a camelid antibody)).

In some embodiments, the antibody molecule (e.g., mono- or bi-specific antibodies) binds to one or more of CD45, CD8, CD18 or CD11a, e.g., it is an IgG, e.g., human IgG4, or an antigen binding domain, e.g., a Fab, a F(ab′)2, Fv, a single chain Fv, that binds to CD45, CD8, CD18 or CD11a. In some embodiments, the antibody molecule is a human, a humanized or a chimeric antibody. In embodiments, the antibody molecule is a recombinant antibody.

In some embodiments, the anti-CD45 antibody is a human anti-CD45 antibody, a humanized anti-CD45 antibody, or a chimeric anti-CD45 antibody. In some embodiments, the anti-CD45 antibody is an anti-CD45 monoclonal antibody. Exemplary anti-CD45 antibodies include antibodies BC8, 4B2, GAP8.3 or 9.4. Antibodies against other immune cell surface targets are also disclosed, e.g., anti-CD8 antibodies, such as OKT8 monoclonal antibodies, anti-CD18 antibodies, such as 1B4 monoclonal antibodies, and anti-CD11a antibodies, such as MHM24 antibodies.

Also encompassed by the present disclosure are antibody molecules having the amino acid sequences disclosed herein, or an amino acid sequence substantially identical thereof), nucleic acid molecules encoding the same, host cells and vectors comprising the nucleic acid molecules.

In one embodiment, the antibody molecule that binds to CD45 is specific to one CD45 isoform or binds to more than on CD45 isoforms, e.g., is a pan-CD45 antibody. In some embodiments, the anti-CD45 antibody molecule binds to CD45RA and CD45RO. In one embodiment, the anti-CD45 antibody molecule is a BC8 antibody. In some embodiments, the BC8 antibody binds to CD45RA and CD45RO. In other embodiments, the anti-CD45 antibody molecule is CD45RO-specific or is a pan-CD45 antibody molecule, e.g., it binds to activated and memory T cells. Additional examples of anti-CD45 antibody molecules includes, but is not limited to, GAP8.3, 4B2, and 9.4.

In one embodiment, the anti-CD45 antibody molecule is a BC8 antibody, e.g., a chimeric or humanized BC8 antibody. In some embodiments, the chimeric BC8 antibody comprises:

(i) the light chain variable amino acid sequence (optionally, further including a kappa light chain sequence) corresponding to the antibody portion of the amino acid sequence shown in SEQ ID NO:1, 2, 3, 4, 7, 21, or 22, or an amino acid sequence substantially identical thereof (e.g., an amino acid sequence at least 85%, 90%, 95% or higher identical to the antibody portion of SEQ ID NO: 1, 2, 3, 4, 7, 21, or 22); and/or

(ii) the heavy chain variable amino acid sequence (optionally, further including a human IgG1 heavy chain sequence or a human IgG4 sequence having an S228P substitution) of the amino acid sequence shown in SEQ ID NO:5, 6, or 8, respectively, or an amino acid sequence substantially identical thereto (e.g., an amino acid sequence at least 85%, 90%, 95% or higher identical to SEQ ID NOs: 5, 6, or 8, respectively).

In other embodiments, the amino acid of SEQ ID NO:1-4, or an amino acid substantially identical thereto (e.g., an amino acid sequence at least 85%, 90%, 95% or higher identical to SEQ ID NO: 1-4) (optionally, further including a kappa light chain sequence), includes, optionally via a linker, an IL-15 cytokine or receptor, e.g., a sushi domain as described herein (e.g., SEQ ID NO: 9 or an amino acid substantially identical thereto (e.g., an amino acid sequence at least 85%, 90%, 95% or higher identical to SEQ ID NO: 9).

In other embodiments, the antibody molecule that binds to CD45 is a 9.4 antibody, e.g., a chimeric or humanized 9.4 antibody. In some embodiments, the chimeric 9.4 antibody comprises:

(i) the light chain variable amino acid sequence (optionally, further including a kappa light chain sequence) corresponding to the antibody portion of the amino acid sequence shown in SEQ ID NO:15, or an amino acid sequence substantially identical thereto (e.g., an amino acid sequence at least 85%, 90%, 95% or higher identical to the antibody portion of SEQ ID NO:15); and/or

(ii) the heavy chain variable amino acid sequence (optionally, further including a human IgG1 heavy chain sequence) of the amino acid sequence shown in SEQ ID NO:14, respectively, or an amino acid sequence substantially identical thereto (e.g., an amino acid sequence at least 85%, 90%, 95% or higher identical to SEQ ID NO: 14, respectively). In embodiments, the 9.4 antibody comprises one, two, or all three CDR1, CDR2 or CDR3 of the light chain variable region, and/or the heavy chain variable region, of the 9.4 antibody, e.g., according to the Kabat definition, or a closely related CDR, e.g., CDRs which have at least one amino acid alteration, but not more than two, three or four alterations (e.g., substitutions, deletions, or insertions, e.g., conservative substitutions) from the CDR sequence of SEQ ID NO:14 or 15.

In other embodiments, the antibody molecule that binds to CD45 is a 4B2 antibody, e.g., a chimeric or humanized 4B2 antibody. In some embodiments, the chimeric 4B2 antibody comprises:

(i) the light chain variable amino acid sequence (optionally, further including a kappa light chain sequence) corresponding to the antibody portion of the amino acid sequence shown in SEQ ID NO:17, or an amino acid sequence substantially identical thereto (e.g., an amino acid sequence at least 85%, 90%, 95% or higher identical to the antibody portion of SEQ ID NO:17); and/or

(ii) the heavy chain variable amino acid sequence (optionally, further including a human IgG1 heavy chain sequence) of the amino acid sequence shown in SEQ ID NO:16, respectively, or an amino acid sequence substantially identical thereto (e.g., an amino acid sequence at least 85%, 90%, 95% or higher identical to SEQ ID NO: 16, respectively). In embodiments, the 4B2 antibody comprises one, two, or all three CDR1, CDR2 or CDR3 of the light chain variable region, and/or the heavy chain variable region, of the 4B2 antibody, e.g., according to the Kabat definition, or a closely related CDR, e.g., CDRs which have at least one amino acid alteration, but not more than two, three or four alterations (e.g., substitutions, deletions, or insertions, e.g., conservative substitutions) from the CDR sequence of SEQ ID NO:16 or 17.

### Antibody Molecules

The immunostimulatory fusion molecules described herein may comprise one or more antibody molecule. For example, the immune cell engager may comprise an antibody molecule. In one embodiment, the antibody molecule binds to a cancer antigen, e.g., a tumor antigen or a stromal antigen. In some embodiments, the cancer antigen is, e.g., a mammalian, e.g., a human, cancer antigen. In other embodiments, the antibody molecule binds to an immune cell antigen, e.g., a mammalian, e.g., a human, immune cell antigen. For example, the antibody molecule binds specifically to an epitope, e.g., linear or conformational epitope, on the cancer antigen or the immune cell antigen.

In an embodiment, an antibody molecule is a monospecific antibody molecule and binds a single epitope. E.g., a monospecific antibody molecule having a plurality of immunoglobulin variable domain sequences, each of which binds the same epitope.

In another embodiment, an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable domain. In an embodiment, a multispecific antibody molecule is a bispecific antibody molecule, a trispecific antibody molecule, or a tetraspecific antibody molecule.

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv or a Fab, or fragment thereof, have binding specificity for a first epitope and a scFv or a Fab, or fragment thereof, have binding specificity for a second epitope.

In an embodiment, an antibody molecule comprises a diabody, and a single-chain molecule, as well as an antigen-binding fragment of an antibody (e.g., Fab, F(ab′)2, and Fv). For example, an antibody molecule can include a heavy (H) chain variable domain sequence (abbreviated herein as VH), and a light (L) chain variable domain sequence (abbreviated herein as VL). In an embodiment an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and mono or bispecific), triabodies (trivalent and mono or multispecific), and chimeric or humanized antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. A preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the disclosure, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R, Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods.

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the disclosure. Antibody molecules generated in a non-human organism, e.g., a rat or mouse, and then modified, e.g., in the variable framework or constant region, to decrease antigenicity in a human are within the disclosure. For example, anti-human CD45 antibodies such as 9.4, 4B2 and BC8 can be humanized using techniques known in the art, for making the tethered fusions disclosed herein.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present disclosure (UK Patent Application GB 2188638A, filed on Mar. 26, 1987; Winter U.S. Pat. No. 5,225,539), the contents of which is expressly incorporated by reference.

Also within the scope of the disclosure are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFv) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target protein. In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., a cytokine molecule as described herein or other chemical or proteinaceous groups). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the disclosure are intended to include derivatized and otherwise modified forms of the antibodies described herein, including immunoadhesion molecules. For example, an antibody molecule can be functionally linked (e.g., by chemical coupling, genetic fusion, noncovalent association or otherwise) to one or more other molecular entities, such as a cytokine molecule, another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking an antibody molecule to one or more proteins, e.g., a cytokine molecule, another antibody molecule (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### Dosing Regimens

A therapeutically effective dose is an amount of immune agonist-loaded T cells (e.g., IL-12 tethered fusion-loaded T cells or IL-15 nanogel-loaded T cells) that is capable of producing a clinically desirable result (i.e., a sufficient amount to induce or enhance a specific T cell immune response against cells overexpressing antigen (e.g., a cytotoxic T cell response) in a statistically significant manner) in a treated human or non-human mammal. As is well known in the medical arts, the dosage for any one patient depends upon many factors, including the patient's size, weight, body surface area, age, the particular therapy to be administered, sex, time and route of administration, general health, and other drugs being administered concurrently. Doses will vary, but acceding to some embodiments of the invention, the dosage for administration of immune agonists-loaded T cells described herein is about 20M cells/m², 40M cells/m², 100M cells/m², 120M cells/m², 200M cells/m², 360M cells/m², 600M cells/m², 1B cells/m², 1.5B cells/m², 10⁶ cells/m², about 5×10⁶ cells/m², about 107 cells/m², about 5×107 cells/m², about 108 cells/m², about 5×108 cells/m², about 109 cells/m², about 5×109 cells/m², about 1010 cells/m², about 5×1010 cells/m², or about 10″ cells/m².

In some embodiments, the IL-12 tethered fusion-loaded T cells and the IL-15 nanogel-loaded T cells are administered at a ratio of either agent to the other agent of about 1:1, 1:2, 1:3, 1:4 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:30, 1:35, 1:40, 1:45, 1:50, 1:55, 1:60, 1:65, 1:70; 1:75, 1:80, 1:85, 1:90, 1:95, 1:100, 1:120, 1:130, 1:140, 1:150, 1:160, 1:170, 1:180, 1; 190, 1:200, 1:500, 1:1000. 1:5000, 1:10,000, 1:100,000, 2:3, 3:4, 2:5, 3:5, 3:10, 7:10, 9:10, 2:15, 4:15, 6:15, 7:15, 8:15, 11:15, 13:15, 14:15, 3:20, 7:20, 9:20, 11:20, 13:20, 17:20, 19:20, 1:25, 2:25, 4:25, 6:25, 7:25, 8:25, 10:25, 11:25, 12:25, 13:25, 14:25, 16:25, 17:25, 18:25, 19:25, 21:25, 22:25, 23:25, or 24:25.

The synergistic combination therapies of the present invention may be dosed a single time, or two or more repeated doses. Such combination therapies can be administered on a daily, weekly, bi-weekly, or monthly basis. In addition or as an alternative, such combination therapies can be administered about every hour, 2 hours, 5 hours, 8 hours, 10, hours, 12 hours, 15 hours, 18 hours, 20 hours, 24 hours, 2 days, 3 days, 5 days, 10 days, 30 days, 60 days, 90 days, 3 weeks, 4 weeks, 6 weeks, 8 weeks, 10 weeks, 12 weeks, 15 weeks, 18 weeks, 24 weeks, 36 weeks, or 52 weeks.

### Nucleic Acids/Vectors/Cells

The disclosure also features nucleic acids comprising nucleotide sequences that encode the immunostimulatory fusion molecules described herein. Further provided herein are vectors comprising the nucleotide sequences encoding an IFMs and the antibody molecule described herein. In one embodiment, the vectors comprise nucleotides encoding the IFMs and the antibody molecules described herein. In one embodiment, the vectors comprise the nucleotide sequences described herein. The vectors include, but are not limited to, a virus, plasmid, cosmid, lambda phage or a yeast artificial chromosome (YAC). Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity. Methods and conditions for culturing the resulting transfected cells and for recovering the antibody molecule produced are known to those skilled in the art, and may be varied or optimized depending upon the specific expression vector and mammalian host cell employed, based upon the present description.

In another aspect, the application features host cells and vectors containing the nucleic acids described herein. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

The disclosure also provides host cells comprising a nucleic acid encoding an antibody molecule as described herein. In one embodiment, the host cells are genetically engineered to comprise nucleic acids encoding the antibody molecule. In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter. The disclosure also provides host cells comprising the vectors described herein. The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Compositions

Compositions, including pharmaceutical compositions, comprising the immunostimulatory fusion molecules and/or protein nanogels are provided herein. A composition can be formulated in pharmaceutically-acceptable amounts and in pharmaceutically-acceptable compositions. The term “pharmaceutically acceptable” means a non-toxic material that does not interfere with the effectiveness of the biological activity of the active ingredients (e.g., biologically-active proteins of the nanoparticles). Such compositions may, in some embodiments, contain salts, buffering agents, preservatives, and optionally other therapeutic agents. Pharmaceutical compositions also may contain, in some embodiments, suitable preservatives. Pharmaceutical compositions may, in some embodiments, be presented in unit dosage form and may be prepared by any of the methods well-known in the art of pharmacy. Pharmaceutical compositions suitable for parenteral administration, in some embodiments, comprise a sterile aqueous or non-aqueous preparation of the nanoparticles, which is, in some embodiments, isotonic with the blood of the recipient subject. This preparation may be formulated according to known methods. A sterile injectable preparation also may be a sterile injectable solution or suspension in a non-toxic parenterally-acceptable diluent or solvent.

Additional compositions include modified cells, such as modified immune cells further comprising one or more tethered fusions proteins on their cell surface. This can be useful for ex vivo preparation of a cell therapy such as an adoptive cell therapy, CAR-T cell therapy, engineered TCR T cell therapy, a tumor infiltrating lymphocyte therapy, an antigen-trained T cell therapy, an enriched antigen-specific T cell therapy, or an NK cell therapy.

In some embodiments, the IFMs and/or nanogels of the present disclosure can be administered directly to a patient in need thereof, e.g., in the form of a nanoparticle or hydrogel or biogel, as agents for specific delivery of therapeutic proteins via receptor mediated binding of receptors unique to specific cells (e.g., CD4 or CD8). Such direct administration can be systemic (e.g., parenteral such as intravenous injection or infusion) or local (e.g., intratumoral, e.g., injection into the tumor microenvironment). The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

In some embodiments, the IFM and/or nanogels of the present disclosure can be used as ex vivo agents to induce activation and expansion of isolated autologous and allogenic cells prior to administration or reintroduction to a patient, via systemic or local administration. For example, the expanded cells can be used in T cell therapies including ACT (adoptive cell transfer) and also with other important immune cell types, including for example, B cells, tumor infiltrating lymphocytes, NK cells, antigen-specific CD8 T cells, T cells genetically engineered to express chimeric antigen receptors (CARs) or CAR-T cells, T cells genetically engineered to express T-cell receptors specific to an tumor antigen, tumor infiltrating lymphocytes (TILs), and/or antigen-trained T cells (e.g., T cells that have been “trained” by antigen presenting cells (APCs) displaying antigens of interest, e.g. tumor associated antigens (TAA)).

### Therapeutic Uses and Methods

The methods and compositions disclosed here have numerous therapeutic utilities, including, e.g., the treatment of cancers and infectious diseases. The present disclosure provides, inter alia, methods for inducing an immune response in a subject with a cancer in order to treat the subject having cancer. Exemplary methods comprise administering to the subject a therapeutically effective amount of any of the immunostimulatory fusion molecules described herein, wherein the IFM has been selected and designed to increase the cell surface availability of a cytokine and consequently potentiate its signaling.

Methods described herein include treating a cancer in a subject by using an IFM, e.g., an IFM and/or a nanoparticle comprising the IFM as described herein, e.g., using a pharmaceutical composition described herein. Also provided are methods for reducing or ameliorating a symptom of a cancer in a subject, as well as methods for inhibiting the growth of a cancer and/or killing one or more cancer cells. In embodiments, the methods described herein decrease the size of a tumor and/or decrease the number of cancer cells in a subject administered with a described herein or a pharmaceutical composition described herein.

In embodiments, the cancer is a hematological cancer. In embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In embodiments, the immunostimulatory fusion molecules and/or protein nanogels (or pharmaceutical compositions thereof) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or immunostimulatory fusion molecules) to be administered can be determined by a physician with consideration of individual differences in tumor size, extent of infection or metastasis, age, weight, and condition of the subject. In embodiments, the pharmaceutical composition described herein can be administered at a dosage of 10⁴ to 10⁹cells/kg body weight, e.g., 10⁵ to 10⁶ cells/kg body weight, including all integer values within those ranges. In embodiments, the pharmaceutical composition described herein can be administered multiple times at these dosages. In embodiments, the pharmaceutical composition described herein can be administered using infusion techniques described in immunotherapy (see, e.g., Rosenberg et al., New Eng. J. of Med. 319:1676, 1988).

In embodiments, the immunostimulatory fusion molecules and/or protein nanogels, or pharmaceutical composition thereof is administered to the subject parenterally. In embodiments, the cells are administered to the subject intravenously, subcutaneously, intratumorally, intranodally, intramuscularly, intradermally, or intraperitoneally. In embodiments, the cells are administered, e.g., injected, directly into a tumor or lymph node. In embodiments, the cells are administered as an infusion (e.g., as described in Rosenberg et al., New Eng. J. of Med. 319:1676, 1988) or an intravenous push. In embodiments, the cells are administered as an injectable depot formulation.

In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Further Combinations

The combination of a tethered fusion and nanogel disclosed herein can be used in further combinations with one or more therapeutic agents or procedure.

In some embodiments, the combination of a tethered fusion and nanogel is administered in combination with radiotherapy.

In some embodiments, the combination of a tethered fusion and nanogel is administered in conjunction with a cell therapy, e.g., a cell therapy chosen from an adoptive cell therapy, CAR-T cell therapy, engineered TCR T cell therapy, a tumor infiltrating lymphocyte therapy, an antigen-trained T cell therapy, or an enriched antigen-specific T cell therapy.

In embodiments, the combination of a tethered fusion and nanogel and the addition therapeutic agent or procedure are administered/performed after a subject has been diagnosed with a cancer, e.g., before the cancer has been eliminated from the subject. In embodiments, the combination of a tethered fusion and nanogel and the additional therapeutic agent or procedure are administered/performed simultaneously or concurrently. For example, the delivery of one treatment is still occurring when the delivery of the second commences, e.g., there is an overlap in administration of the treatments. In other embodiments, the combination of a tethered fusion and nanogel and the additional therapeutic agent or procedure are administered/performed sequentially. For example, the delivery of one treatment ceases before the delivery of the other treatment begins.

In embodiments, further combination therapy can lead to more effective treatment than the combination of a tethered fusion and nanogel or a monotherapy with either agent alone. In embodiments, the further combination of more effective (e.g., leads to a greater reduction in symptoms and/or cancer cells) than the combination of a tethered fusion and nanogel or the further combination alone. In embodiments, the further combination therapy permits use of a lower dose of the tethered fusion, nanogel and/or additional agent normally required to achieve similar effects when administered as a monotherapy. In embodiments, the further combination therapy has a partially additive effect, wholly additive effect, or greater than additive effect.

In one embodiment, the combination of a tethered fusion and nanogel is administered in a further combination with a therapy, e.g., a cancer therapy (e.g., one or more of anti-cancer agents, immunotherapy, photodynamic therapy (PDT), surgery and/or radiation). The terms “chemotherapeutic,” “chemotherapeutic agent,” and “anti-cancer agent” are used interchangeably herein. The administration of the combination of a tethered fusion and nanogel and the therapy, e.g., the cancer therapy, can be sequential (with or without overlap) or simultaneous. Administration of the combination of a tethered fusion and nanogel and the additional agent can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J. Clin. 61:250-281).

### Anti-Cancer Therapies

In other embodiments, the combination of a tethered fusion and nanogel is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the combination of a tethered fusion and nanogel is administered in conjunction with a biologic. Exemplary biologics include, e.g., HERCEPTIN® (trastuzumab); FASLODEX® (fulvestrant); ARIMIDEX® (anastrozole); Aromasin® (exemestane); FEMARA® (letrozole); NOLVADEX® (tamoxifen), AVASTIN® (bevacizumab); and ZEVALIN® (ibritumomab tiuxetan).

### EXAMPLES

### Example 1: Preparation of T Cells for ACT

T cells are isolated from healthy donors. One day old leukopack cells (Biospecialties Inc.) were diluted 1:1 in volume with DPBS and layered on a density cushion (Lymphoprep, Stemcell Tech.) in a 50 ml tube (35 ml of diluted leukopack on top of 15 ml of Lymphoprep). After 30 minutes centrifugation at 800 g, mononuclear cells were harvested at the interface between lymphoprep and DPBS. Cells are washed in 50 ml of DPBS 3 times to remove residual lymphoprep and cell debris. T cells are isolated by sequential magnetic beads sorting using anti-CD3 (or anti-CD8) and anti-CD56 conjugated beads (Miltenyi), respectively, according to the manufacturer's instructions. Briefly, LS columns are equilibrated with 3 ml of ice-cold DPBS while antibody-conjugated beads were incubated with mononuclear cells (30 minutes at +4° C.). After loading the cells in the column, 3 washes with 3 ml of ice-cold DPBS are performed and cells flushed out of the column with 5 ml of ice-cold DPBS.

After isolation, T cells are rested in complete media (CM-T): IMDM (Lonza), Glutamaxx (Life Tech), 20% FBS (Life Tech), 2.5 ug/ml human albumin (Octapharma), 0.5 ug/ml Inositol (Sigma) supplemented with 20 ng/ml of interleukin-2 (IL-2) for at least 2 hours.

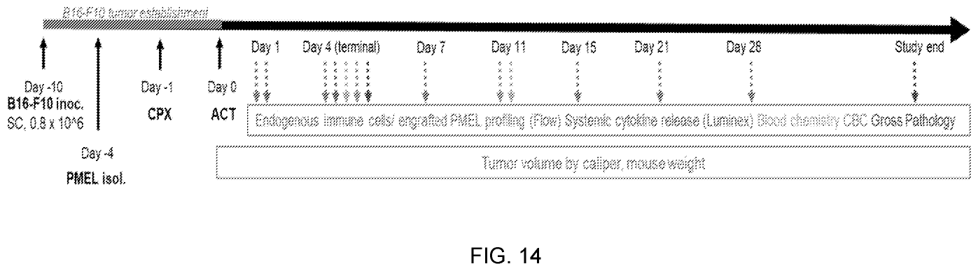

In some embodiments, the T cell is primed to improve or optimize T cell activation. As shown in FIG. 14B, pretreatment with IL-21 showed the most improvement in ACT efficacy, followed by IL-2/IL-7 and IL-7.

### APC Preparation In Vitro

Antigen-presenting cells (APCs), e.g., dendritic cells (DCs) can be prepared in vitro using the methods disclosed herein, as well as those disclosed in PCT Publication No. WO2020/055931, incorporated herein by reference in its entirety. First, moDCs can be generated in vitro from peripheral blood mononuclear cells (PBMCs). Plating of PBMCs in a tissue culture flask permits adherence of monocytes. Treatment of these monocytes with interleukin 4 (IL-4) and granulocyte-macrophage colony stimulating factor (GM-CSF) leads to differentiation to iDCs. Subsequent treatment with tumor necrosis factor (TNF), IL6, IL1,13, and PGE2 further differentiates the iDCs into mDCs.

Monocytes, iDCs and the cells prior to becoming mature Des can be contacted with preselected antigens to be presented on their surface. This can be done in vitro using, in some embodiments, the preloading process disclosed herein. As used herein, preloading refers to a process where monocytes and/or immature DCs are induced to internalize and proteolytically process the peptides into shorter fragments before loading onto major histocompatibility complex (MHC) I and MHC IL Without wishing to be bound by theory, it is believed that most peptides loaded using the preloading process are 8mer-11mer in length (compared to standard initial peptides of 15mer). In contrast, the conventional process refers to the loading of TAA peptides onto previously matured DCs, and is an extracellular method that briefly (typically for 1-3 hr) pulses DCs with peptide with the goal of loading peptides directly onto MHC I and MHC 11 at their original length without intracellular processing. This size difference between peptides loaded using preloading vs. conventional process is significant, because peptides that are presented in tumor MHC I are mostly shorter than 15mer (typically 8-10mer). As such. CD8+ CTLs that are trained by the conventional (i.e. extracellular loading) method using 15mer cannot be expected to bind tumor peptide:MHC due to intrinsic biophysical differences between loading of short (8-10mer) and long (15mer) peptides. Preloading uses intracellular processing of peptides to present peptides that are MHC I allele-specific and thus, can result in a more robust stimulation of a physiologically relevant CTL repertoire that can bind tumor peptide:MHC better and more effectively. Furthermore, using preloading, cells can “customize” the peptide via proteolysis (which may be different across patients), so that the most biologically preferred peptides are loaded regardless of MHC allele. In various embodiments, disclosed herein is a combination composition (mixture of conventionally loaded and preloaded DCs) and methods for making and using the same.

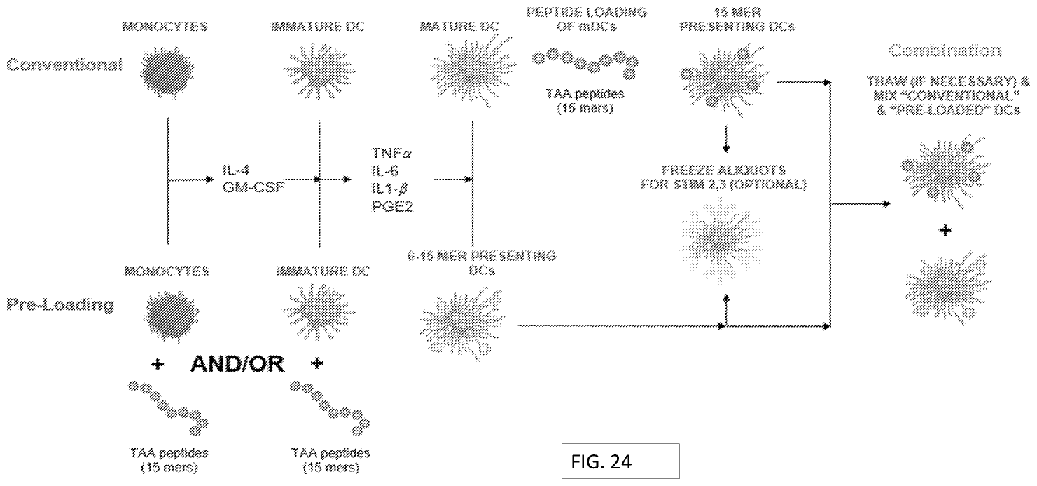

In some embodiments, an APC preparation method of the present disclosure can include the following steps (FIG. 24):

providing a plurality of monocytes;

culturing a first aliquot of the monocytes in a first culture medium comprising cytokines (e.g., IL-4 and GMCSF), thereby inducing differentiation of at least a portion of the first aliquot of monocytes into immature dendritic cells (DCs);

delivering to the monocytes and/or immature DCs a plurality of peptides (e.g., 15 raters) derived from one or more tumor-associated antigens (TAAs) (“TAA peptides”), e.g., by incubation with the TAA peptides, whole TAA protein, or via peptide-conjugated liposomal delivery;

continuing to culture the monocytes and/or immature DCs into a first plurality of mature DCs that present on their surfaces 6-15 mer peptide antigens, preferably 8-11 mer peptide antigens;

culturing a second aliquot of the monocytes and/or a plurality of immature DCs in a second culture medium, thereby inducing differentiation into mature DCs;

loading onto the mature DCs a plurality of the TAA peptides, thereby obtaining a second plurality of mature DCs that present on their surfaces the TAA peptides (e.g., 15 mer peptides); and combining the first plurality of mature DCs and the second plurality of mature DCs at a ratio of about 10:1 to 1:10 (e.g., about 5:1 to 1:5, or about 1:1), thereby generating APCs suitable for downstream uses T cell training).

In some embodiments, the monocytes can be acquired by elutriating PBMCs into at least a lymphocyte-rich fraction and a monocyte-rich fraction, wherein preferably the PBMCs are from a cancer patient in need of cell therapy.

In some embodiments, the peptides can include full-length TAAs and/or TAA fragments. The peptides can be a library of peptides obtained or derived from various TAAs. They can have a length of 8-15 amino acids (8-15mers). The TAAs can be, e.g., selected from PRAME, SSX2, NY-ESO-1, Survivin, and WT-1. In certain embodiments, the TAAs are obtained from the cancer patient in need of treatment in certain embodiments, the TAAs can include viral tumor antigens for HPV⁺ head & neck cancer and/or cervical cancer.

The resulting APCs can display on their cell surface 8-10mer antigens presented by major histocompatibility complex (MHC) I, wherein the 8-10mers are created from antigens and/or peptides that are proteolytically processed by the monocytes and/or iDCs from the peptides.

### MTC Preparation In Vitro

In various embodiments, the APCs prepared in accordance with the methods disclosed herein can be used to expand multi-targeted T cells (MTCs) in vitro. This can be done by, e.g., co-culturing the lymphocyte-rich fraction of the PBMCs with the APCs at a ratio between about 40:1 to about 1:1) to expand MTCs that are reactive to the TAA peptides. Such co-culturing can proceed in the presence of one or more of IL-2, IL-6, IL-7, IL-12, IL-15 and IL-21. In some embodiments, co-culturing can be in the presence of IL-15, IL-12 and optionally one or more of IL-2, IL-21, IL-7 and IL-6, Advantageously, using methods and compositions disclosed herein, the entire process time from PBMCs to MTCs can be shortened to 10-20 days, whereas conventional methods typically require at least 20 days (see, e.g., Putz et al., Methods Mol Med. 2005; 109:71-82, incorporated herein by reference in its entirety). The resulting MTCs can be used in various T-cell therapies as further disclosed herein.

### Cytokine Molecules

The expanded MTCs can be loaded with clusters of cross-linked therapeutic protein monomers (e.g., nanogels) to provide additional therapeutic benefits. Examples of therapeutic protein monomers include, without limitation, antibodies (e.g., IgG, Fab, mixed Fe and Fab), single chain antibodies, antibody fragments, engineered, proteins such as Fe fusions, enzymes, co-factors, receptors, ligands, transcription factors and other regulatory factors, cytokines, chemokines, human serum albumin, and the like. These proteins may or may not be naturally occurring. Other proteins are contemplated and may be used in accordance with the disclosure. Any of the proteins can be reversibly modified through cross-linking to form a cluster or nanogel structure as disclosed in, e.g., U.S. Publication No. 2017/0080104, U.S. Pat. No. 9,603,944, U.S Publication No. 2.014/0081012, PCT Application No, PCT/US17/37249 filed Jun. 13, 2017, and U.S. Provisional Application No. 62/657,218 filed Apr. 13, 2018, all incorporated herein by reference in their entirety. Loaded cells can have many therapeutic applications. For example, loaded MTCs can be used in T cell therapies including adoptive cell therapy.

The therapeutic protein monomers can include one or more cytokine molecules. In embodiments, the cytokine molecule is full length, a fragment or a variant of a cytokine, e.g., a cytokine comprising one or more mutations. In some embodiments the cytokine molecule comprises a cytokine chosen from interleukin-1 alpha (IL-1 alpha), interleukin-1 beta (IL-1_beta), interleukin-2 interleukin-4 interleukin-5 (IL-5), interleukin-6 (IL-6), interleukin-7 (IL-7), interleukin-12 (IL-12), interleukin-15 (IL-15), interleukin-17 (IL-17), interleukin-18 (IL-18), interleukin-21 (IL-21), interleukin-23 (IL-23), interferon (IFN) alpha, IFN beta, IFN gamma, tumor necrosis alpha, GM-CSF, GCSF, or a fragment or variant thereof, or a combination of any of the aforesaid cytokines. In other embodiments, the cytokine molecule is chosen from interleukin-2 interleukin-7 (IL-7), interleukin-12 (IL-12), interleukin-15 interleukin-18 (IL-18), interleukin-21 (IL-21), interleukin-23 (IL-23) or interferon gamma, or a fragment or variant thereof, or a combination of any of the aforesaid cytokines. The cytokine molecule can be a monomer or a dimer.

In embodiments, the cytokine molecule further comprises a receptor domain, e.g., a cytokine receptor domain. In one embodiment, the cytokine molecule comprises an IL-15 receptor, or a fragment thereof (e.g., an extracellular IL-15 binding, domain of an IL-15 receptor alpha) as described herein. In some embodiments, the cytokine molecule is an IL-15 molecule, e.g., IL-15 or an IL-15 superagonist as described herein. As used herein, a “superagonist” form of a cytokine molecule shows increased activity, e.g., by at least 10%, 20%, 30%, compared to the naturally-occurring cytokine. An exemplary superagonist is an IL-15 SA. In some embodiments, the IL-15 SA comprises a complex of IL-15 and an IL-15 binding, fragment of an IL-15 receptor, e.g., IL-15 receptor alpha or an IL-15 binding fragment thereof.

In other embodiments, the cytokine molecule further comprises an antibody molecule, e.g., an immunoglobulin Fab or scFv fragment, a Fab fragment, a FAB2 fragment, or an affibody fragment or derivative, e.g., a sdAb (nanobody) fragment, a heavy chain antibody fragment, e.g., an Fe region, single-domain antibody, a bi-specific or multispecific antibody). In one embodiment, the cytokine molecule further comprises an immunoglobulin Fe or a Fab.

In some embodiments, the cytokine molecule is an IL-2 molecule, e.g., IL-2 or IL-2-Fc. In other embodiments, a cytokine agonist can be used in the methods and compositions disclosed herein. In embodiments, the cytokine agonist is an agonist of a cytokine receptor, e.g., an antibody molecule (e.g., an agonistic antibody) to a cytokine receptor, that elicits at least one activity of a naturally-occurring cytokine. In embodiments, the cytokine agonist is an agonist of a cytokine receptor, e.g., an antibody molecule (e.g., an agonistic antibody) to a cytokine receptor chosen from an IL-15Ra or IL-21R.

To identify MTC reactive to smaller processed peptides in the product, the harvested MTC were incubated with commercially-sourced HLA-A*02:01, fluorophore-conjugated tetramers loaded with selected PRAME-derived 9mer and 10mer peptides. MTC bearing TCR reactive to the 9mer and 10mer-loaded tetramers are identified through flow cytometry based on tetramer staining (FIG. 25). The MART1₂₆₋₃₅₍₂₇₁₎ tetramer is used as a negative control for non-specific binding to tetramer. The study shows that clones that are reactive to immunologically significant 9 and 10mer can be isolated from off-the-shelf, highly diversified pools of peptides.

### Example 2: Exemplary Immunostimulatory Fusion Proteins Comprising IL-12

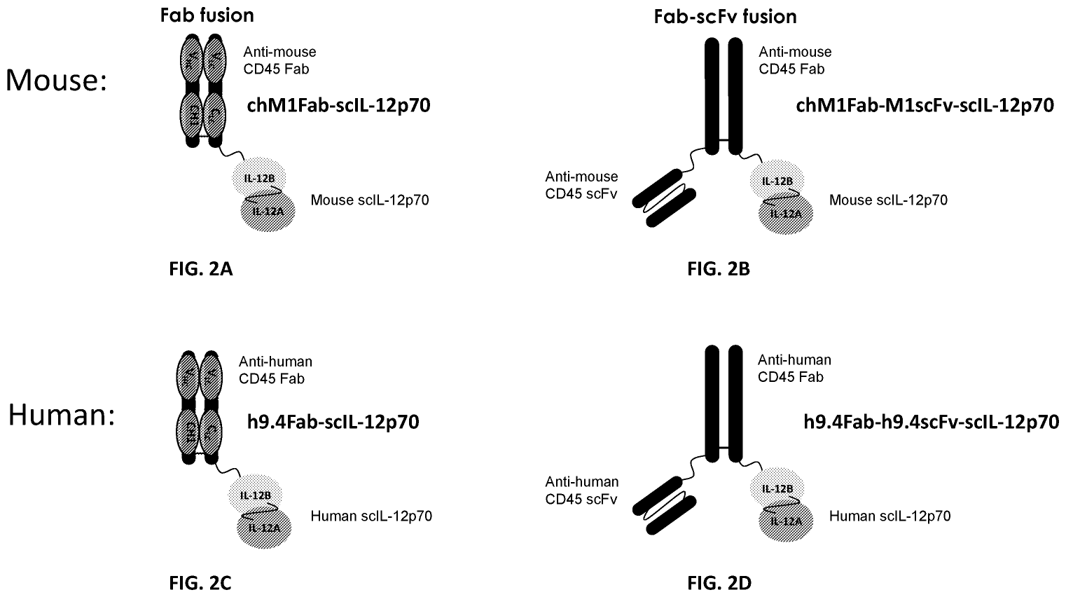

To explore the potential for fusion molecules of IL-12 and an immune-targeted antibody to improve IL-12 biological activity, IFMs comprising IL-12 and monoclonal antibodies are constructed, which target human CD45 an abundant receptor on the surface of immune cells (Cyster et al., EMBO Journal, Vol 10, no 4, 893-902, 1991). Exemplary IL-12 tethered fusions (IL12-TFs) are depicted in FIGS. 2A-2D. IL12-TFs for use on both human or mouse cells have been constructed using either human or mouse IL-12 and antibody fragments specific to either human or mouse CD45. The IL12-TF chM1Fab-sc-IL12p70 comprises an anti-mouse CD45 Fab fragment fused to the mouse single-chain IL-12p70 (FIG. 2A). Mouse single-chain IL-12p70 comprises a genetic fusion between mouse IL-12A and IL-12B. Another IL12-TF for use in mouse cells, chM1Fab-M1scFv-scIL-12p70, comprises a Fab-scFv fusion of anti-mouse CD45 Fab and scFv antibody fragments and a mouse single-chain IL-12p70 (FIG. 2B). Corresponding IL12-TFs for use with human cells have also been constructed: h9.4Fab-scIL-12p70 (FIG. 2C) and h9.4Fab-h9.4scFv-scIL-12p70 (FIG. 2D) comprise a Fab or Fab-scFv fusion specific for human CD45 and a single-chain human IL-12p70. The respective IL-12p70 subunits IL-12A and IL-12B for all four constructs are expressed as a single-chain molecule with the orientation IL-12B-IL-12A, although the converse expression orientation is also possible (e.g. IL-12A-IL-12B). Multiple different flexible linkers joining the IL-12A and IL-12B subunits are possible. IL-12p70 can also be expressed as a heterodimer of IL-12A and IL-12B, which is the natural form of the protein. Various linkers disclosed herein can be used to operably link the anti-CD45 antibody and IL-12, which act to add space therebetween.

### Example 3: Antibody-Mediated Tethering of IL-12 to CD45 Supports Cell Loading of IL-12 and Prolonged Surface Persistence

The ability of IL12-TFs to support the loading of IL-12 onto T cells was evaluated. Briefly, human total CD3 T cells were activated with CD3/CD28 Dynabeads for three days. Beads were removed and cells were incubated with IL-2 for 1 day prior to pulse incubation with h9.4Fab-scIL-12p70 diluted in full medium (RPMI 1640 with 10% FBS). Cells were incubated in biological duplicate with full media (Mock condition) or h9.4Fab-scIL-12p70 for 1 hr at 37° C. and then washed three times will full media (RPMI 1640 with 10% FBS) to remove unbound IL12-TF. Cells were then plated in full medium at a cell density of approximately 200,000 cells/mL and incubated at 37° C., 5% CO2. Surface tethered IL-12 was detected using flow cytometry by immunostaining with a polyclonal anti-human IgG antibody. Cells were counted using CountBright Absolute flow cytometry counting beads. In each case cells were analyzed on a FACSCelesta using Diva Software; data was analyzed using Cytobank.

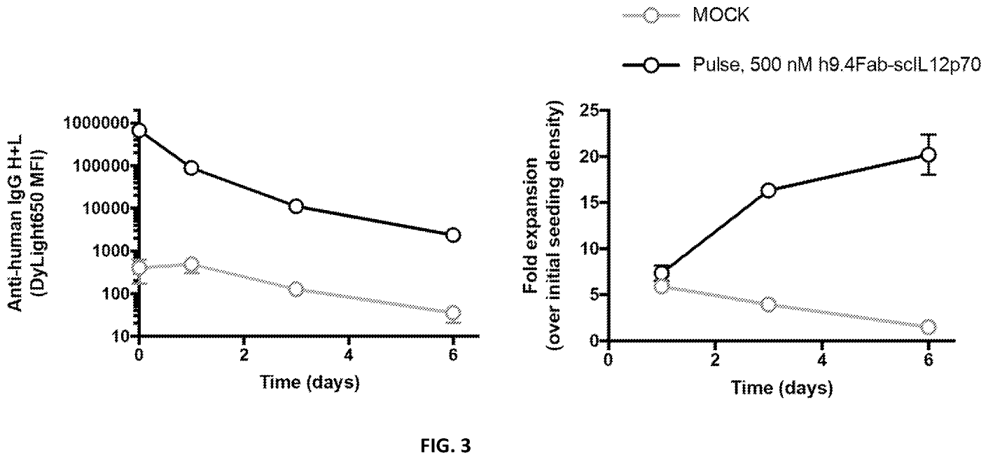

As shown in FIG. 3, pulse incubation of IL-12 fused to an anti-CD45 antibody supports not only significant loading and prolonged persistence of IL-12 on the T cell surface, but also significant T cell expansion.

### Example 4: Activation of STAT4 Phosphorylation in Non-Loaded Target Cells by IL-12 Tethered Fusion

A tethered fusion can activate both the loaded cell and non-loaded target cells (FIG. 4A). As such, an IL-12 tethered fusion was then evaluated for its ability to support activity in human T cells in cis/autocrine, trans, and paracrine manner. Briefly, STAT4 phosphorylation was measured in three separate assays one day after pulse incubation with an IL-12 tethered fusion (h9.4Fab-scIL-12p70) to probe cis, trans, and paracrine activity. Total CD3 T cells were activated as described herein. The activated human T cells were incubated with an IL12-TF (h9.4Fab-scIL-12p70) for 1 hr at 37° C., unbound tethered fusion was removed by washing and cells were seeded at a density of 4E5 cells/mL and incubated overnight at 37° C. and 5% CO2. Non-loaded cells were propagated in full media for an additional day in the absence of cytokine, and were used on the following day as “target” cells for the trans and paracrine assays. For cis-presentation/autocrine activity cells were fixed, permeabilized and immunostained for STAT4 phosphorylation as described above. For trans-presentation evaluation, non-IL12-TF-loaded target cells were labeled with CellTrace Far Red dye (ThermoFisher) in order allow differentiation from IL12-TF-loaded cells using flow cytometry. IL12-TF-loaded cells were mixed with the fluorescently labeled non-loaded cells, pelleted and incubated together for 30 min. Cells were then fixed, permeabilized and immunostained for STAT4 phosphorylation. For transfer/paracrine conditioned media from IL12-TF-loaded cells was recovered one day following pulse incubation and transferred to non-loaded cells, incubated for 30 min, and then fixed permeabilized and immunostained for STAT4 phosphorylation. In all assays cells were analyzed on a FACSCelesta flow cytometer using DiVa software, and data was analyzed using Cytobank. For all assays the IL12-TF induces STAT4 phosphorylation above the background of “mock” pulsed cells, which were pulsed with media not containing a tethered fusion (FIG. 4B). IL-15 has been reported to augment the activity of IL-12 and as shown above, combinations of IL-15 and IL-12 tethered fusions can augment STAT4 phosphorylation; therefore also evaluated was the STAT4 phosphorylation for combined pulse incubation with IL-12 (h9.4Fab-scIL-12p70) and IL-15 (h9.4Fab-IL-15/sushi) tethered fusions in the three assays described here. While the IL-15 tethered fusion did not induce strong STAT4 phosphorylation on its own, STAT4 phosphorylation was augmented by combination of IL-12 and IL-15 tethered fusions in the cis and transferred assays, as shown in FIG. 4B.

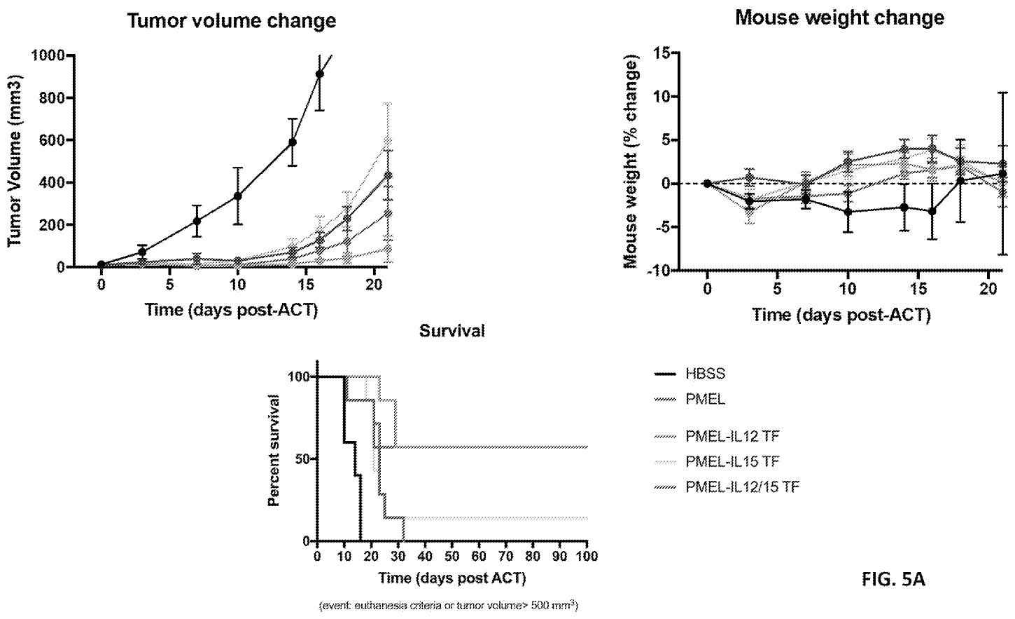

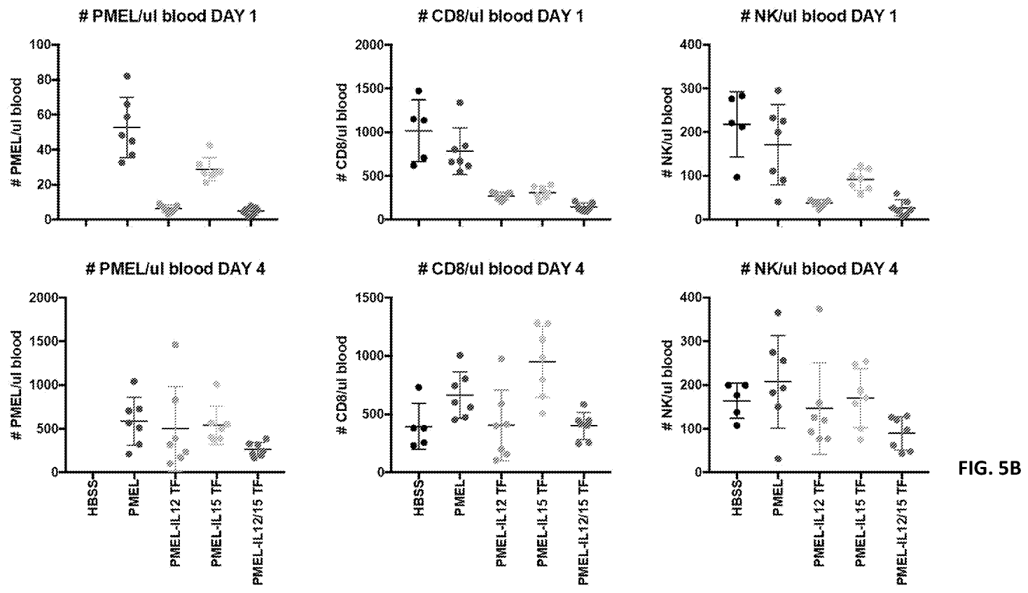

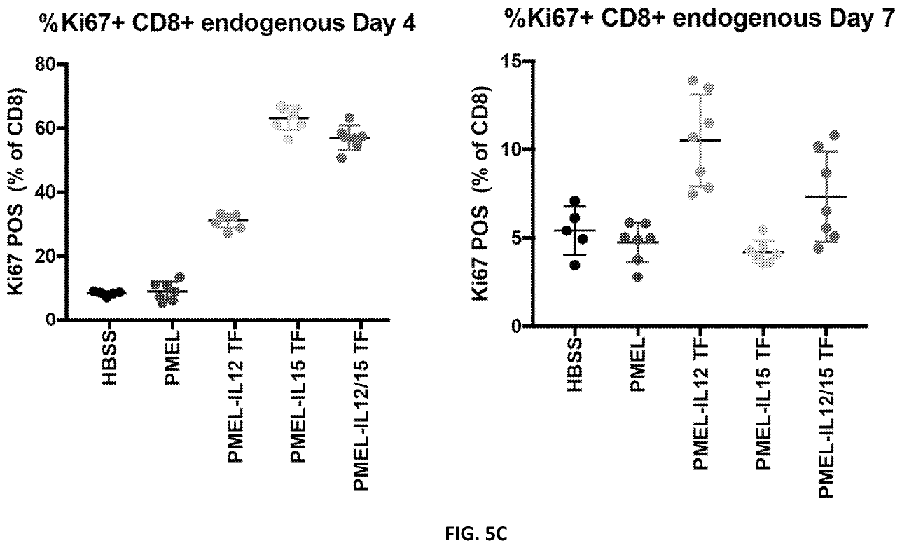

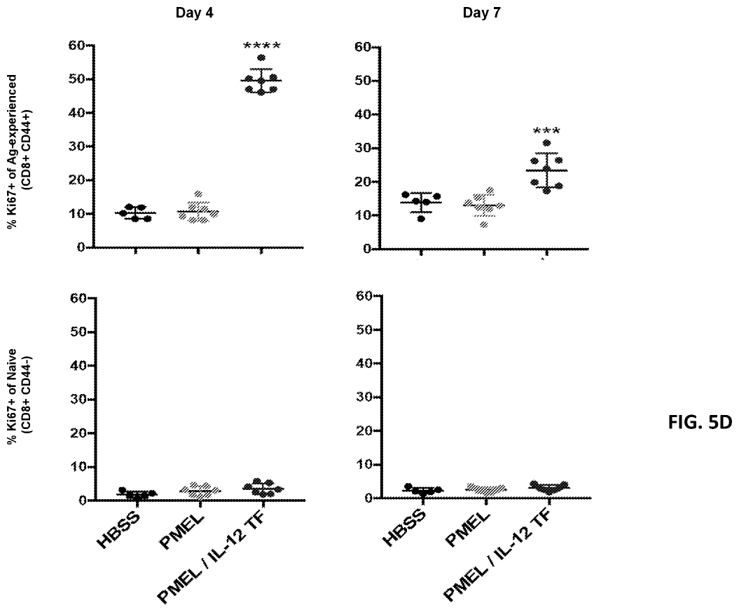

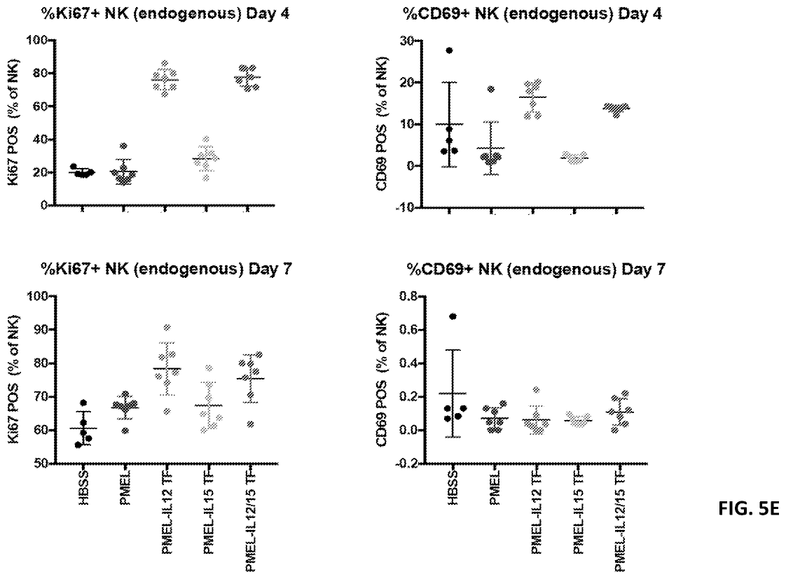

Pmel cells carrying a mouse IL12-TF show signs of activity towards the endogenous immune system. They induce transient lymphopenia of transferred and endogenous immune cells including CD8 T cells and NK cells (FIG. 5B). This is followed by proliferation (as defined by Ki67 positivity) and differentiation of endogenous CD8 T cells (FIG. 5C). Further subdivision of the endogenous CD8 T cells reveals that the proliferating cells are almost exclusively encompassed within the antigen-experienced endogenous CD8 T cell population (by flow cytometry, populations are both negative for the congenic Pmel T cell marker CD90.1 and double-positive for CD8 and CD44), suggesting that the presence of the IL12-TF is activating a specific compartment of the endogenous immune system (FIG. 5D). The transient lymphopenia of endogenous NK cells shown in FIG. 5B is followed by their increased proliferation (via Ki67 positivity) and activation (via CD69 positivity) as shown in FIG. 5E. In conclusion, that tumor-specific T cells carrying IL12-TFs hold the potential to both augment ACT for cancer and prime the endogenous immune system.

### Example 5: IL12-TF Augments Tumor-Specific T Cell Therapy when Either Pre-Loaded onto Adoptively Transferred T Cells or when Solubly Co-Administered

Surface tethered IL-12 was evaluated for the ability to augment adoptive cell therapy (ACT) for cancer. Briefly, C57BL/6J mice were innoculated intradermally with 400,000 B16-F10 melanoma cells. One day prior to adoptive cell therapy with tumor-specific T cells (9 days after inoculation with B16-F10 cells) tumor-bearing mice were treated with 4 mg cyclophosphamide. Separately, CD8 T cells were isolated from Pmel-1 mice, which express a T cell receptor specific for the gp100 antigen in B16-F10 melanoma cells, and activated and expanded as described for T cells herein. Cells were then harvested for ACT and incubated with an IL-12 tethered fusion (chM1Fab-scIL-12p70) at a concentration of 125 nM. Unbound tethered fusion was removed by washing, cells were resuspended in HBSS, and the CD8 Pmel T cells were then adoptively transferred (3E6 cells/mouse) by intravenous (i.v.) injection into the B16-F10 tumor-bearing mice. As controls, mice were also treated with HBSS, CD8 Pmel T cells alone, or the CD8 Pmel T cells followed by a single dose of soluble IL-12p70 (at dose levels of 10, 50, or 250 ng, which corresponds to 0.143, 0.715, and 3.575 pmoles of IL-12p70) or with soluble IL-12 tethered fusion (0.143, 0.715, or 3.575 pmoles of tethered fusion), which was administered intravenously for all conditions. Not wishing to be bound by theory, based on preliminary calculations herein, the highest dose tested so far corresponds to greater than 100-fold amounts of the surface-tethered IL-12 dose.

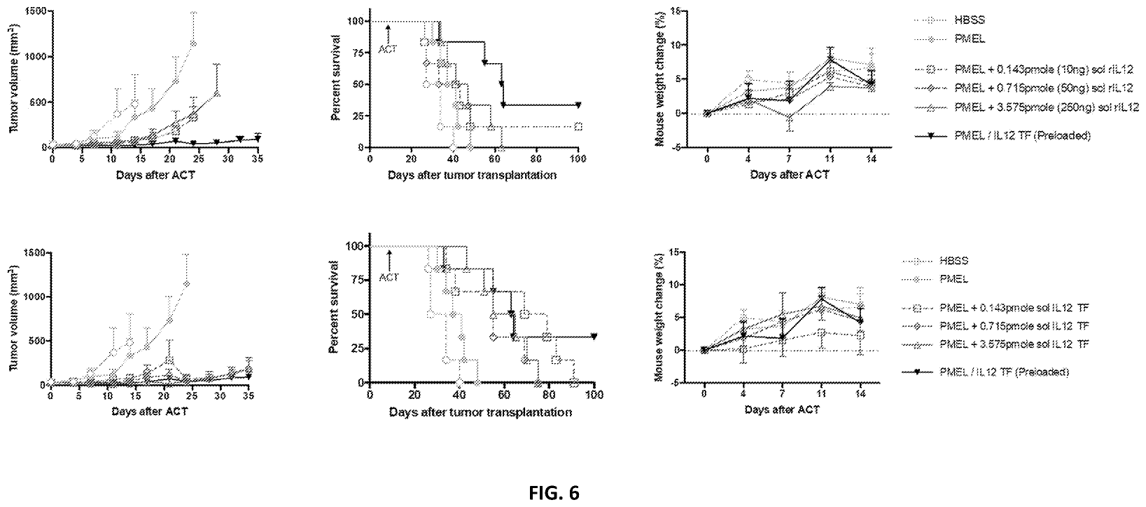

As shown in FIG. 6, both pre-loaded or solubly co-administered IL-12 tethered fusions significantly inhibited tumor growth and supported prolonged survival. In each case the tethered fusions more strongly inhibited tumor growth and prolonged survival than co-administration of free IL-12. Minimal overt toxicity in the form of body weight loss was observed. The data are plotted in two separate figures for clarity; in the second set of figures the HBSS, Pmel only, and Pmel carrying IL12-TF groups are replotted for comparison. Tumor growth kinetics are shown for the first 35 days after ACT or until two mice in a given group die.

### Example 6: IL12-TF Candidate Enables Further Improved Tumor Control with Multiple Cell Doses

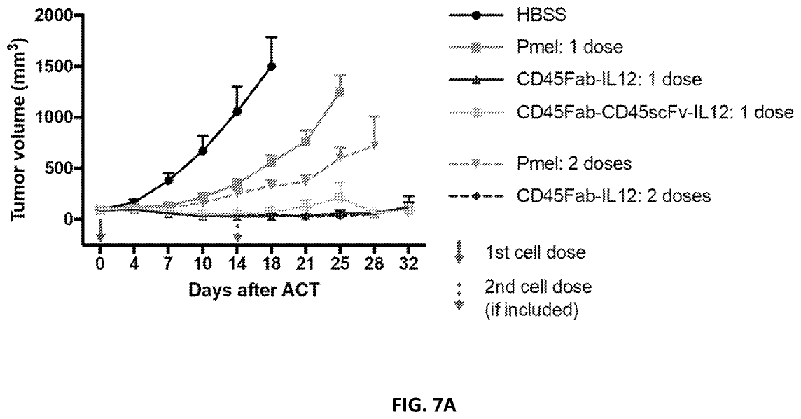

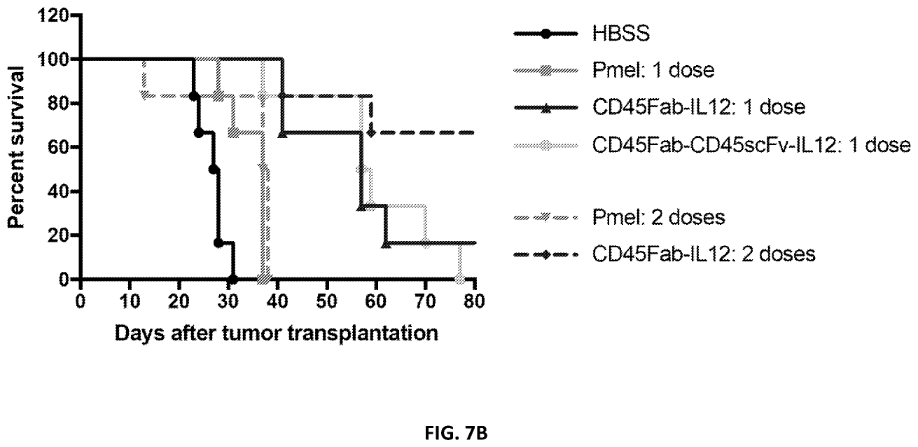

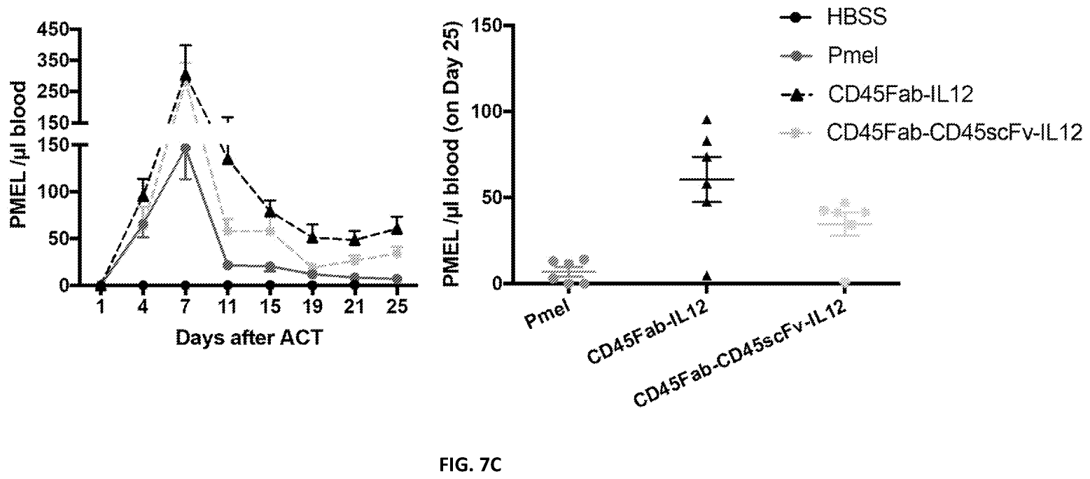

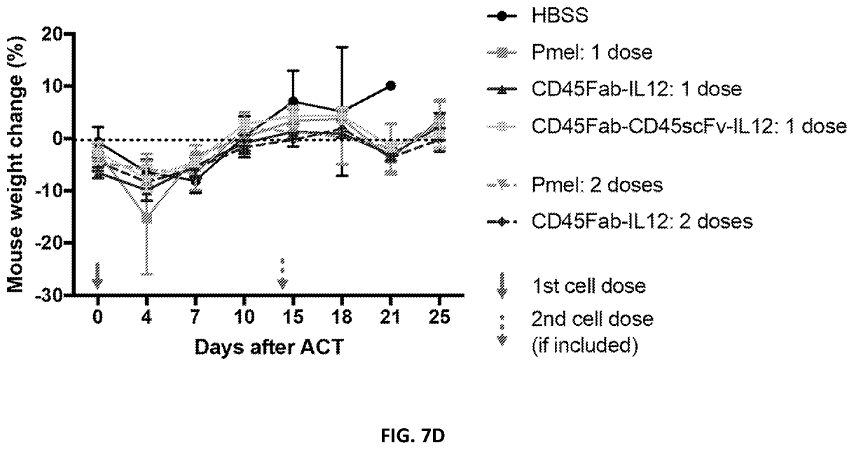

Most cell therapies require preconditioning regimens involving myeloablative chemotherapy prior to ACT for robust anti-tumor responses. This approach, however, has limitations including the inability to administer multiple cell doses due to risks of depleting the activity of previously administered cells by successive rounds of preconditioning chemotherapy. As demonstrated previously, the potential for the IL12-TF to augment tumor-specific T cell therapy in the absence of preconditioning in a solid tumor model. Next, the ability for IL12-TF to further augment anti-tumor control through the use of multiple cell doses was evaluated. Briefly, C57BL/6J mice were inoculated intradermally with 400,000 B16-F10 melanoma cells. Separately, CD8 T cells from Pmel mice were isolated, activated, expanded, and loaded with an IL12-TF (chM1Fab-scIL-12p70) as described herein. Nine days following tumor inoculation mice were treated with the CD8 Pmel T cells by i.v. injection. Lymphodepletion with cyclophosphamide was used one day prior to the first cell dose; the second cell dose was given 14 days after the first dose in the absence of additional lymphodepletion. The ability of an alternative configuration for the IL12-TF (chM1Fab-M1scFv-scIL-12p70) to augment efficacy of a single dose of tumor-specific cell therapy was additionally evaluated. Both of the IL12-TFs (chM1Fab-scIL-12p70 and chM1Fab-M1scFv-scIL-12p70) improved the tumor growth inhibition and survival with a single cell dose loaded ex vivo with the tethered fusions (FIG. 7A). Multiple doses of tumor-specific T cells loaded ex vivo with chM1Fab-scIL-12p70—but not multiple doses of the tumor-specific T cells alone—further augmented anti-tumor survival (FIG. 7B).

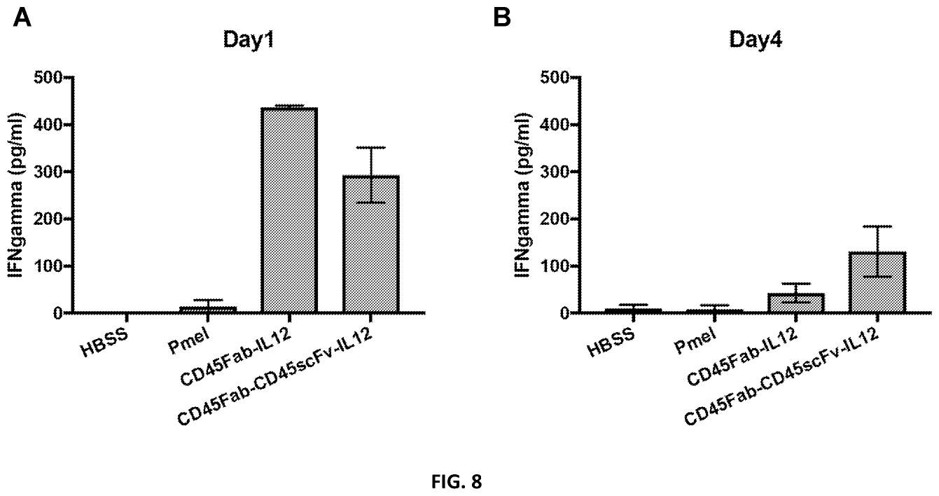

The IL12-TFs increased both the peak expansion of circulating Pmel T cells and the their long-term persistence (FIG. 7C). No signs of overt toxicity in the form of body weight loss was observed (FIG. 7D). Any observed body weight loss appeared to be predominantly driven by lymphodepletion with cyclophosphamide: mice lost approximately 10% body weight in each treatment group (FIG. 7D), while in a previous example, which was conducted in the absence of lymphodepletion, less than 5% body weight loss was observed across all treatment groups (FIG. 5A). In addition, modest levels of systemic IFN-γ (FIG. 8) and CXCL10 (FIG. 9) was observed in plasma one day after ACT with Pmel carrying the IL12-TF; circulating levels returned to baseline within four days of the adoptive cell transfer (FIGS. 8-9).

In summary, demonstrated herein are multiple configurations of antibody-mediated cytokine tethering that enable strong loading and persistence of IL-12 on the T cell surface. Furthermore, surface-tethered IL-12 substantially improves efficacy of adoptively transferred tumor-specific T cells in an aggressive solid tumor model, including better tumor control and survival than >100 fold molar excess of systemically administered IL-12. Efficacy of tumor-specific T cells loaded with an IL12-TF in the absence of lymphodepletion enabled further improved efficacy through administration of multiple cell doses.

Surface-tethered IL-12 also supports activation of the endogenous immune system—including increased proliferation of antigen-experienced CD8 T cells—with an absence of overt toxicities in the form of body weight loss and sustained systemic cytokine release.

In conclusion, cell surface tethered immunostimulatory cytokines are a powerful approach to augment the efficacy of cell therapy for cancer, including for solid tumors. This approach does not require genetic engineering and can be readily incorporated onto cell therapies that are currently under clinical exploration, such as CAR-T, TCR-T, tumor associated antigen-specific T cells, and NK cells.

### Example 7: Tethered Fusion Platform Enables Specific Cell Targeting In Vivo

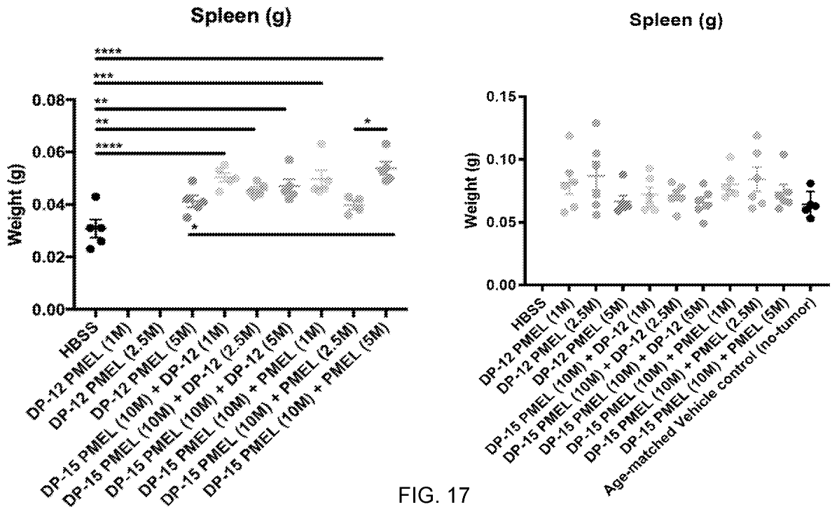

After establishing the ability for selective CD8 T cell loading in vitro using CD8-targeted IL-7 or IL-15 tethered fusions (see Examples above), selective targeting of CD8 T cells was tested in vivo using a CD8-targeted IL-15 IFM. Based on previous observations in human T cells demonstrating improved CD8 affinity using a bivalent Fab-scFv construct (FIG. 17D-FIG. 17F) a mouse CD8-targeted IL-15 variant comprising a similar Fab-scFv antibody configuration (chY169Fab-M1scFv-IL15/sushi) was generated. The CD8-targeting Fab is designed to provide specificity, while the CD45-targeting scFv improves persistence on the targeted cell.

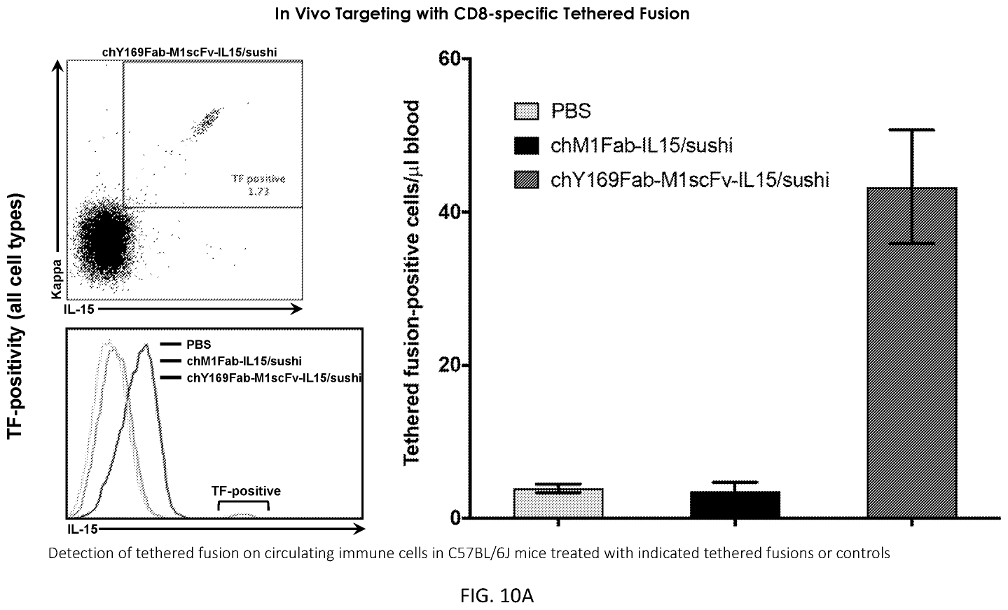

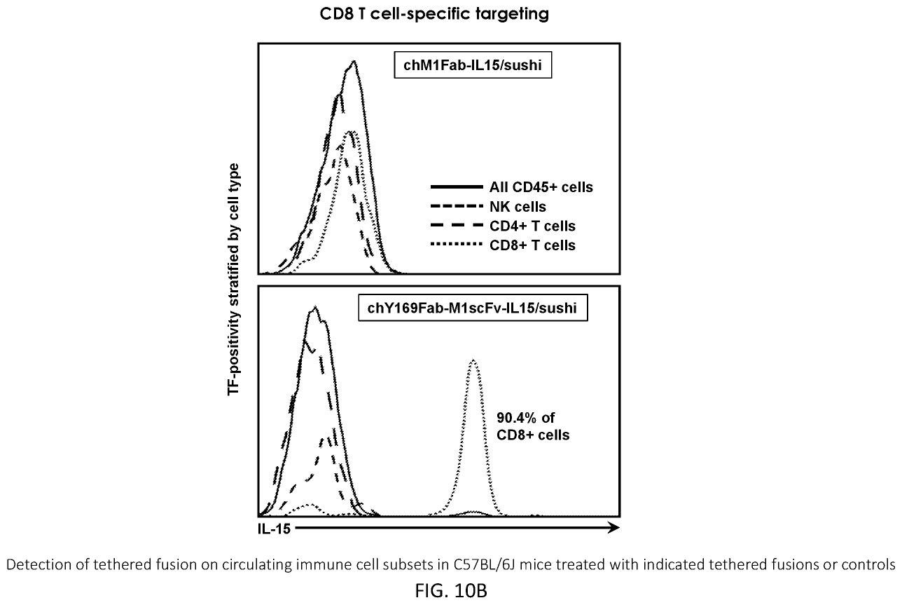

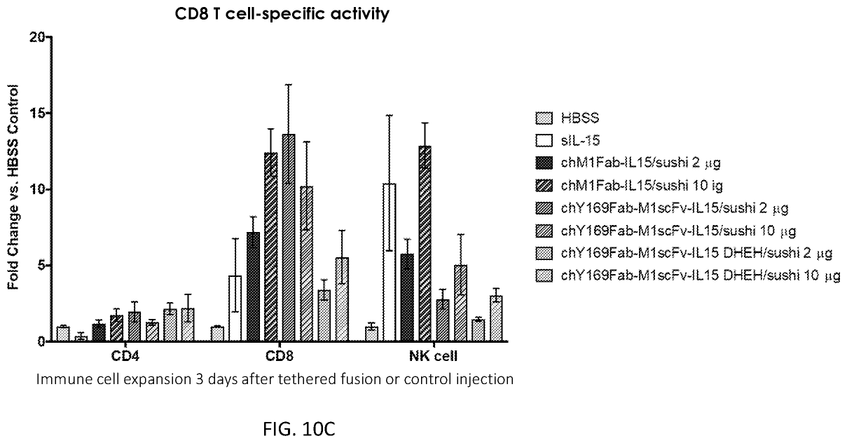

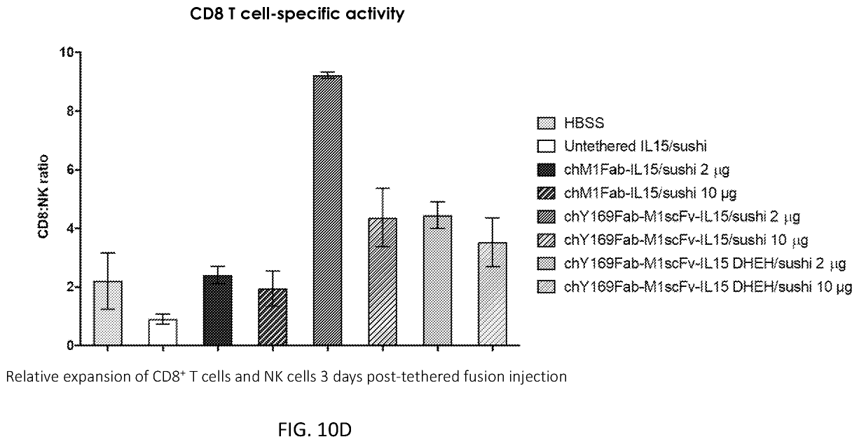

In the first experiment described in this example, whether or not intravenously administered tethered fusion could be specifically target on mouse CD8 T cells in vivo was tested. Two C57BL/6J mice/group were injected with chY169Fab-M1scFv-IL15/sushi (2 μg/mouse), chM1Fab-IL15/sushi (CD45-targeted IFM, 2 μg/mouse), or PBS vehicle control. One hour after injection, blood was drawn and tethered fusion cell surface binding was assessed by flow cytometry. Red blood cells were lysed, and remaining cells were stained with fluorescently conjugated antibodies against kappa and IL-15 for detection of tethered fusion. Antibodies specific for mouse CD4, CD8, NK1.1, and CD45 were additionally included to enable immune cell subset analysis. Tethered fusion surface binding was defined by positivity for both kappa and IL-15. Both the vehicle control and the chM1Fab-IL15/sushi-treated animals had minimal positive staining cells (3.5-3.9/μl), while the chY169Fab-M1scFv-IL15/sushi-treated animals had greater than 10-fold higher concentration of circulating tethered-fusion positive cells (FIG. 10A). The histogram plot in FIG. 10A shows a bulk shift in fluorescence for the chM1Fab-IL15/sushi-treated animals; however, there was not a distinct TF-positive population. Because CD45 is found on all immune cells, it is likely that there was specific binding, but the tethered fusion signal was spread out over a much larger number of cells. For the mice treated with the CD8-targeted IL-15 (chY169Fab-M1scFv-IL15/sushi), while only a small percentage of total cells were positive for tethered fusion (1.73%), the majority of CD8 T cells were positive (FIG. 10B). Furthermore, none of the other analyzed subsets (CD4 T cells, NK cells) exhibited TF-staining Together these data show specific, high-level targeting of CD8 cells by the chY169Fab-M1scFv-IL15/sushi tethered fusion, and non-specific, low-level targeting by the CD45-specific chM1Fab-IL15/sushi tethered fusion.

In a second experiment the effects of a single dose (2 or 10 ug) of CD45- or CD8-targeted IL-15 IFMs on circulating immune cells was evaluated. A non-targeted IL-15/sushi-Fc construct and a CD8-targeted IL-15 variant comprising D61H and E64H mutations (chY169Fab-M1scFv-IL15-DHEH/sushi was included; see Examples above for further description of these mutations). Blood was drawn on Day 3 post-injection, red blood cells were lysed, and remaining cells were stained with fluorescently conjugated antibodies directed against CD45, CD4, CD8 and NK1.1. By Day 3 post-injection there were minimal changes in CD4 T cell numbers for any of the tethered fusion formats or concentrations (FIG. 10C). In contrast, CD8 numbers were increased by all of the tethered fusion formats and concentrations with a range of 3.4-13.6-fold. The effects of the CD45- and CD8-targeting tethered fusions were similar on CD8 cells and were increased relative to non-targeted IL15/sushi-Fc. Corresponding with its reduced biological activity, the DHEH mutant drove less CD8 expansion than the wildtype IL-15 IFMs. While there was no enhancement of CD8 T cell expansion for the CD8-targeting tethered fusion relative to CD45, there were reduced off-target effects on NK cells (as measured by quantification of NK1.1+ cells in circulation). NK cells are also highly sensitive to IL-15, and their numbers were dramatically increased by the IL-15/sushi-Fc and the pan-CD45 targeting chM1Fab-IL15/sushi tethered fusion (6-13-fold expansion relative to HBSS treated group). In contrast, the CD8-targeting chY169Fab-M1scFv-IL15/sushi led to only modest increases (3-5-fold) in NK cell number. The DHEH mutant off-target effects on NK cells was even more attenuated with only 1.5-3-fold expansion relative to the vehicle control. These effects can be seen most clearly by comparing the CD8 T cell to NK cell ratio (FIG. 10D). The non-targeted IL15/sushi actually reduces the ratio of CD8 T cells to NK cells, suggesting that without targeting there is a preference for NK specific activity. While the pan-CD45—targeting tethered fusion increased CD8 T cell numbers significantly, it had comparable effects on NK cell numbers and the CD8:NK ratio is unchanged from vehicle treated mice. The wildtype CD8-targeting tethered fusion drove a substantial increase in the CD8:NK ratio with 9.2- and 4.4-fold increases for the 2 μg and 10 μg doses, respectively. The DHEH mutant did not have as dramatic effects on CD8 T cells, but it also had reduced off-target effects on NK cells, and mice treated with this construct had similar CD8:NK ratios as the wildtype CD8-targeting tethered fusion. In conclusion, IFM targeting can modulate the magnitude and selectivity of CD8 and NK cell effects of IL-15; in particular, IL-15 activity can be biased towards CD8 cells by controlling the targeting (CD8 vs CD45 vs non-targeted), the dose, and activity of IL-15 (via attenuating IL-15 mutations).

Together these experiments indicate that systemic administration of CD8-targeted IL-15 variants can load IL-15 onto CD8 T cells in vivo, can bias IL-15 activity towards these cells, and can further increase circulating levels of CD8 T cells beyond that attainable by treatment with IL-15 constructs described in the art, such as an IL15/sushi-Fc.

### Example 8: Systemic Administration of CD8-Targeted IL-15 Shows Reduced Toxicity

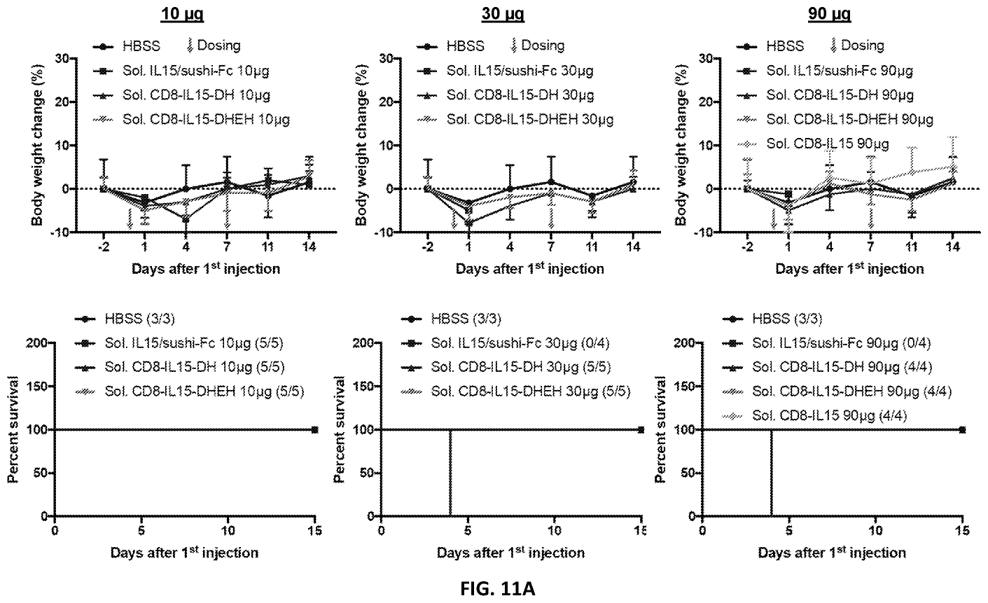

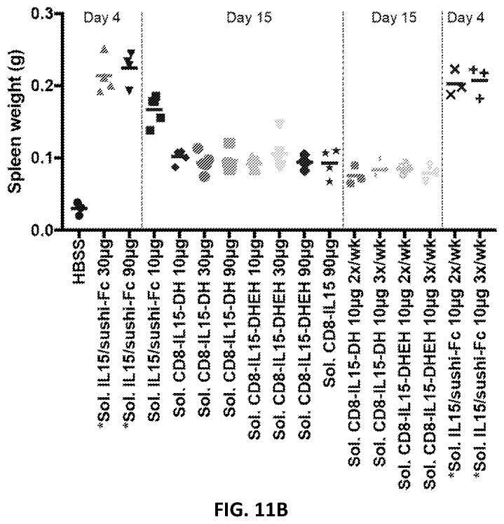

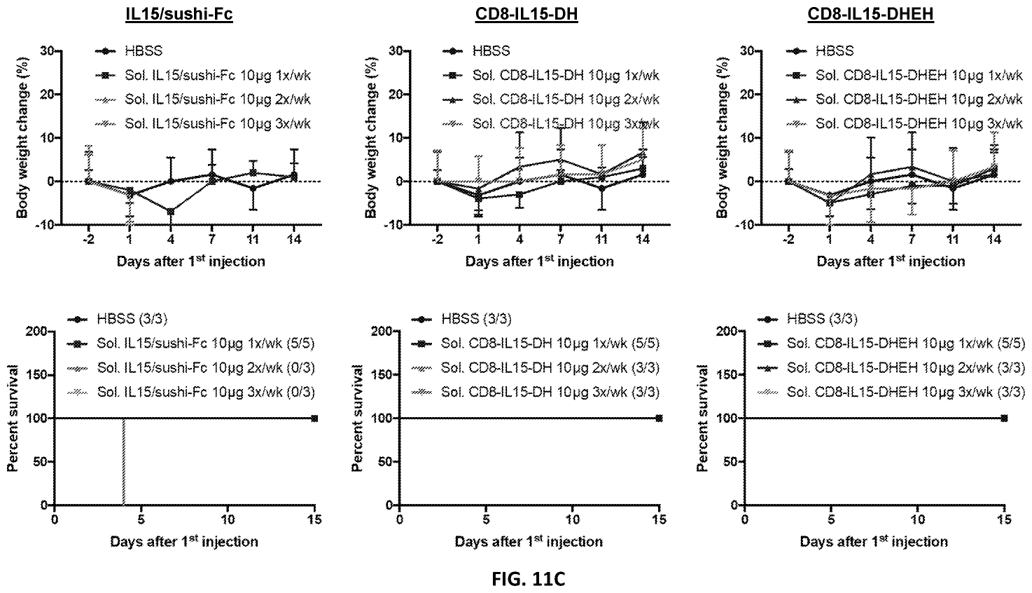

The effects of retargeting IL-15 to CD8 T cells on systemic toxicities was evaluated. Briefly, C57BL/6J mice were given two doses administered once per week by intravenous injection of 10, 30 or 90 μg with an IL15/sushi-Fc construct, which is an extended half-life form of IL-15, or two different CD8-targeted IL-15 variants containing a D61H (DH) or D61H and E64H (DHEH) mutations (chY169Fab-M1scFv-IL15-DH/sushi and chY169Fab-M1scFv-IL15-DHEH/sushi); n=5 mice per group. In addition, a CD8-targeted IFM containing wild-type IL-15, chY169Fab-M1scFv-IL15/sushi was evaluated at the 90 μg dose level. In the above Example, it was demonstrated that the CD8-targeted constructs bias the loading and activity of IL-15 towards CD8 T cells as compared with IL15/sushi-Fc or CD45-targeted construct, and at the 10 μg dose the CD8-targeted constructs induce expansion of circulating CD8 T cells as well or better than 10 μg IL15/sushi-Fc, while inducing lesser expansion of circulating NK cells. Increasing the number of doses per week at a fixed dose-level was also evaluated, in particular, two or three doses per week of 10 μg IL15/sushi-Fc or the CD8-targeted IL-15 IFMs for two weeks (n=3 mice per group) was investigated. FIG. 11A shows no overt toxicity in the form of body weight loss over time for the dose escalation of the CD8-targeted IL-15 variants. The IL15/sushi-Fc construct, however, had a maximum tolerated dose of 10 μg per week for the IL15/sushi-Fc: the 30 and 90 μg doses induced significant toxicity and resulted in death four days post-injection (FIG. 11A, numbers in parentheses in figure legends indicate fraction of surviving mice at the end of the experiment). Spleens were harvested from the dead mice and found splenomegaly in the mice treated with 30 and 90 μg IL15/sushi-Fc (FIG. 11B). By comparison, each of the CD8-targeted IL-15 variants were able to complete the full two-week study, resulted in minimal body weight loss (FIG. 11A), no deceased animals, and lesser spleen enlargement compared to 10 μg IL15/sushi-Fc following the full two-week dosing regimen examined here (FIG. 11B). Furthermore, while IL15/sushi-Fc was tolerated at a dose of 10 μg one-time per week, increasing this dosing to two or three doses per week resulted in significant toxicity and death after second injection (FIG. 11C, numbers in parentheses in figure legends indicate fraction of surviving mice at the end of the experiment). The deceased mice also exhibited splenomegaly (FIG. 11B). By comparison, dosing the CD8-targeted IL-15 variants two or three times per week at a dose of 10 μg did not result in significant body weight loss, deceased animals, (FIG. 11C) or substantial spleen enlargement (FIG. 11B) over the course of the full two-week dosing regimes. In conclusion, retargeting IL-15 to CD8 T cells enables lower toxicity and higher dosing of IL-15 in vivo. Expansion of NK cells by IL-15 has been shown to strongly contribute to IL-15 toxicity in vivo (Guo et al., J Immunol. 2015 Sep. 1; 195(5):2353-64). Without wishing to be bound by theory, it is reasoned that biasing activity of IL-15 away from NK cells in vivo can reduce toxicity and enable stronger dosing against CD8 T cells. This is therapeutically advantageous and significant, given that anti-tumor efficacy of IL-15 can be mediated by CD8 T cells (Xu et al., Cancer Res. 2013 May 15; 73(10):3075-86; Cheng et al., J Hepatol. 2014 December; 61(6):1297-303).

### Example 9: Anti-Cancer Efficacy from Systemic, CD8 Targeted Administration of IL-12

IL-12 is a potent cytokine that induces strong anti-tumor activity in murine tumor models, but has suffered from high toxicity in human clinical trials. IL-12 supports differentiation of CD4 T cells into a Th1 phenotype, increases cytotoxicity of CD8 T cells, and activates NK cells. Clinical trials of IL-12 for cancer therapy, however, have found that effects of IL-12 in human patients has been most prominent on NK cells (Robertson et al., Clin Cancer Res. 1999 January; 5(1):9-16; Bekaii-Saab et al., Mol Cancer Ther. 2009 November; 8(11): 2983-2991). It is possible that the dominant activity of IL-12 on NK cells—coupled with toxicity associated with activating NK cells—limits the ability to effectively deliver biological effects of IL-12 to CD4 and CD8 T cells. To test this hypothesis, a mouse IL-12 IFM targeted to mouse CD8 T cells (chY169Fab-M1scFv-scIL-12p70) was constructed and evaluated its safety and anti-tumor efficacy in a murine melanoma tumor model.

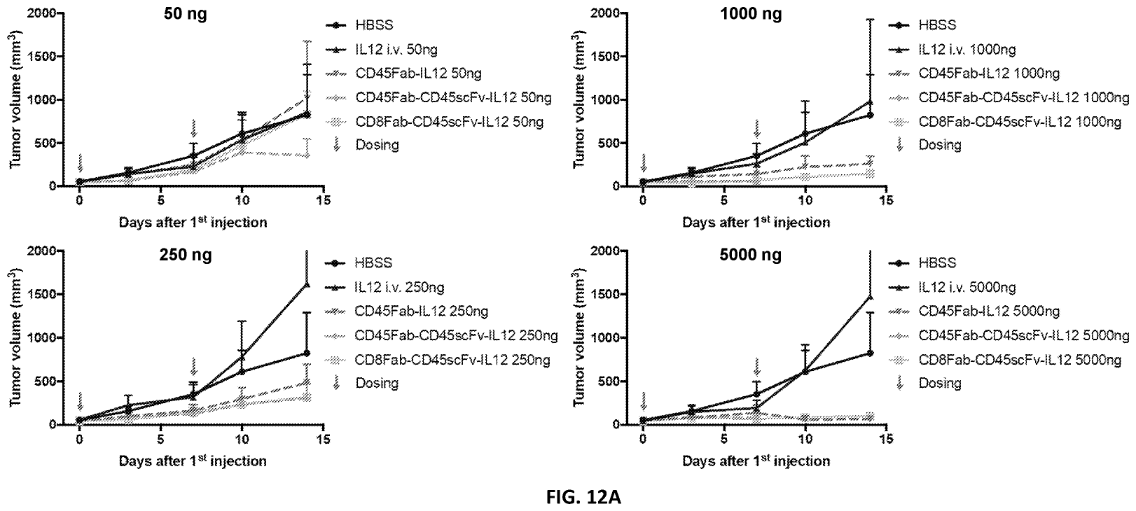

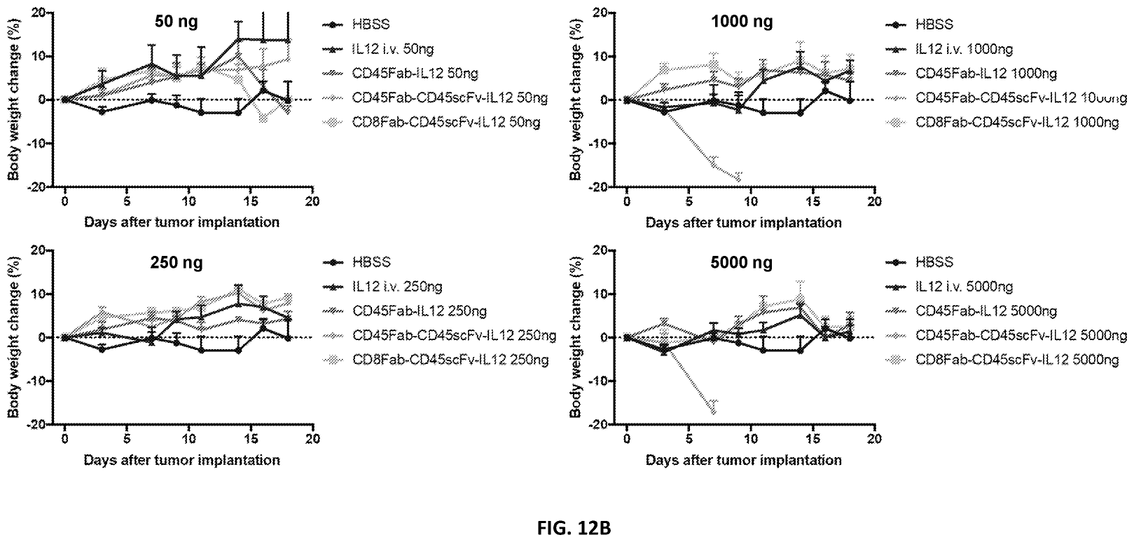

Briefly, B6D2F1/J mice were inoculated by intradermal injection with 400,000 B16-F10 melanoma cells. After 10 days tumor-bearing mice were randomized and treated with two doses (once weekly dosing) of 0.05, 0.25, 1, or 5 μg of recombinant IL-12 (R&D Systems), CD45-targeted IL-12 (chM1Fab-M1scFv-scIL12p70 and chM1Fab-scIL12p70), or CD8-targeted IL-12 by intravenous injection (n=5 mice per group). FIG. 12A demonstrates that weekly injection of the CD45-targeted IL-12 IFMs or the CD8-targeted IL-12 IFM each delivered stronger anti-tumor efficacy than weekly injection of IL-12. The CD8-targeted IL-12 additionally delivered similar tumor growth inhibition as the CD45-targeted IL-12 (FIG. 12A). Notably, mice treated with the Fab-scFv CD45-targeted IL-12 construct suffered toxicity in the form of body weight loss at the 1 and 5 ug dose levels, while mice treated with the Fab-scFv CD8-targeted IL-12 did not exhibit similar toxicities (FIG. 12B). In conclusion, IFMs comprising IL-12 can deliver improved anti-tumor efficacy as compared with IL-12 alone, and that cell-specifically-targeted IL-12 can reduce systemic toxicities.

### Example 10: IL-12 Tethered Fusions

In one aspect, a tethered fusion protein useful in the invention comprises a single chain human IL-12p70 tethered to an anti-CD45 Fab and, optionally, additional comprising an anti-CD45 scFV. The Fab and scFV regions function to target the IL-12 tethered fusion to T cells, particularly normal or functional T cells. The IL-12 TFs can be loaded onto T cells ex vivo for use in adoptive cell therapy or administered systemically to bind to T cells (and other immune cells) in vivo. With either mode of administration, the IL-12 TFs exhibit both autocrine and paracrine activity and have shown to stimulate an immune response.

Two embodiments of the IL-12 TF described herein are depicted in FIGS. 2C-2D.

### IL-12 Tethered Fusion Description

Protein Name: h9.4Fab-scIL-12p70

This protein was made by co-expression of two subunits: HC-h9.4Fab (SEQ ID NO: 79) and LC-h9.4Fab-scIL-12p70 (SEQ ID NO: 82). The resulting protein comprises a fusion of a single-chain human IL-12p70 to the C-terminus of h9.4 Fab fragment light-chain using Linker-1 (SEQ ID NO: 36). The h9.4 Fab is an anti-human CD45R antibody Fab fragment comprising variable-heavy and variable-light chain domains (VH and VL) from h9.4 and constant domains from human (human constant kappa domain and human IgG1-CH1 domain).

Protein Name: h9.4Fab-h9.4scFv-scIL-12p70

This protein was made by co-expression of two subunits: HC-h9.4Fab-h9.4scFv (SEQ ID NO: 80) and LC-h9.4Fab-scIL-12p70 (SEQ ID NO: 82). The resulting protein comprises a fusion of a single-chain human IL-12p70 to the C-terminus of h9.4 Fab fragment light-chain using Linker-1 (SEQ ID NO: 36) and a fusion of an h9.4 scFv to the h9.4 Fab fragment heavy-chain using Linker-1.

### IL-12 Tethered Fusion Sequences

A. SEQ ID NO: 36: Linker-1 (L1) (G₄S)₃ linker

GGGGSGGGGSGGGGS

B. SEQ ID NO: 50: scIL-12p70-BA

Synthetic sequence; IL-12B and IL-12A joined by flexible linker.

IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTDILKDQKE
PKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FCVQVQGKSKREKKDRVFTDKTSATVICRKNASISVRAQDRYYSSSWSEW
ASVPCSGGGSGGGSGGGSGGGSRNLPVATPDPGMFPCLHHSQNLLRAVSN
MLQKARQTLEFYPCTSEEIDHEDITKDKTSTVEACLPLELTKNESCLNSR
ETSFITNGSCLASRKTSFMMALCLSSIYEDLKMYQVEFKTMNAKLLMDPK
RQIFLDQNMLAVIDELMQALNFNSETVPQKSSLEEPDFYKTKIKLCILLH
AFRIRAVTIDRVMSYLNAS

C. SEQ ID NO: 70: Linker-5 (L5) (G₃S)₄ linker

GGGSGGGSGGGSGGGS

D. SEQ ID NO: 79: HC-h9.4Fab

Heavy-chain of a humanized anti-CD45 antibody; contains humanized 9.4 (h9.4) heavy-chain variable domain and the CH1 domain from human IgG1.

EVQLVQSGAEVKKPGASVKVSCKASGYTFTSYSIQWVRQAPGQRLEWIGY
INPSSGYIKYNQHFRGRATLTADRSASTAYMELSSLRSEDTAVYYCARGN
SGSFDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEP KSC

E. SEQ ID NO: 80: HC-h9.4Fab-h9.4scFv

Heavy-chain of a humanized anti-CD45 antibody linked to a humanized anti-CD45 scFv; contains variable domain from h9.4 heavy-chain and the CH1 domain from human IgG1. An h9.4 scFv is genetically fused to the Fab heavy chain C-terminus using a flexible linker (Linker-1, SEQ ID NO: 36).

EVQLVQSGAEVKKPGASVKVSCKASGYTFTSYSIQWVRQAPGQRLEWIGY
INPSSGYIKYNQHFRGRATLTADRSASTAYMELSSLRSEDTAVYYCARGN
SGSFDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCGGGGSGGGGSGGGGSEVQLVQSGAEVKKPG
ASVKVSCKASGYTFTSYSIQWVRQAPGQRLEWIGYINPSSGYIKYNQHFR
GRATLTADRSASTAYMELSSLRSEDTAVYYCARGNSGSFDYWGQGTLVTV
SSGGGGSGGGGSGGGGSGGGGSDIVMTQSPLSLPVTPGEPASISCRSSQS
LLHSSGITYLYWFLQKPGQSPQLLIYRMSNLASGVPDRFSGSGSGTDFTL
KISRVEAEDVGVYYCMQHLEYPFTFGQGTKLEIK

F. SEQ ID NO: 82: LC-h9.4Fab-scIL-12p70

Light-chain of a humanized anti-CD45 antibody; contains variable domain from h9.4 light-chain and human constant kappa domain, a wild-type single-chain human IL-12p70 (SEQ ID NO: 50) genetically fused to antibody light-chain C-terminus using a flexible linker (Linker-1, SEQ ID NO: 36); single-chain human IL-12p70 comprises a genetic fusion of human IL-12A and IL-12B using a flexible linker (Linker-5; SEQ ID NO: 80).

DIVMTQSPLSLPVTPGEPASISCRSSQSLLHSSGITYLYWFLQKPGQSPQ
LLIYRMSNLASGVPDRFSGSGSGTDFTLKISRVEAEDVGVYYCMQHLEYP
FTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGECGGGGSGGGGSGGGGSIWELKKDVYVVELDWY
PDAPGEMVVLTCDTPEEDGITWTLDQSSEVLGSGKTLTIQVKEFGDAGQY
TCHKGGEVLSHSLLLLHKKEDGIWSTDILKDQKEPKNKTFLRCEAKNYSG
RFTCWWLTTISTDLTFSVKSSRGSSDPQGVTCGAATLSAERVRGDNKEYE
YSVECQEDSACPAAEESLPIEVMVDAVHKLKYENYTSSFFIRDIIKPDPP
KNLQLKPLKNSRQVEVSWEYPDTWSTPHSYFSLTFCVQVQGKSKREKKDR
VFTDKTSATVICRKNASISVRAQDRYYSSSWSEWASVPCSGGGSGGGSGG
GSGGGSRNLPVATPDPGMFPCLHHSQNLLRAVSNMLQKARQTLEFYPCTS
EEIDHEDITKDKTSTVEACLPLELTKNESCLNSRETSFITNGSCLASRKT
SFMMALCLSSIYEDLKMYQVEFKTMNAKLLMDPKRQIFLDQNMLAVIDEL
MQALNFNSETVPQKSSLEEPDFYKTKIKLCILLHAFRIRAVTIDRVMSYL
NAS

### Example 11: IL-15 Nanogels

In an embodiment, an IL-15 nanogel comprises IL-15-Fc monomers, a degradable chemical crosslinker, and a cationic block copolymer. The IL-15 nanogels are minimally biologically active when formed into nanogels but are active upon release of the IL-15-Fc monomers resulting from crosslinker degradation in vivo.

More particularly, IL-15 nanogels comprise crosslinked IL-15-Fc monomers coated with a cationic block copolymer of PEG-polylysine (PK30) to promote cell adhesion. The key elements of the IL-15 nanogels include (1) IL-15-Fc monomers, (2) a degradable chemical crosslinker, and (3) a cationic block copolymer consisting of PEG-polylysine (referred to as PK30).

### IL-15-Fc Monomer

According to an embodiment, the IL-15-Fc monomer is a sushi-Fc fusion homodimer protein with two associated IL-15 proteins. The primary sequence for the IL-15 protein is a wild type human IL-15. The Sushi-Fc protein is a fusion of the sushi domain of wild type IL-15 receptor subunit alpha (IL-15R□) to the N-terminus of a modified IgG2 Fc protein. The primary sequence for the Fc region is composed of the CH2 and CH3 hinge regions from human IgG2 with IgG4 mutations (PAPIEK-IgG2 mutated to PSSIEK-IgG4) to minimize Fc gamma receptor and complement mediated effector function. Two sushi domains are fused to each Fc protein. One IL-15 non-covalently binds to each sushi domain due to high affinity ionic and hydrophobic interactions. The IL-15-Fc monomers are manufactured from CHO cells with the IL-15 and sushi-Fc proteins each coded on a plasmid under a separate promoter.

### Crosslinker

In one embodiment, the CL17 crosslinker [bis(2-((((2,5-dioxopyrrolidin-1-yl)oxy)carbonyl)oxy)ethyl) succinate] is a bifunctional crosslinking reagent (shown below). The crosslinker has multiple reactive sites which serve two separate purposes. The N-hydroxysuccinimide carbonate groups at each end of the crosslinker reacts with amines to join IL-15-Fc monomers while the ester group at the center of the molecule can be cleaved by hydrolysis. More specifically, during nanogel synthesis, the activated carbonate groups on the crosslinker react with free lysines on IL-15-Fc to form carbamate linkages resulting in crosslinking of monomers into multimers. IL-15 nanogels are stable in solution at physiological pH. However, in vivo, the ester group will be subject to hydrolysis resulting in release of thiol modified IL-15-Fc monomer. The pendant thiol still attached to lysine on IL-15-Fc will undergo a fast intramolecular cyclization liberating intact native protein with concomitant formation of 1-3, oxathiolon-2-one. As such, the crosslinker and residual groups are self-eliminating, the crosslinker completely disassociates leaving IL-15-Fc monomer in the same state as it was prior to the nanogel formation.

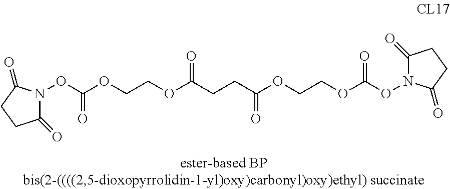 [SMILES: O=C(CCC(=O)OCCOC(=O)ON1C(=O)CCC1=O)OCCOC(=O)ON1C(=O)CCC1=O]

### Block Copolymer PK30

Due to the reaction of cationic lysine residues, the crosslinking of IL-15-Fc with the CL17 crosslinker results in a net negative charge of the resulting IL-15 nanogels which inhibit cell attachment. In a first step, nanogels are complexed with PK30 (Methoxy-poly(ethylene glycol)n-block-poly(L-lysine hydrochloride), PEG-polylysine, as shown below) via electrostatic interactions to drive cell attachment. PK30 is a linear amphiphilic block copolymer which has a poly(L-lysine hydrochloride) block and a non-reactive PEG block. The block copolymer contains approximately 114 PEG units (MW approximately 5000 Da) and 30 lysine units (MW approximately 4900 Da). The poly-L-lysine block provides a net cationic charge at physiological pH and renders the nanogel with a net positive charge after association.

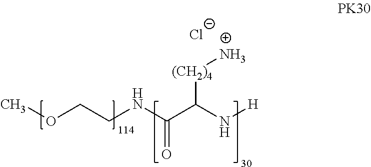

### Example 12: Formation of Protein Nanogel with Polycationic Polymer on Surface

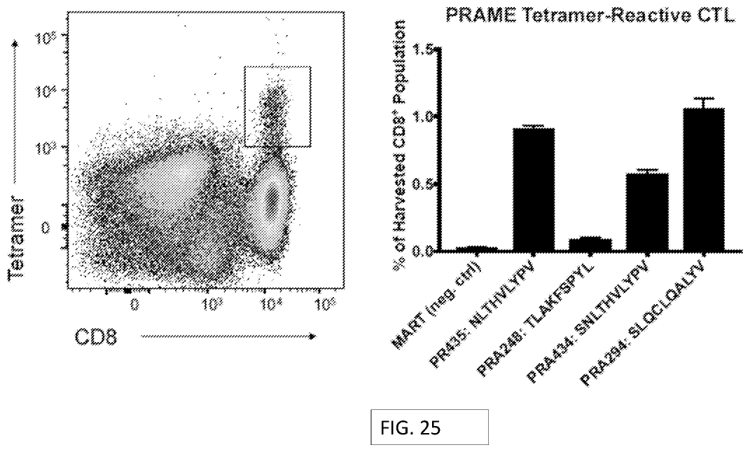

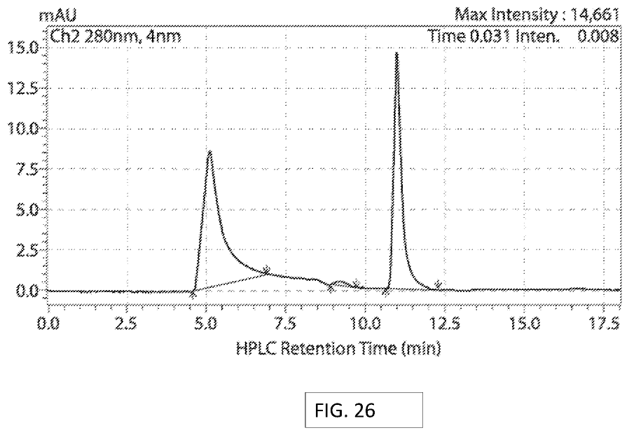

Protein nanogels comprising a protein nanogel with cationic polymer surface are formed as follows. IL-15^{WT}/sushi-Fc at a concentration of 15 mg/mL are cross-linked into protein nanoparticles using 25-fold molar excess of the degradable crosslinker specified in Formula IV. After 30 min incubation at room temperature the reaction is diluted 10-fold with DPBS to a final cytokine concentration of 1.5 mg/mL. Protein nanogels are then purified from linker leaving groups (which comprise molecular fragments of the linker that are removed as part of the cross-linking reaction) and unreacted linker by buffer exchange into DPBS using a Zeba column (7,000 or 40,000 MW cut-off, available from Thermo-Fisher). Zeba columns are used according to the manufacturer's instructions, including equilibrating the column in DPBS by three consecutive washes with DPBS to facilitate buffer exchange, followed by application of the reaction products. Buffer-exchanged protein nanogels at a cytokine concentration of approximately 1-1.5 mg/mL are then conjugated with a polyethylene glycol-polylysine (PEG-polyK) block copolymer: PEG5k-polyK30 (Alamanda Polymers cat. no. 050-KC030), which is a block co-polymer comprising 5 kiloDalton (kD) polyethylene glycol (PEG5k) and a 30 amino acid polylysine polymer (polylysine30 or polyK30), or PEG5k-polyK200 (Alamanda Polymers ca. no. 050-KC200). PEG5k-polyK30 or PEG5k-polyK200 are reconstituted to 10 mg/mL in DPBS and added to protein nanogels at a final block copolymer concentration of 50 ug/mL and incubated at room temperature for 30 min. Size and polydispersity of surface functionalized nanoparticles are analyzed by dynamic light scattering (DLS) at 90 degrees angle on a NanoBrook Omni particle sizer (NanoBrook Instruments Corp.), Relative conversion to nanoparticle are evaluated by size-exclusion chromatography using a BioSep™ SEC-s4000 column (Phenomenex Inc.) on a Prominence HPLC system with PBS (pH 7.2) as eluent (flow rate 0.5 mL/min) equipped with a photodiode array (Shimadzu Corp.), see FIG. 26.

The final IL-15 nanogels are diluted with an equal volume of Hank's Balanced Salt Solution (HBSS) to a final concentration of approximately 0.5-0.75 mg/mL for use in downstream assays such as association with activated primary T cells.

### Example 13: Association of Protein Nanogel to T Cell and Cryopreservation

Protein nanogels are associated with activated human T cells. Briefly, IL-15^{WT}/sushi-Fc protein nanogels surface functionalized with a polycationic polymer (PEG5k-polyK30) are prepared as described in Example 12. To support downstream flow cytometric analysis, the nanogels are generated using 3 mass % Alexa-647-labeled IL-15^{WT}/sushi-Fc and 97 mass % unlabeled IL-15^{WT}/sushi-Fc. IL-15^{WT}/sushi-Fc are fluorescently labeled using an Alexa-Fluor-647 labeling kit according to the manufacturer's instructions (ThermoFisher, cat. no. A20186, 100 ug scale kit; or cat. no. A20173, 1 mg scale kit). All other steps for protein nanogel synthesis were performed as described in Example 12.

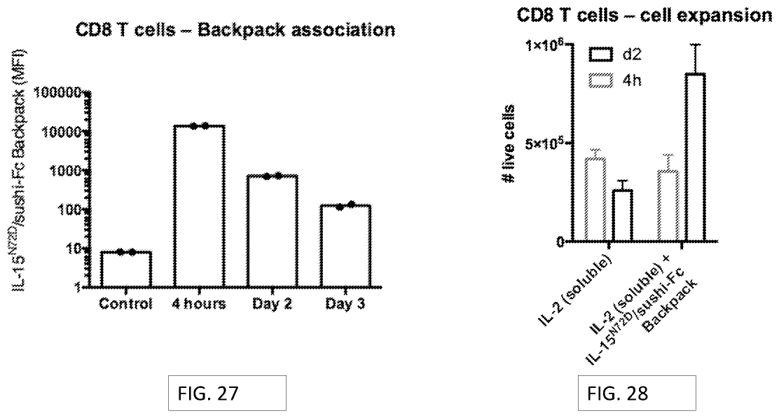

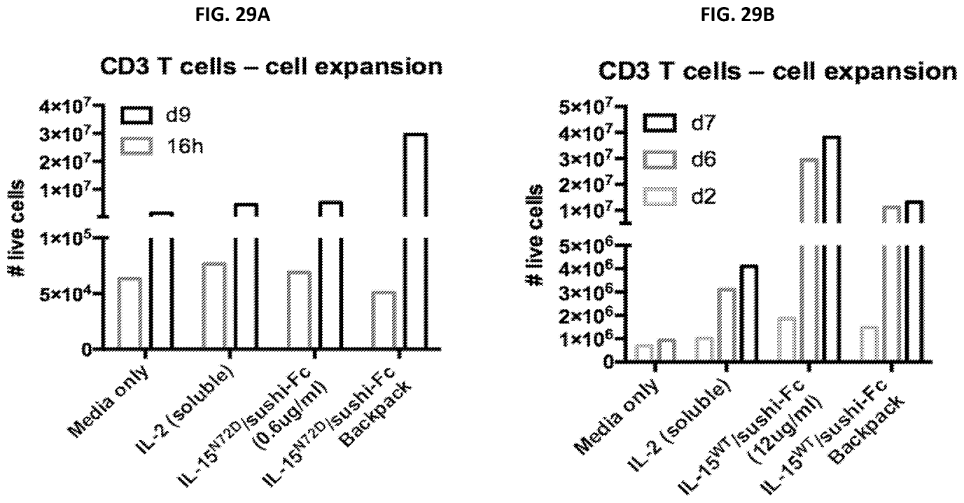

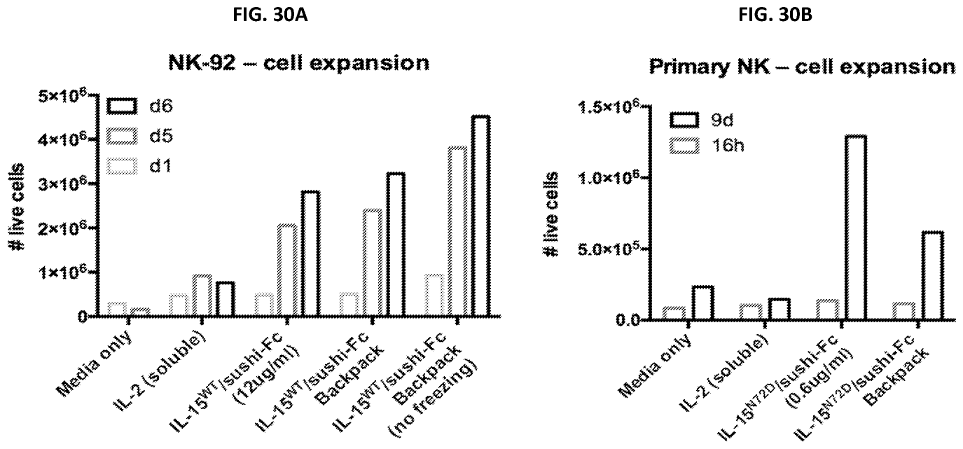

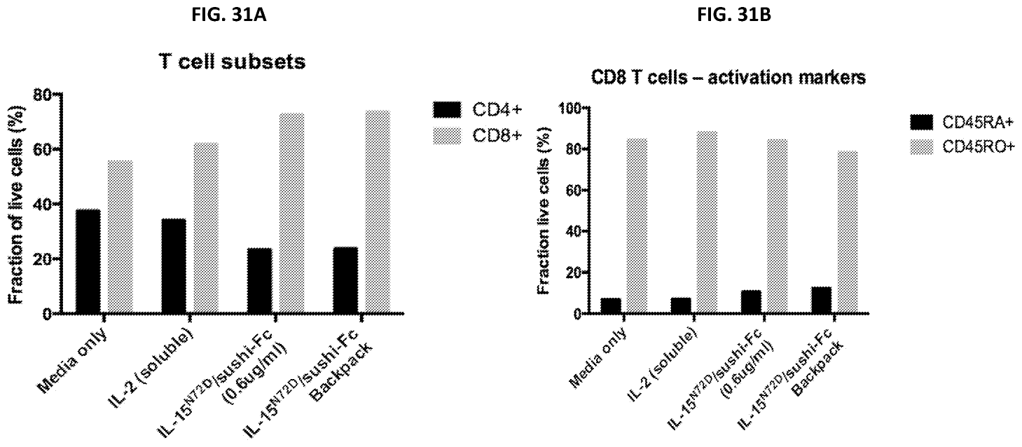

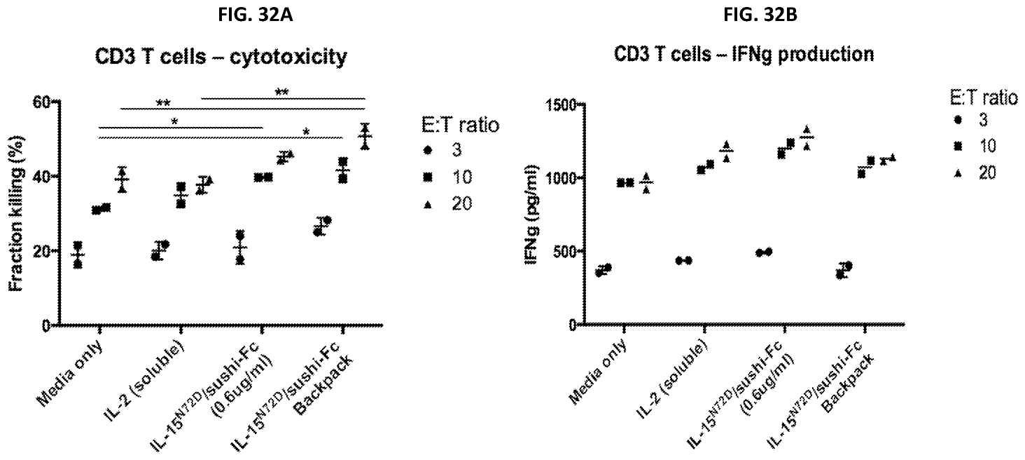

Activated T cells are washed with DPBS and incubated for 1 hr at 37° C. at a final cell density of approximately 10⁸ cells/mL with IL-15 nanogels at an equivalent cytokine concentration of approximately 0.5-0.75 mg/mL. The solution is mixed every 10-15 min by inversion or gentle vortexing. Cells are then resuspended in cell freezing media containing FBS with 5% dimethyl sulfoxide (DMSO) or serum-free freezing media (Bambanker, Lymphotec, Inc. cat. no. BB02) as specified, at a density of 10⁷cells/mL, and transferred to cryogenic vials to be frozen in a Mr. Frosty™ freezing container (Nalgene) as described by the manufacturer. Cells were then cultured in CM-T with 20 ng/mL IL-2 at 37° C. and 5% CO₂. Association of immune agonists formulated as tethered fusions and/or nanogels with the T cells was monitored by flow cytometry using a FACSCelesta™ flow cytometer with FACSDiva™ software (BD Biosciences), and revealed persistent association of such immune agoings with the T cells following the freezing and thawing (FIG. 27).

### Example 14: IL-15 Nanogel Provides Autocrine Stimulation and Expansion of T Cells after Adoptive Transfer Driven by Controlled Concentrated Release of IL-15

Interleukin 15, a powerful stimulator of CD8 and NK cell expansion is capable of driving anti-tumor activity of adoptively transferred T cells. However, systemic delivery does not safely provide sufficient doses to drive T cell expansion engraftment and anti-tumor activity.

High levels of IL-15 in the blood of cancer patients is associated with successful clinical responses (Kochenderfer et al., Lymphoma remissions caused by anti-CD19 chimeric antigen receptor T cells are associated with high serum interleukin-15 levels. J. Clinical Oncology (2017) 35(16):1803-1813). The IL-15 nanogel loaded T-cells disclosed herein are autologous T cells that carry tightly controlled doses of IL-15, which is slowly released over a 7-14 day period for directed autocrine activation of infused T cells without affecting endogenous T cells. One example is IL-15 nanogel loaded cytotoxic T cells (CTLs) that are tumor antigen primed using a novel dendritic cell priming sequence.

In conclusion, IL-15 nanogel cell loading is robust and tunable giving a controlled IL-15 dose per cell. The design of the IL-15 nanogel technology provides slow and controllable release of IL-15 resulting in autocrine stimulation and sustained cell expansion in adoptive T cell therapy. In contrast to systemically delivered IL-15, IL-15 nanogel Priming induces orders of magnitude lower systemic IFNg levels, endogenous CD8 and NK cell expansion, due to lack of systemic exposure. A fully closed, semi-automated cell process reproducibly generates several billion antigen-directed human CTLs with ˜20% reactivity and 95% T cell purity from healthy donors despite ultra low frequency (<1%) precursors. Human CTLs are highly dependent on IL-15 nanogel priming technology for cell survival and expansion in vivo.

### Example 15: Pharmacological Activity of IL-15 Nanogel-Loaded PMEL T Cells

In one embodiment, IL-15 nanogel is a multimer of human IL-15 receptor α-sushi-domain-Fc fusion homodimers with two associated IL-15 molecules (IL15-Fc), connected by a cleavable crosslinker (Linker-2), and non-covalently coated with a polyethylene glycol (PEG)-polylysine30 block copolymer (PK30). Specifically, an IL-15 nanogel is a multimer of human IL15-Fc monomers, connected by a biodegradable crosslinker and non-covalently coated with a polyethylene glycol (PEG)-polylysine30 block copolymer (PK30). IL15-Fc monomers consist of two subunits, each consisting of an effector attenuated IgG2 Fc variant fused with an IL-15 receptor α-sushi-domain noncovalently bound to a molecule of IL-15. IL-15 nanogel-loaded T cells are generated via a loading process in which target cells are co-incubated with IL-15 nanogel at high concentrations. Through this process, IL-15 nanogels becomes associated with the cell via electrostatic interactions and is internalized to create intracellular reservoirs of IL-15 nanogel. From these reservoirs, IL-15 nanogel slowly releases bioactive IL15-Fc by hydrolysis of the crosslinker. This extended release of IL15-Fc promotes proliferation and survival of IL-15 nanogel-loaded T cells, providing a targeted, controllable and time-dependent immune stimulus.

The objective of this study was to test the pharmacological activity of IL-15 nanogel-loaded PMEL T cells in C57BL/6J mice with and without orthotopically placed B16-F10 melanoma tumors. Control groups included vehicle control, PMEL cells alone and PMEL cells+IL15-Fc, administered in a separate injection (10 μg, maximum tolerated dose, MTD).

### B16-F10 Tumor Establishment and Tumor Measurements

B16-F10 melanoma tumor cells (0.2×10⁶) were injected intra-dermally into the shaved right flank of female C57BL/6 mice (Jackson Labs) on study day −12. The body weights were recorded and tumor dimensions (length [L] and width [W], defined in the list of abbreviations) were measured with calipers 2 to 3 times per week. Tumor volumes were calculated using the formula: W²×L×π/6.

### Isolation and Expansion of PMEL Cells

PMEL cells were isolated from the spleens and lymph nodes (inguinal, axillary and cervical) of 14 female transgenic PMEL mice (Jackson Laboratories, Bar Harbor, Me.). The spleens and lymph nodes were processed with a GentleMACS Octo Dissociator (Miltenyi Biotech, Auburn, Calif.) and passed through a 40 μm strainer. The cells were washed by centrifugation and the CD8a+ cells were purified using an IMACS naïve CD8a⁺ isolation kit (Miltenyi Biotech,) and a MultiMACS cell 24 block (Miltenyi Biotech) and separator (Miltenyi Biotech) with 18 columns following the manufacturer's protocol. The non-CD8a⁺ cells were removed by an affinity column and the CD8a⁺ T-cells were collected in the column eluate. The purity of CD8a+ cells was confirmed by flow cytometry.

Upon isolation (D0) purified CD8a+ cells from PMEL mice were plated into ten, 6-well tissue culture plates coated with anti-CD3 and anti-CD28 at a density of 5×10⁶ cells/well and incubated for 24 hr at 37° C. and 5% CO2. Murine IL-2 (20 ng/mL) and murine IL-7 (0.5 ng/mL) were added 24 hr post plating (D1). On D2 and D3, the cells were counted and diluted to a concentration of 0.2×10⁶ cells/mL with fresh media containing murine IL-21 (10 ng/mL). The cells were collected on D4 to obtain a total of 100×10⁶ PMEL cells/mL in 28 mL of vehicle control.

### Preparation of IL-15 Nanogel-Loaded PMEL T Cells

Five mL of PMEL cells (100×10⁶ cells/mL) were mixed with 5.5 mL of IL-15 nanogel (1.36 mg/ml) and incubated with rotation for 1 hr at 37° C. to create IL-15 nanogel-loaded PMEL cells. IL-15 nanogel-loaded PMEL cells were washed (3×, first with medium and then twice with HBSS) by centrifugation (500 g) and counted. IL-15 nanogel-loaded PMEL cells were resuspended at a concentration of 50×10⁶ cells/mL. The mice in Groups 5A and 5B were injected with 200 μL of this preparation for a total of 10×10⁶ IL-15 nanogel-loaded PMEL cells per mouse. PMEL cells (15 mL at 100×10⁶ cells/mL) were mixed with 15 mL of HBSS, incubated with rotation for 1 hr at 37° C., washed (3×, first with medium and then twice with HBSS) by centrifugation (500 g) and counted. PMEL cells were resuspended at a concentration of 50×10⁶ cells/mL. The mice in Groups 2A and 2B were injected IV with 200 μL of this preparation for a total of 10×10⁶ PMEL cells per mouse. The mice in Groups 3A and 3B were injected IV with 200 μL of this preparation for a total of 10×10⁶ PMEL cells per mouse, and received a retro-orbital injection of IL15-Fc (10 μg/mouse in 50 μl HBSS; lot #TS0). Based on an average loading efficiency of 39%, the total amount of IL15-Fc associated with 10×10⁶ PMEL cells is 58.5 μg, which is 5.85-fold higher than the amount delivered systemically by injection of IL15-Fc (10 μg) in Groups 3A and 3B.

### Fc-IL-15 ELISA

An Fc-IL15 Enzyme-Linked Immunosorbent Assay (ELISA) was used to determine the IL15 Fc concentration in the samples collected at 2 hr, D1, 2, 4 and 10 post-dose. ELISA plates (were coated overnight at 4° C. with Goat Anti-human IgG Fc Capture Antibody. Plates were washed and blocked with reagent diluent for at least 2 hours at 30° C. Plates were washed, samples (diluted in reagent diluent) and IL15-Fc standards (in duplicate, 31 to 2000 pg/mL, in reagent diluent) were added to the wells, and plates were incubated for 1 hour at 37° C. Plates were washed followed by addition of biotin-anti-IL15 detection Antibody was added and incubated for 1 hour at 37° C. Plates were washed and incubated with Streptavidin-HRP for 20 min at 37° C. Plates were washed followed by addition of 3,3′,5,5′-Tetramethylbenzidine (TMB) Substrate Solution and incubated for 20 min at room temperature in the dark until the reaction was stopped. Plates were read on a microplate reader (450 nm).

The assay was run twice. For the first run, samples were evaluated at the following dilutions: 1:20000 for the 2 hr time point, 1:5000 for the D1 time point, and 1:250 for the D2, D4 and D10 time points. For the second run, samples from groups 3A and 3B, were diluted 1:5000 for the D1 time point, 1:250 for the D2 time point and 1:25 for the D4 and D10 time points. Samples from groups 1A and 1B, 2A and 2B and 5A and 5B were diluted 1:25 for all the time points analyzed. The data is reported for the second run. However, because the samples for the 2 hr time point were exhausted for the second run, and given that IL15-Fc concentrations at 24 hr were similar in groups 3A and 3B across the two runs, the 2 hr values from the first run were included with the other data points from the second run for the purpose of calculating pharmacokinetic (PK) parameters.

The lower limit of quantitation (LLOQ) in blood was 310 ng/ml for the 1:20000 dilution, 77.5 ng/ml for the 1:5000 dilution, 3.875 ng/ml for the 1:250 dilution and 0.3875 ng/ml for the 1:25 dilution.

Serum Cytokine Levels in Serum from Mice

ThermoFisher ProcartaPlex mouse high sensitivity panel 5plex Cat. #EPXS0S0-22199-901 kits were used according to manufacturer's protocol and samples were analyzed on a Bio-Plex 200 system. Serum was thawed on ice, and 20 μL of serum were tested for IFN-γ, TNF-α, IL-2, IL-4 and IL-6 levels. In a few samples, 20 μL of serum were not available, so a smaller volume was utilized. Dilution factors were adjusted, to calculate concentrations according to the standard curves. Statistical analysis was carried out in GraphPad Prism.

### Clinical Chemistry

Clinical chemistry parameters were measured on serum samples. FIG. 9 shows clinical chemistry parameters where statistically significant changes were observed for the naïve mice at D1 and D4 post-dose. At D1 post-dose, a significant reduction (p<0.05) in Albumin levels was observed in the PMEL+IL15-Fc group relative to the IL-15 nanogel-loaded PMEL group as well as in the Blood Urea Nitrogen (BUN) levels compared to both vehicle control and IL-15 nanogel-loaded PMEL (p<0.05 for both). At D4 post-dose, the PMEL+IL15-Fc group showed significantly reduced Albumin (p<0.05 compared to all the other treatment groups), total protein (p<0.05 compared to vehicle control), Glucose (p<0.05 compared to the IL-15 nanogel-loaded PMEL), Albumin/Globulin (ALB/GLOB) ratio (p<0.05 compared to vehicle control, and p<0.01 compared to PMEL and IL-15 nanogel-loaded PMEL). Additionally, the PMEL+IL15-Fc group showed a significant increase (p<0.05 compared to vehicle control and IL-15 nanogel-loaded PMEL) in Cholesterol levels. All treatment groups showed a trend toward a reduction in Calcium levels compared to vehicle control, which was statistically significant with the PMEL group (p<0.05). The IL-15 nanogel-loaded PMEL group showed statistically significant changes in Total Bilirubin (p<0.05 compared to vehicle control and PMEL) and Phosphorus (p<0.05 compared to PMEL).

FIG. 10 shows clinical chemistry parameters where statistically significant changes were observed for the tumor-bearing mice at D1 and D4 post-dose. At D1 post-dose, the only statistically significant change in clinical chemistry was a reduction in Bilirubin—conjugated, observed with both the PMEL+IL15-Fc and with the IL-15 nanogel-loaded PMEL group (p<0.05 compared to vehicle control for both). At D4 post-dose, statistically significant increases in Albumin (p<0.05 compared to vehicle control), Total Protein (p<0.01 compared to vehicle control) and Bicarbonate TCO2 (p<0.05 compared to vehicle control) were seen with the PMEL group. Additionally, a statistically significant increase in Globulin was observed with the PMEL group (p<0.001 compared to vehicle control; and p<0.05 compared to DP-15 PMEL) and with the PMEL+IL15-Fc group (p<0.05 compared to vehicle control).

### Systemic Cytokine Release

Using a Luminex 5-plex kit, serum cytokines (IFN-γ, IL-2, IL-4, IL-6, and TNFα) were measured at 2 hr, 24 hr and 96 hr post-dose. In the naïve non-tumor bearing mice, the levels of IFN-γ in the PMEL+IL15-Fc group were 12.8±3.7 pg/mL, while IFN-γ was below the lower limit of quantitation (LLOQ=0.06 pg/mL) in the IL-15 nanogel-loaded PMEL group (FIG. 11). In the tumor-bearing mice, there was on average a 41-fold higher IFN-γ concentration in the PMEL+IL15-Fc group (20.5±0.5 pg/mL) compared to the IL-15 nanogel-loaded PMEL group (0.5±0.1 pg/mL). Higher levels of IL-2, IL-6, and TNFα were also seen in the PMEL+IL15-Fc group compared to the other groups.

### Pharmacokinetics of IL15-Fc in the Blood

A sandwich ELISA (anti-Fc capture antibody followed by anti-IL15 detection antibody) was used to measure IL15-Fc in the blood of mice injected with PMEL+IL15-Fc (10 μg) and IL-15 nanogel-loaded PMEL (carrying 58.5 ug of IL15-Fc).

The pharmacokinetics (PK) of a single dose administration of IL-15 nanogel-loaded PMEL and PMEL+IL15-Fc were determined for a composite animal in naïve and tumor-bearing mouse. For the PMEL+IL15-Fc group, maximum concentration (Cmax) was attained at 2 hr post dose administration in both naïve and tumor-bearing mice. In the IL-15 nanogel-loaded PMEL group, the first concentration measured was at 24 hr (the 2 hr samples were initially measured at a non-optimal dilution and no IL15-Fc was detected, and there was not sufficient sample available to repeat the measurement with ideal dilution). Tumor-bearing mice attained slightly lower concentrations than the naïve mice. The calculated mean t½ for IL15-Fc in the PMEL+IL15-Fc group was 28.9 hr and 7.12 hr in tumor bearing mice and non-tumor bearing mice, respectively.

The IL15-Fc concentrations at the 24 hr timepoint were compared between the PMEL+IL15-Fc and IL-15 nanogel-loaded PMEL groups. The total IL15-Fc concentration was higher in the PMEL+IL15-Fc (10 μg) group than in the IL-15 nanogel-loaded PMEL group (58.5 ug of IL15-Fc), approximately 3488-fold higher in the naïve mice and 3299-fold higher in the tumor bearing mice. Composite IL15-Fc PK parameters are summarized in Table 1 and the mean (SD) IL15-Fc PK profiles are depicted in FIG. 12.

TABLE 1
Composite IL15-Fc PK parameters for the PMEL + IL15-Fc group, in naive and
tumor-bearing mice (Mug dose of IL15-Fc)

| | | | T1/2 | Cmax | Tmax | Clast | Tlast | AUClast | AUCINF |
|---|---|---|---|---|---|---|---|---|---|
| Animal | Compound | Group | (hr) | (ng/mL) | (hr) | (ng/mL) | (hr) | (hr*ng/mL) | (hr*ng/mL) |
| Composite | IL15-Fc | Non- | 7.12 | 6931 | 2 | 3.64 | 96 | 202387 | 202424 |
| | | tumor | | | | | | | |
| | | bearing | | | | | | | |
| | | Tumor | 28.9 | 7300 | 2 | 0.448 | 240 | 156335 | 156353 |
| | | Bearing | | | | | | | |

### Inhibition of Tumor Growth

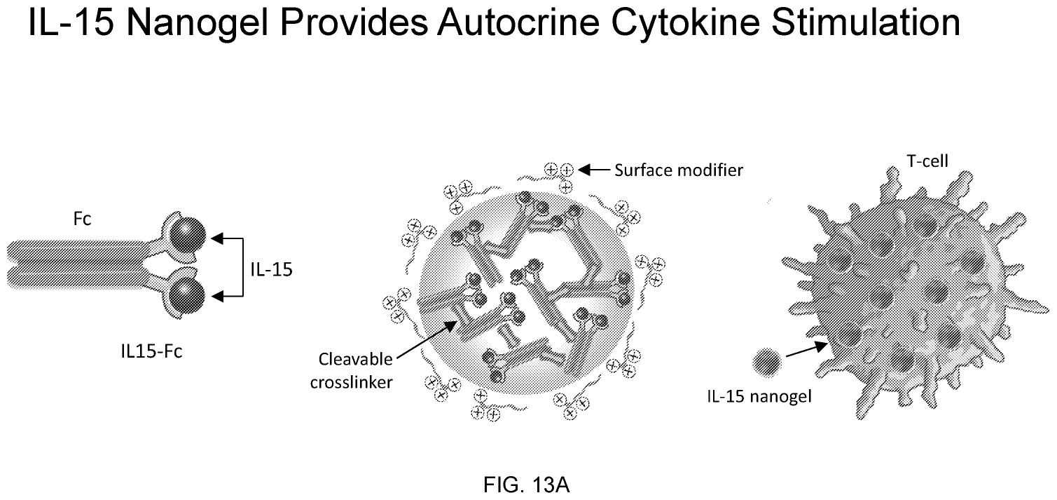

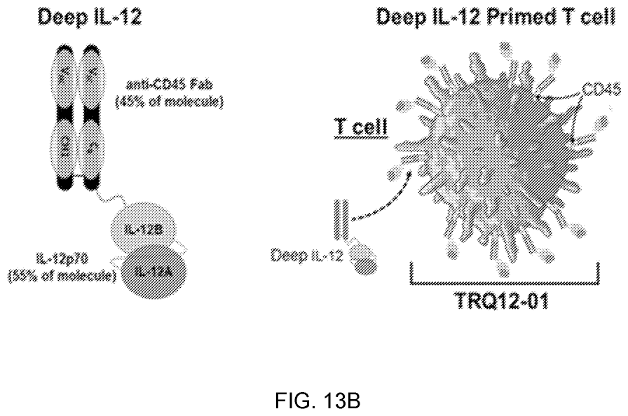

On D0 (the day of dosing) tumors had reached an average volume of approximately 140 mm³. A statistically significant inhibition of tumor growth was observed at D4 post-dose in all treatment groups compared to vehicle control (p<0.0001), and this difference became more pronounced over time (FIG. 13, left panel). On study D16 there were only 2/5 animals remaining in the vehicle control group (the others were sacrificed due to extensive tumor burden) but 4/5 animals remaining in each of the treatment groups. Tumor volumes in the vehicle control group were significantly (p<0.0001) different from all other groups. Tumor volumes in the PMEL group were significantly (p<0.05) larger than those in the IL-15 nanogel-loaded PMEL and PMEL+IL15-Fc groups. The inhibition of tumor growth in the PMEL+IL15-Fc and IL-15 nanogel-loaded PMEL groups were not different from each other on D16 (FIG. 13, left and right panels). Tumors were weighed post-sacrifice (n=2-5, each group, each time point) on D1, 4, 10 and 16 post-dose. Tumor weights are shown in FIG. 14.

Some animals were found moribund or dead prior to the study-specified endpoints. These included mice in the vehicle control (4 total: 1 on D9, 1 on D10 and 2 on D14), in the PMEL group (2 total: 1 on D2, and 1 on D6), in the PMEL+IL15-Fc group (2 total: 1 on D9 and 1 on D11) and in the IL-15 nanogel-loaded PMEL group (2 total: 1 on D9 and 1 on D16). These were not considered related to treatment since they were distributed across groups with the highest numbers (n=4) in the vehicle control. Finally, there was no difference in animals found moribund or dead associated with the IL-15 nanogel-loaded PMEL group compared to PMEL.

Major findings of the study are summarized below.
1. IL-15 nanogel-loaded PMEL cells were well tolerated at the administered dose of 10×10⁶ cells.
2. Both PMEL, PMEL+IL15-Fc and IL-15 nanogel-loaded PMEL cells resulted in tumor growth inhibition compared to vehicle control. Inhibition was higher with PMEL+IL15-Fc and IL-15 nanogel-loaded PMEL cells compared to PMEL.
3. No toxicologically relevant clinical chemistry parameter changes were observed with either PMEL or IL-15 nanogel-loaded PMEL cells. Some changes were observed with PMEL+IL-15 Fc.
4. No changes in serum IFN-γ, TNF-α or IL-6 were detected with PMEL or IL-15 nanogel-loaded PMEL cells at any time point. Significant changes in serum IFN-γ and TNF-α were observed with PMEL+IL15-Fc at 24 hr. IL-6 was increased with PMEL+IL15-Fc at 2 hr (Non-tumor-bearing (naïve) mice only) and 24 hr.
5. The serum levels of IL15-Fc in the IL-15 nanogel-loaded PMEL group were over 3000-fold lower compared to the levels detected in the PMEL+IL15-Fc group, corresponding to no weight loss, no significant changes in CBCs and in endogenous immune cells (CD8⁺, NK1.1⁺ and CD4⁺ cells), reduced IFN-γ serum levels and associated pharmacological changes compared to the PMEL+IL15-Fc group.

### Example 16: Combining IL-12 Tethered Fusion-Loaded and IL-15 Nanogel-Loaded T Cells Leverages Different Mechanisms to Enhance Anti-Tumor Activity

Background: Interleukin-15 (IL-15) and Interleukin-12 (IL-12) play different roles as immunomodulators. IL-15 induces T cell memory and supports survival, activation and proliferation of CD8⁺ T and NK cells. IL-12 promotes T cell cytotoxicity and innate immune responses in the tumor microenvironment. Both cytokines have been explored as cancer immunotherapies, but clinical success has been limited due to severe side effects. To limit systemic toxicities, T cell therapy comprising surface-loaded immune agonsists, as described herein was developed. Multi-targeted T cells (MTC) specific for multiple tumor antigens are generated from patient apheresis. Cytokines are tethered to MTCs to support MTC persistence and activity following adoptive transfer into patients, while limiting systemic cytokine exposure. This study evaluates the combination of cytotoxic T lymphocytes (CTL) surface-loaded with IL-15 nanogel and IL-12 tethered fusion to leverage their complementary biology for superior efficacy.
Methods: CTLs reactive against MART-1 antigen were generated from healthy donors (MART-1 CTLs). Next, expansion and cytotoxicity of MART-1 CTLs loaded with IL-12 tethered fusion, IL-15 nanogel or both against MART-1 expressing SKMEL-5 melanoma cells were assessed. In addition, murine PMEL CD8⁺ T cells reactive against the B16-F10 melanoma antigen gp100 were loaded with IL-12 tethered fusion, IL-15 nanogel or both and evaluated for in vitro expansion, activation and cytotoxicity against B16-F10 melanoma cells, as well as for anti-tumor activity in B16-F10 tumor-bearing mice.
Results: Loading with IL-15 nanogel promoted MART-1 CTL proliferation and preserved antigen reactivity over time. IL-12 tethered fusion loaded MART-1 CTLs displayed enhanced IFN-γ secretion and cytotoxicity, particularly at low effector:target ratios. Combination of MART-1 CTLs loaded with IL-12 tethered fusion and IL-15 nanogel further enhanced T cell expansion, IFN-γ secretion and cytotoxicity. Similarly, combination of murine PMEL T cells loaded with IL-12 tethered fusion and IL-15 nanogel resulted in persistent T cell activation, improved memory, and enhanced cytotoxicity over individually loaded T cells. Coadministration of IL-12 tethered fusion and IL-15 nanogel loaded PMEL T cells to B16-F10 melanoma-bearing mice was well-tolerated, with minimal and reversible body weight loss, and elicited superior anti-tumor activity.
Conclusions: Modular tethering of IL-12 tethered fusion and IL-15 nanogel to T cells exert their different mechanisms of action as immunomodulators, unexpectedly resulting in a synergistic effect, with increased anti-tumor activity without notable toxicity in preclinical models.
1. Combination of IL-15 Nanogel-Loaded PMEL T Cells with IL-12 Tethered Fusion-Loaded PMEL T Cells Results in Unexpected Synergistic Effects

Interleukin-15 (IL-15) activates and expands both CD8⁺ T cells and NK cells but not immunosuppressive T_{reg} cells. Thus, IL-15 is an attractive asset for cancer immunotherapy, but its systemic administration is limited by immune activation and toxicities. To limit IL-15 systemic exposure, the IL-15 nanogel, a multimer of chemically crosslinked IL-15/IL-15 Rα/Fc heterodimers (IL15-Fc) disclosed herein was developed. IL-15 nanogel is loaded onto tumor reactive T cells prior to adoptive cell transfer (ACT). This novel therapeutic approach enables IL-15 nanogel loading into cells at concentrations unachievable with systemic IL15-Fc, causes autocrine T cell activation and expansion, yet limits systemic exposure and associated toxicities. The anti-tumor activity of T cell therapies has been limited by insufficient T cell expansion and activation. Disclosed herein is a combination therapy comprising a IL-15 nanogel combined with a IL-12 tethered fusion to overcome these limitations.

Specifically, as illustrated in FIG. 13A, IL-15 nanogel (may be referred to as DP-15 or Deep IL-15) refers to a multimer of human IL-15 receptor α-sushi-domain-Fc fusion homodimers with two associated IL-15 molecules (IL15-Fc), connected by a cleavable crosslinker (see, e.g., PCT Application No. PCT/US2018/049594, incorporated herein by reference), and non-covalently coated with a polyethylene glycol (PEG)-polylysine₃₀ block copolymer (PK30). More specifically, IL-15 nanogel is a multimer of human IL15-Fc monomers, connected by a hydrolysable crosslinker (CL17) and non-covalently coated with a polyethylene glycol (PEG)-polylysine30 block copolymer (PK30). IL15-Fc monomers consist of two subunits, each consisting of an effector attenuated IgG2 Fc variant fused with an IL-15 receptor α-sushi-domain noncovalently bound to a molecule of IL-15. IL-15 nanogel-loaded T cells are generated via a loading process in which target cells are co-incubated with IL-15 nanogel at high concentrations. Through this process, IL-15 nanogel becomes associated with the cell via electrostatic interactions and is internalized to create intracellular reservoirs of IL-15 nanogel. From these reservoirs, IL-15 nanogel slowly releases bioactive IL15-Fc by hydrolysis of the crosslinker. This extended release of IL15-Fc promotes proliferation and survival of IL-15 nanogel-loaded T cells, providing a targeted, controllable and time-dependent immune stimulus.

As shown in FIG. 13B, IL-12 tethered fusion (may be referred to as DP-12 or Deep IL-12) consists of an IL-12 p70 molecule fused to an anti-CD45 antibody antigen-binding fragment (Fab). Through this Fab, IL-12 tethered fusion is tethered to CD45 molecules on the surface of the MTCs. T cells carrying surface-tethered IL-12 tethered fusion (IL-12 tethered fusion-loaded T cells) leverage the ability of the cytokine IL-12 to augment immune responses in several different and complementary ways. These include the differentiation or expansion of interferon-γ (IFN-γ) producing T helper 1 (T_{H}1) cells, cytotoxic T lymphocytes (CTLs) and natural killer (NK) cells; the induction of antigen presentation to CTLs by major histocompatibility complex class 1 (MHCI) molecules; reprogramming of immunosuppressive myeloid cells; and anti-angiogenic effects. IL-12 tethered fusion-loaded T cells transport IL-12 tethered fusion into tumors, where it can act in a paracrine manner

The goal of this study is to (1) evaluate the in vitro cytotoxicity of human and mouse T cells loaded with IL-12 tethered fusion, IL-15 nanogel or a combination of both (as mixed cell populations, T cells loaded with either IL-12 tethered fusion or with IL-15 nanogel; or in a condition where both cytokines are loaded on the same cells, co-loaded); (2) evaluate the in vivo anti-tumor activity of the combination of IL-15 nanogel loaded PMEL T cells (“DP-15 PMEL”) and IL-12 tethered fusion loaded PMEL T cells (“DP-12 PMEL”).

For the in vivo evaluation of the combination, the following groups were evaluated, as indicated in Table 2 below: a vehicle control (G1), a dose escalation of a single dose of IL-12 tethered fusion-loaded PMEL T cells (1, 2.5 and 5×10⁶) (G2-4), three combination groups where DP-12 PMEL T cells (1, 2.5 or 5×10⁶) were added to a constant amount of IL-15 nanogel-loaded PMEL T cells (10×10⁶) (G5-7). Additionally, three combination groups where PMEL T cells (1, 2.5 or 5×10⁶) were added to a constant amount of IL-15 nanogel-loaded PMEL T cells (10×10⁶) (G8-10) served as control for groups 5-7.

TABLE 2
Treatment groups

| Group | N | Treatment | Route | Dose level | Frequency |
|---|---|---|---|---|---|
| 1 | 20 | Vehicle control | IV | 200 μL HBSS | Once, D0 |
| | | (HBSS) | | | |
| 2 | 15 | IL-12 tethered | IV | 1 × 10⁶ T cells in 100 | Once, D0 |
| | | fusion-loaded | | μL HBSS | |
| | | PMEL T cells | | | |
| 3 | 15 | IL-12 tethered | IV | 2.5 × 10⁶ T cells in | Once, D0 |
| | | fusion-loaded | | 100 μL HBSS | |
| | | PMEL T cells | | | |
| 4 | 20 | IL-12 tethered | IV | 5 × 10⁶ T cells in 100 | Once, D0 |
| | | fusion-loaded | | μL HBSS | |
| | | PMEL T cells | | | |
| 5 | 20 | IL-15 nanogel- | IV | 10 × 10⁶ T cells in 100 | Once, D0 |
| | | loaded PMEL | | μL HBSS | |
| | | T cells | | | |
| | | IL-12 tethered | IV | 1 × 10⁶ T cells in1200 | Once, D0 |
| | | fusion-loaded | | μL HBSS | |
| | | PMEL T cells | | | |
| 6 | 20 | IL-15 nanogel- | IV | 10 × 10⁶ T cells in 100 | Once, D0 |
| | | loaded PMEL | | μL HBSS | |
| | | T cells | | | |
| | | IL-12 tethered | IV | 2.5 × 10⁶ T cells in | Once, D0 |
| | | fusion-loaded | | 100 μL HBSS | |
| | | PMEL T cells | | | |
| 7 | 20 | IL-15 nanogel- | IV | 10 × 10⁶ T cells in 100 | Once, D0 |
| | | loaded PMEL | | μL HBSS | |
| | | T cells | | | |
| | | IL-12 tethered | IV | 5 × 10⁶ T cells in 100 | Once, D0 |
| | | fusion-loaded | | μL HBSS | |
| | | PMEL T cells | | | |
| 8 | 20 | IL-15 nanogel- | IV | 10 × 10⁶ T cells in 100 | Once, D0 |
| | | loaded PMEL | | μL HBSS | |
| | | T cells | | | |
| | | PMEL T cells | IV | 1 × 10⁶ T cells in 100 | Once, D0 |
| | | | | μL HBSS | |
| 9 | 19 | IL-15 nanogel- | IV | 10 × 10⁶ T cells in 100 | Once, D0 |
| | | loaded PMEL | | μL HBSS | |
| | | T cells | | | |
| | | PMEL T cells | IV | 2.5 × 10⁶ T cells in | Once, D0 |
| | | | | 100 μL HBSS | |
| 10 | 19 | IL-15 nanogel- | IV | 10 × 10⁶ T cells in 100 | Once, D0 |
| | | loaded PMEL | | μL HBSS | |
| | | T cells | | | |
| | | PMEL T cells | IV | 5 × 10⁶ T cells in 100 | Once, D0 |
| | | | | μL HBSS | |

Readouts included anti-tumor activity, body weight changes, flow cytometry on blood to evaluate changes in endogenous immune cells (CD4, CD8, NK and Treg) and transferred PMEL T cells (Enumeration, Phenotype, Activation, Proliferation), serum blood chemistry (Day 4 and Day 11 post dosing), Complete Blood Counts (CBC, Day 1 and Day 4 post dosing), and systemic cytokine release (Luminex; Day 1 and Day 4 post-dosing). In addition, gross pathology was evaluated on 4-5 mice/group (except for groups 2 and 3) at Day 4 post dosing and at study end (D39, 6 mice/group from the treatment groups still on study). A study timeline is shown in FIG. 14.

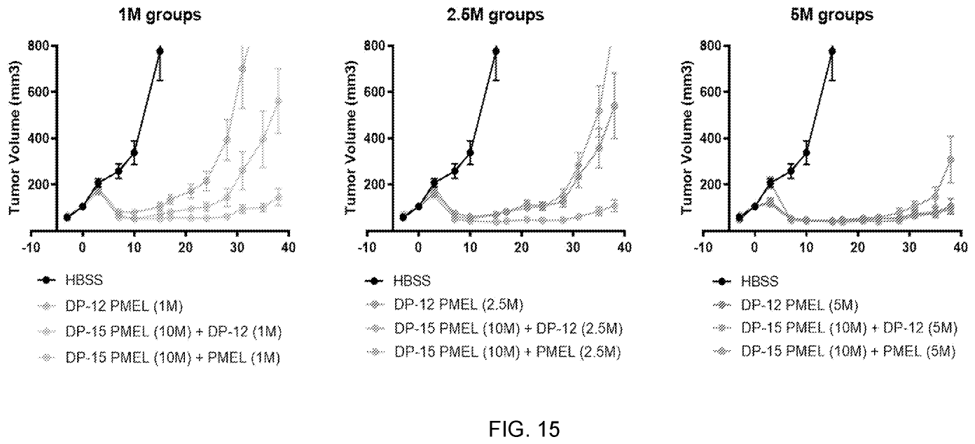

As shown in FIG. 15, the anti-tumor activity of the combination groups where IL-15 nanogel-loaded PMEL T cells (10×10⁶) were co-administered with IL-12 tethered fusion-loaded PMEL T cells dosed at 1, 2.5 and 5×10⁶ showed significantly improved anti-tumor activity compared to IL-15 nanogel-loaded PMEL T cells co-administered with PMEL T cells at the corresponding doses of 1, 2.5 and 5×10⁶ T cells (FIG. 15). However, in the case of the combination group at the highest IL-12 tethered fusion-loaded PMEL T cells dose (5×10⁶), the combination was equally efficacious as IL-12 tethered fusion-loaded PMEL T cells single agent (FIG. 15, right graph).

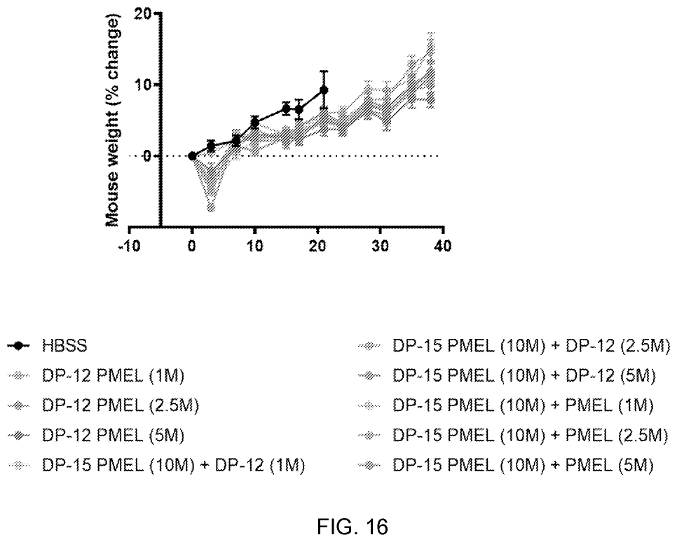

The combination was well tolerated with only a minor weight loss observed at Day 3 post dose, which fully reversed to baseline levels by Day 7 post dosing (FIG. 16).

Gross pathology evaluation at Day 4 post dosing and at end of study (Day 39) showed no gross lesions in any of the treatment groups. Lung and brain weights in all treatment groups were comparable to the vehicle control treated mice. There was an increase in spleen weight with the combination groups compared to vehicle control (FIG. 17, left panel). Spleen weights in the treatment groups were comparable across groups at study end, and similar to those of age-matched vehicle control treated mice (FIG. 17, right panel).

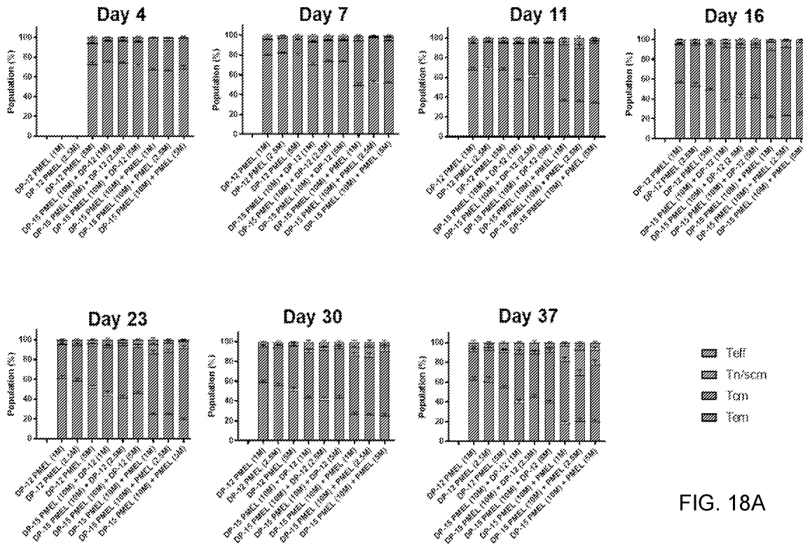

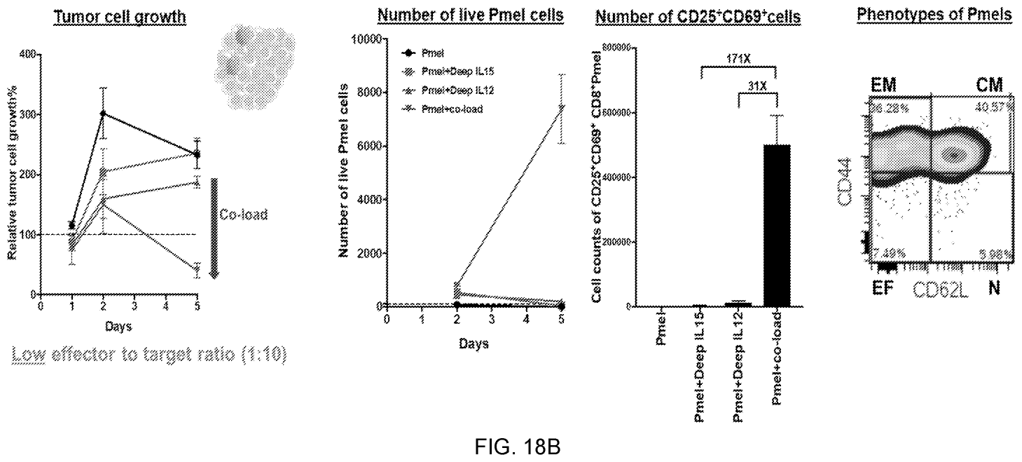

As shown in FIG. 18A, no statistically significant changes were observed in clinical chemistry parameters between the combination groups (5-7) and the combination control groups at matched cell doses (8-10), neither at D4 nor at D11 post-dosing. Flow cytometry analysis showed no significant differences between the combination groups (5-7) relative to the combination control groups at matched cell doses (8-10) in terms of overall PMEL T cells engraftment or endogenous T cells (CD4, CD8, NK and Tregs). Analysis of the phenotype of PMEL T cells over time showed a relative increase in effector memory T cells (Tem) in the combination groups (5-7) compared to the combination control groups (8-10) at correspondent cell doses.

In the presence of antigen, co-loading of IL-12 tethered fusion and IL-15 nanogel on PMEL T cells resulted in persistent T cell activation and improved memory phenotype and enhanced cytotoxicity in vitro (FIG. 18B).

### Materials and Methods

### IL-12 Tethered Fusion and IL-15 Nanogel Preparation

An IL-12 tethered fusion was constructed and used to prime PMEL T cells in accordance the previous Examples. IL-15 nanogel was synthesized by incubation of IL15-Fc with a crosslinking reagent.

### B16-F10 Tumor Establishment and Tumor Measurements

B16-F10 melanoma tumor cells (0.8×10⁶) were injected subcutaneously into the shaved right flank of female C57BL/6 mice (Jackson Labs) on study day −10. B16-F10 tumor-bearing mice were treated with cyclophosphamide (4 mg/mouse) one day prior to dosing The body weights were recorded and tumor dimensions (length [L] and width [W], defined in the list of abbreviations) were measured with calipers 2 to 3 times per week. Tumor volumes were calculated using the formula: W²×L×π/6.

### Isolation and Expansion of PMEL Cells

PMEL cells were isolated from the spleens and lymph nodes (inguinal, axillary and cervical) of 12 (7 female and 5 male) transgenic PMEL mice (Jackson Laboratories, Bar Harbor, Me.). Spleens and lymph nodes were processed with a GentleMACS Octo Dissociator (Miltenyi Biotech, Auburn, Calif.) and passed through a 40 μm strainer. Cells were washed by centrifugation and CD8a⁺ cells purified using an IMACS naïve CD8a⁺ isolation kit (Miltenyi Biotech) and a MultiMACS cell 24 block (Miltenyi Biotech) and separator (Miltenyi Biotech) following the manufacturer's protocol. The purity of CD8a⁺ cells was confirmed by flow cytometry.

Upon isolation (D0), 250×10\hat{ }6 purified PMEL T cells were resuspended at 1.0×10⁶/mL in Roswell Park Memorial Institute 1640 media (RPMI-1640) with 10% Fetal Bovine Serum (FBS), Penicillin/Streptomycin (Pen/Strep) (1%), L-glutamine (1%), Insulin/Transferrin/Selenium (ITS; 1%) and β-mercaptoethanol (BME, 50 μM), and plated into six, 6-well tissue culture plates (5×10⁶/well) coated with anti-CD3 and anti-CD28. Cells were incubated for 24 hr at 37° C. and 5% CO₂. Murine IL-2 (20 ng/mL) and murine IL-7 (0.5 ng/mL) were added 24 hr post plating (D1). On D2 and D3, the cells were counted and diluted to a concentration of 0.2×10⁶ cells/mL with fresh media containing murine IL-21 (25 ng/mL). The cells were collected on D4 and resuspended at 100×10\hat{ }6 or 20×10⁶ PMEL T cells/mL in PMEL T cell medium.

### Preparation of IL-15 Nanogel-Loaded PMEL T Cells

PMEL cells (100×10⁶ cells/mL) were mixed with an equal volume of IL-15 nanogel (1.36 mg/ml) and incubated with rotation for 1 hr at 37° C. to create IL-15 nanogel-loaded PMEL cells. IL-15 nanogel-loaded PMEL cells were washed (3×, first with medium and then twice with HBSS) by centrifugation (500 g) and counted. IL-15 nanogel-loaded PMEL cells were resuspended at a final concentration of 100×10⁶ cells/mL to be injected in Groups 5-10 (100 ul/mouse, corresponding to 10×10\hat{ }6 IL-15 nanogel-loaded PMEL T cells).

### Preparation of IL-12 Tethered Fusion-Loaded PMEL T Cells

PMEL T cells (20.0×10⁶ cells/mL) were mixed with an equal volume of mouse IL-12 tethered fusion (250 nM) and incubated with rotation for 30 min at 37° C. to create IL-12 tethered fusion-loaded PMEL T cells. IL-12 tethered fusion-loaded PMEL T cells were washed (3×, twice with medium and then once with HBSS) by centrifugation (500 g) and counted. IL-12 tethered fusion-loaded PMEL T cells were then resuspended at 10, 25 and 50×10\hat{ }6 IL-12 tethered fusion-loaded PMEL T cells for injection in groups 2-7 as indicated in the table above; 100 ul/mouse).

### Blood Collection

In-life blood samples (˜80 μL) were collected by submandibular bleeds. Terminal blood collections (D4) were carried out through cardiac punctures after CO₂ asphyxiation.

### Whole Blood Samples

For flow cytometry and CBC analysis, whole blood was collected in EDTA-coated tubes (Greiner Bio-One, Monroe, N.C.). For CBC analysis, blood samples were shipped to IDEXX laboratories (Grafton, Mass.).

### Serum Preparation (for Luminex and Blood Chemistry)

For serum samples blood was collected into tubes containing a clot activator (Greiner Bio-One, Monroe, N.C.). Tubes were centrifuged at 10,000 g, the serum supernatant was transferred into prelabeled cryotubes (Greiner Bio-One, Monroe, N.C.) and stored at −80° C. for future analysis. For blood chemistry samples were shipped to IDEXX laboratories (Grafton, Mass.).

### Flow Cytometry Staining

50 μl of blood from each animal were transferred into one well of a 96-deep well plate. The red blood cells were lysed using a hypotonic buffer and washed 2×. Counting beads were added to the blood during the red blood cell lysis step. The remaining cells were washed 3× in Staining buffer (0.5% BSA, 2 mM EDTA in PBS). The cells were resuspended in a master mix containing the staining antibodies (1:100 dilution in Staining buffer). The reagents used in the flow cytometry protocol are listed below in Table 3. The cells were incubated with the antibody mixture for 10 min at room temperature, protected from light.

For intracellular antigen staining, the cells were washed 3× in Staining buffer, resuspended in Fixation/Permeabilization Solution (Thermo Fisher Scientific, Waltham, Mass.), and incubated overnight (4° C.). The next day, samples were centrifuged and washed 3× in Permeabilization buffer. Antibodies for the intracellular Ki67 and FoxP3 markers were incubated with the permeabilized cells for 30 min at room temperature, protected from light and washed 2× in Staining buffer.

Flow cytometry data was collected on a FACSCelesta (Becton-Dickinson Franklin Lakes, N.J.) and analyzed in Flowjo.

TABLE 3
Flow cytometry reagents

| Species reactivity | Cell marker | Fluorophore | Manufacturer |
|---|---|---|---|
| Mouse | CD45 | BV510 | Biolegend |
| Mouse | CD4 | AF488 | Biolegend |
| Mouse | CD8a | APC-Cy7 | Biolegend |
| Mouse | CD90.1 | AF700 | Biolegend |
| Mouse | NK1.1 | BV711 | Biolegend |
| Mouse | CD44 | BV605 | Biolegend |
| Mouse | CD62L | BV786 | Biolegend |
| Mouse | CD25 | APC | Biolegend |
| Mouse | CD69 | PerCP-Cy5.5 | Biolegend |
| Mouse | Ki67 | PE-Cy7 | ThermoFisher |
| Mouse | FoxP3 | PE | ThermoFisher |
| Mouse | Fc-block | N/A | Biolegend |
| N/A | Dead cells-Zombie | BV421 | Biolegend |
| | Violet | | |

2. IL-15 Nanogel-Loaded MART-1 T Cells Synergize with IL-12 Tethered Fusion-Loaded MART-1 T Cells

Human T cells were trained using dendritic cells (DCs) presenting an immunodominant peptide from MART-1 to generate MART-1-Targeted T cells. The trained T cells were loaded with human IL-12 tethered fusion and IL-15 nanogel to generate IL-12 tethered fusion-loaded and IL-15 nanogel-loaded MART-1-targeted T cells and then tested for cytotoxicity against SKMEL5, a MART-1 expressing human cancer cell line, either alone or combined 1:1 IL-12 tethered fusion:IL-15 nanogel. MART-1-targeted T cell co-loaded with both IL-12 tethered fusion and IL-15 nanogel were also tested. Cytotoxicity at multiple effector:target ratios was measured by colorimetric live cell quantification and the T cells were characterized by flow cytometry to track the number and antigen reactivity of T cells in co-culture with SKMEL-5 cells compared to monoculture.

On Day 0 MART-1 cells were 82.6% specific. The majority of the cells had effector memory phenotypes (CD45RO+ CCR7−). Antigen-specific MART-1 MTCs were highly activated (CD25+ CD69+) on Day0 comparing to non-specific MTCs (data not shown).

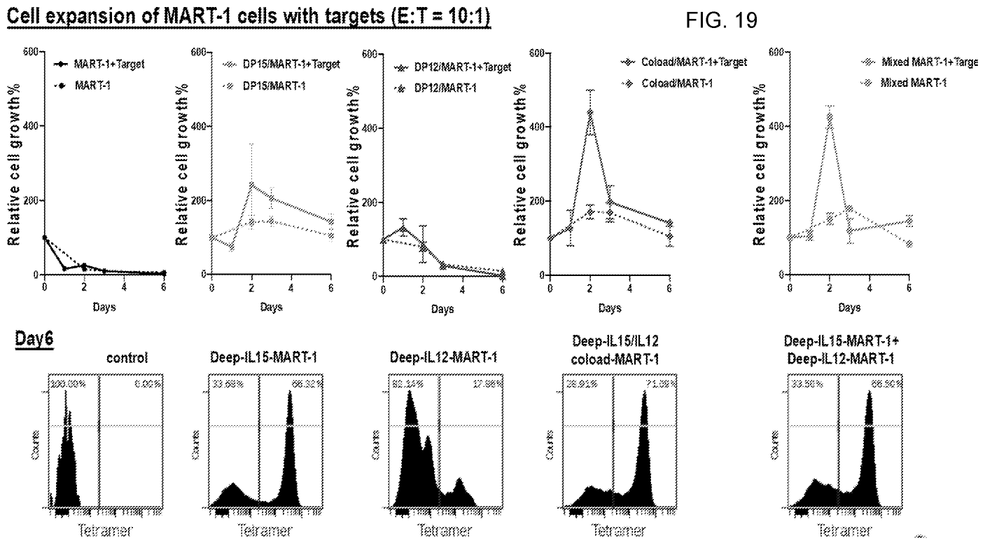

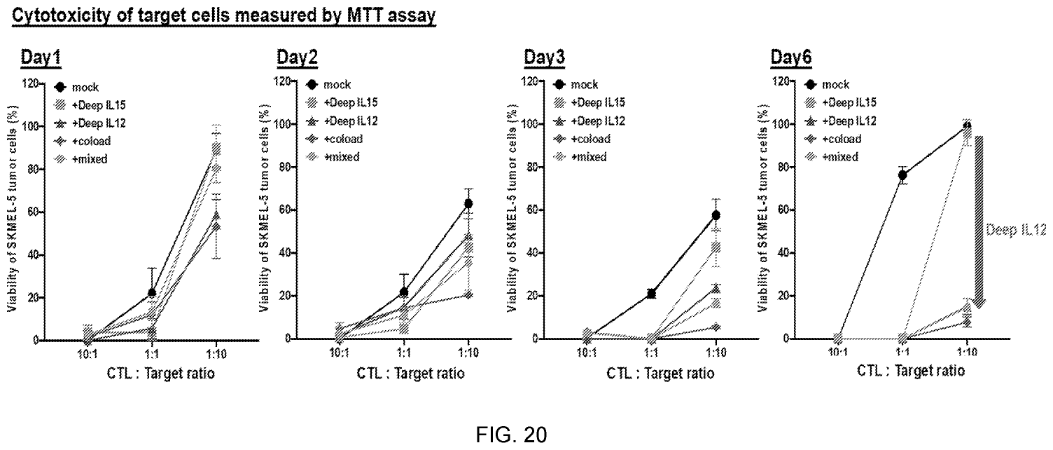

Over the time course (0-6 days) mono-, combination, and co-loaded therapies promote MART-1 cell proliferation upon antigen exposure. IL-15 nanogel, combined (mixed) and co-loaded treatments preserved antigen-specificity during cell expansion, while IL-12 tethered fusion preserved antigen-specificity by a smaller amplitude of effect (FIG. 19). IL-12 tethered fusion, combined (mixed) and co-loaded groups showed much enhanced SKMEL-5 cytotoxicity especially with a low E:T ratio and at later time points (FIG. 20).

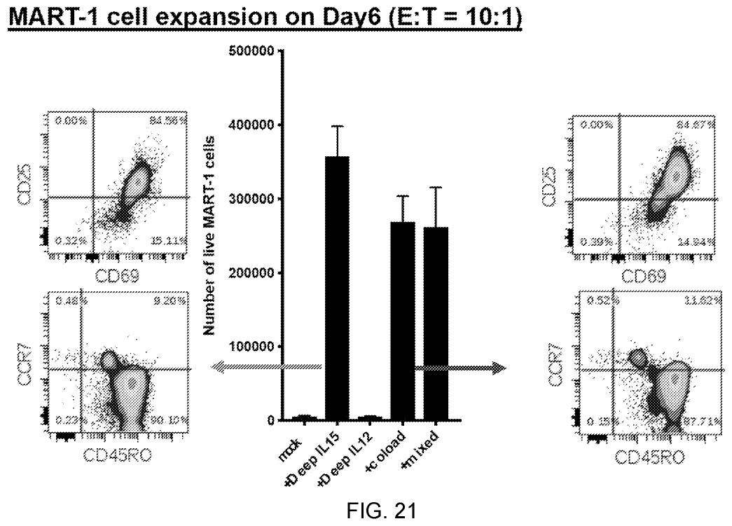

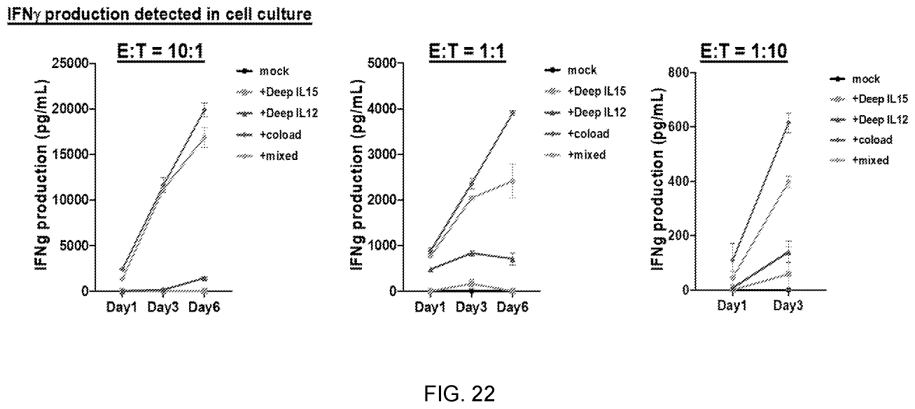

Cell proliferation driven by IL-15 nanogel, combined (mixed) and co-loaded treatments lead to similar T cell phenotypes and activation states. IL-15 nanogel, combined (mixed) and co-loaded groups had similar cell expansion profile, activation state and phenotypes (FIG. 21). To begin to understand the much higher cytotoxicity in IL-12 tethered fusion/coload/mixed groups interferon gamma (IFN-gamma) secreted in the cell culture supernatants was quantified (FIG. 22). IL-12 tethered fusion, combined (mixed) and co-loaded groups had enhanced IFN-gamma production, while IL-15 nanogel loaded MART-1 cells produced low level of IFN-gamma. Combined (mixed) and co-loaded MART-1 cells continued to produce IFN-gamma between Day1 and Day6 even at E:T 10:1 ratio where all tumor cells were killed on Day 1.

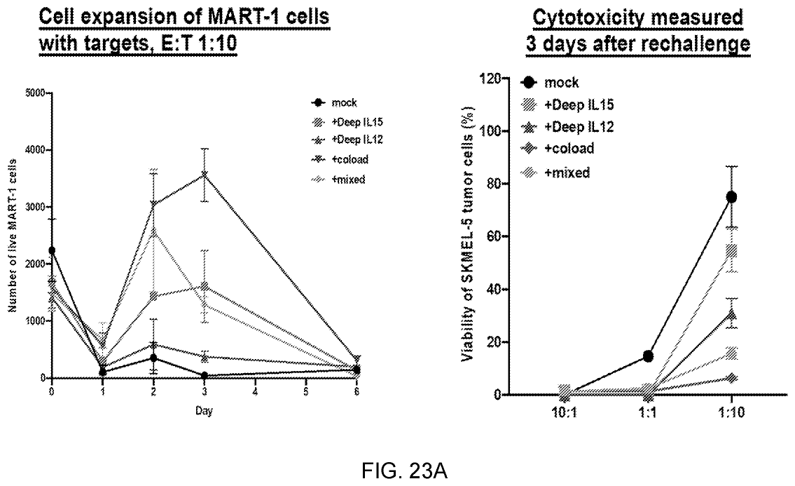

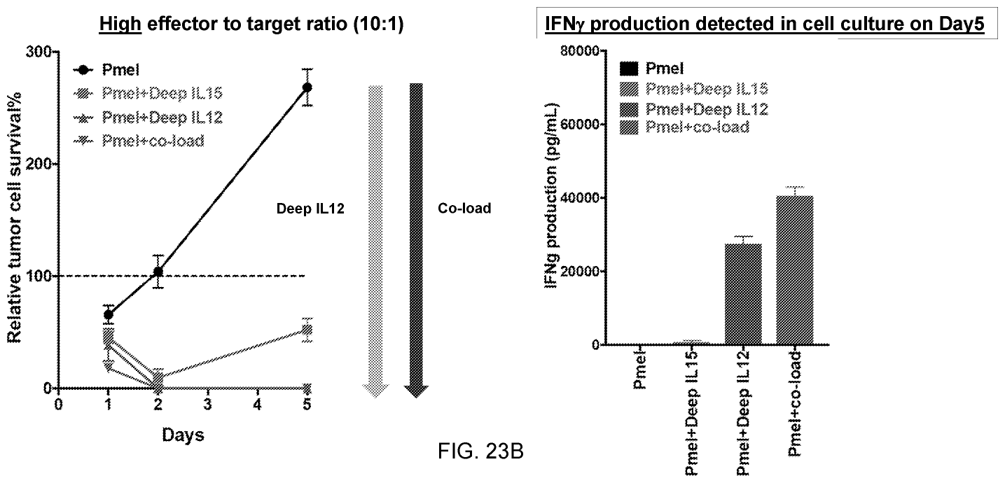

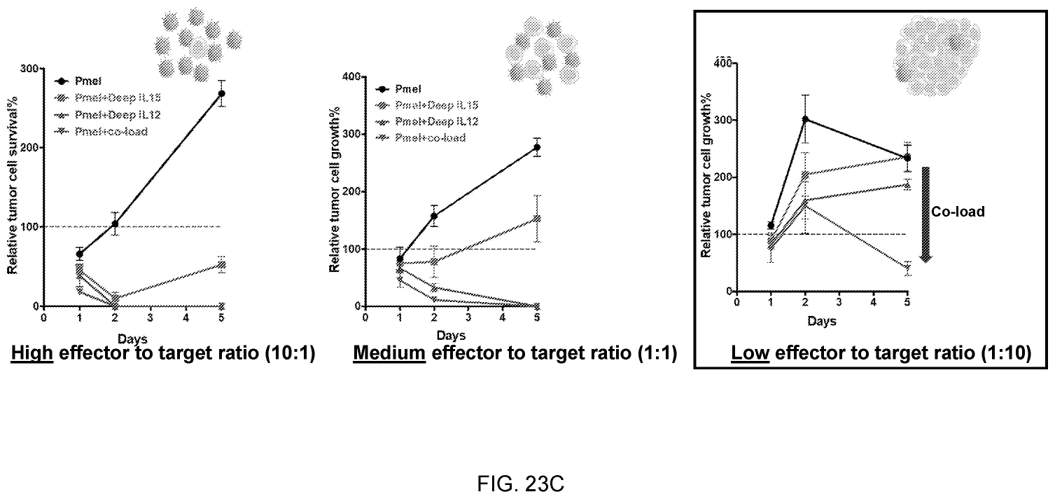

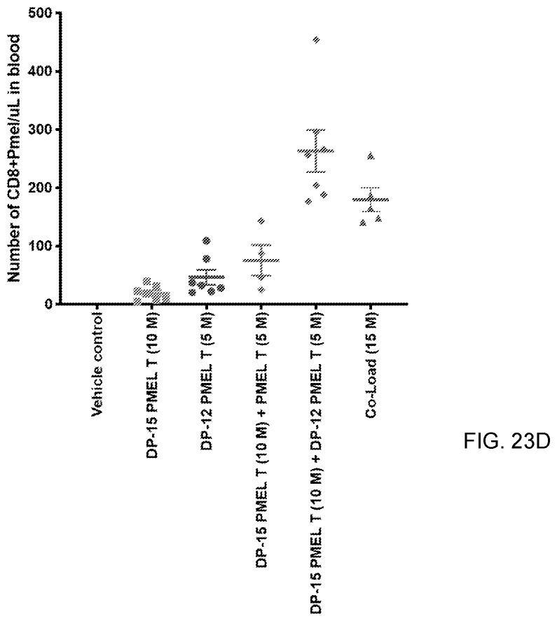

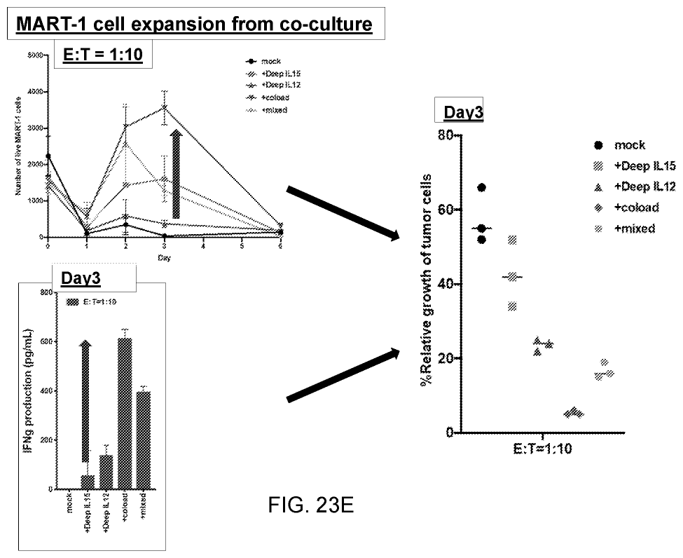

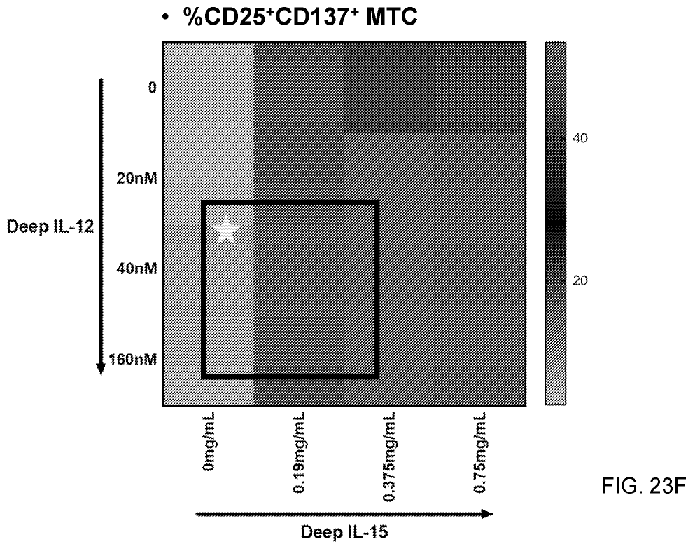

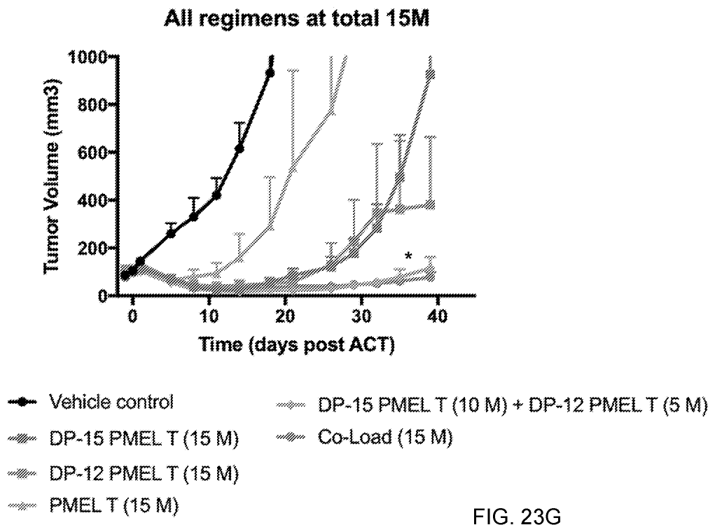

Under multiple E:T ratios and treatment groups nearly complete SKMEL-5 killing was observed early, by Day 1 or Day 3. Cell proliferation peaked on Day 3 (FIG. 23A), and reactive MART-1 cell population was maintained during expansion (FIG. 21). On Day 3 the surface-loaded MART-1 T cells have >80% effector memory phenotypes and have >80% CD25+CD69+ activation state. Day 3 of co-culture T cells were removed from the co-culture plate and transferred to fresh SKMEL-5 culture, to re-challenge them for cytotoxicity quantification. Three days after re-challenge, especially with a low E:T ratio, combined (mixed) and co-loaded MART-1 cells showed enhanced SKMEL-5 cytotoxicity compared to either IL-12 tethered fusion or IL-15 nanogel-loaded T cells alone.

FIG. 23B: IL-12 tethered fusion drives cytotoxicity of Pmel cells. Co-load treatment improves cytotoxicity of IL15 nanogel-loaded Pmel cells. As shown in FIG. 23B, complete tumor elimination was achieved in IL-12 tethered fusion and co-load groups by Day2. IL-12 tethered fusion drives IFNg production and cytotoxic activities. Tumor outgrowth was observed in control and IL-15 nanogel group by Day5.

FIG. 23C: Co-load mediated target cell cytotoxicity at low E:T ratio. As shown in FIG. 23C, IL-15 nanogel loses long-term cytotoxicity advantage as the E:T ratio decreases. IL15 nanogel+IL12 TF co-load condition shows induced persistent cytotoxicity advantage over mono-therapy.

FIG. 23D: Combo IL-15 nanogel+IL-12 TF: improved activity relative to individual agents. As shown in FIG. 23D, IL-15 nanogel, IL-12 TF, and antigen presentation showed surprising enhancement of PMEL T cells long term persistence in circulation. Co-load (15M) and combination group (IL-15 nanogel 10M+IL-12TF 5M) show comparable anti-tumor activity. Combination groups show improved activity compared to the individual agents.

FIG. 23E: Combination treatment enables persistent cell expansion of antigen-specific cells and enhances cytotoxicity. As shown in FIG. 23E, IL-15 nanogel rescues antigen-specific cell expansion from IL-12 TF loaded MTCs. IL-12 TF drives IFNg production and enhances cytotoxicity in IL-15 nanogel loaded cells.

FIG. 23F: Beneficial synergistic effect was observed on co-loaded cells at low level of IL-15 nanogel and IL-12 TF. As shown in FIG. 23F, determining the optimal loading doses of IL-15 nanogel and IL-12 TF for co-load samples, lower doses of each monotherapy might be enough to reach the same synergistic effect.

FIG. 23G: Combo and co-load show improved activity relative to IL-12 TF and IL-15 nanogel at same total cell numbers (15 M). * IIL-12 TF 15M group: variability is driven by 1 mouse w earlier tumor escape than others.

### Materials and Methods

### MART-1-Targeted T Cell Generation

Healthy donor apheresis was collected and shipped by HemaCare, and these cells are subsequently referred to as CTL075. Apheresis was separated by size and density into six fractions using an Elutra device (Terumo BCT). The T cell rich fraction (fraction 3) was volume reduced using a Sepax C-Pro device (GE) into HBSS, frozen in 20% HBSS and 80% CryoStor10 (CS10, BioLife Solutions) in a controlled rate freezer (CRF) and then transferred to liquid nitrogen.

A monocyte rich fraction was identified via cellometer as the Elutra fraction(s) with highest CD14⁺ cell percentage as determined by flow cytometry. Following identification and counting using AO/PI staining and cellometer acquisition, live cells were washed into monocyte differentiation media (RPMI-1640 with 2% human AB serum and 1% GlutaMAX). Monocyte rich cells were counted and 1.16×10⁸ cells/bag were transferred into cell differentiation bags (Miltenyi). The volume was brought up to 20 mL using monocyte differentiation media with IL-4 (750 IU/mL) and GM-CSF (500 IU/mL). Next, all cells were placed in an incubator at 37° C. and 5% CO₂ overnight.

After 24 h, monocyte rich cells were matured into mature dendritic cells (mDC) by adding 80 mL of monocyte differentiation media containing a maturation cocktail of IL-1B (1400 IU/mL), IL-6 (1100 IU/mL), TNF-α (1000 IU/mL), and PGE-2 (0.352 μg/ml). Cells were next incubated at 37° C./5% CO₂ for 48 h.

Mature DCs were harvested by centrifugation at 500 g for 5 min and washed into T cell media (CTS AIM-V SFM [Thermo Fisher, South San Francisco, Calif.] with 5% human AB serum and 1% GlutaMAX). Cells were then diluted to 5.0×10⁶ cells/mL, and reconstituted MART-1 peptide (ELAGIGILTV; New England Peptide, Gardner, Mass.) was added to the culture at 0.1 μM. The cells were placed in the incubator for 1 h. The remaining mDCs were pelleted by centrifugation, washed, re-pelleted, and frozen in 20% HBSS and 80% CS10.

At this time, cells from the T cell rich fraction were thawed manually into T cell media, centrifuged to remove DMSO and transferred to a PL07 bag (Origen Biomedical, Austin, Tex.). The cells were then diluted with T cell media to 1.0×10⁶ cells/mL, and priming cytokines were added. Next, mDCs were added to the T cells at a ratio of 1:10 mDC to T cells, and cells were incubated at 37° C./5% CO₂ for 4 days.

Following the four-day incubation, cells were counted, pelleted and resuspended in fresh T cell media. Cells were then diluted to 1.0×10⁶ cells/mL in T cell media with priming cytokines, placed into a PL07 bag and incubated at 37° C./5% CO₂ for 3 days. On Day 7 of the T cell culture, the cells were pelleted, resuspended in fresh T cell media, diluted to 1.0×10⁶ cells/mL, seeded into a PL07 bag, and expansion cytokines were added. The previously frozen mDCs were thawed manually into T cell media, loaded with MART-1 peptide as described above, and placed directly into the T cell culture at a ratio of 1:10 mDC to T cells. The culture was then incubated at 37° C./5% CO₂ until Day 11.

On Day 11, cells were counted, and cultures were adjusted to 1.0×10⁶ cells/mL in T cell media with expansion cytokines before continued incubation at 37° C./5% CO₂. On Day 14, the cells were collected, counted, and washed into T cell media for IL-12 tethered fusion loading and co-culture assays.

Human IL-15 Nanogel and IL-12 Tethered Fusion Loading onto MART-1-Targeted T Cells

Human IL-12 tethered fusion and IL-15 nanogel loading onto MART-1-targeted T cells was carried out at 20-40×10⁶ cell scales in 1.5 ml Eppendorf tubes. The MART-1-targeted T cells were harvested by centrifugation at 500×g 5 min, resuspended at 1×10⁸ cells/mL in HBSS and mixed 1:1 with IL-12 tethered fusion or IL-15 nanogel at 2× the final loading concentration. IL-15 nanogel was loaded in HBSS solution at a final concentration of 50×10⁶/mL of cells, 0.375 mg/mL of IL-15 nanogel and incubated at 37 C for 1 hr. Onto MART-1-Targeted T Cells IL-12 tethered fusion was loaded in HBSS+10% HSA solution at a final concentration of 25×106/mL of cells, IL-12 tethered fusion at 125 nM, and incubated at 37 C for 30 minutes. Onto IL-15 nanogel-loaded MART-1-Targeted T Cells IL-12 tethered fusion was loaded in HBSS+10% HSA solution at a final concentration of 25×10⁶/mL of cells, IL-12 tethered fusion at 125 nM, and incubated at 37 C for 30 minutes to create co-loaded IL-12 tethered fusion/IL-15 nanogel T cells. Next, cells were washed by an initial dilutive wash, centrifuged at 500 g 5 min and washed a second time with T cell AIMS media. Co-loaded T cells were diluted 1:4 in T cell media, counted, and adjusted to 5×10⁴ cells/mL in T cell media prior to serial dilutions and seeding in the co-culture assay.

Assessment of MART-1 Specificity, IL-12 tethered fusion & IL-15 nanogel Cell Surface Loading, and T Cell Number by Flow Cytometry

IL-12 tethered fusion or IL-15 nanogel was loaded onto MART-1-targeted T cells on Day 0 as described above. After loading, triplicates of 1×10⁴ T cells per loading condition were transferred to 96-well plates. Two such plates were prepared.

One plate was stained with:
BV421 CD25 (0.5 uL/sample)
BV711 CD69 (0.5 uL/sample)
PE/Cy7 CD62L (0.5 uL/sample)
FITC CD4 (0.5 uL/sample)
BV605 CD45RO (0.5 uL/sample)
BV785 CD8 (0.5 uL/sample)
HLA-A*02:01 MART-1 tetramer (1:100 dilution) (MBL, Woburn, Mass.)
PercpCy5.5 IL137 (1:100 dilution)
AF647 anti-IL12 (1:100 dilution)
AF700 CD3 (0.5 uL/sample)
APC-Cy7 CCR7 (1 uL/sample)
Zombie Aqua (1:500 dilution)

Reagents were from Biolegend (Biolegend, San Diego, Calif.) unless otherwise noted. Fluorescence Minus One controls were done for CD25, CD69, CD62L, CD137, CCR7 and CD45RO.

The other plate was stained with:
FITC CD4 (0.5 uL/sample)
BV711 CD8 (0.5 uL/sample)
BV785 CD8 (0.5 uL/sample)
PE IL15 (1:100 dilution)
AF647 anti-IL12 (1:100 dilution)
AF700 CD3 (0.5 uL/sample)
Zombie Aqua (1:500 dilution)

At Day 1, 3 or 4 & 6 time points of co-culture or parallel monoculture in flat bottom 96-well plates, all the T cells in media supernatant were transferred to new U-bottom 96-well plates both from a plate of T cell monoculture and a plate of co-culture with target cells. Flat bottom plates were used for MTT assay (see below). U-bottom plates were spun at 500×g for 5 min. Media supernatants were collected for IFNγ ELISA (see below):
BV421 CD25 (0.5 uL/sample)
PE/Cy7 CD62L (0.5 uL/sample)
FITC CD4 (0.5 uL/sample)
BV711 CD8 (0.5 uL/sample)
BV605 CD45RO (0.5 uL/sample)
BV785 CD8 (0.5 uL/sample)
PE tetramer (1:100 dilution)
PercpCy5.5 IL137 (1:100 dilution)
AF647 anti-IL12 (1:100 dilution)
AF700 CD3 (0.5 uL/sample)
Zombie Aqua (1:500 dilution)

5 μL of CountBright counting beads (LifeTechnologies) per well in staining buffer (PBS with 2 mM EDTA and 2% FBS) were added after washing off antibody stain.

Samples were analyzed on a FACSCelesta flow cytometer (BectonDickinson) using FACSDiva software (Becton Dickinson).

### Assessment of Tumor Cell Lysis

The MART-1-positive human cancer cell line SKMEL-5 was maintained per ATCC recommendations. For direct assessment of antigen-specific T cell cytotoxicity, SKMEL-5 cells were plated in target cell culture media (DMEM with 10% FBS, Pen/Strep) at 1×10⁴cells/well in 96-well plates. After overnight incubation at 37° C. covered with microporous film, target cell media was removed, and 200 μL/well of T cell suspension in T cell media were added in serial dilutions resulting in Effector:Target ratios of 1:1, 1:2, 1:5, and 1:10 in one experiment and 10:1, 1:1, and 1:10 in a replicate experiment. A target cell only condition was included to serve as a negative control. Effector cell conditions were: (1) MART-1-specific T cells alone, (2) IL-12 tethered fusion-loaded MART-1-specific T cells (3) IL-15 nanogel-loaded MART-1-specific T cells (4) 1:1 combination of IL-12 tethered fusion-loaded MART-1-specific T cells and IL-15 nanogel-loaded MART-1-specific T cells, and (5) IL-12 tethered fusion/IL-15 nanogel-co-loaded MART-1-specific T cells. Target cells only with 10% DMSO in the media served as a negative control without T cell cytotoxicity. A MART-1-targeted T cell monoculture plate with 200 μL/well of the same T cell suspensions was also plated. Following the addition of T cells, plates were spun at 100 g for 1 min and incubated at 37° C. covered with microporous film. Technical triplicates were used for the colorimetric cytotoxicity studies.

After 1, 3 or 4, and 6 days, T-cell containing media supernatants were transferred to new 96-well plate and used for flow analysis (as described above). Target cells were washed carefully once with PBS. A solution of 0.5 mg/ml MTT (3-(4,5-dimethylthiazol-2-yl)-2.5-diphenyltetrazolium bromide, Sigma) in target cell media (100 μL/well) was added, and the plates were incubated at 37° C. for 1.5 h while purple formazan crystals formed in live cells. After 1.5 h, the media with MTT was removed, and the cells were washed carefully with PBS. DMSO (100 μL/well) was added to dissolve formazan crystals. Absorbance at 570 nm was read on a Tecan plate reader. To assess target cell number, the background signal (from 0% live DMSO in culture media wells) was subtracted and values were normalized to the 100% live values from wells only containing SKMEL-5 target cells (but no T cells).

For the re-challenge assay condition, after 4 days T-cell containing media supernatants were transferred to new flat bottom plate with SKMEL-5 to re-challenge the T cells with new tumor co-culture targets. Duplicate plates still existed on that day to continue the original time course out to day 6.

### Assessment of IFNγ Secretion

At the Day 1, 4 & 6 timepoints, culture media supernatant removed from cells pelleted for flow cytometry (see above) was saved for ELISA quantification of IFNγ, via the human IFNγ Quantikine ELISA Kit (R&D Systems, Minneapolis, Minn.) according to the manufacturer's instructions. Samples were diluted 1:4 and 1:10 in supplied diluent to concentrations within the linear range of the assay. Absorbance was measured on a Tecan Infinite M200 plate reader.

Flow cytometry data was analyzed with FlowJo v10 software (BD Biosciences, San Jose, Calif.), and graphs were prepared in GraphPad Prism v7.0. ELISA analysis was done in GraphPad Prism v7.0.

### Modifications

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference. The PCT International Application Nos. PCT/US2018/040777, PCT/US2018/040783, PCT/US2018/040786, PCT/US2018/049594, PCT/US2018/049596 and PCT/US2019/050492 are all incorporated herein by reference in its entirety.

1. A therapeutic (e.g., cancer immunotherapy) composition comprising: a first immune cell having a surface loaded with a plurality of protein nanogels and a second immune cell having a surfaced loaded with a plurality of immunostimulatory fusion molecules (IFMs).

2. The composition of claim 1, wherein each protein nanogel comprises a plurality of therapeutic protein monomers reversibly cross-linked to one another via a plurality of biodegradable cross-linkers, wherein the protein nanogel has a size between 30 nm and 1000 nm in diameter measured by dynamic light scattering, wherein the cross-linker degrades, after administration into a subject in need thereof, under physiological conditions so as to release the therapeutic protein monomers from the protein nanogel, wherein optionally the protein nanogel further comprises a surface modification such as polycation so as to allow the protein nanogel to associate with the first immune cell.

3. The composition of claim 1, wherein each IFM is engineered to contain an immunostimulatory cytokine molecule and a targeting moiety (e.g., an antibody or an antigen-binding fragment thereof) having an affinity to an antigen on the surface of the second immune cell, wherein the immunostimulatory cytokine molecule is operably linked to targeting moiety.

4. The composition of claim 1, wherein the first immune cell and the second immune cell are the same cell.

5. The composition of claim 1, wherein the first immune cell and the second immune cell are different cells, and are provided and administered separately (e.g., sequentially) to a patient in need of, e.g., cancer immunotherapy.

6. The composition of claim 2, wherein the therapeutic protein monomers include one or more cytokine molecules and/or one or more costimulatory molecules, wherein:
(i) the one or more cytokine molecules are selected from IL-15, IL-2, IL-7, IL-10, IL-12, IL-18, IL-21, IL-23, IL-4, IL-1alpha, IL-1beta, IL-5, IFNgamma, TNFa, IFNalpha, IFNbeta, GM-CSF, or GCSF; and
(ii) the one or more costimulatory molecules are selected from CD137, OX40, CD28, GITR, VISTA, anti-CD40, or CD3.

7. The composition of claim 3, wherein in the IFM, the immunostimulatory cytokine molecule is selected from one or more of IL-15, IL-2, IL-6, IL-7, IL-12, IL-18, IL-21, IL-23, or IL-27 or variant forms thereof, and wherein the antigen is selected from one or more of CD45, CD4, CD8, CD3, CD11a, CD11b, CD11c, CD18, CD25, CD127, CD19, CD20, CD22, HLA-DR, CD197, CD38, CD27, CD196, CXCR3, CXCR4, CXCR5, CD84, CD229, CCR1, CCR5, CCR4, CCR6, CCR8, CCR10, CD16, CD56, CD137, OX40, or GITR.

8. A method for providing cancer immunotherapy, comprising:
administering to a patient in need thereof a plurality of immune cells each loaded with a first plurality of protein nanogels and a second plurality of immunostimulatory fusion molecules (IFMs).

9. A method for providing cancer immunotherapy, comprising:
administering to a patient in need thereof a first plurality of immune cells each loaded with a plurality of protein nanogels; and
administering to the patient a second plurality of immune cells each loaded with a plurality of immunostimulatory fusion molecules (IFMs).

10. The method of claim 8 or 9, wherein the cancer immunotherapy is for treatment of cancer selected from breast, prostate, lung, ovarian, cervical, skin, melanoma, colon, stomach, liver, esophageal, kidney, throat, thyroid, pancreatic, testicular, and bone cancer, leukemia, chronic lymphocytic leukemia, basal cell carcinoma, biliary tract cancer, bladder cancer, brain and central nervous system (CNS) cancer, choriocarcinoma, colorectal cancer, connective tissue cancer, endometrial cancer, eye cancer, head and neck cancer, gastric cancer, intraepithelial neoplasm, larynx cancer, lymphoma; neuroblastoma; lip, tongue, mouth and pharynx cancer; retinoblastoma; rhabdomyosarcoma; rectal cancer; sarcoma; skin cancer; thyroid cancer; and uterine cancer.

11. A method for inducing the synergistic expansion of human CD8⁺ T cells in a human immunotherapeutic regimen, said regimen consisting of co-administering at least two immune agonists, the first immune agonist comprising a T cell loaded with an IL-12 tethered fusion, and the second immune agonist comprising a T cell loaded with an IL-15 nanogel, wherein the co-administration of such immune agonists results in a synergistic expansion of said human CD8⁺ T cells.

12. The method of claim 11, wherein the T cell loaded with the IL-12 tethered fusion, the T cell loaded with the IL-15 nanogel, or both T cells, are specific to one or more tumor-associated antigens.

13. The method of claim 12, wherein the tumor-associated antigen is one expressed by a cancer selected from breast, prostate, lung, ovarian, cervical, skin, melanoma, colon, stomach, liver, esophageal, kidney, throat, thyroid, pancreatic, testicular, brain, and bone cancer, leukemia, chronic lymphocytic leukemia, basal cell carcinoma, biliary tract cancer, bladder cancer, brain and central nervous system (CNS) cancer, choriocarcinoma, colorectal cancer, connective tissue cancer, endometrial cancer, eye cancer, head and neck cancer, gastric cancer, intraepithelial neoplasm, larynx cancer, lymphoma; neuroblastoma; lip, tongue, mouth and pharynx cancer; retinoblastoma; rhabdomyosarcoma; rectal cancer; sarcoma; skin cancer; thyroid cancer; and uterine cancer.

14. The method of claim 11, wherein the IL-12 tethered fusion comprises a humanized anti-CD45 antibody or an antibody fragment selected from a Fab, F(ab)₂, Fd, and a Fv.

15. The method of claim 11, wherein the IL-15 nanogel comprises a plurality of crosslinked IL-15-Fc fusion protein monomers.

16. A method for the treatment of cancer, comprising the concurrent administration to a mammal in need thereof a synergistic, therapeutically effective amount of two immune agonists, the first immune agonist comprising a T cell loaded with an IL-12 tethered fusion, and the second immune agonist comprising a T cell loaded with an IL-15 nanogel.

17. The method of claim 16, wherein said cancer is a solid tumor.

18. The method of claim 16, wherein said cancer treatment further comprises an anti-proliferative cytotoxic agent either alone or in combination with radiation therapy.

19. The method of claim 16, wherein the first and second immune agonists are administered in a ratio of either immune agonists to the other immune agonists of 1:1, 1:2, 1:3, 1:4 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:30, 1:35, 1:40, 1:45, 1:50, 1:55, 1:60, 1:65, 1:70; 1:75, 1:80, 1:85, 1:90, 1:95, 1:100, 1:120, 1:130, 1:140, 1:150, 1:160, 1:170, 1:180, 1; 190, 1:200, 1:500, 1:1000, 1:5000, 1:10,000, 1:100,000, 2:3, 3:4, 2:5, 3:5, 3:10, 7:10, 9:10, 2:15, 4:15, 6:15, 7:15, 8:15, 11:15, 13:15, 14:15, 3:20, 7:20, 9:20, 11:20, 13:20, 17:20, 19:20, 1:25, 2:25, 4:25, 6:25, 7:25, 8:25, 10:25, 11:25, 12:25, 13:25, 14:25, 16:25, 17:25, 18:25, 19:25, 21:25, 22:25, 23:25, or 24:25.

20. The method of claim 16, wherein at least one of the first and second immune agonists is administered in a dosage of about 20 million cells/m², 40 million cells/m², 100 million cells/m², 120 million cells/m², 200 million cells/m², 360 million cells/m², 600 million cells/m², 1 billion cells/m², 1.5 billion cells/m², 10⁶ cells/m², about 5×10⁶ cells/m², about 10⁷ cells/m², about 5×10⁷ cells/m², about 10⁸ cells/m², about 5×10⁸ cells/m², about 10⁹ cells/m², about 5×10⁹ cells/m², about 10¹⁰ cells/m², about 5×10¹⁰ cells/m², or about 10¹¹ cells/m².


SEQUENCE LISTING (131 sequences)

SEQ ID NO: 1 - protein, 298 aa, Unknown
  Synthetic
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSITCPPPMSVEHADIWVKSYSLYSRERYICNSGFKRKAGTSSLTECVLNKATNVAHWTTPSLKCIR

SEQ ID NO: 2 - protein, 298 aa, Unknown
  Synthetic
ITCPPPMSVEHADIWVKSYSLYSRERYICNSGFKRKAGTSSLTECVLNKATNVAHWTTPSLKCIRGGGGSGGGGSGGGGSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 347 aa, Unknown
  Synthetic
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - protein, 347 aa, Unknown
  Synthetic
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 224 aa, Unknown
  Synthetic
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 6 - protein, 448 aa, Unknown
  Synthetic
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 218 aa, Unknown
  Synthetic
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 577 aa, Unknown
  Synthetic
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 9 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 10 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 11 - protein, 114 aa, Unknown
  Synthetic
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 12 - protein, 297 aa, Unknown
  Synthetic
ITCPPPMSVEHADIWVKSYSLYSRERYICNSGFKRKAGTSSLTECVINKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 297 aa, Unknown
  Synthetic
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 220 aa, Unknown
  Synthetic
QVQLQQLGAELARPGASVKMSCKASGYTFTSYSIQWVKQRPGQGLEWIGYINPSSGYIKYNQHFRDRATLTADRSSSTAYMQLSSLTSEDSAVYYCARGNSGSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 15 - protein, 348 aa, Unknown
  Synthetic
DIVMTQAAPSVPVTPGESLSISCRSSKSLLHSSGITYLYWFLQRPGQSPQLLIYRMSNLASGVPDRFSGSGSGTAFTLRISRVEAEDVGVYYCMQHLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 16 - protein, 220 aa, Unknown
  Synthetic
QVQLQQSGAELARPGASVKMSCKASGYTFTSYTMQWVKQRPGQGLEWIGYINPSSGYIKYNQKFKDKVTLTADKSSTTAYMQLSRLTSEDSAVYYCARRGSYFFDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 17 - protein, 348 aa, Unknown
  Synthetic
DIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVEYPFTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 18 - protein, 224 aa, Unknown
  Synthetic
EVQLVESGGGLVQPGGSLRLSCAASGFDFSRYWMSWVRQAPGKGLEWIGEINPTSSTINFADSVKGRFTISRDNAKNSLYLQMNSVRDEDTAVYYCARGNYYRYGDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 19 - protein, 347 aa, Unknown
  Synthetic
EIVLTQSPATLSLSLGERATISCRASKSVSTSGYSYLHWYQQKPGQAPKLLIYLASNRATGVPARFSGSGPGTDFTLTISSLEPEDFATYYCQHSRELPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 20 - protein, 224 aa, Unknown
  Synthetic
EVQLVESGGGLVQPGGSLRLSCAASGFDFSRYWMSWVRQAPGKGLEWIGEINPTSSTINFADSVKGRFTISRDNAKNSLYLQMNSVRDEDTAVYYCARGSGGGSGDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 21 - protein, 340 aa, Unknown
  Synthetic
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 22 - protein, 344 aa, Unknown
  Synthetic
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTSPPSPAPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 23 - protein, 121 aa, Unknown
  Synthetic
EVQLVESGGGLVQPGGSLRLSCAASGFDFSRYWMSWVRQAPGKGLEWIGEINPTSSTINFADSVKGRFTISRDNAKNSLYLQMNSVRDEDTAVYYCARGNYYRYGDAMDYWGQGTLVTVSS

SEQ ID NO: 24 - protein, 111 aa, Unknown
  Synthetic
EIVLTQSPATLSLSLGERATISCRASKSVSTSGYSYLHWYQQKPGQAPKLLIYLASNRATGVPARFSGSGPGTDFTLTISSLEPEDFATYYCQHSRELPFTFGQGTKLEIK

SEQ ID NO: 25 - protein, 121 aa, Unknown
  Synthetic
EVQLVESGGGLVQPGGSLRLSCAASGFDFSRYWMSWVRQAPGKGLEWIGEINPTSSTINFADSVKGRFTISRDNAKNSLYLQMNSVRDEDTAVYYCARGSGGGSGDAMDYWGQGTLVTVSS

SEQ ID NO: 26 - protein, 224 aa, Unknown
  Synthetic
EVQLQQPGAELMRPGASVKLSCKASGYSFTGHWMNWVRQRPGQGLEWIGMIHPSDSETRLNQKFKDKATLTVDKSSSSAYMQLSSPTSEDSAVYYCARGIYFYGTTYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 27 - protein, 343 aa, Unknown
  Synthetic
DVQITQSPSYLAASPGETISINCRASKTISKYLAWYQEKPGKTNKLLIYSGSTLQSGIPSRFSGSGSGTDFTLTISSLEPEDFAMYYCQQHNEYPLTFGTGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 28 - protein, 223 aa, Unknown
  Synthetic
DVKLVESGGDLVKLGGSLKLSCAASGFTFSDYYMSWVRQTPEKRLELVAAIDNDGGSISYPDTVKGRFTISRDNAKNTLYLQMSSLRSEDTALYYCARQGRLRRDYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 29 - protein, 347 aa, Unknown
  Synthetic
DIVLTQSPASLAVSLGQRATISCRASESVDSYGNSFMHWYQQKPGQPPKLLIYRASNLESGIPARFSGSGSRTDFTLTINPVEADDVATYYCQQSNEDPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 30 - protein, 221 aa, Unknown
  Synthetic
QVQLQQSGAELVKPGASVKLSCTASGFNIKDTYIHFVRQRPEQGLEWIGRIDPANDNTLYASKFQGKATITADTSSNTAYMHLSSLTSGDTAVYYCGRGYGYYVFDHWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 31 - protein, 343 aa, Unknown
  Synthetic
DVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 32 - protein, 380 aa, Unknown
  Synthetic
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSGGGGSGGGGSGGGTGDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRSGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSAAAHHHHHH

SEQ ID NO: 33 - protein, 22 aa, Unknown
  Synthetic
MDMRVPAQLLGLLLLWLPGAKC

SEQ ID NO: 34 - protein, 19 aa, Unknown
  Synthetic
MELGLCWVFLVAILEGVQC

SEQ ID NO: 35 - protein, 30 aa, Unknown
  Synthetic
MAPRRARGCRTLGLPALLLLLLLRPPATRG

SEQ ID NO: 36 - protein, 15 aa, Unknown
  Synthetic
GGGGSGGGGSGGGGS

SEQ ID NO: 37 - protein, 8 aa, Unknown
  Synthetic
GGGSGGGS

SEQ ID NO: 38 - protein, 12 aa, Unknown
  Synthetic
DKTHTSPPSPAP

SEQ ID NO: 39 - protein, 11 aa, Unknown
  Synthetic
RSGSGGGGSLQ

SEQ ID NO: 40 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 41 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 42 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 43 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 44 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 45 - protein, 132 aa, Homo sapiens
GQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 46 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 47 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 48 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 49 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 50 - protein, 519 aa, Unknown
  Synthetic
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 51 - protein, 519 aa, Unknown
  Synthetic
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 52 - protein, 61 aa, Homo sapiens
CPPPMSVEHADIWVKSYSLYSRERYICNSGFKRKAGTSSLTECVLNKATNVAHWTTPSLKC

SEQ ID NO: 53 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 55 - protein, 228 aa, Unknown
  Synthetic
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 56 - protein, 293 aa, Unknown
  Synthetic
ITCPPPMSVEHADIWVKSYSLYSRERYICNSGFKRKAGTSSLTECVLNKATNVAHWTTPSLKCIRERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 57 - protein, 121 aa, Unknown
  Synthetic
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSS

SEQ ID NO: 58 - protein, 111 aa, Unknown
  Synthetic
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 59 - protein, 117 aa, Unknown
  Synthetic
QVQLQQLGAELARPGASVKMSCKASGYTFTSYSIQWVKQRPGQGLEWIGYINPSSGYIKYNQHFRDRATLTADRSSSTAYMQLSSLTSEDSAVYYCARGNSGSFDYWGQGTTLTVSS

SEQ ID NO: 60 - protein, 112 aa, Unknown
  Synthetic
DIVMTQAAPSVPVTPGESLSISCRSSKSLLHSSGITYLYWFLQRPGQSPQLLIYRMSNLASGVPDRFSGSGSGTAFTLRISRVEAEDVGVYYCMQHLEYPFTFGGGTKLEIK

SEQ ID NO: 61 - protein, 117 aa, Unknown
  Synthetic
QVQLQQSGAELARPGASVKMSCKASGYTFTSYTMQWVKQRPGQGLEWIGYINPSSGYIKYNQKFKDKVTLTADKSSTTAYMQLSRLTSEDSAVYYCARRGSYFFDFWGQGTSVTVSS

SEQ ID NO: 62 - protein, 112 aa, Unknown
  Synthetic
DIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVEYPFTFGGGTKLEVK

SEQ ID NO: 63 - protein, 205 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 64 - protein, 172 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIKPAASSPSSNNTAATTAAIVPGSQLMPSKSPSTGTTEISSHESSHGTPSQTTAKNWELTASASHQPPGVYPQGHSDTT

SEQ ID NO: 65 - protein, 62 aa, Unknown
  Synthetic
CPPPMSVEHADIWVKSYSLYSRERYICNSGFKRKAGTSSLTECVINKATNVAHWTTPSLKCI

SEQ ID NO: 66 - protein, 65 aa, Unknown
  Synthetic
ITCPPPMSVEHADIWVKSYSLYSRERYICNSGFKRKAGTSSLTECVINKATNVAHWTTPSLKCIR

SEQ ID NO: 67 - protein, 232 aa, unknown; identical to SEQ ID NO: 53
  Synthetic

SEQ ID NO: 68 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 69 - protein, 224 aa, Unknown
  Synthetic
EVQLVESGGGLVQPGGSLRLSCAASGYSFTGHWMNWVRQAPGKGLEWVGMIHPSDSETRYNQKFKDRFTISVDKSKNTLYLQMNSLRAEDTAVYYCARGIYFYGTTYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 70 - protein, 16 aa, Unknown
  Synthetic
GGGSGGGSGGGSGGGS

SEQ ID NO: 71 - protein, 20 aa, Unknown
  Synthetic
GGGGSGGGGSGGGGSGGGGS

SEQ ID NO: 72 - protein, 523 aa, Unknown
  Synthetic
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 73 - protein, 523 aa, Unknown
  Synthetic
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 74 - protein, 107 aa, Homo sapiens [sequence omitted]

SEQ ID NO: 75 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 76 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 77 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 78 - protein, 106 aa, Homo sapiens
SQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 79 - protein, 220 aa, Unknown
  Synthetic
EVQLVQSGAEVKKPGASVKVSCKASGYTFTSYSIQWVRQAPGQRLEWIGYINPSSGYIKYNQHFRGRATLTADRSASTAYMELSSLRSEDTAVYYCARGNSGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 80 - protein, 484 aa, Unknown
  Synthetic
EVQLVQSGAEVKKPGASVKVSCKASGYTFTSYSIQWVRQAPGQRLEWIGYINPSSGYIKYNQHFRGRATLTADRSASTAYMELSSLRSEDTAVYYCARGNSGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSEVQLVQSGAEVKKPGASVKVSCKASGYTFTSYSIQWVRQAPGQRLEWIGYINPSSGYIKYNQHFRGRATLTADRSASTAYMELSSLRSEDTAVYYCARGNSGSFDYWGQGTLVTVSSGGGGSGGGGSGGGGSGGGGSDIVMTQSPLSLPVTPGEPASISCRSSQSLLHSSGITYLYWFLQKPGQSPQLLIYRMSNLASGVPDRFSGSGSGTDFTLKISRVEAEDVGVYYCMQHLEYPFTFGQGTKLEIK

SEQ ID NO: 81 - protein, 487 aa, Unknown
  Synthetic
EVQLVQSGAEVKKPGASVKVSCKASGYTFTSYSIQWVRQAPGQRLEWIGYINPSSGYIKYNQHFRGRATLTADRSASTAYMELSSLRSEDTAVYYCARGNSGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSEVQLVESGGGLVQPGGSLRLSCAASGFDFSRYWMSWVRQAPGKGLEWIGEINPTSSTINFADSVKGRFTISRDNAKNSLYLQMNSVRDEDTAVYYCARGNYYRYGDAMDYWGQGTLVTVSSGGGGSGGGGSGGGGSGGGGSEIVLTQSPATLSLSLGERATISCRASKSVSTSGYSYLHWYQQKPGQAPKLLIYLASNRATGVPARFSGSGPGTDFTLTISSLEPEDFATYYCQHSRELPFTFGQGTKLEIK

SEQ ID NO: 82 - protein, 753 aa, Unknown
  Synthetic
DIVMTQSPLSLPVTPGEPASISCRSSQSLLHSSGITYLYWFLQKPGQSPQLLIYRMSNLASGVPDRFSGSGSGTDFTLKISRVEAEDVGVYYCMQHLEYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 83 - protein, 226 aa, Unknown
  Synthetic
QVNLLQSGAALVKPGASVKLSCKASGYTFTDYYIHWVKQSHGKSLEWVAYINPDRDYTNYNEKFKSKATLTVDKSTNTAYMELSRLTSEDSATFYCTRRLYDGAYYYSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 84 - protein, 755 aa, Unknown
  Synthetic
DIQMTQSPHSLSASLGETVSIECLASEGISNYLAWYQQKPGKSPQLLIYYGSRLQDGVPSRFSGSGSGTQYSLRITNMQPEDEGVYYCQQGYKYPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 85 - protein, 343 aa, Unknown
  Synthetic
DIQMTQSPHSLSASLGETVSIECLASEGISNYLAWYQQKPGKSPQLLIYYGSRLQDGVPSRFSGSGSGTQYSLRITNMQPEDEGVYYCQQGYKYPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 86 - protein, 491 aa, Unknown
  Synthetic
QVNLLQSGAALVKPGASVKLSCKASGYTFTDYYIHWVKQSHGKSLEWVAYINPDRDYTNYNEKFKSKATLTVDKSTNTAYMELSRLTSEDSATFYCTRRLYDGAYYYSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSQVNLLQSGAALVKPGASVKLSCKASGYTFTDYYIHWVKQSHGKSLEWVAYINPDRDYTNYNEKFKSKATLTVDKSTNTAYMELSRLTSEDSATFYCTRRLYDGAYYYSWFAYWGQGTLVTVSSGGGGSGGGGSGGGGSGGGGSDIQMTQSPHSLSASLGETVSIECLASEGISNYLAWYQQKPGKSPQLLIYYGSRLQDGVPSRFSGSGSGTQYSLRITNMQPEDEGVYYCQQGYKYPYTFGAGTKLELK

SEQ ID NO: 87 - protein, 227 aa, Unknown
  Synthetic
EVKLQESGGGLVQPGRSLKLSCAASGFNFNDYWMGWVRQAPGKGLEWIGEINKDSSTINYTPSLKDKFTISRDNAQNTLYLQMSKLGSEDTAIYYCARARGMMVLIIPHYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 88 - protein, 342 aa, Unknown
  Synthetic
DIVLTQSPAMAMSPGERITISCRASESVSTRMHWYQQKPGQQPKLLIYGASNLESGVPARFSGSGSGTDFTLTIDPVEANDTATYFCQQSWYDPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 89 - protein, 492 aa, Unknown
  Synthetic
EVKLQESGGGLVQPGRSLKLSCAASGFNFNDYWMGWVRQAPGKGLEWIGEINKDSSTINYTPSLKDKFTISRDNAQNTLYLQMSKLGSEDTAIYYCARARGMMVLIIPHYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSQVNLLQSGAALVKPGASVKLSCKASGYTFTDYYIHWVKQSHGKSLEWVAYINPDRDYTNYNEKFKSKATLTVDKSTNTAYMELSRLTSEDSATFYCTRRLYDGAYYYSWFAYWGQGTLVTVSSGGGGSGGGGSGGGGSGGGGSDIQMTQSPHSLSASLGETVSIECLASEGISNYLAWYQQKPGKSPQLLIYYGSRLQDGVPSRFSGSGSGTQYSLRITNMQPEDEGVYYCQQGYKYPYTFGAGTKLELK

SEQ ID NO: 90 - protein, 754 aa, Unknown
  Synthetic
DIVLTQSPAMAMSPGERITISCRASESVSTRMHWYQQKPGQQPKLLIYGASNLESGVPARFSGSGSGTDFTLTIDPVEANDTATYFCQQSWYDPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 91 - protein, 249 aa, Unknown
  Synthetic
EVQLVQSGAEVKKPGASVKVSCKASGYTFTSYSIQWVRQAPGQRLEWIGYINPSSGYIKYNQHFRGRATLTADRSASTAYMELSSLRSEDTAVYYCARGNSGSFDYWGQGTLVTVSSGGGGSGGGGSGGGGSGGGGSDIVMTQSPLSLPVTPGEPASISCRSSQSLLHSSGITYLYWFLQKPGQSPQLLIYRMSNLASGVPDRFSGSGSGTDFTLKISRVEAEDVGVYYCMQHLEYPFTFGQGTKLEIK

SEQ ID NO: 92 - protein, 252 aa, Unknown
  Synthetic
EVQLVESGGGLVQPGGSLRLSCAASGFDFSRYWMSWVRQAPGKGLEWIGEINPTSSTINFADSVKGRFTISRDNAKNSLYLQMNSVRDEDTAVYYCARGNYYRYGDAMDYWGQGTLVTVSSGGGGSGGGGSGGGGSGGGGSEIVLTQSPATLSLSLGERATISCRASKSVSTSGYSYLHWYQQKPGQAPKLLIYLASNRATGVPARFSGSGPGTDFTLTISSLEPEDFATYYCQHSRELPFTFGQGTKLEIK

SEQ ID NO: 93 - protein, 250 aa, Unknown
  Synthetic
QVNLLQSGAALVKPGASVKLSCKASGYTFTDYYIHWVKQSHGKSLEWVAYINPDRDYTNYNEKFKSKATLTVDKSTNTAYMELSRLTSEDSATFYCTRRLYDGAYYYSWFAYWGQGTLVTVSSGGGGSGGGGSGGGGSGGGGSDIQMTQSPHSLSASLGETVSIECLASEGISNYLAWYQQKPGKSPQLLIYYGSRLQDGVPSRFSGSGSGTQYSLRITNMQPEDEGVYYCQQGYKYPYTFGAGTKLELK

SEQ ID NO: 94 - protein, 117 aa, Unknown
  Synthetic
EVQLVQSGAEVKKPGASVKVSCKASGYTFTSYSIQWVRQAPGQRLEWIGYINPSSGYIKYNQHFRGRATLTADRSASTAYMELSSLRSEDTAVYYCARGNSGSFDYWGQGTLVTVSS

SEQ ID NO: 95 - protein, 112 aa, Unknown
  Synthetic
DIVMTQSPLSLPVTPGEPASISCRSSQSLLHSSGITYLYWFLQKPGQSPQLLIYRMSNLASGVPDRFSGSGSGTDFTLKISRVEAEDVGVYYCMQHLEYPFTFGQGTKLEIK

SEQ ID NO: 96 - protein, 121 aa, Unknown; identical to SEQ ID NO: 23
  Synthetic

SEQ ID NO: 97 - protein, 111 aa, Unknown; identical to SEQ ID NO: 24
  Synthetic

SEQ ID NO: 98 - protein, 123 aa, Unknown
  Synthetic
QVNLLQSGAALVKPGASVKLSCKASGYTFTDYYIHWVKQSHGKSLEWVAYINPDRDYTNYNEKFKSKATLTVDKSTNTAYMELSRLTSEDSATFYCTRRLYDGAYYYSWFAYWGQGTLVTVSS

SEQ ID NO: 99 - protein, 107 aa, Unknown
  Synthetic
DIQMTQSPHSLSASLGETVSIECLASEGISNYLAWYQQKPGKSPQLLIYYGSRLQDGVPSRFSGSGSGTQYSLRITNMQPEDEGVYYCQQGYKYPYTFGAGTKLELK

SEQ ID NO: 100 - protein, 124 aa, Unknown
  Synthetic
EVKLQESGGGLVQPGRSLKLSCAASGFNFNDYWMGWVRQAPGKGLEWIGEINKDSSTINYTPSLKDKFTISRDNAQNTLYLQMSKLGSEDTAIYYCARARGMMVLIIPHYFDYWGQGVMVTVSS

SEQ ID NO: 101 - protein, 106 aa, Unknown
  Synthetic
DIVLTQSPAMAMSPGERITISCRASESVSTRMHWYQQKPGQQPKLLIYGASNLESGVPARFSGSGSGTDFTLTIDPVEANDTATYFCQQSWYDPWTFGGGTKLELK

SEQ ID NO: 102 - protein, 753 aa, Unknown
  Synthetic
DIVMTQSPLSLPVTPGEPASISCRSSQSLLHSSGITYLYWFLQKPGQSPQLLIYRMSNLASGVPDRFSGSGSGTDFTLKISRVEAEDVGVYYCMQHLEYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 103 - protein, 431 aa, Unknown
  Synthetic
DIVMTQSPLSLPVTPGEPASISCRSSQSLLHSSGITYLYWFLQKPGQSPQLLIYRMSNLASGVPDRFSGSGSGTDFTLKISRVEAEDVGVYYCMQHLEYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 104 - protein, 540 aa, Unknown
  Synthetic
DIVMTQSPLSLPVTPGEPASISCRSSQSLLHSSGITYLYWFLQKPGQSPQLLIYRMSNLASGVPDRFSGSGSGTDFTLKISRVEAEDVGVYYCMQHLEYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 105 - protein, 4 aa, Unknown
  Synthetic
GFSG

SEQ ID NO: 106 - protein, 4 aa, Unknown
  Synthetic
GKVS

SEQ ID NO: 107 - protein, 4 aa, Unknown
  Synthetic
GWIG

SEQ ID NO: 108 - protein, 4 aa, Unknown
  Synthetic
GKKW

SEQ ID NO: 109 - protein, 4 aa, Unknown
  Synthetic
GAYM

SEQ ID NO: 110 - protein, 8 aa, Unknown
  Synthetic
VPLSLYSG

SEQ ID NO: 111 - protein, 8 aa, Unknown
  Synthetic
GPQGIAGQ

SEQ ID NO: 112 - protein, 8 aa, Unknown
  Synthetic
VPMSMRGG

SEQ ID NO: 113 - protein, 8 aa, Unknown
  Synthetic
IPVSLRSG

SEQ ID NO: 114 - protein, 8 aa, Unknown
  Synthetic
RPFSMIMG

SEQ ID NO: 115 - protein, 8 aa, Unknown
  Synthetic
VPLSLTMG

SEQ ID NO: 116 - protein, 8 aa, Unknown
  Synthetic
IPESLRAG

SEQ ID NO: 117-118 - skipped (2 entries)

SEQ ID NO: 119 - protein, 8 aa, Unknown
  Synthetic
PQGIAGQR

SEQ ID NO: 120 - protein, 7 aa, Unknown
  Synthetic
PLGIAGR

SEQ ID NO: 121 - protein, 5 aa, Unknown
  Synthetic
GPLGP

SEQ ID NO: 122 - protein, 5 aa, Unknown
  Synthetic
GPIGP

SEQ ID NO: 123 - protein, 5 aa, Unknown
  Synthetic
SGGGS

SEQ ID NO: 124 - protein, 4 aa, Unknown
  Synthetic
GGGS

SEQ ID NO: 125 - protein, 5 aa, Unknown
  Synthetic
GGGGS

SEQ ID NO: 126 - protein, 13 aa, Unknown
  Synthetic
DKTHTCPPSCAPE

SEQ ID NO: 127 - protein, 18 aa, Unknown
  Synthetic
EPKSSDKTHTSPPSPAPE

SEQ ID NO: 128 - protein, 8 aa, Unknown
  Synthetic
SVESPPSP

SEQ ID NO: 129 - protein, 12 aa, Unknown
  Synthetic
ERKSSVESPPSP

SEQ ID NO: 130 - protein, 7 aa, Unknown
  Synthetic
PPSPSSP

SEQ ID NO: 131 - protein, 12 aa, Unknown
  Synthetic
ESKYGPPSPSSP